(19) United States
(12) Patent Application Publication
(10) Pub. No.: US 2024/0150356 A1
(43) Pub. Date: May 9, 2024

(54) Pyrimidine amide compounds and use thereof

(71) Applicant: ALPHALA CO., LTD., Taipei City (TW)

(72) Inventors: Cheng-Ho CHUNG, Taipei City (TW); Shi-Liang TSENG, Taipei City (TW); Bo-Sian LIOU, Taipei City (TW)

(21) Appl. No.: 17/766,822

(22) PCT Filed: Oct. 20, 2020
(86) PCT No.: PCT/US2020/056480

§ 371 Date: Apr. 6, 2022

Related U.S. Application Data
(60) Provisional application No. 62/924,214, filed on Oct. 22, 2019.

(51) Int. Cl. C07D 487/04; C07D 473/34; C07D 495/04

(52) U.S. Cl. CPC C07D 487/04; C07D 473/34; C07D 495/04

(57) ABSTRACT

Disclosed are compounds of formula (I) below or a stereoisomer, a tautomer, or a pharmaceutically acceptable salt thereof:

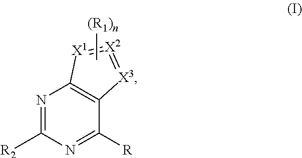

in which each of variables each of variables R, R₁, R₂, X₁, X₂, X₃ and n is defined herein. Also disclosed is a method for treating a cancer with a compound of formula (I) or a salt thereof and a pharmaceutical composition containing same.

This application claims the benefit of filing date of U.S. Provisional Application Ser. No. 62/924,214, filed Oct. 22, 2019, which is hereby incorporated by reference.

### FIELD OF THE DISCLOSURE

The present disclosure relates to compounds that can inhibit the growth of turner cells, pharmaceutical compositions comprising the compounds, and the uses of the compounds or compositions.

### BACKGROUND

In recent years, it is found that foods or food additives, and environmental pollutions may be a cause or catalyst for promoting cancer in recent years. Not coincidentally, the same event is happening as well in the developed countries and around the world, and the high incidence rates of cancers is an alarming sign. According to the data published by the American Cancer Society, cancer is being proved to be the most significant threat to public health.

The general methods for treating cancer include surgery, radiotherapy, chemotherapy and immune therapy. In recent years, several therapeutic agents for cancer treatments through new anti-cancer mechanisms have been developed, and it has been proven that the survival rate of patients can be increased by treating them with these therapeutic agents. It is known that the therapeutic agents can treat cancers through, for example, inhibition of cell cycle progression, angiogenesis, farnesyl transferase, and tyrosine kinases.

Although it is known that certain agents exhibit therapeutic effects on cancer, these agents still have their limitations. For example, some anti-cancer drugs only have therapeutic effects on specific cancers, e.g. the anti-cancer drugs for lung cancer treatment do not necessarily show effects on breast cancer treatment. In addition, the therapeutic effects of the anti-cancer drugs also depend on the locations of tumor cells, genetic variations of patients, and the side effects of drugs. Furthermore, cancer cells may spread from its original sites to another organ of the patients via the lymphatic system or blood circulatory systems, thereby causing metastatic cancers.

Since the risk of developing cancer generally increases with age, the occurrence rates of cancer are increased as longer lifespan and as mass lifestyle changes. Hence, there is a long unfulfilled need to provide new agents for cancer treatment and prevention.

### SUMMARY

The present disclosure relates to certain compounds that can inhibit the growth of tumor cells.

An aspect of this disclosure is drawn to the compounds of formula (I) below or a stereoisomer, a tautomer, or a pharmaceutically acceptable salt thereof:

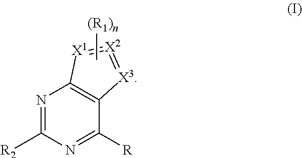

In this formula, each of X₁, X₂ and X₃ independently is C, N, O or S, with the proviso that no more than two of X₁, X₂ and X₃ are N, O or S; each of R₁ independently is selected from the group consisting of hydrogen, halogen, alkyl, alkyloxy, cycloalkyl, heterocycloalkyl, aryl or heteroaryl, wherein each of alkyl, alkyloxy, cycloalkyl, heterocycloalkyl, aryl and heteroaryl is optionally substituted with one to three moieties selected from the group consisting of halogen, hydroxyl, nitro, cyano, —NRₐR_{b}, —C(═O)R_{c}, —C(═O)OR_{d}, heterocycloalkyl optionally substituted with one to four halogen, aryl optionally substituted with one to three halogen or —NRₐR_{b}, alkyl optionally substituted with one to three halogen, and alkyloxy optionally substituted with one to three halogen or alkyloxy, in which each of Rₐ and R_{b} independently is hydrogen, alkyl or acrylamide, and each of R_{c} and R_{d} independently is hydrogen, alkenyl, or alkyl optionally substituted with one to three halogen or alkyloxy optionally further substituted with alkyloxy, one of R and R₂ is

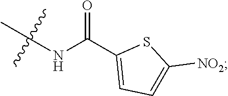 [SMILES: *NC(=O)c1ccc([N+](=O)[O-])s1]

the other of R and R₂ is —OR₃, —NHR₄, —SR₅, alkenyl, alkynyl, aryl, heteroaryl, cycloalkyl heterocycloalkyl, aryl fused with heterocycloalkyl, cycloalkenyl or cycloamine, wherein each of alkenyl, alkynyl, aryl, heteroaryl, cycloalkyl, heterocycloalkyl and cycloalkenyl is optionally substituted with one to three moieties selected from the group consisting of halogen, hydroxyl, nitro, cyano, —NRₑR_{f}, —C(═O)R_{g}, —C(═O)ORₕ, —SO₂Rᵢ, —OSiRⱼ, alkenyl, heterocycloalkyl, aryl optionally substituted with alkyl optionally substituted with one to three halogen or alkyloxy optionally substituted with one to three halogen, alkyl optionally substituted with one to three halogen, —NRₑR_{f} or —ORₖ, and alkyloxy optionally substituted with one to four halogen, hydroxyl, —NRₑR_{f} or alkyloxy optionally further substituted with alkyloxy, in which each of Rₑ and R_{f} independently is hydrogen or alkyl optionally substituted with alkyloxy, each of R_{g} and Rₕ independently is hydrogen, alkyl or alkenyl, Rᵢ is alkyl, Rⱼ is alkyl, and Rₖ is hydrogen, aryl, or alkyl optionally substituted with alkyloxy optionally further substituted with alkyloxy; each of R₃ and R₅ independently is alkyl, cycloalkyl or aryl, wherein alkyl, cycloalkyl or aryl is optionally substituted with one to four moieties selected from the group consisting of halogen, alkyl optionally substituted with one to four halogen, alkyloxy optionally substituted with one to three halogen or alkyloxy, —NRₗRₘ, and heterocycloalkyl, in which each of Rₗ and Rₘ independently is hydrogen or alkyl; R₄ is alkyl, cycloalkyl or —SO₂Rₐ, in which Rₐ is hydrogen, alkyl or aryl, and each of alkyl or aryl is optionally substituted with one to three halogen; and n is 1, 2 or 3.

The term “alkyl” herein refers to a straight or branched hydrocarbon group, containing 1-12 carbon atoms (e.g., C₁-C₁₀, C₁-C₈ and C₁-C₆). Examples include methyl, ethyl, n-propyl, i-propyl, n-butyl, i-butyl, and t-butyl.

The term “alkenyl” herein refers to linear or branch hydrocarbon groups with at least one double bond, and includes, for example, linear or branch C₂₋₁₂ hydrocarbon groups with at least one double bond, linear or branch C₂₋₈ hydrocarbon groups with at least one double bond, or linear or branch C₂₋₆ hydrocarbon groups with at least one double bond. Examples of the alkenyl include, but are not limited to vinyl, propenyl or butenyl.

The term “alkynyl” herein refers to a straight or branched monovalent or bivalent hydrocarbon containing 2-20 carbon atoms (e.g., C₂-C₁₆, C₂-C₁₂, C₂-C₈, C₂-C₆, and C₂-C₄) and one or more triple bonds. Examples of alkynl include, but are not limited to, ethynyl, ethynylene, 1-propynyl, 1- and 2-butynyl, and 1-methyl-2-butynyl.

The term “cycloalkyl” refers to a saturated and partially unsaturated monocyclic, bicyclic, tricyclic, or tetracyclic hydrocarbon group having 3-12 (e.g., 3-10 and 3-7) carbon atoms. Examples include cyclopropyl, cyclobutyl, cyclopentyl, cyclopentenyl, cyclohexyl, cyclohexenyl, cycloheptyl, and cyclooctyl.

The term “heterocycloalkyl” refers to a nonaromatic 5-4 membered monocyclic, 8-12 membered bicyclic, or 11-14 membered tricyclic ring system having one or more heteroatoms (e.g., O, N, P, and S). Examples include piperazinyl, imidazolidinyl azepanyl, pyrrolidinyl, dihydrothiadiazolyl, dioxanyl, morpholinyl, tetrahydropuranyl, and tetrahydrofuranyl.

The term “cycloalkenyl” includes cyclic unsaturated hydrocarbon groups, which includes 3 to 18 carbon atoms (C₃₋₁₈), 3 to 12 carbon atoms (C₃₋₁₂) or 3 to 8 carbon atoms (C₃₋₈). Examples of the cycloalkenyl include, but are not limited to cyclopentenyl, cyclohexenyl or cycloheptenyl.

The term “alkoxy” or “alkyloxy” refers to an —O-alkyl group. Examples include methoxy, ethoxy, propoxy, and isopropoxy.

The term “halogen” refers to a fluoro, chloro, bromo, or iodo radical.

The term “amino” refers to a radical derived from amine, which is unsubstituted or mono-/di-substituted with alkyl, aryl, cycloalkyl, heterocycloalkyl, or heteroaryl.

The term “aryl” refers to a 6-carbon monocyclic, 10-carbon bicyclic, 14-carbon tricyclic aromatic ring system. Examples of aryl groups include phenyl, naphthyl, and anthracenyl.

The term“heteroaryl” refers to an aromatic 5-8 membered monocyclic, 8-12 membered bicyclic, or 11-14 membered tricyclic ring system having one or more heteroatoms (e.g., O, N, P, and S). Examples include thiophenyl, triazolyl, oxazolyl, thiadiazolyl, tetrazolyl, pyrazolyl, pyridyl, furyl, imidazolyl, benzimidazolyl, pyrimidinyl, thienyl, quinolinyl, indolyl, thiazolyl, and benzothiazolyl.

Alkyl, alkenyl, alkynyl, cycloalkyl, cycloalkenyl, heterocycloalkyl, alkyloxy, aryl, and heteroaryl mentioned herein include both substituted and unsubstituted moieties. Possible substituents on cycloalkyl, heterocycloalkyl, alkoxy, aryl, and heteroaryl include, but are not limited to, C₁₋₆ alkyl, C₂₋₆ alkenyl, C₂₋₆ alkynyl, C₃₋₁₂ cycloalkyl, C₃₋₁₂ cycloalkenyl, C₁₋₁₂ heterocycloalkyl, C₁₋₂ heterocycloalkenyl, C₁₋₆ alkoxy, aryl, aryloxy, heteroaryl, heteroaryloxy, amino, C₁₋₆ alkylamino, C₁₋₂₀ dialkylamino, arylamino, diarylamino, C₁₋₆ alkylsulfonamino, arylsulfonamino, C₁₋₆ alkylimino, arylimino, C₁₋₆ alkylsulfonimino, arylsulfonimino, hydroxyl, halo, thio, C₁₋₆ alkylthio, arylthio, C₁₋₆ alkylsulfonyl, arylsulfonyl, acylamino, aminoacyl, aminothioacyl, amido, amidino, guanidine, ureido, thioureido, cyano, nitro, nitroso, azido, acyl, thioacyl, acyloxy, carboxyl, and carboxylic ester. On the other hand, possible substituents on alkyl include all of the above-recited substituents except C₁₋₆ alkyl. Cycloalkyl, heterocycloalkyl, aryl, and heteroaryl can also be fused with each other.

In addition to the compounds of formula (I) described above, their pharmaceutically acceptable salts and solvates, where applicable, are also covered by this disclosure. A salt can be formed between an anion and a positively charged group (e.g., amino) on a compound. Examples of a suitable anion include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. A salt can also be formed between a cation and a negatively charged group. Examples of a suitable cation include sodium ion, potassium ion, magnesium ion, calcium ion, and an ammonium cation such as tetramethylammonium ion. A salt further includes those containing quaternary nitrogen atoms. A solvate refers to a complex formed between an active compound and a pharmaceutically acceptable solvent. Examples of a pharmaceutically acceptable solvent include water, ethanol, isopropanol, ethyl acetate, acetic acid, and ethanolamine.

Another aspect of this disclosure is a pharmaceutical composition for treating a cancer.

The pharmaceutical composition contains one of the compounds of formula (I) described above or its pharmaceutically acceptable salt and a pharmaceutically acceptable carrier, excipient or diluent.

This disclosure also covers use of such a composition for the manufacture of a medicament for treating a cancer.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. Oral solid dosage forms can be prepared by spray dried techniques, hot melt extrusion strategy, micronization, and nano milling technologies.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters, fluorocarbons, and/or other solubilizing or dispersing agents known in the art. A composition having an active compound can also be administered in the form of suppositories for rectal administration.

The carrier, the excipient and the diluent in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples of other carriers include colloidal silicon oxide, magnesium stearate, cellulose, sodium lauryl sulfate, and D&C Yellow #10.

Still within the scope of the present disclosure is a method of treating a cancer.

The method includes administering to a subject in need thereof an effective amount of a compound of formula (I) or a pharmaceutically acceptable salt thereof.

The above-described compounds or a pharmaceutical composition containing one or more of them can be administered to a subject orally, parenterally, by inhalation spray, topically, rectally, nasally, buccally, or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

The term “treating”, “treat” or “treatment” refers to application or administration of the compound to a subject with the purpose to cure, alleviate, relieve, alter, remedy, improve, or affect the disease, the symptom, or the predisposition. “An effective amount” refers to the amount of the compound which is required to confer the desired effect on the subject. Effective amounts vary, as recognized by those skilled in the art, depending on route of administration, excipient usage, and the possibility of co-usage with other therapeutic treatments such as use of other active agents.

The details of one or more embodiments of the disclosure are set forth in the description below. Other features, objects, and advantages of the disclosure will be apparent from the description and from the claims.

### DETAILED DESCRIPTION

One embodiment of the present disclosure is the compounds of formula (I) or a stereoisomer, a tautomer, or a pharmaceutically acceptable salt thereof:

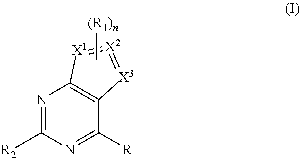

in which each of variables each of variables R, R₁, R₂, X₁, X₂, X₃ and n is defined as in the SUMMARY section.

R₁ can be produced by condensing various hydrazine derivatives into a fused aromatic heterocyclic five-membered ring. In addition, it can also be constructed by Mitsunobu reaction or by simple substitution reaction. The Mitsunobu reaction uses triphenylphosphine and azodicarboxylate (such as diethyl azodicarboxylate (DEAD) or diisopropyl azodicarboxylate (DIAD) to convert alcohols into various functional groups (such as the organic reaction of ester). And alcohol reacts with phosphine to form a good leaving group, and then undergoes stereochemical conversion in the classic S_{N}2 way of nucleophile substitution to form carbon-carbon bond derivatives.

R₂ can be synthesized by Suzuki, and the Sonogaslura reaction is a cross-coupling reaction used to form carbon-carbon bonds in organic synthesis. They use a palladium catalyst as a catalyst to form carbon-carbon bonds between the terminal alkynes and the aryl heteroaryl or vinyl halide. And it is substituted by NR, OR, SR nucleophile in heteroaromatic substitution.

Another embodiment of the present disclosure is the compound of the aforesaid embodiment or a stereoisomer, a tautomer, or a pharmaceutically acceptable salt thereof, wherein

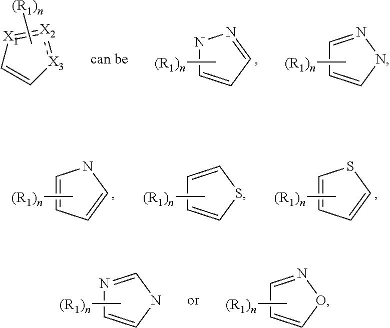

in which n can be 1 or 2.

Another embodiment of the present disclosure is the compound of any one of the aforesaid embodiments or a stereoisomer, a tautomer, or a pharmaceutically acceptable salt thereof, wherein

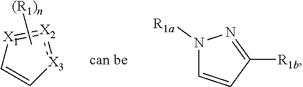

in which each of R₁ₐ and R_{1b} independently can be selected from the group consisting of hydrogen, halogen, alkyl, alkyloxy, cycloalkyl, heterocycloalkyl, aryl or heteroaryl, wherein each of alkyl, alkyloxy, cycloalkyl, heterocycloalkyl, aryl and heteroaryl is optionally substituted with one to three moieties selected from the group consisting of halogen, hydroxyl, nitro, cyano, —NRₐR_{b}, —C(═O)R_{c}, —C(═O)OR_{d}, heterocycloalkyl optionally substituted with one to four halogen, aryl optionally substituted with one to three halogen or —NRₐR_{b}, alkyl optionally substituted with one to three halogen, and alkyloxy optionally substituted with one to three halogen or alkyloxy, in which each of Rₐ and R_{b} independently is hydrogen, alkyl or acrylamide, and each of R_{c} and R_{d} independently is hydrogen, alkenyl, or alkyl optionally substituted with one to three halogen or alkyloxy optionally further substituted with alkyloxy.

Another embodiment of the present disclosure is the compound of any one of the aforesaid embodiments or a stereoisomer, a tautomer, or a pharmaceutically acceptable salt thereof, wherein R_{1b} can be hydrogen or alkyl.

Another embodiment of the present disclosure is the compound of any one of the aforesaid embodiments or a stereoisomer, a tautomer, or a pharmaceutically acceptable salt thereof, wherein R_{1b} can be hydrogen.

Another embodiment of the present disclosure is the compound of any one of the aforesaid embodiments or a stereoisomer, a tautomer, or a pharmaceutically acceptable salt thereof, wherein R₁ₐ can be alkyl, aryl, heteroaryl, cycloalkyl or heterocycloalkyl, wherein each of alkyl, cycloalkyl, heterocycloalkyl, aryl and heteroaryl is optionally substituted with one to three moieties selected from the group consisting of F, Cl, hydroxyl, cyano, —NRₐR_{b}, —C(═O)R_{c}, —C(═O)OR_{d}, heterocycloalkyl optionally substituted with one to four F or Cl, aryl optionally substituted with one to three F, Cl or —NRₐR_{b}, alkyl optionally substituted with one to three F, Cl, and alkyloxy optionally substituted with one to three F, Cl or alkyloxy, in which Rₐ is hydrogen or alkyl, R_{b} is alkyl or acrylamide, R_{c} is alkyl or alkenyl, R_{d} is alkyl optionally substituted with one to three F, Cl or alkyloxy optionally further substituted with alkyloxy.

Another embodiment of the present disclosure is the compound of any one of the aforesaid embodiments or a stereoisomer, a tautomer, or a pharmaceutically acceptable salt thereof, wherein R₁ₐ can be alkyl, benzyl, phenyl, pyridinyl, pyrrolidinyl, cyclopentyl, cyclohexyl, cycloheptyl or piperidinyl, wherein alkyl is optionally substituted with hydroxyl, cyano, dialkylamino, heterocycloalkyl optionally substituted with one or two F, or alkyloxy optionally substituted with alkyloxy; benzyl is optionally substituted with one F; phenyl is optionally substituted with one or two F, Cl, —NRₐR_{b}, alkyl optionally substituted with three F, or alkyloxy optionally substituted with one to three F; pyridinyl is optionally substituted with alkyl optionally substituted with three F; pyrrolidinyl is substituted with alkyl, —C(═O)R_{c} or —C(═O)OR_{d}; cyclopentyl, cyclohexyl and cycloheptyl is optionally substituted with one or two F, Cl or alkyl; and piperidinyl is optionally substituted with alkyl, —C(═O)R_{c}, or —C(═O)OR_{d}; wherein Rₐ is hydrogen, R_{b} is acrylamide, R_{c} is alkyl or alkenyl, R_{d} is alkyl optionally substituted with one to three F, Cl or alkyloxy optionally further substituted with alkyloxy.

Another embodiment of the present disclosure is the compound of any one of the aforesaid embodiments or a stereoisomer, a tautomer, or a pharmaceutically acceptable salt thereof, wherein R₁ₐ can be benzyl, phenyl, pyridinyl, pyrrolidinyl, cyclopentyl, cyclohexyl, cycloheptyl or piperidinyl; wherein benzyl is optionally substituted with one F; phenyl is optionally substituted with one or two F, Cl, —NRₐR_{b}, alkyl optionally substituted with three F, or alkyloxy optionally substituted with one to three F; pyridinyl is optionally substituted with alkyl optionally substituted with three F; pyrrolidinyl is substituted with alkyl, —C(═O)R_{c} or —C(═O)OR_{d}; cyclopentyl, cyclohexyl and cycloheptyl is optionally substituted with one or two F or alkyl; and piperidinyl is optionally substituted with alkyl, —C(═O)R_{c}, or —C(═O)OR_{d}; wherein Rₐ is hydrogen, R_{b} is acrylamide, R_{c} is alkyl or alkenyl, R_{d} is alkyl optionally substituted with one to three F or alkyloxy optionally further substituted with alkyloxy.

Another embodiment of the present disclosure is the compound of any one of the aforesaid embodiments or a stereoisomer, a tautomer, or a pharmaceutically acceptable salt thereof, wherein R can be

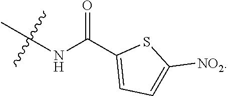 [SMILES: *NC(=O)c1ccc([N+](=O)[O-])s1]

Another embodiment of the present disclosure is the compound of any one of the aforesaid embodiments or a stereoisomer, a tautomer, or a pharmaceutically acceptable salt thereof, wherein R₂ can be —OR₃, —NHR₄, —SR₅, alkenyl, alkynyl, aryl, heteroaryl, cycloalkyl, heterocycloalkyl, aryl fused with heterocycloalkyl or cycloalkenyl, wherein each of alkenyl, alkynyl, aryl, heteroaryl, cycloalkyl, heterocycloalkyl and cycloalkenyl is optionally substituted with one to three moieties selected from the group consisting of F, Cl, hydroxyl, cyano, —NRₑR_{f}, —C(═O)R_{g}, —C(═O)ORₕ, —SO₂Rᵢ, —OSiRⱼ, alkenyl, heterocycloalkyl, aryl optionally substituted with alkyl optionally substituted with one to three F, Cl or alkyloxy optionally substituted with one to three F, Cl, alkyl optionally substituted with one to three F, Cl, —NRₑR_{f} or —ORₖ, and alkyloxy optionally substituted with one to four F, Cl, hydroxyl, —NRₑR_{f} or alkyloxy optionally further substituted with alkyloxy, in which each of Rₑ and R_{f} independently is hydrogen or alkyl optionally substituted with alkyloxy, each of R_{g} and Rₕ independently is hydrogen, alkyl or alkenyl, Rᵢ is alkyl, Rⱼ is alkyl, and Rₖ is hydrogen, aryl, or alkyl optionally substituted with alkyloxy optionally further substituted with alkyloxy; each of R₃ and R₅ independently is alkyl, cycloalkyl or aryl, wherein alkyl, cycloalkyl or aryl is optionally substituted with one to four moieties selected from the group consisting of F, Cl, alkyl optionally substituted with one to four F or Cl, alkyloxy optionally substituted with one to three F, Cl or alkyloxy, —NRₗRₘ, and heterocycloalkyl, in which each of Rₗ and Rₘ independently is hydrogen or alkyl; and R₄ is alkyl, cycloalkyl or —SO₂Rₘ, in which Rₒ is hydrogen, alkyl or aryl, and each of alkyl or aryl is optionally substituted with one to three F or Cl.

Another embodiment of the present disclosure is the compound of any one of the aforesaid embodiments or a stereoisomer, a tautomer, or a pharmaceutically acceptable salt thereof, wherein R₂ can be —OR₃, —NHR₄, —SR₅, styryl, phenylethnyl, cyclohexyl, cyclohexenyl, phenyl, benzodioxolyl, benzodioxinyl, benzofuranyl, furanyl, thiophenyl, xazolyl, imidazole, pyrazolyl, oxadiazolyl, azetidinyl, pyrrolidinyl, piperidinyl, morpholinyl, piperazinyl, pyrimidinyl, or pyridinyl, wherein styryl is optionally substituted with alkyl substituted with one to three F or alkoxy; phenylethynyl is optionally substituted with alkyl optionally substituted with one to three F or alkoxy optionally substituted with one to three F; cyclohexyl is optionally substituted with one to three alkyl; cyclohexenyl is optionally substituted with one to three alkyl; phenyl is optionally substituted with one to three moieties selected from the group consisting of F, Cl, cyano, dialkylamino, —C(═O)R_{g}, —C(═O)ORₕ, —SO₂Rᵢ, alkyl optionally substituted with one to three F, dialkylamin or alkyloxy, and alkyloxy optionally substituted with one to three F or alkyloxy optionally further substituted with alkyloxy; thiophenyl is optionally substituted with Cl or alkyl; xazolyl is optionally substituted with one or two alkyl; pyrazolyl is optionally substituted with alkyl optionally substituted with one to three F; oxadiazolyl is optionally substituted with alkyl; azetidinyl is optionally substituted with one or two F or —OSiRⱼ; pyrrolidinyl is optionally substituted with hydroxyl, cyano, alkenyl, —C(═O)ORₕ, one to two F, or alkyl optionally substituted with hydroxyl, phenoxy or alkyloxy optionally substituted with alkyloxy optionally further substituted with alkyloxy; piperidinyl is optionally substituted with alkyl, one or two F, —C(═O)R_{g}; morpholinyl is optionally substituted with one to three alkyl; piperazinyl is optionally substituted with alkyl, and pyridinyl is optionally substituted with one or two moieties selected from the group consisting of F, Cl, cyano, piperidinyl, morpholinyl, —NRₑR_{f}, alkyl optionally substituted with three to four F, and alkyloxy optionally substituted with one to four F, hydroxyl, dialkylamino or alkyloxy optionally further substituted with alkyloxy; in which Rₑ is alkyl, R_{f} is alkyl optionally substituted with alkyloxy, R_{g} is hydrogen, alkyl or alkenyl, Rₕ is hydrogen or alkyl, Rᵢ is alkyl, and Rⱼ is alkyl; R₃ is phenyl, cycloalkyl or alkyl, wherein phenyl is optionally substituted with F, morpholinyl, alkyloxy optionally substituted with one to three F, or alkyl optionally substituted with one to three F; and alkyl is optionally substituted with one to four F, morpholinyl, dialkylamino, or alkyloxy optionally substituted with alkyloxy, R₄ is alkyl, cycloalkyl or —SO₂Rₙ, in which Rₙ is phenyl optionally substituted with one to three Cl; and R₅ is phenyl optionally substituted with Cl.

Another embodiment of the present disclosure is the compound of any one of the aforesaid embodiments or a stereoisomer, a tautomer, or a pharmaceutically acceptable salt thereof, wherein R₂ can be

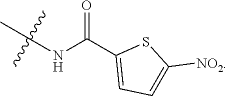 [SMILES: *NC(=O)c1ccc([N+](=O)[O-])s1]

Another embodiment of the present disclosure is the compound of any one of the aforesaid embodiments or a stereoisomer, a tautomer, or a pharmaceutically acceptable salt thereof, wherein R can be phenyl or morpholinyl, and phenyl is optionally substituted with alkyl or alkyloxy optionally substituted with one to three F.

Another embodiment of the present disclosure is the compound of any one of the aforesaid embodiments or a stereoisomer, a tautomer, or a pharmaceutically acceptable salt thereof, wherein

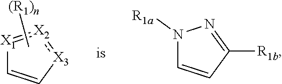

in which R₁ₐ is alkyl and R_{1b} is hydrogen; R is

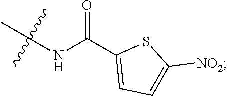 [SMILES: *NC(=O)c1ccc([N+](=O)[O-])s1]

and R₂ is styryl or phenyl, in which phenyl is substituted with alkyl substituted with one to three F or alkyloxy substituted with one to three F; and styryl is substituted with alkyl substituted with one to three F.

Another embodiment of the present disclosure is the compound of any one of the aforesaid embodiments or a stereoisomer, a tautomer, or a pharmaceutically acceptable salt thereof, wherein

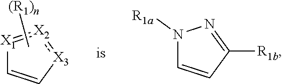

in which R_{1b} is hydrogen, and R₁ₐ is phenyl optionally substituted with one or two F, Cl, acrylamido, alkyl optionally substituted with three F or alkyloxy optionally substituted with three F; R is

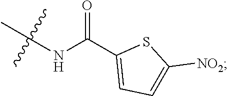 [SMILES: *NC(=O)c1ccc([N+](=O)[O-])s1]

and R₂ is —OR₃, phenyl, benzodioxolyl, thiophenyl, pyrazolyl, pyrrolidinyl, pyrimidinyl, or pyridinyl, wherein phenyl is optionally substituted with one or two moieties selected from the group consisting of F, Cl, cyano, alkyl substituted with one to three F, and alkyloxy substituted with one to three F, pyrazolyl is optionally substituted with alkyl; pyrrolidinyl is substituted with —C(═O)ORₕ, one to two F, or alkyl substituted with hydroxyl, phenoxy or alkyloxy; pyridinyl is optionally substituted with one or two moieties selected from the group consisting of F, Cl, cyano, piperidinyl, alkyl, and alkyloxy optionally substituted with dialkylamino or alkyloxy optionally further substituted with alkyloxy; R₃ is alkyl optionally substituted with dialkylamino; and Rₕ is alkyl.

Another embodiment of the present disclosure is the compound of any one of the aforesaid embodiments or a stereoisomer, a tautomer, or a pharmaceutically acceptable salt thereof, wherein

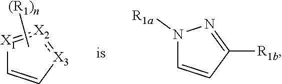

in which R_{1b} is hydrogen, and R₁ₐ is pyridinyl substituted with alkyl substituted with one to three F; R is

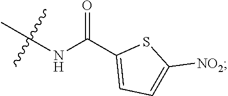 [SMILES: *NC(=O)c1ccc([N+](=O)[O-])s1]

and R₂ is phenyl substituted with one or two F or Cl.

Another embodiment of the present disclosure is the compound of any one of the aforesaid embodiments or a stereoisomer, a tautomer, or a pharmaceutically acceptable salt thereof, wherein

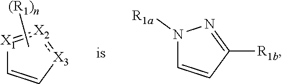

in which R_{1b} is hydrogen, R₁ₐ is piperidinyl substituted with —C(═O)OR_{d}, and R_{d} is alkyl; R is

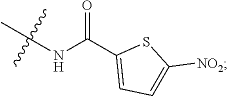 [SMILES: *NC(=O)c1ccc([N+](=O)[O-])s1]

and R₂ is phenyl substituted with Cl or —C(═O)ORₕ, in which Rₕ is alkyl.

Another embodiment of the present disclosure is the compound of any one of the aforesaid embodiments or a stereoisomer, a tautomer, or a pharmaceutically acceptable salt thereof, wherein

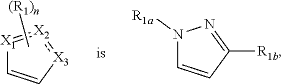

in which R_{1b} is hydrogen, and R₁ₐ is cyclohexyl optionally substituted with one or two F or alkyl; R is

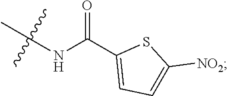 [SMILES: *NC(=O)c1ccc([N+](=O)[O-])s1]

and R₂ is phenyl or pyridinyl, wherein phenyl is substituted with one or two F, Cl, or —C(═O)ORₕ, in which Rₕ is alkyl; and pyridinyl is substituted with F, Cl or alkyloxy.

Another embodiment of the present disclosure is the compound of any one of the aforesaid embodiments or a stereoisomer, a tautomer, or a pharmaceutically acceptable salt thereof, wherein

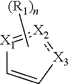

can be

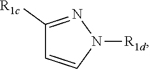

in which R_{1c} and R_{1d} are respectively alkyl.

Another embodiment of the present disclosure is the compound of any one of the aforesaid embodiments or a stereoisomer, a tautomer, or a pharmaceutically acceptable salt thereof, wherein R can be

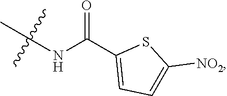 [SMILES: *NC(=O)c1ccc([N+](=O)[O-])s1]

and R₂ is phenyl, wherein phenyl is optionally substituted with F, alkyl optionally substituted with one to three F or alkyloxy optionally substituted with one to three F.

Another embodiment of the present disclosure is the compound of any one of the aforesaid embodiments or a stereoisomer, a tautomer, or a pharmaceutically acceptable salt thereof, wherein

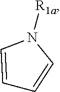

can be

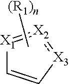

in which R_{1c} is alkyl.

Another embodiment of the present disclosure is the compound of any one of the aforesaid embodiments or a stereoisomer, a tautomer, or a pharmaceutically acceptable salt thereof, wherein R can be

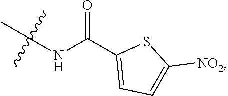 [SMILES: *NC(=O)c1ccc([N+](=O)[O-])s1]

and R₂ is phenyl optionally substituted with alkyl.

Another embodiment of the present disclosure is the compound of any one of the aforesaid embodiments or a stereoisomer, a tautomer, or a pharmaceutically acceptable salt thereof, wherein

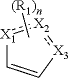

can be

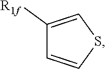

in which R_{1f} is alkyl or aryl optionally substituted with halogen or alkyl.

Another embodiment of the present disclosure is the compound of any one of the aforesaid embodiments or a stereoisomer, a tautomer, or a pharmaceutically acceptable salt thereof, wherein R_{1f} can be alkyl, R₂ can be

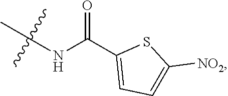 [SMILES: *NC(=O)c1ccc([N+](=O)[O-])s1]

and R can be phenyl, in which phenyl is optionally substituted with alkoxy optionally substituted with one to three F.

Another embodiment of the present disclosure is the compound of any one of the aforesaid embodiments or a stereoisomer, a tautomer, or a pharmaceutically acceptable salt thereof, wherein R_{1f} can be alkyl or phenyl optionally substituted with F or alkyl, R can be

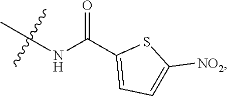 [SMILES: *NC(=O)c1ccc([N+](=O)[O-])s1]

and R₂ can be —OR₃, pyrrolidinyl or phenyl, in which pyrrolidinyl is optionally substituted with one or two F, phenyl is optionally substituted with one to three moieties selected from the group consisting of F, Cl dialkylamino, alkyl optionally substituted with one to three F, and alkoxy optionally substituted with one to three F, and R₃ is phenyl optionally substituted with alkyl.

Another embodiment of the present disclosure is the compound of any one of the aforesaid embodiments or a stereoisomer, a tautomer, or a pharmaceutically acceptable salt thereof, wherein

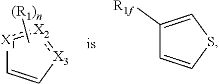

in which R_{1f} is alkyl; R is

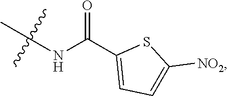 [SMILES: *NC(=O)c1ccc([N+](=O)[O-])s1]

and R₂ is phenyl, in which phenyl is substituted with alkyl substituted with one to three F or alkyloxy substituted with ore to three F.

Another embodiment of the present disclosure is the compound of any one of the aforesaid embodiments or a stereoisomer, a tautomer, or a pharmaceutically acceptable salt thereof, wherein

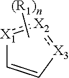

can be

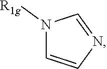

in which R_{1g} can be cycloalkyl or heterocycloalkyl optionally substituted with —C(═O)OR_{d}, and R_{d} is alkyl.

Another embodiment of the present disclosure is the compound of any one of the aforesaid embodiments or a stereoisomer, a tautomer, or a pharmaceutically acceptable salt thereof, wherein Rig can be piperidinyl or cyclohexyl, R can be

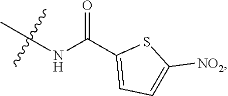 [SMILES: *NC(=O)c1ccc([N+](=O)[O-])s1]

and R₂ can be phenyl optionally substituted with F or pyridinyl optionally substituted with F or alkyloxy.

Another embodiment of the present disclosure can be a compound selected from the group consisting of Compounds 1-1 to 1-33, Compounds 2-1 to 2-8, Compounds 3-1 to 3-12, Compounds 4-1 to 4-22, Compounds 5-1 to 5-57, Compounds 6-1 to 6-145, Compounds 7-1 to 7-19, Compounds 8-1 to 8-16, Compounds 9-1 to 9-13 and Compounds 10-1 to 10-72, which are listed in the following Tables 1 to 10; or a stereoisomer, a tautomer, or a pharmaceutically acceptable salt thereof.

A further another embodiment of the present disclosure can be a compound selected from the group consisting of compounds 1-4 to 1-5, compound 1-11, compound 1-22, compound 1-27, compound 4-2, compound 4-5, compound 5-6, compound 5-18, compounds 5-28 to 5-33, compound 5-35, compound 5-39, compound 5-40, compound 5-42, compound 5-44, compound 5-45, compound 5-47, compound 5-49, compound 5-56, compound 64, compound 6-11, compound 6-18, compound 6-19, compounds 6-24 to 6-28, compound 6-35, compound 6-36, compound 6-39, compound 6-43, compound 6-45, compound 6-46, compounds 6-50 to 6-54, compounds 6-57 to 6-58, compound 6-66, compound 6-69, compound 6-71, compound 6-75, compound 6-85, compounds 6-89 to 6-91, compound 6-104, compound 6-106, compound 6-108, compounds 6-110 to 6-112, compounds 6-114 to 6-121, compound 6-125, compounds 6-127 to 6-128, compound 6-130, compound 6-133, compound 6-135, compound 6-137, compound 7-1, compound 7-3, compound 7-6, compound 7-13, compound 7-19, compound 9-3, compound 9-5, compound 10-13, compound 10-14, compound 10-18, compound 10-23, compound 10-32, compound 10-40, compound 10-55, compound 10-57, and compound 10-64; or a stereoisomer, a tautomer, or a pharmaceutically acceptable salt thereof.

The compounds of the present disclosure may contain asymmetric or chiral centers, and exist in different stereoisomeric forms. Unless specified otherwise, all stereoisomeric forms of the compounds of the present disclosure as well as mixtures thereof, including racemic mixtures are within the scope of the present disclosure. In addition, the compounds of the present disclosure may also exist in different geometric and positional isomers. For example, both the cis- and trans-forms, as well as mixtures of the compound with a double bond or a fused ring, are also within the scope of the present disclosure.

Diastereomeric mixtures can be separated into their individual diastereoisomers by any methods, such as by chromatography and/or fractional crystallization. Enantiomers can be separated by use of a chiral HPLC column or by converting the enantiomeric mixture into a diastereomeric mixture by reaction with an appropriate optically active compound to separate the diastereoisomers and convert the individual diastereoisomers into pure enantiomers. The specific stereoisomers may be synthesized by converting one stereoisomer into the other by asymmetric transformation, by using an optically active starting material or by asymmetric synthesis using optically active reagents, catalysts, substrates or solvents.

Also within the present disclosure is a pharmaceutical composition, comprising: (1) the compound of the present disclosure, or a stereoisomer, a tautomer, or a pharmaceutically acceptable salt thereof, and (2) a pharmaceutically acceptable carrier, excipient or diluent. The composition may also comprise at least one additional pharmaceutical agent such as anti-cancer agents. The compound or the pharmaceutically acceptable salt thereof or the composition of the present disclosure may be used in the manufacture of a medicament of inhibiting the growth of tumor cells or treating cancer.

Also within the present disclosure is a method for treating a cancer, which includes the step of administering to the subject in need thereof an effective amount of the compound of the present disclosure or a stereoisomer, a tautomer, or a pharmaceutically acceptable salt thereof.

Further covered by the present disclosure a method of inhibiting a growth of tumor cells, which includes the step of administering to a subject in need thereof an effective amount of the compound of the present disclosure or a stereoisomer, a tautomer, or a pharmaceutically acceptable salt thereof.

In the present disclosure, the aforesaid subject can be mammal, for example, human.

In the present disclosure, the compound of the present disclosure or a stereoisomer, a tautomer, or a pharmaceutically acceptable salt thereof can inhibit the growth of tumor cells to achieve the purpose of treating a cancer. Examples of the cancer include, but am not limited to, gastric cancer, colon cancer, colorectal cancer, breast cancer, lung cancer, prostate cancer, bladder cancer, pancreatic cancer, liver cancer, uterine cancer, cervical caner, endometrial cancer, esophageal cancer, leukemia, lymphoma, kidney cancer, osteosarcoma, ovarian cancer, skin cancer, small intestine cancer, thymus cancer, thyroid cancer, nervous system cancers, bone cancer, brain cancer, or head and neck cancer.

The compounds or a stereoisomer, a tautomer, or a pharmaceutically acceptable salt thereof of the present disclosure may be administered in combination with at least one additional pharmaceutical agent such as anti-cancer agent. The administration formulation can be, for example, (a) a single formulation comprising the compound of the present disclosure or a stereoisomer, a tautomer, or a pharmaceutically acceptable salt thereof, a pharmaceutically acceptable carrier, excipient or diluent and at least one additional pharmaceutical agent; or (b) two formulations administered simultaneously or sequentially and in any order, wherein one comprises the compound of the present disclosure or a stereoisomer, a tautomer, or a pharmaceutically acceptable salt thereof, a pharmaceutically acceptable carrier, excipient or diluent and the other one comprises at least one additional pharmaceutical agent.

Suitable anti-cancer agents may include Herceptin, Rituximab, Docetaxel, Capecitabine, Cetuximnab, Gefitinib, PD-1, Sorafenib tosylate or Imatinib, but the present disclosure is not limited thereto. Any other anti-cancer agents known in the art can also be used in the present disclosure.

Methods for synthesizing the compounds of formula (I) are well known in the art. See, for example, R. Larock, Comprehensive Organic Transformations (2^{nd} Ed., VCH Publishers 1999); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994). L. Paquette, ed., Encyclopedia of Reagents for Organic Synthesis (2^{nd} ed., John Wiley and Sons 2009); P. Roszkowski, J. K. Maurin, Z. Czarnocki “Enantioselective synthesis of (R)-(−)-praziquantel (PZQ)” Tetrahedron: Asymmetry 17 (2006) 1415-1419; and L. Hu, S. Magesh, L. Chen, T. Lewis, B. Munoz, L. Wang “Direct inhibitors of keap1-nrf2 interaction as antioxidant inflammation modulators,” WO2013/067036.

The compounds of formula (I) thus prepared can be initially screened using in vitro assays, e.g., NCI-60 screening platform or MTS method. They can be subsequently evaluated using in vivo assays known in the field. The selected compounds can be further tested to verify their efficacy in disease related efficacy and adverse effects models. Based on the results, an appropriate dosage range and administration route can be determined.

The following embodiments are made to clearly exhibit the above-mentioned and other technical contents, features and/or effects of the present disclosure. Through the exposition by means of the specific embodiments, people would further understand the technical means and effects the present disclosure adopts to achieve the above-indicated objectives. Moreover, as the contents disclosed herein should be readily understood and can be implemented by a person skilled in the art, all equivalent changes or modifications which do not depart from the concept of the present disclosure should be encompassed by the appended claims.

### Example

Without further elaboration, it is believed that one skilled in the art can, based on the above description, utilize the present disclosure to its fullest extent. The following specific examples are therefore to be construed as merely illustrative, and not limitative of the remainder of the disclosure in any way whatsoever. All publications cited herein are incorporated by reference in their entirety.

Described below are the procedures used to synthesize the exemplary compounds of the present disclosure.

Unless otherwise stated, all starting materials used were commercially available and used as supplied. Reactions requiring anhydrous conditions were performed in flame-dried glassware and cooled under an argon or nitrogen atmosphere. Unless otherwise stated, reactions were carried out under argon or nitrogen and monitored by analytical thin-layer chromatography performed on glass-backed plates (5 cm_10 cm) precoated with silica gel 60 F254 as supplied by Merck. Visualization of the resulting chromatograms was done by looking under an ultraviolet lamp (λ=254 nm), followed by dipping in an nBuOH solution of Ninhydnn (0.3% w/v) containing acetic acid (3% v/v) or ethanol solution of phosphomolybdic acid (2.5% w/v) and charring by heat gun. Solvents for reactions were dried under an argon or nitrogen atmosphere prior to use as follows: THF, Toluene, and DCM were dried by the column of Dried molecular Sieve 5A (LC technology solution Inc). DMF dried by calcium hydride or anhydrous is commercial available. Flash chromatography was used routinely for purification and separation of product mixtures using RediSep Rf Silica Gel Disposable Flash Columns, Gold® 20-40/40-60 microns silica gel and Reusable RediSep Rf Gold® C18 Reversed Phase columns, 20-40 microns supplied by RediSep. Eluent systems are given in volume/volume concentrations. ¹³C and ¹H NMR spectra were recorded on Bruker AVIII (400 MHz). Chloroform-d or dimethyl sulfoxide-d6 and CD₃OD was used as the solvent and TMS (δ 0.00 ppm) as an internal standard. Chemical shift values are reported in ppm relative to the TMS in delta (S) units. Multiplicities are recorded as s (singlet), br s (broad singlet), d (doublet), t (triplet), q (quartet), dd (doublet of doublet), dt (doublet of triplet), m (multiplet). Coupling constants (J) are expressed in Hz. Electrospray mass spectra (ESMS) were recorded using a Thermo LTQ XL mass spectrometer. Spectral data were recorded as m/z values.

In the preparation of compounds of the present invention, protection of remote functionality (e.g., primary or secondary amine) of intermediates may be necessary. The need for such protection may vary depending on the nature of the remote functionality and the conditions of the preparation methods. Suitable amino protecting groups (NHPg) include, for example, acetyl, trifluoroacetyl, tbutoxycarbonyl (BOC), 9-fluorenylmethyleneoxycarbonyl (Fmoc) and benzyloxycarbonyl (CBz). Similarly, a “hydroxyl protecting group” refers to a substituent of a hydroxy group that blocks or protects the hydroxy functionality. Suitable hydroxyl protecting groups (OPg) include, for example, allyl, acetyl, silyl, benzyl, paramethoxy benzyl, trityl, and the like. The need for such protection is readily determined by one skilled in the art.

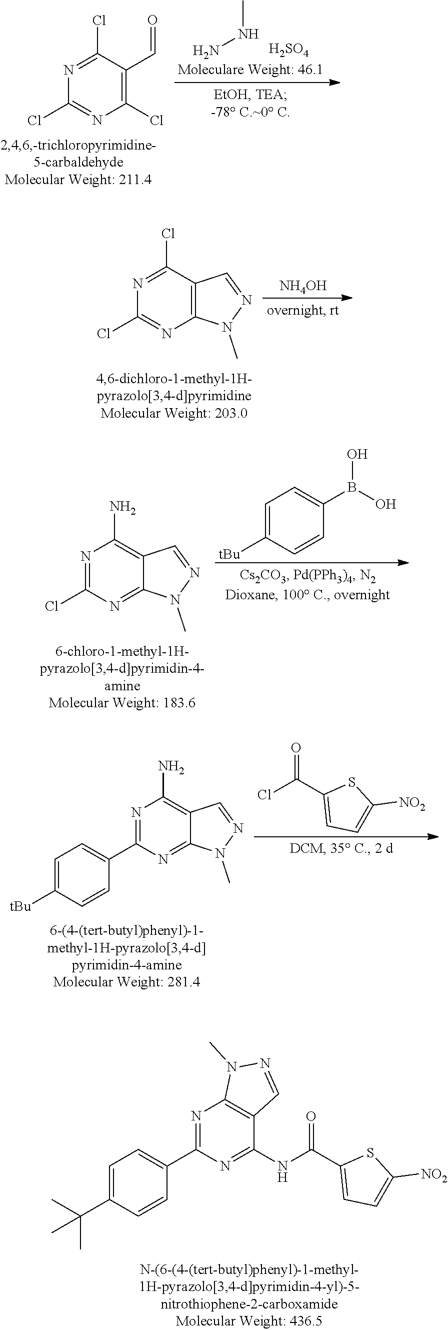

### Experimental Procedure

### Step 1: Cyclization

To a solution of the 2,4,6-trichloropyrimidine-5-carbaldehyde (3.71 g, 17.5 mmol) dissolved in EtOH (50 ml) cooled to −78° C. was added methyl hydrazine (0.93 ml, 17.5 mmol) and TEA (8 ml). The mixture was stirred for 30 minutes at −78° C., and then 2 hr at 0° C. The solution was concentrated in vacuum without heating. To the reduced volume solution EtOAc was added and the solution washed with a saturated NaHCO₃ solution and concentrated in vacuum without heating. Filtration over a small silica gel plug (2:1=EtOAc:Hex) and concentration afforded the desired product 4,6-Dichloro-1-methyl-1H-pyrazolo[3,4-d]pyrimidine as a yellow solid (2.84 g, 80%).

### Step 2: Amination

To a suspension of 4,6-Dichloro-1-methyl-1H-pyrazolo[3,4-d]pyrimidine (2.84 g, 14 mmol) in the reaction flask and then add THF (56 ml) to wait for the solid to dissolve completely. Then add 20 g 30% ammonium solution and react at room temperature (25° C.) for 24 h and poured 60 ml water into the solution, filtration by suction to afford the 6-chloro-1-methyl-1H-pyrazolo[3,4-d]pyrimidin-4-amine (2.17 g, 85%) as a yellow solid powder.

### Step 3: Suzuki Coupling Reaction

To a suspension of 6-chloro-1-methyl-1H-pyrazolo[3,4-d]pyrimidin-4-amine (1.84 g, 10 mmol), 4-(tert-butyl)phenyl boronic acid (2.67 g, 15 mmol), Tetrakis (triphenylphosphine) palladium (0) (1.16 g, 1 mmol), 1,4-dioxane (40 ml), water (2 ml) and aqueous Cesium carbonate solution (2.0 M, 20 ml, 40 mmol) in a 100 ml glass flask was deoxygenated using 6 alternate vacuum/Nitrogen flush cycles. The reaction mixture was heated to 100° C. for 16 hrs. After cooling to room temperature, poured the Celite powder into the flask and stirred for 10 min at room temperature then filter by suction and the filtrate was partitioned between ethyl acetate (80 ml) and saturated aqueous sodium bicarbonate solution (30 ml). The layers were separated and the organic layer was washed with brine (30 ml), dried over sodium sulfate, filtered, and concentrated under reduced pressure. The residue was purified by chromatography on silica gel (0-25% n-Hexanes/Ethyl acetate, linear gradient) to afford 6-(4-(tert-butyl)phenyl)-1-methyl-1H-pyrazolo[3,4-d]pyrimidin-4-amine (2.25 g, 80%) as a yellow powder.

### Step 4: Amidation Reaction

### N-(6-(4-(tert-butyl)phenyl)-1-methyl-1H-pyrazolo[3,4-d]pyrimidin-4-yl)-5-nitrothiophene-2-carboxamide formation by amidation of carbonyl acid

To a solution of 6-(4-(tert-butyl)phenyl)-1-methyl-1H-pyrazolo[3,4-d]pyrimidin-4-amine (843 mg, 3.00 mmol) in pyridine (6 ml), was added 5-nitrothiophene-2-carbonyl chloride (900 mg, 4.68 mmol). The reaction mixture was stirred at ambient temperature for 15 hr. Water was added and the product extracted with ethyl acetate. The organic layer was separated, dried (anhydrous sodium sulfate) and evaporated in vacuo. The product was purified by column chromatography on silica gel (hexane-ethyl acetate gradient eluent) and recrystallization with hexane and acetone to yield the title compound. Yield 980 mg (75%). N-(6-(4-(tert-butyl)phenyl)-1-methyl-1H-pyrazolo[3,4-d]pyrimidin-4-yl)-5-nitrothiophene-2-carboxamide as a yellow powder.

### Experimental Procedure

### Cyclization Via Condensation and Closure Ring Reactions

To a solution of the 2,4,6-trichloropyrimidine-5-carbaldehyde (3.7 g, 17.5 mmol) dissolved in 50 ml EtOH and cooled to 0° C. by ice bath was added phenylhydrazine hydrochloride (2.53 g, 17.5 mmol) and 8 ml TEA. The mixture was stirred for 30 minutes at 0° C. and spontaneous to ambient temperature. The solution was concentrated in vacuum without heating. To the reduced volume solution EtOAc was added and the solution washed with a saturated NaHCO₃ solution and concentrated in vacuum without heating. Filtration over a small silica gel plug (EtOAc:Hex=2:1) and concentration afforded the desired product 4,6-dichloro-1-phenyl-1H-pyrazolo[3,4-d] pyrimidine as a yellow solid (3.94 g, 85%).

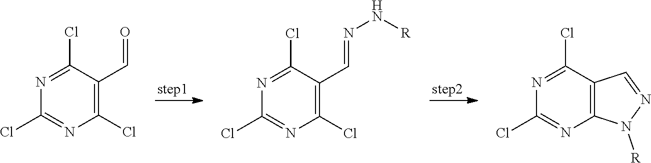

| Step1 condition | | | | Step2 condition | | | |
|---|---|---|---|---|---|---|---|
| hydrazine (1 eq.) | Solvent | Temp. (° C.) | Time | Solvent | Base*^{a} | Temp. (° C.) | Time |
| Methylhydrazine sulfate | EtOH | −78 | 20 min | EtOH | 3 eq | −78 to 0 | 1 h |
| iso-Propylhydrazine | EtOH | −78 | 20 min | EtOH | 3 eq | −78 to 0 | 1 h |
| hydrochloride | | | | | | | |
| tert-Butylhydrazine | EtOH | −78 | 20 min | EtOH | 3 eq | −78 to 0 | 1 h |
| hydrochloride | | | | | | | |
| 2-Hydroxyethyl | EtOH | −78 | 20 min | EtOH | 3 eq | −78 to 0 | 1 h |
| hydrazine | | | | | | | |
| Cyanoethylhydrazine | EtOH | −78 | 20 min | EtOH | 3 eq | −78 to 0 | 1 h |
| Dimethylaminoethyl | EtOH | −78 | 20 min | EtOH | 3 eq | −78 to 0 | 1 h |
| hydrazine dihydrochloride | | | | | | | |
| Phenylhydrazine | EtOH | −78 | 20 min | EtOH | 3 eq | −78 to 0 | 1 h |
| hydrochloride | | | | | | | |
| 4-tert-Butylphenyl | EtOH | −78 | 30 min | EtOH | 3 eq | −78 to 0 | 1 h |
| hydrazine hydrochloride | | | | | | | |
| 2,4- | Dioxane | 0 | 1 h | Dioxane | 3 eq | 100 | 5 d |
| Dichlorophenylhydrazine | | | | | | | |
| hydrochloride | | | | | | | |
| 3,4- | Dioxane | 0 | 1 h | Dioxane | 3 eq | 100 | 3 d |
| Dichlorophenylhydrazine | | | | | | | |
| hydrochloride | | | | | | | |
| 4-Chlorophenylhydrazine | Dioxane | 0 | 1 h | Dioxane | 3 eq | 0 to rt | 1 d |
| hydrochloride | | | | | | | |
| 3-Chloro-6- | ACN | −10 | 16 h | ACN | none | reflux | 16 h |
| hydrazinopyridazine | | | | | | | |
| 2-Hydraziny1-5-(trifluoro | DMF | rt | 1 d | Dioxane | 0 | 110 | 1 d |
| methyl)pyridine | | | | | | | |
| 3-Chlorophenylhydrazine | Dioxane | rt | 1 d | Dioxane | 1.3 eq | reflux | 2 d |
| hydrochloride | | | | | | | |
| 3-Fluorophenylhydrazine | ACN | rt | 1 d | ACN | 1 eq | reflux | 1 d |
| hydrochloride | | | | | | | |
| 4-Fluorophenylhydrazine | DMF | rt | 1 d | Dioxane | none | 100 | 1 d |
| hydrochloride | | | | | | | |
| 4-(Trifluoromethyl)phenyl | Dioxane | rt | 1 d | Dioxane | 1 eq | reflux | 1 d |
| hydrazine | | | | | | | |
| 4-Trifluoromethoxyphenyl | DMF | rt | 1 d | Dioxane | 0 | 100 | 1 d |
| hydrazine hydrochloride | | | | | | | |
| 4-Nitrophenylhydrazine | ACN | rt | 1 d | ACN | 1 eq | reflux | 4 d |
| hydrochloride | | | | | | | |
| 4-methoxyphenylhydrazine | Dioxane | 0 ~ rt | 12 h | Dioxane | 0 | reflux | 4 h |
| hydrochloride | | | | | | | |

*^{a}: Use TEA or DIPEA as base

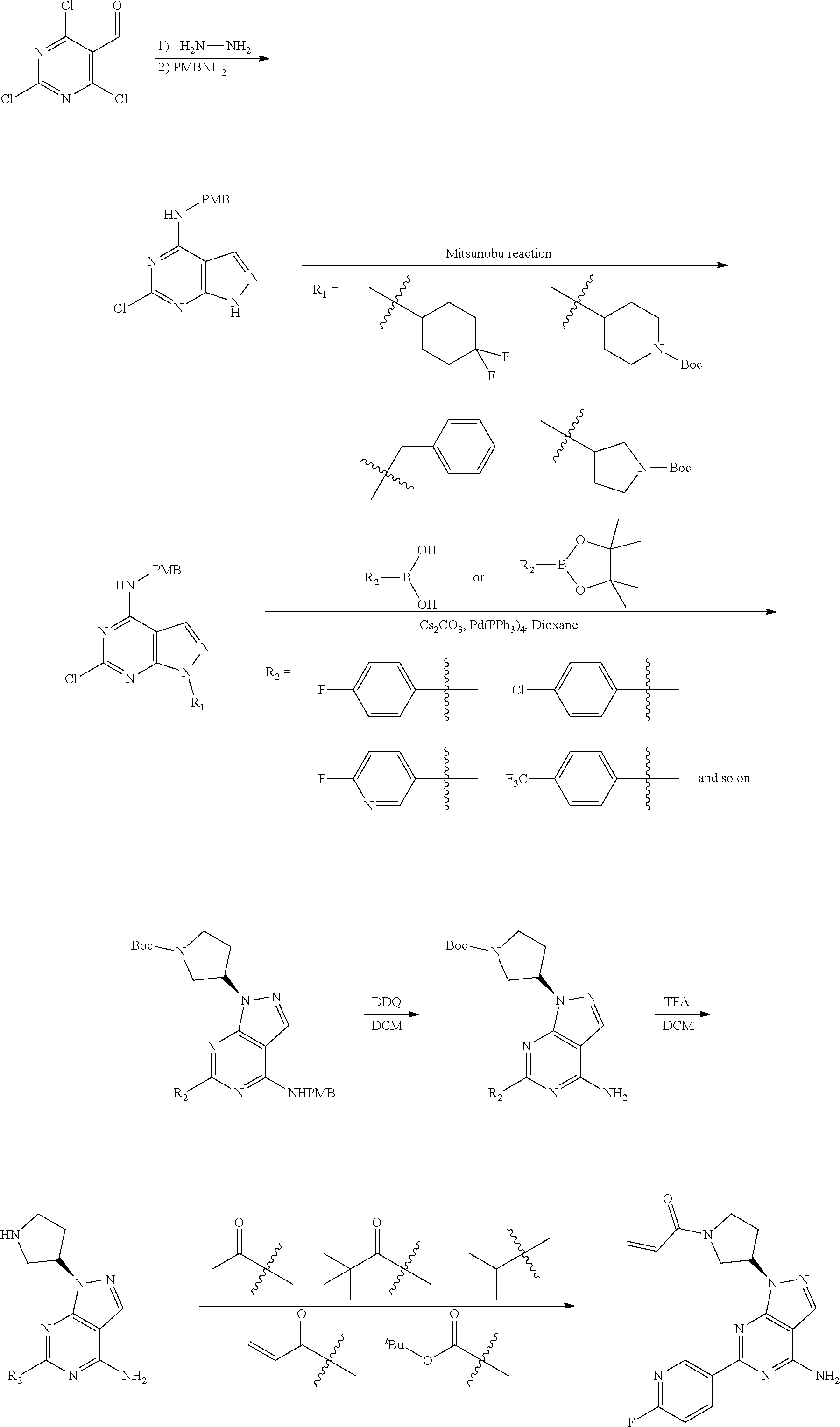

N—C Bond Formation with Mitsunobu Reaction

### Formation of tert-butyl 4-(6-chloro-4-((4-methoxybenzyl)amino)-1H-pyrazolo[3,4-d] pyrimidin-1-yl)piperidine-1-carboxylate

To a flame dried round bottom flask under N₂ was added 6-chloro-N-(4-methoxy benzyl)-1H-pyrazolo[3,4-d]pyrimidin-4-amine (4.35 g, 15 mmol), t-butyl 4-hydroxy-1-piperidinecarboxylate (3.96 g, 20 mmol), and PPh₃ (5.24 g, 20 mmol) in anhydrous tetrahydrofuran (175 ml). The mixture was cooled to 0° C. and diisopropyl azodicarboxylate (DIAD) (4.0 ml, 20 mmol) in tetrahydrofuran (30 ml) was added dropwise. The mixture was allowed to warm to room temperature and stirred overnight. The reaction was then filtered and taken up in DCM. The insoluble material was filtered off. The filtrate was concentrated, taken up in DCM, and put in the freezer for 5 hr. The crystals that formed were filtered off and the filtrate was purified by silica gel chromatography (5-12% methanol/DCM) to give pure tert-butyl 4-(6-chloro-4-((4-methoxybenzyl)amino)-1H-pyrazolo[3,4-d]pyrimidin-1-yl)piperidine-1-carboxylate (4.78 g, 70%).

### Suzuki Coupling Reaction

### Formation of 4-(6-(4-chlorophenyl)4-((4-m)ethoxybenzyl)amino)-1H-pyrazolo[3,4-d]pyrimidin-1-yl)piperidine-1-carboxylate

To a suspension solution of tert-butyl 4-(6-chloro-4-((4-methoxybenzyl)amino)-1H-pyrazolo[3,4-d]pyrimidin-1-yl)piperidine-1-carboxylate (4.73 g, 10 mmol), 4-chlorophenyl boronic acid (1.87 g, 12 mmol), Tetrakis (triphenylphosphine) palladium (0) (1.16 g, 1 mmol), 1,4-dioxane (100 ml), water (40 ml), and aqueous Cesium carbonate solution (1.0 M, 40 ml, 40 mmol) in a 100 ml glass flask was deoxygenated using 6 alternate vacuum/Nitrogen flush cycles. The reaction mixture was heated to 90° C. for 16 hrs. After cooling to room temperature, poured the Celite powder into the flask and stirred for 10 min at room temperature then filter by suction and the filtrate was partitioned between ethyl acetate (160 ml) and saturated aqueous sodium bicarbonate solution (80 ml). The layers were separated and the organic layer was washed with brine (80 ml), dried over sodium sulfate, filtered, and concentrated under reduced pressure. The residue was purified by chromatography on silica gel (0-35% n-Hexanes/Ethyl acetate, linear gradient) to afford tert-butyl 4-(6-(4-chloro phenyl)-4-((4-methoxybenzyl)amino)-1H-pyrazolo[3,4-d]pyrimidin-1-yl)piperidine-1-carboxylate (4.23 g, 77%) as a yellow powder.

### Deprotection by DDQ

### Formation of tert-butyl 4-(4-amino-6-(4-chlorophenyl)-1H-pyrazolo[3,4-d] pyrimidin-1-yl)piperidine-1-carboxylate

To a mixture of tert-butyl 4-(6-(4-chlorophenyl)-4-((4-methoxybenzyl)amino)-1H-pyrazolo[3,4-d]pyrimidin-1-yl)piperidine-1-carboxylate (4.12 g, 7.5 mmol) and DDQ (2.04 g, 9.0 mmol) in the reaction flask, poured 125 ml DCM, 25 ml H₂O, and stir overnight at room temperature. After the reaction is over, then add DCM and NaHCO₃ (aq) to extract, use DCM (150 ml×3) to extract the water layer, use anhydrous MgSO₄ to remove water and use Celite to filter and concentrate, then use silica gel column chromatography (10% EA/DCM) to afford tert-butyl 4-(4-amino-6-(4-chlorophenyl)-1H-pyrazolo[3,4-d]pyrimidin-1-yl)piperidine-1-carboxylate 2.54 g (yield 79%) as a white solid.

### Deprotection by TFA

### Formation of (S)-6-(6-fluoropyridin-3-yl)-1-(pyrrolidin-3-yl)-1H-pyrazolo[3,4-d]pyrimidin-4-amine

To a solution of tert-butyl (S)-3-(4-amino-6-(6-fluoropyridin-3-yl)-1H-pyrazolo[3,4-d]pyrimidin-1-yl)pyrrolidine-1-carboxylate (2.00 g, 5 mmol) in 20 ml DCM then added 5 ml TFA dropwised at room temperature for 1 h. After the reaction is judged by LCMS, concentrate to remove the solvent, add EA and adjusted to pH=11 by addition of 10% NaOH, use EA (100 ml×3) to extract the water layer, use anhydrous MgSO₄ to remove water from the collected organic layer, and filter it concentrate to obtain (S)-6-(6-fluoropyridin-3-yl)-1-(pyrrolidin-3-yl)-1H-pyrazolo[3,4-d]pyrmidin-4-amine 1.28 g (86%) as yellow solid.

Amidation with Acyloyl Chloride

### Formation of (S)-1-(3-(4-amino-6-(6-fluoropyridin-3-yl)-1H-pyrazolo[3,4-d] pyrimidin-1-yl)pyrrolidin-1-yl)prop-2-en-1-one

Add TEA (2.02 g, 20 mmol) and acryloyl chloride (1.00 g, 11 mmol) to a solution of (S)-6-(6-fluoropyridin-3-yl)-1-(pyrrolidin-3-yl)-1H-pyrazolo [3,4-d]pyrimidin-4-amine (1.20 g, 4 mmol) in 50 ml THF at 0° C. by ice bath, stir the resulting mixture for 50 minutes and quench the mixture by 8 ml MeOH then extraction and concentrate the mixture and purify by silica gel column chromatography (0-10% MeOH in DCM) to obtain (S)-1-(3-(4-amino-6-(6-fluoropyridin-3-yl)-1H-pyrazolo[3,4-d]pyrimidin-1-yl)pyrrolidin-1-yl)prop-2-en-1-one 1.06 g (75%) as pale yellow solid.

### Amination

To a suspension solution of 4,6-Dichloro-1-phenyl-1H-pyrazolo[3,4-d]pyrimidine (3.71 g, 14 mmol) in the reaction flask and then add 60 ml THF to wait for the solid to dissolve completely. Then add 20 g, 30% ammonium solution and react at room temperature (25° C.) for 24 h. Poured 60 ml water into the solution, filtration by suction to afford the 6-chloro-1-methyl-1H-pyrazolo[3,4-d]pyrimidin-4-amine (2.93 g, 85%) as a yellow solid powder.

### N-Alkylation

### 6-(3,3-difluoropyrolidin-1-yl)-1-phenyl-1H-pyrazolo[3,4-d]pyrimidin-4-amine formation

A suspension solution of 6-chloro-1-phenyl-1H-pyrazolo[3,4-d]pyrimidin-4-anine (2.46 g, 10 mmol), 3,3-Difluoropyrrolidine hydrochloride (2.15 g, 15 mmol), 1,4-dioxane (50 ml), Cesium carbonate (13.03 g, 40 mmol) in a 100 ml glass flask. The reaction mixture was heated to 100° C. for 16 hrs, then cooling to ambient temperature, and filter by suction and the filtrate was removed and extract with ethyl acetate (60 ml) three time. The layers were separated and the organic layer was washed with brine (30 ml), dried over sodium sulfate, filtered, and concentrated under reduced pressure. The residue was purified by chromatography on silica gel (n-Hexanes/0-30% Ethyl acetate, linear gradient) to afford 6-(3,3-difluoropyrrolidin-1-yl)-1-phenyl-1H-pyrazolo[3,4-d] pyrimidin-4-amine (2.66 g, 83%) as a pale yellow powder.

### Amidation

### N-(6-(3,3-difluoropyrrolidin-1-yl)-1-phenyl-1H-pyrazolo[3,4-d]pyrimidin-4-yl)-5-nitrothiophene-2-carboxamide formation by amidation of carbonyl acid

To a solution of 6-(3,3-difluoropyrrolidin-1-yl)-1-phenyl-1H-pyrazolo[3,4-d]pyrimidin-4-amine (948 mg, 3.00 mmol) and triethyamine (2.06 ml) in 30 ml THF, was added 5-nitrothiophene-2-carbonyl chloride (900 mg, 4.68 mmol). The reaction mixture was stirred at ambient temperature for 15 hr. Water was added and the product extracted with ethyl acetate. The organic layer was separated, dried (anhydrous sodium sulfate) and evaporated in vacuo. The product was purified by column chromatography on silica gel (hexane-ethyl acetate gradient) and recrystallization (DCM and MeOH) to yield the title compound. Yield 1060 mg (75%) N-(6-(4-(tert-butyl)phenyl)-1-methyl-1H-pyrazolo [3,4-d]pyrimidin-4-yl)-5-nitrothiophene-2-carboxamide as a yellow powder.

### Alkoxylation

### 6-(4-fluorophenoxy)-1-phenyl-1H-pyrazolo[3,4-d]pyrimidin-4-amine formation

To a suspension solution of 6-chloro-1-phenyl-1H-pyrazolo[3,4-d]pyrimidin-4-amine (2.46 g, 10 mmol), 4-fluorophenol (1.68 g, 15 mmol), 1,4-dioxane (50 ml), Cesium carbonate (13.03 g, 40 mmol) in a 100 ml glass flask. The reaction mixture was heated to 100° C. for 16 hrs. After cooling to room temperature, then filter by suction and the filtrate was removed and extract with ethyl acetate (60 ml*3). The layers were separated and the organic layer was washed with brine (30 ml), dried over sodium sulfate, filtered, and concentrated under reduced pressure. The residue was purified by chromatography on silica gel (n-Hexanes/0-25% Ethyl acetate, linear gradient) to afford 6-(4-fluorophenoxy)-1-phenyl-1H-pyrazolo[3,4-d]pyrimidin-4-amine (2.60 g, 81%) as a pale yellow powder.

### Amidation

### N-(6-(4-fluorophenoxy)-1-phenyl-1H-pyrazolo[3,4-d]pyrimidin-4-yl)-5-nitrothiophene-2-carboxamide formation by Amidation of Carbonyl Acid

To a solution of 6-(3,3-difluoropyrrolidin-1-yl)-1-phenyl-1H-pyrazolo[3,4-d]pyrimidin-4-amine (948 mg, 3.00 mmol) and triethyamine (2.06 ml) in 30 ml THF, was added 5-nitrothiophene-2-carbonyl chloride (900 mg, 4.68 mmol). The reaction mixture was stirred at ambient temperature for 15 hr. Water was added and the product extracted with ethyl acetate. The organic layer was separated, dried (anhydrous sodium sulfate) and evaporated in vacuo. The product was purified by column chromatography on silica gel (hexane-ethyl acetate gradient eluent) and recrystallization with DCM and MeOH to yield the title compound. Yield 1100 mg (77%) N-(6-(4-fluorophenoxy)-1-phenyl-1H-pyrazolo [3,4-d]pyrimidin-4-yl)-5-nitrothiophene-2-carboxamide formation as a yellow powder.

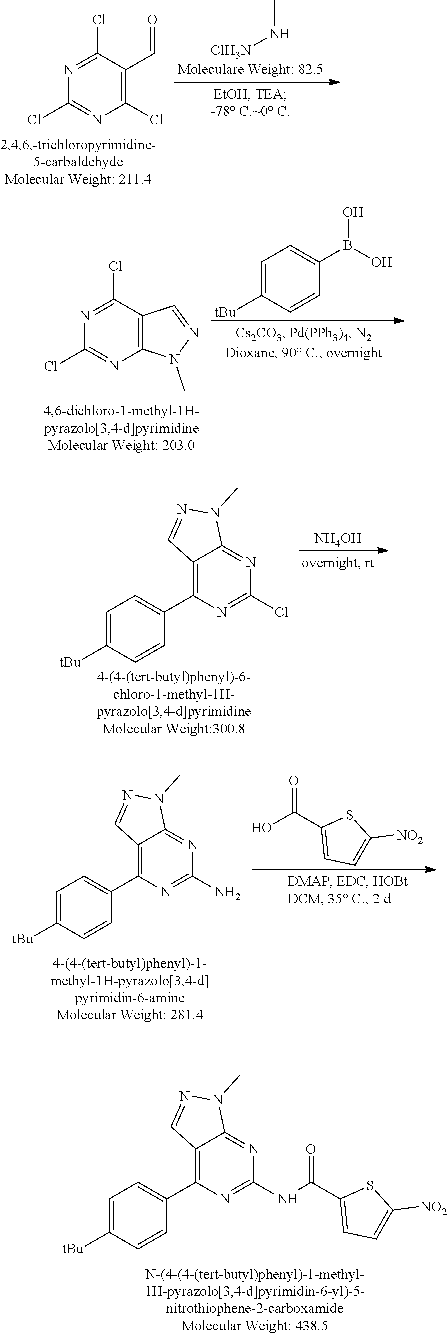

### Experimental Procedure

To a solution of the 2,4,6-trichloropyrimidine-5-carbaldehyde (3.7 g, 17.5 mmol) dissolved in EtOH (50 ml) cooled to −78° C. was added methyl hydrazine (0.93 ml, 17.5 mmol) and TEA (8 ml). The mixture was stirred for 30 minutes at −78° C. then 2 hr at 0° C. The solution was concentrated in vacuum without hearing. To the reduced volume solution EtOAc was added and the solution washed with a saturated NaHCO₃ solution and concentrated in vacuum without heating. Filtration over a small silica gel plug (2:1 EtOAc:Hex) and concentration afforded the desired product, 4,6-Dichloro-1-methyl-1H-pyrazolo[3,4-d]pyrimidine as a yellow solid (2.84 g, 80%)

### 4-(4-(tert-butyl)phenyl)-6-chloro-1-methyl-1H-pyrazolo[3,4-d]pyrimidine formation Suzuki coupling reaction

A suspension of 4,6-dichloro-1-methyl-1H-pyrazolo[3,4-d]pyrimidine (2.03 g, 10 mmol), 4-(tert-butyl)phenyl boronic acid (2.67 g, 15 mmol), Tetrakis (triphenylphosphine) palladium (0) (1.16 g, 1 mmol), 1,4-dioxane (40 ml), water (2 ml), and aqueous Cesium carbonate solution (2.0 M, 20 ml, 40 mmol) in a 100 mL glass flask was deoxygenated using 5 alternate vacuum/Nitrogen flush cycles. The reaction mixture was heated to 90° C. for 15 hrs. After cooling to room temperature, poured the Celite powder into the flask and stirred for 10 mm at room temperature then filter by suction and the filtrate was partitioned between ethyl acetate (80 ml) and saturated aqueous sodium bicarbonate solution (30 ml). The layers were separated and the organic layer was washed with brine (30 ml), dried over sodium sulfate, filtered, and concentrated under reduced pressure. The residue was purified by chromatography on silica gel (0-25% n-Hexanes/Ethyl acetate, linear gradient) to afford 4-(4-(tert-butyl)phenyl)-6-chloro-1-methyl-1H-pyrazolo[3,4-d]pyrimidine (2.55 g, 85%) as a yellow powder.

### 4-(4-(tert-butyl)phenyl)-1-methyl-1H-pyrazolo[3,4-d]pyrimidin-6-amine

### Amination

To a suspension of 4,6-Dichloro-1-methyl-1H-pyrazolo[3,4-d]pyrimidine (2.11 g, 7 mmol) in the reaction flask and then add 32 ml THF to wait for the solid to dissolve completely. Then add 10 g, 30% ammonium solution and react at room temperature (25° C.) for 24 h. Poured 60 ml water into the solution, filtration by suction to afford the 4-(4-(tert-butyl)phenyl)-1-methyl-1H-pyrazolo[3,4-d]pyrimidin-6-amine (1.58 g, 80%) as a yellow solid powder.

### Amidation

### N-(4-(4-(tert-butyl)phenyl)-1-methyl-1H-pyrazolo[3,4-d]pyrimidin-6-yl)-5-nitrothiophene-2-carboxamide formation by amidation of carbonyl acid

To a solution of 4-(4-(ter-butyl)phenyl)-1-methyl-1H-pyrazolo[3,4-d]pyrimidin-6-amine (843 mg, 3.00 mmol) in pyridine (6 ml), was added 5-nitrothiophen-2-carbonyl chloride (900 mg, 4.68 mmol). The reaction mixture was stirred at ambient temperature for 15 hr. Water was added and the product extracted with ethyl acetate. The organic layer was separated, dried (anhydrous sodium sulfate) and evaporated in vacuo. The product was purified by column chromatography on silica gel (hexane-ethyl acetate gradient) and recrystallization (hexane and acetone) to yield the title compound. Yield 1021 mg (78%) N-(4-(4-(tert-butyl)phenyl)-1-methyl-1H-pyrazolo[3,4-d]pyrimidin-6-yl)-5-nitrothiophene-2-carboxamide as a yellow powder.

### Evaluation of Compounds of Formula (I) in In Vitro MTS Assays

The cell viability measurement is based on the NCI-60 screening methodology (Nat. Rev. Cancer 6, 813-823, 2006). Briefly, cells are inoculated into 96-well plates at the optimal plating density. After 24 h, one of the two plates for Hep3B cell line is processed to determine a time zero cell viability (Tz) by MTS assay (Promega). Compounds are added over a 2-fold serial dilution to provide a total five drug concentrations plus DMSO control. Plates are incubated for a further 2 days, then measured cell viability by MTS assay [control growth (C) and test growth in the presence of drug at the five concentration levels (Ti)]. The LC₅₀ is calculated from [(Ti−Tz)/Tz]×100=−50, which is the drug concentration resulting in a 50% reduction at the end of the drug treatment as compared to that at the beginning.

The compounds prepared in Tables 1-10 were tested in three in vitro assays, and the results are shown in Tables 1-10 for Hep3B and Table 11 for SW480 and NCI-H460 shown below. Herein, Hep3B refers to hepatocellular carcinoma cell line, SW480 refers to colon adenocarcinoma cell line, and NCI-H460 refers to human lung cancer cell line.

In the compounds shown in Tables 1 to 10, the detail synthesized procedures of some compounds are not repeated again if the synthesized procedures thereof are similar to those of the forgoing compounds.

Shown in following Tables 1 to 10 are the structures and in vitro activities of exemplary compounds of formula (I). Most of the disclosed compounds were found to inhibit the growth of Hep3B cells (hepatocellular carcinoma cell line).

TABLE 1

| | 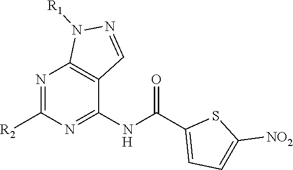 | | |
|---|---|---|---|
| | | | Hep3B |
| Compound | R₁ | R₂ | LC₅₀ |
| 1-1 | Methyl | 4-(tert-butyl)phenyl | 1.80 |
| 1-2 | Methyl | 4-ethoxyphenyl | 3.24 |
| 1-3 | Methyl | benzofuran-2-yl | NA |
| 1-4 | Methyl | 4-(trifluoromethyl)phenyl | NA |
| 1-5 | Methyl | 4-(trifluoromethoxy)phenyl | NA |
| 1-6 | Methyl | 3-(trifluoromethoxy)phenyl | 1.47 |
| 1-7 | Methyl | 4-(tert-butoxy)phenyl | 1.51 |
| 1-8 | Methyl | 2-(trifluoromethoxy)phenyl | 2.53 |
| 1-9 | Methyl | 4-(tert-pentyl)phenyl | 1.50 |
| 1-10 | Methyl | 4-(dimethylamino)phenyl | >5 |
| 1-11 | Methyl | 4-(trifluoromethyl)styryl | 1.71 |
| 1-12 | Methyl | 4-methoxystyryl | >5 |
| 1-13 | Methyl | 4-(trifluoromethyl) | 2.03 |
| | | phenyl)ethynyl | |
| 1-14 | Methyl | 4- | 1.67 |
| | | (trifluoromethoxy) | |
| | | phenyl)ethynyl | |
| 1-15 | Methyl | phenoxy | NA |
| 1-16 | Methyl | 4-fluorophenoxy | NA |
| 1-17 | Methyl | 4-(trifluoromethoxy)phenoxy | NA |
| 1-18 | Methyl | 3-(trifluoromethyl)phenoxy | 1.99 |
| 1-19 | Methyl | 2-(dimethylamino)ethoxy | NA |
| 1-20 | Methyl | 4-morpholinophenoxy | NA |
| 1-21 | Methyl | morpholino | NA |
| 1-22 | Isopropyl | 4-(trifluoromethoxy)phenyl | 0.73 |
| 1-23 | Isopropyl | 4-(tert-butyl)phenyl | 1.57 |
| 1-24 | Isopropyl | 4-(trifluoromethyl)phenyl | 0.90 |
| 1-25 | tert-butyl | 4-(tert-butyl)phenyl | 4.11 |
| 1-26 | tert-butyl | 4-(trifluoromethyl)phenyl | 0.79 |
| 1-27 | tert-butyl | 4-(trifluoromethoxy)phenyl | 0.61 |
| 1-28 | 2-hydroxyethyl | 4-(trifluoromethoxy)phenyl | 4.03 |
| 1-29 | 2-cyanoethyl | 4-(trifluoromethoxy)phenyl | NA |
| 1-30 | 2-morpholinoethyl | 4-(trifluoromethoxy)phenyl | 1.70 |
| 1-31 | 2-(dimethylamino) | 4-(trifluoromethoxy)phenyl | 1.86 |
| | ethyl | | |
| 1-32 | 2-(3,3- | 4-(trifluoromethoxy)phenyl | 0.77 |
| | difluoropyrrolidin- | | |
| | 1-yl)ethyl | | |
| 1-33 | 2-(2-ethoxyethoxy) | 4-fluorophenyl | NA |
| | ethyl | | |

### Compound 1-1

### N-(6-(4-(tert-butyl)phenyl)-1-methyl-1H-pyrazolo[3,4-d]pyrimidin-4-yl)-5-nitrothiophene-2-carboxamide

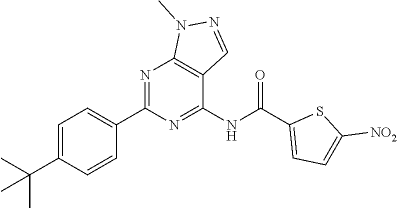 [SMILES: Cn1ncc2c(NC(=O)c3ccc([N+](=O)[O-])s3)nc(-c3ccc(C(C)(C)C)cc3)nc21]

¹H NMR (400 MHz, CDCl₃): δ 15.33 (br. s., 1H), 8.36 (br. s., 1H), 8.13-8.26 (m, 2H), 7.90 (d, J=4.4 Hz, 1H), 7.77 (d, J=4.4 Hz, 1H), 7.58 (d, J=8.3 Hz, 2H), 4.13 (s, 3H), 1.37 (s, 9H). MS(M+1) 437.

### Compound 1-2

### N-(6-(4-ethoxyphenyl)-1-methyl-1H-pyrazolo[3,4-d]pyrimidin-4-yl)-5-nitrothiophene-2-carboxamide

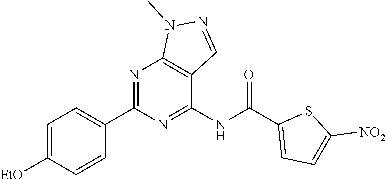 [SMILES: CCOc1ccc(-c2nc(NC(=O)c3ccc([N+](=O)[O-])s3)c3cnn(C)c3n2)cc1]

¹H NMR (400 MHz, DMSO-d₆): δ 11.82 (br. s., 1H), 8.43-4.54 (m, J=8.8 Hz, 2H), 8.30-8.40 (m, 2H), 8.24 (d, J=4.4 Hz, 1H), 7.02-7.13 (m, 2H), 4.13 (q, J=7.2 Hz, 2H), 4.07 (s, 3H), 1.37 (t, J=6.8 Hz, 3H). MS(M+1): 425.

### Compound 1-3

### N-(6-(benzofuran-2-yl)-1-methyl-1H-pyrazolo[3,4-d]pyrimidin-4-yl)-5-nitrothiophene-2-carboxamide

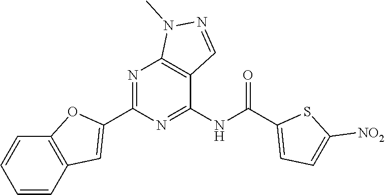 [SMILES: Cn1ncc2c(NC(=O)c3ccc([N+](=O)[O-])s3)nc(-c3cc4ccccc4o3)nc21]

¹H NMR (400 MHz, DMSO-d₆): δ 12.18 (br s., 1H), 8.29-8.41 (m, 2H), 8.24 (d, J=4.4 Hz, 1H), 7.79-7.89 (m, 2H), 7.76 (d, J=7.8 Hz, 1H), 7.47 (t, J=7.8 Hz, 1H), 7.35 (t, J=7.6 Hz, 1H), 4.10 (s, 3H). MS(M+1): 421.

### Compound 1-4

### N-(1-methyl-6-(4-(trifluoromethyl)phenyl)-1H-pyrazolo[3,4-d]pyrimidin-4-yl)-5-nitrothiophene-2-carboxamide

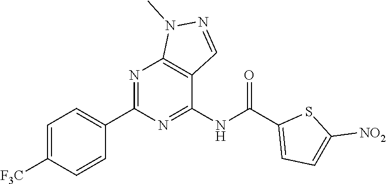 [SMILES: Cn1ncc2c(NC(=O)c3ccc([N+](=O)[O-])s3)nc(-c3ccc(C(F)(F)F)cc3)nc21]

¹H NMR (400 MHz, DMSO-d₆): δ 11.99 (br s., 1H), 8.69-8.77 (m, J=8.3 Hz, 2H), 8.40 (s, 1H), 8.36 (d, J=4.4 Hz, 1H), 8.24 (d, J=4.4 Hz, 1H), 7.89-7.99 (m, J=8.3 Hz, 2H), 4.11 (s, 3H). MS(M+1): 449.

### Compound 1-5

### N-(1-methyl-6-(4-(trifluoromethoxy)phenyl)-1H-pyrazolo[3,4-1]pyrimidin-4-yl)-5-nitrothiophene-2-carboxamide

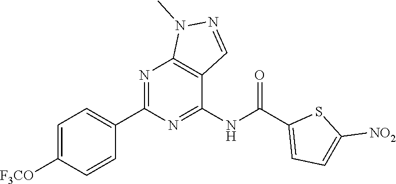 [SMILES: Cn1ncc2c(NC(=O)c3ccc([N+](=O)[O-])s3)nc(-c3ccc(OC(F)(F)F)cc3)nc21]

¹H NMR (400 MHz, DMSO-d₆): δ 11.94 (br. s., 1H), 8.57-8.68 (m, 2H), 8.32-8.41 (m, 2H), 8.24 (d, J=4.4 Hz, 1H), 7.50-7.61 (m, J=8.3 Hz, 2H), 4.09 (s, 3H). MS(M+H): 465

### Compound 1-6

### N-(1-methyl-6-(3-(trifluoromethoxy)phenyl)-1H-pyrazolo[3,4-d]pyrimidin-4-yl)-5-nitrothiophene-2-carboxamide

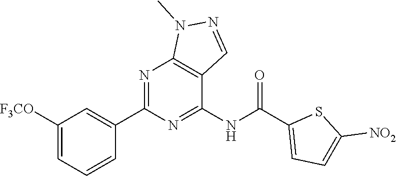 [SMILES: Cn1ncc2c(NC(=O)c3ccc([N+](=O)[O-])s3)nc(-c3cccc(OC(F)(F)F)c3)nc21]

¹H NMR (400 MHz, DMSO-d₆): δ 11.85 (br. s., 1H), 8.55 (d, J=7.8 Hz, 1H), 8.42 (s, 1H), 8.38 (s, 1H), 8.34 (d, J=4.4 Hz, 1H), 8.23 (d, J=4.4 Hz, 1H), 7.71 (t, J=8.1 Hz, 1H), 7.55-7.60 (m, 1H), 4.08 (s, 3H). MS(M+1): 465.

### Compound 1-7

### N-(6-(4-(tert-butoxy)phenyl)-1-methyl-1H-pyrazolo[3,4-d]pyrimidin-4-yl)-5-nitrothiophene-2-carboxamide

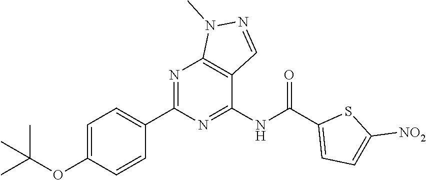 [SMILES: Cn1ncc2c(NC(=O)c3ccc([N+](=O)[O-])s3)nc(-c3ccc(OC(C)(C)C)cc3)nc21]

¹H NMR (400 MHz, DMSO-d₆): δ 11.88 (br. s., 1H), 8.41-8.50 (m, J=8.8 Hz, 2H), 8.32-8.40 (m, 2H), 8.24 (d, J=4.4 Hz, 1H), 7.08-7.19 (m, 2H), 4.08 (s, 3H), 1.39 (s, 9H). MS(M+1): 453.

### Compound 1-8

### N-(1-methyl-6-(2-(trifluoromethoxy)phenyl)-1H-pyrazolo[3,4-d]pyrimidin-4-yl)-5-nitrothiophene-2-carboxamide

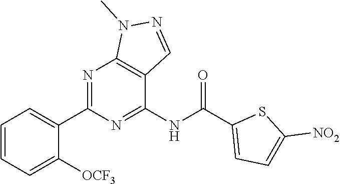 [SMILES: Cn1ncc2c(NC(=O)c3ccc([N+](=O)[O-])s3)nc(-c3ccccc3OC(F)(F)F)nc21]

¹H NMR (400 MHz, DMSO-d₆): δ 12.09 (br. s., 1H), 8.43 (s, 1H), 8.34 (d, J=4.4 Hz, 1H), 8.22 (d, J=4.4 Hz, 1H), 8.13 (dd, J=7.8, 2.0 Hz, 1H), 7.64-7.72 (m, 1H), 7.48-7.64 (m, 2H), 4.05 (s, 3H). MS(M+1): 465.

### Compound 1-9

### N-(1-methyl-6-(4-(tert-pentyl)phenyl)-1H-pyrazolo[3,4-d]pyrimidin-4-yl)-5-nitrothiophene-2-carboxamide

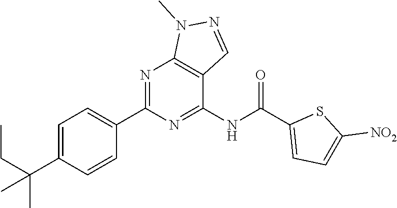 [SMILES: CCC(C)(C)c1ccc(-c2nc(NC(=O)c3ccc([N+](=O)[O-])s3)c3cnn(C)c3n2)cc1]

¹H NMR (400 MHz, DMSO-d₆): δ 11.93 (br s., 1H), 8.46 (d, J=8.3 Hz, 2H), 8.31-8.43 (m, 2H), 8.25 (d, J=4.4 Hz, 1H), 7.52 (d, J=8.3 Hz, 2H), 4.09 (s, 3H), 1.60-1.76 (m, 2H), 1.31 (s, 6H), 0.66 (t, J=7.3 Hz, 3H). MS(M+1): 451.

### Compound 1-10

### N-(6-(4-(dimethylamino)phenyl)-1-methyl-1H-pyrazolo[3,4-d]pyrimidin-4-yl)-5-nitrothiophene-2-carboxamide

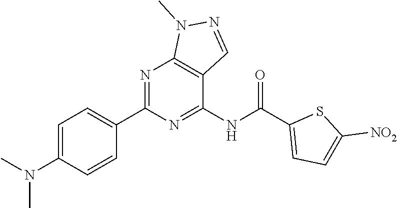 [SMILES: CN(C)c1ccc(-c2nc(NC(=O)c3ccc([N+](=O)[O-])s3)c3cnn(C)c3n2)cc1]

¹H NMR (400 MHz, DMSO-d₆): δ 11.73 (s, 1H), 8.32-8.45 (m, 3H), 8.27 (s, 1H), 8.24 (d, J=4.4 Hz, 1H), 6.82 (d, J=8.8 Hz, 2H), 4.04 (s, 3H), 3.03 (s, 6H). MS(M+1): 424.

### Compound 1-1

### (E)-N-(1-methyl-6-(4-(trifluoromethyl)styryl)-1H-pyrazolo[3,4-d]pyrimidin-4-yl)-5-nitrothiophene-2-carboxamide

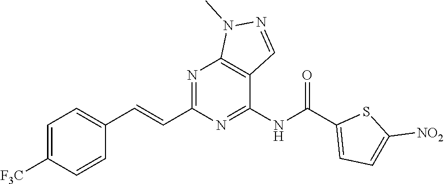 [SMILES: Cn1ncc2c(NC(=O)c3ccc([N+](=O)[O-])s3)nc(/C=C/c3ccc(C(F)(F)F)cc3)nc21]

¹H NMR (400 MHz, DMSO-d₆): δ 11.98 (br. s., 1H), 8.28-8.38 (m, 2H), 8.20 (d, J=4.4 Hz, 1H), 8.07 (d, J=16.1 Hz, 1H), 7.89-7.98 (m, J=8.3 Hz, 2H), 7.72-7.80 (m, J=8.3 Hz, 2H), 7.41 (d, J=16.1 Hz, 1H), 4.03 (s, 3H). MS(M+1): 475.

### Compound 1-12

### (E)-N-(6-(4-methoxystyryl)-1-methy-1H-pyrazolo[3,4-d]pyrimidin-4-yl)-5-nitrothiophene-2-carboxamide

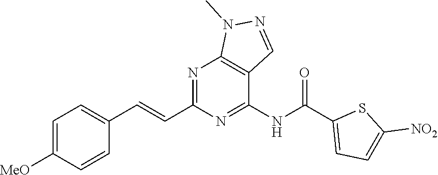 [SMILES: COc1ccc(/C=C/c2nc(NC(=O)c3ccc([N+](=O)[O-])s3)c3cnn(C)c3n2)cc1]

¹H NMR (400 MHz, DMSO-d₆): δ 11.92 (br. s., 1H), 8.32 (s, 2H), 8.22 (d, J=4.4 Hz, 1H), 8.03 (d, J=16.1 Hz, 1H), 7.60-7.73 (m, 2H), 7.16 (d, J=16.1 Hz, 1H), 6.88-7.08 (m, 2H), 4.03 (s, 3H), 3.81 (s, 3H). MS(M+1): 437.

### Compound 1-13

### N-(1-methyl-6-((4-(trifluoromethyl)phenyl)ethynyl)-1H-pyrazolo[3,4-d]pyrimidin-4-yl)-5-nitrothiophene-2-carboxamide

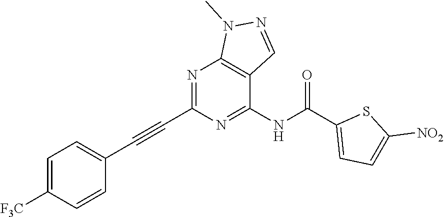 [SMILES: Cn1ncc2c(NC(=O)c3ccc([N+](=O)[O-])s3)nc(C#Cc3ccc(C(F)(F)F)cc3)nc21]

¹H NMR (400 MHz, DMSO-d₆): δ 12.28 (s, 1H), 8.40 (s, 1H), 8.34 (d, J=4.4 Hz, 1H), 8.18 (d, J=4.4 Hz, 1H), 7.72-7.92 (m, 4H), 4.03 (s, 3H). MS(M+1): 473.

### Compound 1-14

### N-(1-methyl-6-((4-(trifluoromethoxy)phenyl)ethynyl)-1H-pyrazolo[3,4,d]pyrimidin-4-yl)-5-nitrothiophene-2-carboxamide

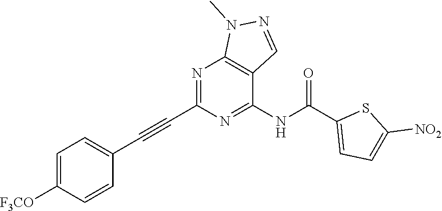 [SMILES: Cn1ncc2c(NC(=O)c3ccc([N+](=O)[O-])s3)nc(C#Cc3ccc(OC(F)(F)F)cc3)nc21]

¹H NMR (400 MHz, DMSO-d₆): δ 12.32 (br. s., 1H), 8.42 (s, 1H), 8.35 (d, J=4.4 Hz, 1H), 8.21 (d, J=4.4 Hz, 1H), 7.77-7.86 (m, 2H), 7.49 (d, J=7.8 Hz, 2H), 4.05 (s, 3H). MS(M+1): 489.

### Compound 1-15

### N-(1-methyl-6-phenoxy-1H-pyrazolo[3,4-d]pyrimidin-4-yl)-5-nitrothiophene-2-carboxamide

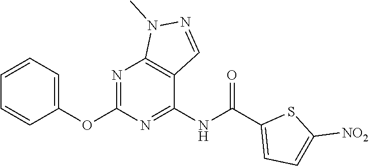 [SMILES: Cn1ncc2c(NC(=O)c3ccc([N+](=O)[O-])s3)nc(Oc3ccccc3)nc21]

¹H NMR (400 MHz, DMSO-d₆): δ 12.17 (s, 1H), 8.31-8.34 (m, 2H), 8.21 (d, J=4.4 Hz, 1H), 7.43-7.50 (m, 2H), 7.24-7.32 (m, 3H), 3.80 (s, 3H). MS (M+1): 397.

### Compound 1-16

### N-(6-(4-fluorophenoxy)-1-methyl-1H-pyrazolo[3,4-d]pyrimidin-4-yl)-5-nitrothiophene-2-carboxamide

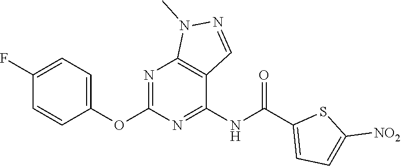 [SMILES: Cn1ncc2c(NC(=O)c3ccc([N+](=O)[O-])s3)nc(Oc3ccc(F)cc3)nc21]

¹H NMR (400 MHz, DMSO-d₆): δ 12.14 (s, 1H), 8.29-8.33 (m, 2H), 8.20 (d, J=4.9 Hz, 1H), 7.24-7.38 (m, 5H), 3.79 (s, 3H). MS (M+1): 415.

### Compound 1-17

### N-(1-methyl-6(4-(trifluoromethoxy)phenoxy)-1H-pyrazolo[3,4-d]pyrimidin-4-yl)-5-nitrothiophene-2-carboxamide

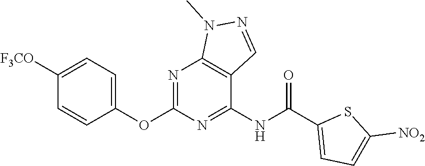 [SMILES: Cn1ncc2c(NC(=O)c3ccc([N+](=O)[O-])s3)nc(Oc3ccc(OC(F)(F)F)cc3)nc21]

¹H NMR (400 MHz, DMSO-d₆): δ 12.15 (s, 1H), 8.33 (s, 1H), 8.31 (d, J=4.9 Hz, 1H), 8.20 (d, J=4.4 Hz, 1H), 7.46 (s, 4H), 3.81 (s, 3H). MS (M+1): 481.

### Compound 1-18

### N-(1-methyl-6-(3-(trifluoromethyl)phenoxy)-1H-pyrazolo[3,4-d]pyrimidin-4-yl)-5-nitrothiophene-2-carboxamide

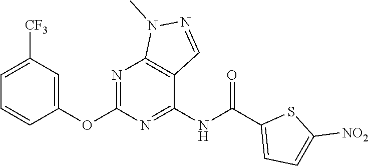 [SMILES: Cn1ncc2c(NC(=O)c3ccc([N+](=O)[O-])s3)nc(Oc3cccc(C(F)(F)F)c3)nc21]

¹H NMR (400 MHz, DMSO-d₆): δ 12.20 (s, 1H), 8.34 (s, 1H), 8.31 (d, J=4.9 Hz, 1H), 8.21 (d, J=4.4 Hz, 1H), 7.62-7.77 (m, 4H), 3.80 (s, 3H). MS (M+1): 465.

### Compound 1-19

### N-(6-(2-(dimethylamino)ethoxy)-1-methyl-1H-pyrazolo[3,4-d]pyrimidin-4-yl)-5-nitrothiophene-2-carboxamide

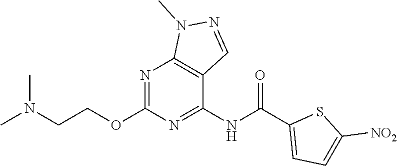 [SMILES: CN(C)CCOc1nc(NC(=O)c2ccc([N+](=O)[O-])s2)c2cnn(C)c2n1]

¹H NMR (400 MHz, DMSO-d₆): δ 8.28-8.33 (m, 2H), 8.22 (d, J=4.4 Hz, 1H), 4.71-4.79 (m, 2H), 3.95 (s, 3H), 3.52-3.60 (m, 2H), 2.87 (s, 6H). MS (M+1): 392.

### Compound 1-20

### N-(1-methyl-6-(4-morpholinophenoxy)-1H-pyrazolo[3,4,d]pyrimidin-4-yl)-5-nitrothiophene-2-carboxamide

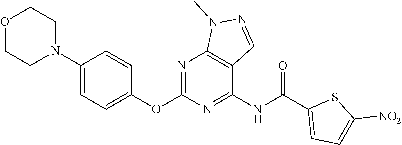 [SMILES: Cn1ncc2c(NC(=O)c3ccc([N+](=O)[O-])s3)nc(Oc3ccc(N4CCOCC4)cc3)nc21]

¹H NMR (400 MHz, DMSO-d₆): δ 12.12 (br. s., 1H), 8.31 (d, J=4.4 Hz, 1H), 8.30 (s, 1H), 8.20 (d, J=4.4 Hz, 1H), 7.12-7.19 (m, J=9.3 Hz, 2H), 6.96-7.03 (m, J=9.3 Hz, 2H), 3.80 (s, 3H), 3.72-3.78 (m, 4H), 3.08-3.15 (m, 4H). MS (M+1): 482.

### Compound 1-21

### N-(1-methyl-6-morpholino-1H-pyrazolo[3,4-d]pyrimidin-4-yl)-5-nitrothiophene-2-carboxamide

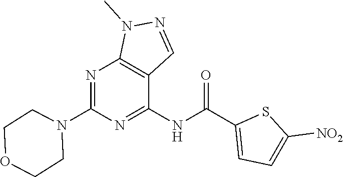 [SMILES: Cn1ncc2c(NC(=O)c3ccc([N+](=O)[O-])s3)nc(N3CCOCC3)nc21]

¹H NMR (400 MHz, DMSO-d₆): δ 11.36 (br. s., 1H), 8.27 (d, J=4.4 Hz, 1H), 8.20 (d, J=4.4 Hz, 1H), 8.07 (s, 1H), 3.77-3.88 (m, 7H), 3.66-3.72 (m, 4H). MS(M+1): 390.

### Compound 1-22

### N-(1-isopropyl-6-(4-(trifluoromethoxy)phenyl)-1H-pyrazolo[3,4-d]pyrimidin-4-yl)-5-nitrothiophene-2-carboxamide

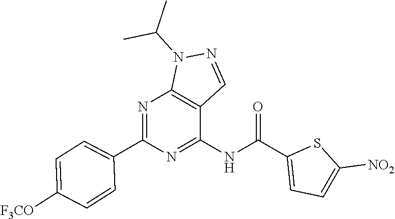 [SMILES: CC(C)n1ncc2c(NC(=O)c3ccc([N+](=O)[O-])s3)nc(-c3ccc(OC(F)(F)F)cc3)nc21]

¹H NMR (400 MHz, DMSO-d₆): δ 11.94 (s, 1H), 8.53-8.74 (m, 2H), 8.34-8.50 (m, 2H), 8.24 (d, J=4.4 Hz, 1H), 7.57 (d, J=7.8 Hz, 2H), 5.13-5.43 (m, 1H), 1.55 (d, J=6.8 Hz, 6H). MS(M+1): 493.

### Compound 1-23

### N-(6-(4-(tert-butyl)phenyl)-1-isopropyl-1H-pyrazolo[3,4-d]pyrimidin-4-yl)-5-nitrothiophene-2-carboxamide

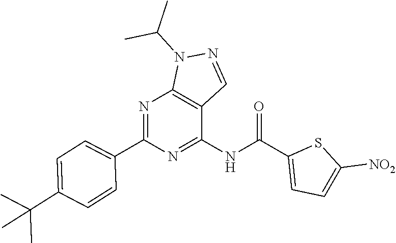 [SMILES: CC(C)n1ncc2c(NC(=O)c3ccc([N+](=O)[O-])s3)nc(-c3ccc(C(C)(C)C)cc3)nc21]

¹H NMR (400 MHz, DMSO-d₆): δ 11.90 (s, 1H), 8.43-8.53 (m, J=8.3 Hz, 2H), 8.31-8.43 (m, 2H), 8.25 (d, J=4.4 Hz, 1H), 7.47-7.67 (m, J=8.3 Hz, 2H), 5.18-5.37 (m, 1H), 1.56 (d, J=6.8 Hz, 6H), 1.35 (s, 9H). MS(M+1): 465.

### Compound 1-24

### N-(1-isopropyl-6-(4-(trifluoromethyl)phenyl)-1H-pyrazolo[3,4-d]pyrimidin-4-yl)-5-nitrothiophene-2-carboxamide

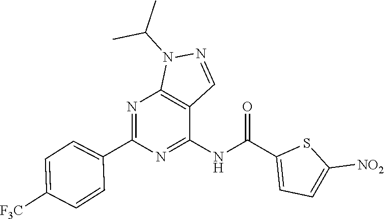 [SMILES: CC(C)n1ncc2c(NC(=O)c3ccc([N+](=O)[O-])s3)nc(-c3ccc(C(F)(F)F)cc3)nc21]

¹H NMR (400 MHz, DMSO-d₆): δ 11.95 (s, 1H), 8.62-8.78 (m, J=8.3 Hz, 2H), 8.30-8.44 (m, 2H), 8.22 (d, J=4.4 Hz, 1H), 7.86-7.99 (m, J=8.3 Hz, 2H), 5.29 (quin, J=6.7 Hz, 1H), 1.56 (d, J=6.8 Hz, 6H). MS(M+1): 477.

### Compound 1-25

### N-(1-(tert-butyl)-6-(4-(tert-butyl)phenyl)-1H-pyrazolo[3,4,d]pyrimidin-4-yl)-5-nitrothiophene-2-carboxamide

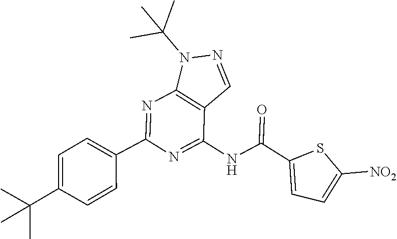 [SMILES: CC(C)(C)c1ccc(-c2nc(NC(=O)c3ccc([N+](=O)[O-])s3)c3cnn(C(C)(C)C)c3n2)cc1]

¹H NMR (400 MHz, DMSO-d₆): δ 11.91 (br. s., 1H), 8.36-8.58 (m, J=8.3 Hz, 2H), 8.17-8.36 (m, 3H), 7.51-7.76 (m, J=8.3 Hz, 2H), 1.84 (s, 9H), 1.35 (s, 9H).

MS(M+1): 479.

### Compound 1-26

### N-(1-(tert-butyl)-6-(4-(trifluoromethyl)phenyl)-1H-pyrazolo[3,4,d]pyrimidin-4-yl)-5-nitrothiophene-2-carboxamide

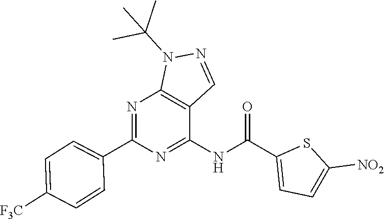 [SMILES: CC(C)(C)n1ncc2c(NC(=O)c3ccc([N+](=O)[O-])s3)nc(-c3ccc(C(F)(F)F)cc3)nc21]

¹H NMR (400 MHz, DMSO-d₆): δ 11.95 (s, 1H), 8.59-8.76 (m, J=7.8 Hz, 2H), 8.29-8.42 (m, 2H), 8.23 (d, J=4.4 Hz, 1H), 7.87-8.11 (m, J=8.3 Hz, 2H), 1.84 (s, 9H). MS(M+1): 491.

### Compound 1-27

### N-(1-(tert-butyl)-6-(4-(trifluoromethoxy)phenyl)-1H-pyrazolo[3,4-d]pyrimidin-4-yl)-5-nitrothiophene-2-carboxamide

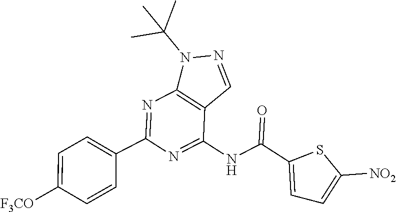 [SMILES: CC(C)(C)n1ncc2c(NC(=O)c3ccc([N+](=O)[O-])s3)nc(-c3ccc(OC(F)(F)F)cc3)nc21]

¹H NMR (4007 MHz, DMSO-d₆): δ 11.87 (br. s., 1H), 8.59 (d, J=8.8 Hz, 2H), 8.34 (d, J=4.4 Hz, 1H), 8.29 (d, J=1.5 Hz, 1H), 8.18-8.24 (m, 1H), 7.50-7.60 (m, 2H), 1.82 (s, 9H). MS(M+1): 507.

### Compound 1-28

### N-(1-(2-hydroxyethyl)-6-(4-(trifluoromethoxy)phenyl)-1H-pyrazolo[3,4-d]pyrimidin-4-yl)-5-nitrothiophene-2-carboxamide

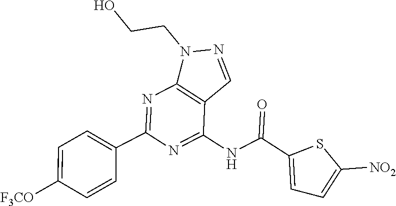 [SMILES: O=C(Nc1nc(-c2ccc(OC(F)(F)F)cc2)nc2c1cnn2CCO)c1ccc([N+](=O)[O-])s1]

¹H NMR (400 MHz, DMSO-d₆): δ 11.95 (br. s., 1H), 8.59-8.69 (m, 2H), 8.41 (s, 1H), 8.37 (d, J=4.4 Hz, 1H), 8.25 (d, J=4.4 Hz, 1H), 7.57 (d, J=7.8 Hz, 2H), 4.90 (t, J=5.6 Hz, 1H), 4.55 (t, J=5.6 Hz, 2H), 3.92 (d, J=5.9 Hz, 2H). MS(M+1) 495.

### Compound 1-29

### N-(1-(2-cyanoethyl)-6-(4-(trifluoromethoxy)phenyl)-1H-pyrazolo[3,4-d]pyrimidin-4-yl)-5-nitrothiophene-2-carboxamide

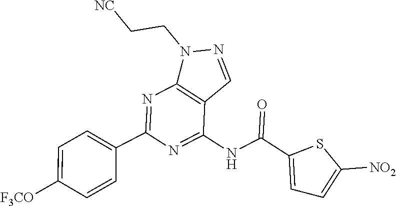 [SMILES: N#CCCn1ncc2c(NC(=O)c3ccc([N+](=O)[O-])s3)nc(-c3ccc(OC(F)(F)F)cc3)nc21]

¹H NMR (400 MHz, DMSO-d₆): δ 12.01 (br. s., 1H), 8.57-8.73 (m, 2H), 8.47 (s, 1H), 8.37 (d, J=4.9 Hz, 1H), 8.25 (d, J=4.4 Hz, 1H), 7.49-7.64 (m, J=8.3 Hz, 2H), 4.71-4.83 (m, 2H), 3.25 (t, J=6.4 Hz, 2H). MS(M+1): 504.

### Compound 1-30

### N-(1-(2-morpholinoethyl)-6-(4-(trifluoromethoxy)phenyl)-1H-pyrazolo[3,4-d]pyrimidin-4-yl)-5-nitrothiophene-2-carboxamide

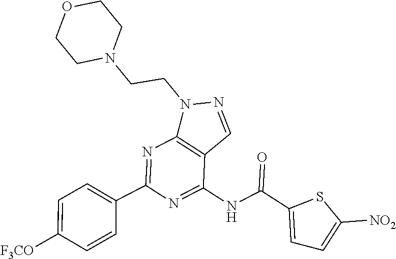 [SMILES: O=C(Nc1nc(-c2ccc(OC(F)(F)F)cc2)nc2c1cnn2CCN1CCOCC1)c1ccc([N+](=O)[O-])s1]

¹H NMR (400 MHz, DMSO-d₆): δ 11.95 (br. s., 1H), 8.57-8.68 (m, 2H), 8.40 (s, 1H), 8.36 (d, J=4.4 Hz, 1H), 8.25 (d, J=4.9 Hz, 1H), 7.46-7.64 (m, J=8.3 Hz, 2H), 4.64 (t, J=6.1 Hz, 2H), 3.37-3.50 (m, 4H), 2.84 (t, J=6.1 Hz, 2H), 2.45-2.55 (m, 4H).

MS(M+1): 564

### Compound 1-31

### N-(1-(2-(dimethylamino)ethyl)-6-(4-(trifluoromethoxy)phenyl)-1H-pyrazolo[3,4-d]pyrimidin-4-yl)-5-nitrothiophene-2-carboxamide

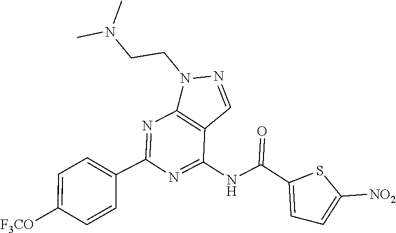 [SMILES: CN(C)CCn1ncc2c(NC(=O)c3ccc([N+](=O)[O-])s3)nc(-c3ccc(OC(F)(F)F)cc3)nc21]

¹H NMR (400 MHz, DMSO-d₆): δ 12.08 (br. s., 1H), 10.38 (br. s., 1H), 8.61-8.78 (m, 2H), 8.47-8.58 (mi, 1H), 8.37-8.47 (m, 1H), 8.26 (d, J=4.4 Hz, 1H), 7.58 (d, J=7.8 Hz, 2H), 4.95 (t, J=6.1 Hz, 2H), 3.71 (t, J=6.1 Hz, 2H), 2.87 (s, 6H). MS(M+1): 558.

### Compound 1-32

### N-(1-(2-(3,3-difluoropyrrolidin-1-yl)ethyl)-6-(4-(trifluoromethoxy)phenyl)-1H-pyrazolo[3,4-d]pyrimidin-4-yl)-5-nitrothiophene-2-carboxamide

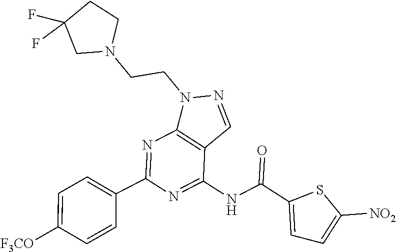 [SMILES: O=C(Nc1nc(-c2ccc(OC(F)(F)F)cc2)nc2c1cnn2CCN1CCC(F)(F)C1)c1ccc([N+](=O)[O-])s1]

¹H NMR (400 MHz, DMSO-d₆): δ 11.95 (br. s., 1H), 8.54-8.73 (m, 2H), 8.41 (s, 1H), 8.36 (d, J=4.4 Hz, 1H), 8.25 (d, J=4.4 Hz, 1H), 7.50-7.61 (m, 2H), 4.62 (t, J=6.1 Hz, 2H), 2.92-3.06 (m, 4H), 2.77 (t, J=7.1 Hz, 2H), 2.01-2.19 (m, 2H) MS(M+1): 584.

### Compound 1-33

### N-(1-(2-(2-ethoxyethoxy)ethyl)-6-(4-fluorophenyl)-1H-pyrazolo[3,4-d]pyrimidin-4-yl)-5-nitrothiophene-2-carboxamide

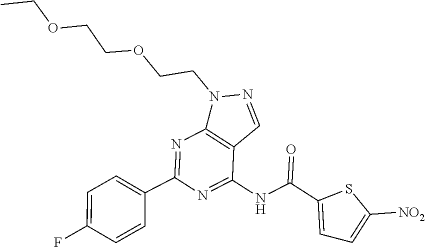 [SMILES: CCOCCOCCn1ncc2c(NC(=O)c3ccc([N+](=O)[O-])s3)nc(-c3ccc(F)cc3)nc21]

¹H NMR (400 MHz, DMSO-d₆): δ 11.86 (s, 1H), 8.58-8.54 (m, 2H), 8.38-8.34 (m, 2H), 8.22 (d, J=4.4 Hz, 1H), 7.38 (t, J=8.8 Hz, 2H), 4.64 (t, J=5.6 Hz, 2H), 3.95 (t, J=5.6 Hz, 2H), 3.54-3.51 (m, 2H), 3.35-3.32 (m, 2H), 3.25 (q. J=6.8 Hz, 2H), 0.93 (t, J=6.8 Hz, 3H). MS(M+1): 501. Yellow solid.

TABLE 2

| | | | |
|---|---|---|---|
| | (A) | | (B) |
| 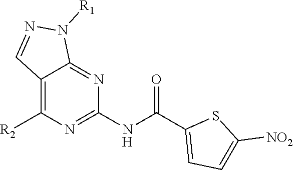 | | 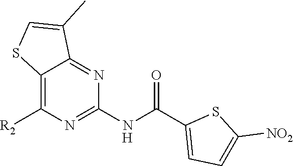 | |

| | | | |
|---|---|---|---|
| Compound | R1 | R2 | Hep3B LC₅₀ |

Formula (A)

| | | | |
|---|---|---|---|
| 2-1 | Me | 4-(tert-butyl)phenyl | 3.13 |
| 2-2 | Me | 4-(trifluoromethoxy)phenyl | NA |
| 2-3 | Isopropyl | 4-(trifluoromethoxy)phenyl | 4.02 |
| 2-4 | tert-butyl | 4-(trifluoromethoxy)phenyl | 3.05 |
| 2-5 | tert-butyl | 4-(tert-butyl)phenyl | 12.88 |
| 2-6 | Me | morpholino | NA |

Formula (B)

| | | | |
|---|---|---|---|
| 2-7 | — | 3-(trifluoromethoxy)phenyl | 3.43 |
| 2-8 | — | 4-(trifluoro methoxy)phenyl | 1.71 |

### Compound 2-1

### N-(4-(4-(tert-but)phenyl)-1-methyl-1H-pyrazolo[3,4-d]pyrimidin-6-yl)-5-nitrothiophene-2-carboxamide

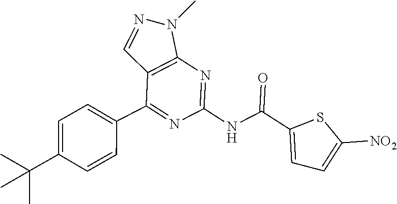 [SMILES: Cn1ncc2c(-c3ccc(C(C)(C)C)cc3)nc(NC(=O)c3ccc([N+](=O)[O-])s3)nc21]

¹H NMR (400 MHz, DMSO-d₆): δ 11.66 (s, 1H), 8.66 (s, 1H), 8.27-8.35 (m, 2H), 8.17-8.27 (m, 2H), 7.61-7.70 (m, 2H), 4.04 (s, 3H), 1.36 (s, 9H). MS(M+1): 437.

### Compound 2-2

### N-(1-methyl-4-(4-(trifluoromethoxy)phenyl)-1H-pyrazolo[3,4-d]pyrimidin-6-yl)-5-nitrothiophene-2-carboxamide

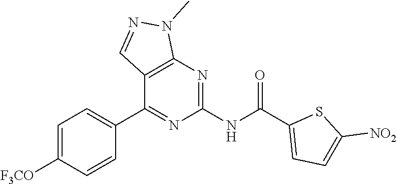 [SMILES: Cn1ncc2c(-c3ccc(OC(F)(F)F)cc3)nc(NC(=O)c3ccc([N+](=O)[O-])s3)nc21]

¹H NMR (400 MHz, DMSO-d₆): δ 11.72 (s, 1H), 8.69 (s, 1H), 8.43-8.52 (m, 2H), 8.17-8.28 (m, 2H), 7.57-7.67 (m, J=8.3 Hz, 2H), 4.04 (s, 3H). MS(M+1): 465.

### Compound 2-3

### N-(1-isopropyl-4-(4-(trifluoromethoxy)phenyl)-1H-pyrazolo[3,4-d]pyrimidin-6-yl)-5-nitrothiophene-2-carboxamide

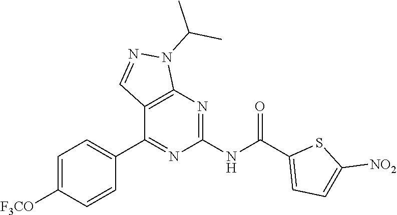 [SMILES: CC(C)n1ncc2c(-c3ccc(OC(F)(F)F)cc3)nc(NC(=O)c3ccc([N+](=O)[O-])s3)nc21]

¹H NMR (400 MHz, DMSO-d₆): δ 11.71 (s, 1H), 8.68 (s, 1H), 8.37-8.57 (m, 2H), 8.11-8.29 (m, 2H), 7.51-7.71 (m, 2H), 5.01-5.24 (m, 1H), 1.54 (d, J=6.8 Hz, 6H).

MS(M+1): 493.

### Compound 2-4

### N-(1-(tert-butyl)-4-(4-(trifluoromethoxy)phenyl)-1H-pyrazolo[3,4,d]pyrimidin-6-yl)-5-nitrothiophene-2-carboxamide

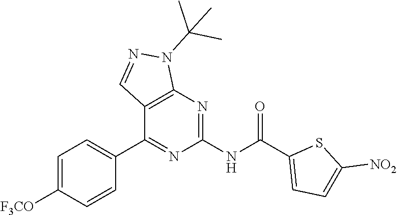 [SMILES: CC(C)(C)n1ncc2c(-c3ccc(OC(F)(F)F)cc3)nc(NC(=O)c3ccc([N+](=O)[O-])s3)nc21]

¹H NMR (400 MHz, DMSO-d₆): δ 11.63 (s, 1H), 8.58 (s, 1H), 8.42 (d, J=8.8 Hz, 2H), 8.14-8.34 (m, 2H), 7.63 (d, J=7.8 Hz, 2H), 1.80 (s, 9H). MS(M+1): 507.

### Compound 2-5

### N-(1-(tert-butyl)-4-(4-(tert-butyl)phenyl)-1H-pyrazolo[3,4-d]pyrimidin-6-yl)-5-nitrothiophene-2-carboxamide

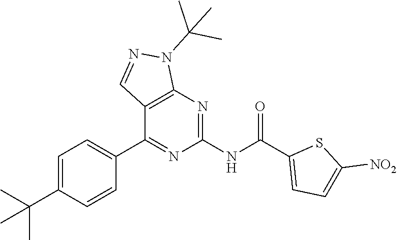 [SMILES: CC(C)(C)c1ccc(-c2nc(NC(=O)c3ccc([N+](=O)[O-])s3)nc3c2cnn3C(C)(C)C)cc1]

¹H NMR (400 MHz, DMSO-d₆): δ 11.58 (s, 1H), 8.55 (s, 1H), 8.11-8.32 (m, 4H), 7.57-7.72 (m, 2H), 1.80 (s, 9H), 1.36 (s, 9H). MS(M+1): 479.

### Compound 2-6

### N-(1-methyl-4-morpholino-1H-pyrazolo[3,4-d]pyrimidin-6-yl)-5-nitrothiophene-2-carboxamide

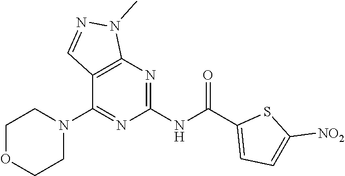 [SMILES: Cn1ncc2c(N3CCOCC3)nc(NC(=O)c3ccc([N+](=O)[O-])s3)nc21]

¹H NMR (400 MHz, DMSO-d₆): δ 11.10 (s, 1H), 8.25 (s, 1H), 8.16 (d, J=3.4 Hz, 2H), 3.93 (d, J=4.9 Hz, 4H), 3.88 (s, 3H), 3.68-3.78 (m, 4H). MS(M+1): 390.

### Compound 2-7

### N-(7-methyl-(3-(trifluoromethoxy)phenyl)thieno[3,2-d]pyrimidin-2-yl)-5-nitrothiophene-2-carboxamide

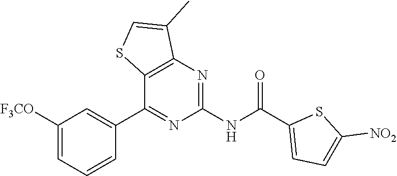 [SMILES: Cc1csc2c(-c3cccc(OC(F)(F)F)c3)nc(NC(=O)c3ccc([N+](=O)[O-])s3)nc12]

¹H NMR (400 MHz, DMSO-d₆): δ 11.73 (br. s., 1H), 8.27 (d, J=1.5 Hz, 1H), 8.18-8.26 (m, 3H), 8.14 (s, 1H), 7.84 (t, J=8.1 Hz, 1H), 7.63-7.73 (m, 1H), 2.47 (d, J=1.0 Hz, 3H). MS(M+1): 481.

### Compound 2-8

### N-(7-methyl-4-(4-(trifluoromethoxy)phenyl)thieno[3,2-d]pyrimidin-2-yl)-5-nitrothiophene-2-carboxamide

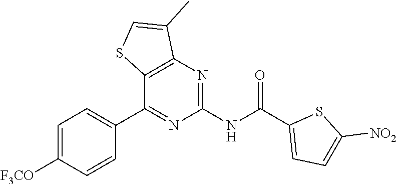 [SMILES: Cc1csc2c(-c3ccc(OC(F)(F)F)cc3)nc(NC(=O)c3ccc([N+](=O)[O-])s3)nc12]

¹H NMR (400 MHz, DMSO-d₆): δ 11.74 (br. s., 1H), 8.31-8.38 (m, 2H), 8.27 (d, J=1.0 Hz, 1H), 8.22 (q, J=4.4 Hz, 2H), 7.69 (d, J=7.8 Hz, 2H), 2.47 (d, J=1.0 Hz, 3H), MS(M+1): 481.

TABLE 3

| | | | |
|---|---|---|---|
| | (A) | | (B) |
| 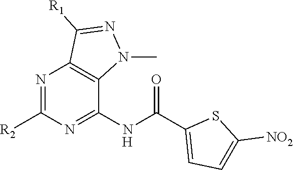 | | 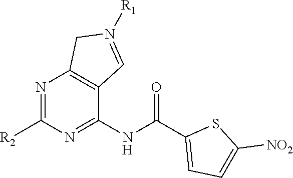 | |

| | | | |
|---|---|---|---|
| | | | Hep3B |
| Compound | R₁ | R₂ | LC₅₀ |

Formula (A)

| | | | |
|---|---|---|---|
| 3-1 | Methyl | 4-(trifluoromethyl)phenyl | 2.22 |
| 3-2 | Methyl | 3-(trifluoromethyl)phenyl | 0.86 |
| 3-3 | Methyl | 4-(trifluoromethoxy)phenyl | 1.92 |
| 3-4 | Methyl | 3-(trifluoromethoxy)phenyl | 1.51 |
| 3-5 | n-propyl | 4-(trifluoromethoxy)phenyl | NA |
| 3-6 | n-propyl | 4-(trifluorometbyl)phenyl | NA |
| 3-7 | n-propyl | 4-(tert-butyl)phenyl | NA |
| 3-8 | n-propyl | Phenyl | NA |
| 3-9 | n-propyl | 4-fluorophenyl | NA |

Formula (B)

| | | | |
|---|---|---|---|
| 3-10 | Methyl | 4-(tert-butyl)phenyl | 10 |
| 3-11 | Ethyl | 4-(tert-butyl)phenyl | 7.64 |
| 3-12 | Isopropyl | 4-(tert-butyl)phenyl | >10 |

### Compound 3-1

### N-(1,3-dimethyl-5-(4-(trifluoromethyl)phenyl)-1H-pyrazolo[4,3-d]pyrimidin-7-yl)-5-nitrothiophene-2-carboxamide

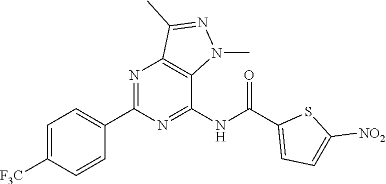 [SMILES: Cc1nn(C)c2c(NC(=O)c3ccc([N+](=O)[O-])s3)nc(-c3ccc(C(F)(F)F)cc3)nc12]

¹H NMR (400 MHz, DMSO-d₆): δ 11.89 (br. s., 1H), 8.61 (br. s., 2H), 8.07-8.26 (m, 2H), 7.91 (d, J=7.8 Hz, 2H), 4.20 (s, 3H), 2.72 (s, 3H). MS(M+1): 463.

### Compound 3-2

### N-(1,3-dimethyl-5-(3-(trifluoromethyl)phenyl)-1H-pyrazolo[4,3-d]pyrimidin-7-yl)-5-nitrothiophene-2-carboxamide

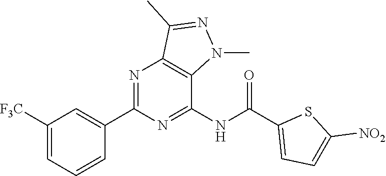 [SMILES: Cc1nn(C)c2c(NC(=O)c3ccc([N+](=O)[O-])s3)nc(-c3cccc(C(F)(F)F)c3)nc12]

¹H NMR (400 MHz, DMSO-d₆): δ 11.91 (br. s., 1H), 8.69 (br. s., 2H), 8.10-8.35 (m, 2H), 7.87 (d, J=7.8 Hz, 1H), 7.79 (t, J=7.8 Hz, 1H), 4.19 (s, 3H), 2.72 (s, 3H).

MS(M+1) 463.

### Compound 3-3

### N-(1,3-dimethyl-5-(4-(trifluoromethoxy)phenyl)-1H-pyrazolo[4,3-d]pyrimidin-7-yl)-5-nitrothiophene-2-carboxamide

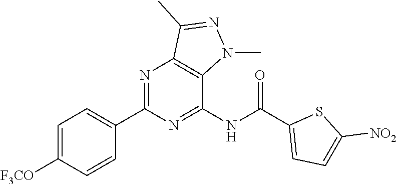 [SMILES: Cc1nn(C)c2c(NC(=O)c3ccc([N+](=O)[O-])s3)nc(-c3ccc(OC(F)(F)F)cc3)nc12]

¹H NMR (400 MHz, DMSO-d₆): δ 11.87 (br. s., 1H), 8.50 (br. s., 2H), 8.04-8.29 (m, 2H), 7.53 (d, J=7.8 Hz, 2H), 4.18 (s, 3H), 2.70 (s, 3H). MS(M+1): 479.

### Compound 3-4

### N-(1,3-dimethyl-5-(3-(trifluoromethoxy)phenyl)-1H-pyrazolo[4,3-d]pyrimidin-7-yl)-5-nitrothiophene-2-carboxamide

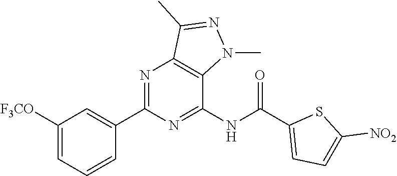 [SMILES: Cc1nn(C)c2c(NC(=O)c3ccc([N+](=O)[O-])s3)nc(-c3cccc(OC(F)(F)F)c3)nc12]

¹H NMR (400 MHz, DMSO-d₆): δ 11.84 (br. s., 1H), 8.46 (br. s., 1H), 8.30 (br. s., 1H), 8.06-8.26 (m, 2H), 7.68 (t, J=7.6 Hz, 1H), 7.50 (d, J=6.8 Hz, 1H), 4.19 (s, 3H), 2.71 (s, 3H), MS(M+1): 479.

### Compound 3-5

### N-(1-methyl-3-propyl-5-(4-(trifluoromethoxy)phenyl)-1H-pyrazolo[4,3-d]pyrimidin-7-yl)-5-nitrothiophene-2-carboxamide

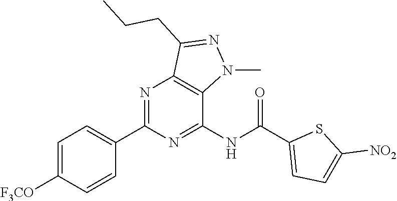 [SMILES: CCCc1nn(C)c2c(NC(=O)c3ccc([N+](=O)[O-])s3)nc(-c3ccc(OC(F)(F)F)cc3)nc12]

¹H NMR (400 MHz, DMSO-d₆): δ8.42 (d, J=7.3 Hz, 2H), 8.21 (d, J=4.4 Hz, 1H), 8.02 (br. s., 1H), 7.57 (d, J=8.8 Hz, 2H), 4.20 (br. s., 3H), 2.97 (t, J=7.3 Hz, 2H), 1.87 (dq, J=14.8, 7.6 Hz, 2H), 1.0) (t, J=7.3 Hz, 3H). MS(M+1): 507.

### Compound 3-6

### N-(1-methyl-3-propyl-5-(4-(trifluoromethyl)phenyl)-1H-pyrazolo[4,3-d]pyrimidin-7-yl)-5-nitrothiophene-2-carboxamide

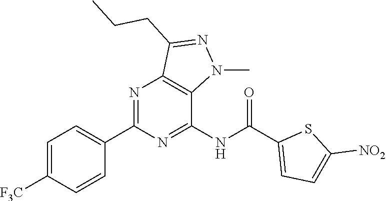 [SMILES: CCCc1nn(C)c2c(NC(=O)c3ccc([N+](=O)[O-])s3)nc(-c3ccc(C(F)(F)F)cc3)nc12]

¹H NMR (400 MHz, DMSO-d₆): δ 12.13 (br. s., 1H), 8.54 (br. s., 2H), 8.25 (d, J=4.4 Hz, 1H), 8.13 (br. s., 1H), 7.96 (d, J=8.3 Hz, 2H), 4.16 (br. s., 3H), 3.00 (t, J=7.3 Hz, 2H), 2.00-1.80 (m, 2H), 1.01 (t, J=7.3 Hz, 3H). MS(M+1): 491.

### Compound 3-7

### N-(5-(4-tert-butyl)phenyl)-1-methyl-3-propyl-1H-pyrazolo[4,3-d]pyrimidin-7-yl)-5-nitrothiophene-2-carboxamide

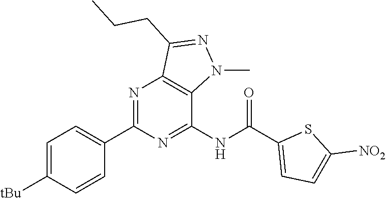 [SMILES: CCCc1nn(C)c2c(NC(=O)c3ccc([N+](=O)[O-])s3)nc(-c3ccc(C(C)(C)C)cc3)nc12]

¹H NMR (400 MHz, DMSO-d₆): δ 8.21 (br. s., 2H), 8.05-8.17 (m, 1H), 7.75 (s, 1H), 7.56 (br. s., 2H), 4.26 (br. s., 3H), 2.91 (t, J=7.3 Hz, 2H), 1.77-1.94 (m, 2H), 1.34 (s, 9H), 0.99 (4, J=7.3 Hz, 3H). MS(M+1) 479.

### Compound 3-8

### N-(1-methyl-5-phenyl-3-propyl-1H-pyrazolo[4,3-d]pyrimidin-7-yl)-5-nitrothiophene-2-carboxamide

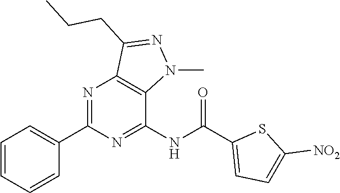 [SMILES: CCCc1nn(C)c2c(NC(=O)c3ccc([N+](=O)[O-])s3)nc(-c3ccccc3)nc12]

¹H NMR (400 MHz, DMSO-d₆): δ 8.26 (br. s., 2H), 8.01-8.22 (m, 1H), 7.92 (br s., 1H), 7.58 (d, J=6.8 Hz, 3H), 4.27 (br. s., 3H), 2.95 (t, J=7.6 Hz, 2H), 1.86 (sxt, J=7.5 Hz, 2H), 0.99 (t, J=7.3 Hz, 3H). MS(M+1): 423. Yellow solid.

### Compound 3-9

### N-(5-(4-fluorophenyl)-1-methyl-3-propyl-1H-pyrazolo[4,3-d]pyrimidin-7-yl)-5-nitrothiophene-2-carboxamide

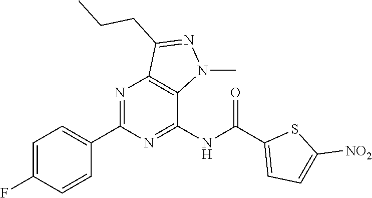 [SMILES: CCCc1nn(C)c2c(NC(=O)c3ccc([N+](=O)[O-])s3)nc(-c3ccc(F)cc3)nc12]

¹H NMR (400 MHz, DMSO-d₆): δ 8.31 (br. s., 2H), 8.22 (d, J=4.4 Hz, 1H), 8.03 (br. s., 1H), 7.44 (br. s., 2H), 4.22 (br. s., 3H), 2.% (t, J=7.3 Hz, 2H), 1.86 (sxt, J=7.3 Hz, 2H), 1.00 (t, J=7.3 Hz, 3H). MS(M+1): 441. Yellow solid.

### Compound 3-10

### N-(2-(4-(tert-butyl)phenyl)4-methyl-6H-pyrrolo[3,4,d]pyrimidin-4-yl)-5-nitrothiophene-2-carboxamide

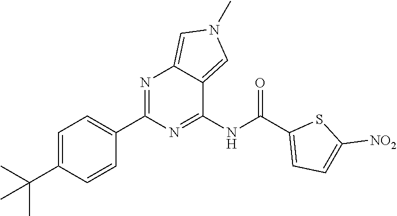 [SMILES: Cn1cc2nc(-c3ccc(C(C)(C)C)cc3)nc(NC(=O)c3ccc([N+](=O)[O-])s3)c2c1]

¹H NMR (400 MHz, DMSO-d₆): δ 8.07-8.13 (m, J=7.8 Hz, 2H), 8.05 (d, J=4.4 Hz, 1H), 7.89 (d, J=2.0 Hz, 1H), 7.85 (br. s., 1H), 7.66-7.73 (m, J=8.3 Hz, 2H), 7.43 (d, J=2.0 Hz, 1H), 4.14 (s, 3H), 1.40 (s, 9H). MS (M+1): 436. Pale yellow solid.

### Compound 3-11

### N-(2-(4-(tert-butyl)phenyl)-6-ethyl-6H-pyrrolo[3,4-d]pyrimidin-4-yl)-5-nitrothiophene-2-carboxamide

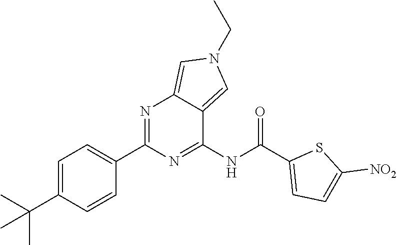 [SMILES: CCn1cc2nc(-c3ccc(C(C)(C)C)cc3)nc(NC(=O)c3ccc([N+](=O)[O-])s3)c2c1]

¹H NMR (400 MHz, DMSO-d₆): δ 14.36 (br. s., 1H), 8.24 (s, 1H), 8.13 (br. s., 1H), 7.88-8.08 (m, 2H), 7.80 (br. s., 1H), 7.44-7.73 (m, 3H), 4.30 (d, J=6.8 Hz, 2H), 1.49 (t, J=7.1 Hz, 3H), 1.23-1.41 (m, 9H). MS(M+1): 450. Yellow solid.

### Compound 3-12

### N-(2-(4-(tert-butyl)phenyl)-6-isopropyl-6H-pyrrolo[3,4-d]pyrimidin-4-yl)-5-nitrothiophene-2-carboxamide

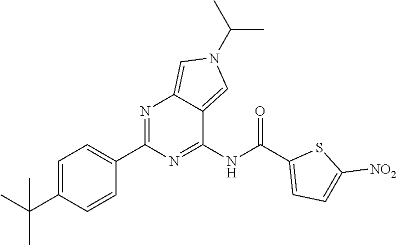 [SMILES: CC(C)n1cc2nc(-c3ccc(C(C)(C)C)cc3)nc(NC(=O)c3ccc([N+](=O)[O-])s3)c2c1]

¹H NMR (400 MHz, DMSO-d₆): δ 14.24 (s, 1H), 8.17 (d, J=4.4 Hz, 1H), 8.07 (br. s., 2H), 7.99 (br. s., 1H), 7.94 (br. s., 1H), 7.72 (d, J=2.0 Hz, 1H), 7.62 (d, J=8.3 Hz, 2H), 4.71 (quin, J=6.6 Hz, 1H), 1.56 (d, J=6.4 Hz, 6H), 1.34 (s, 9H). MS(M+1): 464. Yellow solid.

TABLE 4

| | | | |
|---|---|---|---|
| | (C) | | (D) |
| 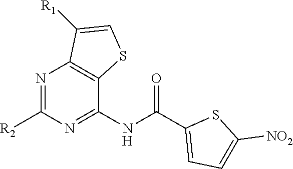 | | 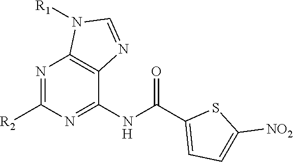 | |

Hep3B

| | | | |
|---|---|---|---|
| Compound | R1 | R2 | LC₅₀ (uM) |

Formula (C)

| | | | |
|---|---|---|---|
| 4-1 | Methyl | 4-(tert-butyl)phenyl | 1.15 |
| 4-2 | Methyl | 4-(trifluoromethyl)phenyl | 0.97 |
| 4-3 | Methyl | 3-(trifluorometbyl)phenyl | 0.88 |
| 4-4 | Methyl | 4-(trifluoromethoxy)phenyl | 0.89 |
| 4-5 | Methyl | 3-(trifluoromethoxy)phenyl | 0.79 |
| 4-6 | Methyl | 3,5-bis(trifluoromethyl) | 0.42 |
| | | phenyl | |
| 4-7 | Methyl | 3-fluorophenyl | 1.28 |
| 4-8 | Methyl | 4-fluorophenyl | 1.70 |
| 4-9 | Methyl | 3,4,5-trifluorophenyl | >10 |
| 4-10 | Methyl | 4-(dimethylamino)phenyl | >10 |
| 4-11 | Methyl | 3,3-difluoropyrrolidin-1-yl | 3.18 |
| 4-12 | Methyl | 4-(tert-butyl)phenoxy | 1.34 |
| 4-13 | Methyl | 2-fluoro-5-(trifluoromethyl) | 1.70 |
| | | phenyl | |
| 4-14 | Methyl | 2-fluoro-4-(trifluoromethyl) | 1.35 |
| | | phenyl | |
| 4-15 | Phenyl | 4-fluorophenyl | 0.62 |
| 4-16 | Phenyl | 4-chloro-3-fluorophenyl | 0.56 |
| 4-17 | Phenyl | 2-fluoro-5-(trifluoromethyl) | 0.86 |
| | | phenyl | |
| 4-18 | 3-fluorophenyl | 4-fluorophenyl | 0.49 |
| 4-19 | 4-(tert-butyl)phenyl | 4-fluorophenyl | 0.67 |

Formula (D)

| | | | |
|---|---|---|---|
| 4-20 | tert-butyl 4-piperidine- | 4-fluorophenyl | >1.25 |
| | 1-carboxylate | | |
| 4-21 | Cyclohexyl | 6-fluoropyridin-3-yl | NA |
| 4-22 | Cyclohexyl | 6-ethoxypyridin-3-yl | NA |

### Synthesis of 2-chloro-7-methylthieno[3,2-d]pyrimidin-4-amine

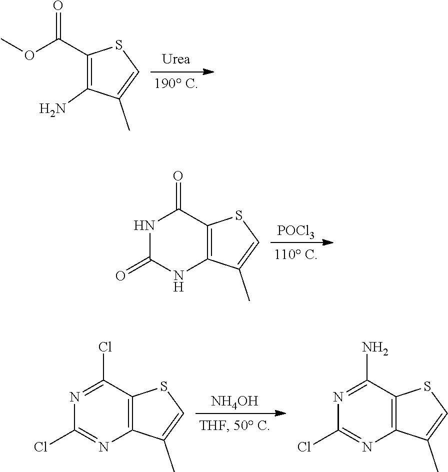

### Synthesis of 2-chloro-7-methylthieno[3,2-d]pyrimidin-4-amine by three steps

To a solution of methyl 3-amino-4-methylthiophene-2-carboxylate(20.6 g, 120 mmol) was added 36 g (600 mmol) of urea, and the resulting mixture was heated at 200° C. for 1.5 hours. The mixture was allowed to resume room temperature, and DMF (360 ml) was added thereto, followed by heating under reflux for one hour. After completion of the reaction, ice water was added to the reaction mixture, and crystals thus precipitated were filtered to give (19.8 g, 90%) of 7-methylthieno[3,2-d]-pyrimidine-2,4(1H,3H)-dione.

To a solution of 7-methylthieno[3,2-d]-pyrimidine-2,4(1H,3H)-dione (18.3 g, 100 mmol) were added 153.0 g (1 mol) of phosphorus oxychloride and the resulting mixture was subjected to heating under reflux for 8 hours. After completion of the reaction, ice water was added to the reaction mixture, and crystals thus precipitated were filtered to give (15.4 g, 70%) of 2,4-dichloro-7-methylthieno[3,2-d]pyrimidine.

To a suspension solution of 2,4-dichloro-7-methylthieno[3,2-d]pyrimidine (8.8 g, 40 mmol) in the reaction flask and then add 100 ml THF to wait for the solid to dissolve completely. Then add 100 g, 30% ammonium solution and react at room temperature for 24 h. Poured 60 ml water into the solution, filtration by suction to afford the 2-chloro-7-methylthieno[3,2-d]pyrimidin-4-amine (6.8 g, 85%) as a yellow solid powder.

### Compound 4-1

### N-(2-(4-tert-butyl)phenyl)-7-methylthieno[3,2-d]pyrimidin-4-yl)-5-nitrothiophene-2-carboxamide

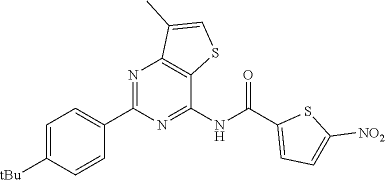 [SMILES: Cc1csc2c(NC(=O)c3ccc([N+](=O)[O-])s3)nc(-c3ccc(C(C)(C)C)cc3)nc12]

¹H NMR (400 MHz, DMSO-d₆): δ 11.97 (br. s., 1H), 8.46 (d, J=8.3 Hz, 2H), 8.24 (d, J=4.4 Hz, 1H), 8.08 (d, J=1.5 Hz, 1H), 7.59 (d, J=8.3 Hz, 2H), 3.28 (br. s., 3H), 1.35 (s, 9H), MS(M+1): 453.

### Compound 4-2

### N-(7-methyl-2-(4-(trifluoromethyl)phenyl)thieno[3,2-d]pyrimidin-4-yl)-5-nitrothiophene-2-carboxamide

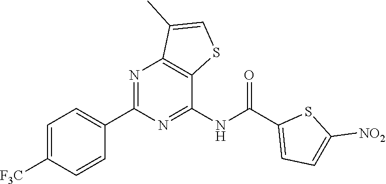 [SMILES: Cc1csc2c(NC(=O)c3ccc([N+](=O)[O-])s3)nc(-c3ccc(C(F)(F)F)cc3)nc12]

¹H NMR (400 MHz, DMSO-d₆): δ 12.06 (br. s., 1H), 8.68-8.80 (m, J=7.8 Hz, 2H), 8.32 (d, J=4.4 Hz, 1H), 8.25 (d, J=4.4 Hz, 1H), 8.14 (d, J=1.5 Hz, 1H), 7.91-8.02 (m, J=8.3 Hz, 2H), 2.53 (d, J=1.0 Hz, 3H). MS(M+1): 465

### Compound 4-3

### N-(7-methyl-2-(3-(trifluoromethyl)phenyl)thieno[3,2-1]pyrimidin-4-yl)-5-nitrothiophene-2-carboxamide

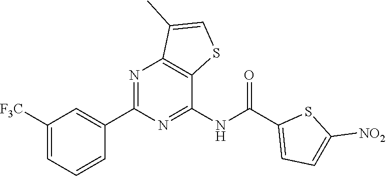 [SMILES: Cc1csc2c(NC(=O)c3ccc([N+](=O)[O-])s3)nc(-c3cccc(C(F)(F)F)c3)nc12]

¹H NMR (400 MHz, DMSO-d₆): δ 12.01 (br. s., 1H), 8.73-8.93 (m, 2H), 8.32 (d, J=4.4 Hz, 1H), 8.25 (d, J=4.4 Hz, 1H), 8.14 (d, J=1.0 Hz, 1H), 7.93 (d, J=7.8 Hz, 1H), 7.84 (t, J=7.6 Hz, 1H), 2.52 (d, J=1.0 Hz, 3H). MS(M+1): 465.

### Compound 4-4

### N-(7-methyl-2-(4-(trifluoromethoxy)phenyl)thieno[3,2-d]pyrimidin-4-yl)-5-nitrothiophene-2-carboxamide

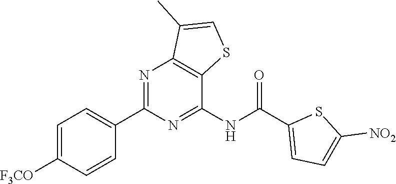 [SMILES: Cc1csc2c(NC(=O)c3ccc([N+](=O)[O-])s3)nc(-c3ccc(OC(F)(F)F)cc3)nc12]

¹H NMR (400 MHz, DMSO-d₆): δ 12.03 (br. s., 1H), 8.50-8.78 (m, 2H), 8.16-8.38 (m, 2H), 8.11 (d, J=1.0 Hz, 1H), 7.57 (d, J=7.8 Hz, 2H), 2.49 (br. s., 3H). MS(M+1): 481.

### Compound 4-5

### N-(7-methyl-2-(3-(trifluoromethoxy)phenyl)thieno[3,2-d]pyrimidin-4-yl)-5-nitrothiophene-2-carboxamide

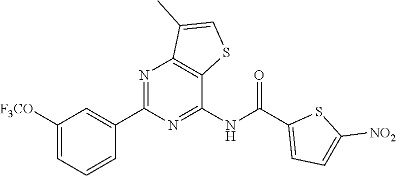 [SMILES: Cc1csc2c(NC(=O)c3ccc([N+](=O)[O-])s3)nc(-c3cccc(OC(F)(F)F)c3)nc12]

¹H NMR (400 MHz, DMSO-d₆): δ 11.97 (s, 1H), 8.58 (d, J=7.8 Hz, 1H), 8.43 (s, 1H), 8.31 (d, J=4.4 Hz, 1H), 8.25 (d, J=4.4 Hz, 1H), 8.14 (d, J=1.0 Hz, 1H), 7.73 (t, J=8.1 Hz, 1H), 7.52-7.60 (m, 1H), 2.51 (d, J=1.0 Hz, 7H). MS(M+1): 481.

### Compound 4-6

### N-(2-(3,5-bis(trifluoromethyl)phenyl)-7-methylthieno[3,2-d]pyrimidin-4-yl)-5-nitrothiophene-2-carboxamide

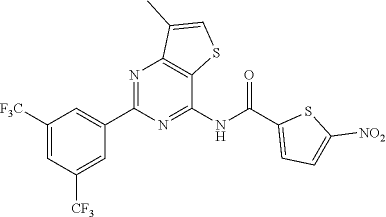 [SMILES: Cc1csc2c(NC(=O)c3ccc([N+](=O)[O-])s3)nc(-c3cc(C(F)(F)F)cc(C(F)(F)F)c3)nc12]

¹H NMR (400 MHz, DMSO-d₆): δ 12.01 (br. s., 1H), 9.07 (s, 2H), 8.30 (d, J=4.4 Hz, 2H), 8.24 (d, J=4.4 Hz, 1H), 8.15 (d, J=1.0 Hz, 1H), 2.52 (s, 2H). MS(M+1): 533.

### Compound 4-7

### N-(2-(3-fluorophenyl)-7-methylthieno[3,2-d]pyrimidin-4-yl)-5-nitrothiophene-2-carboxamide

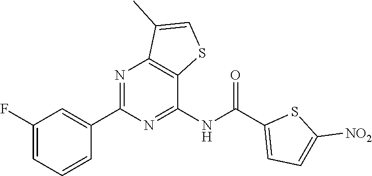 [SMILES: Cc1csc2c(NC(=O)c3ccc([N+](=O)[O-])s3)nc(-c3cccc(F)c3)nc12]

¹H NMR (400 MHz, DMSO-d₆): δ 11.94 (br. s., 1H), 8.39 (d, J=7.8 Hz, 1H), 8.18-8.33 (m, 3H), 8.11 (d, J=1.0 Hz, 1H), 7.63 (td, J=8.1, 6.4 Hz, 1H), 7.33-7.45 (m, 1H), 2.51 (s, 3H). MS(M+1): 415.

### Compound 4-8

### N-(2-(4-fluorophenyl)-7-ethylthieno[3,2-d]pyrimidin-4-yl)-5-nitrothiophene-2-carboxamide

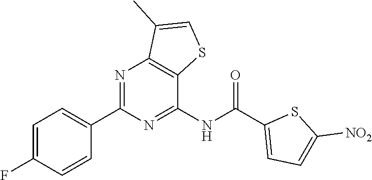 [SMILES: Cc1csc2c(NC(=O)c3ccc([N+](=O)[O-])s3)nc(-c3ccc(F)cc3)nc12]

¹H NMR (400 MHz, DMSO-d₆): δ 11.95 (br. s., 1H), 8.50-8.66 (m, 2H), 8.30 (d, J=4.4 Hz, 1H), 8.24 (d, J=4.4 Hz, 1H), 8.10 (d, J=1.0 Hz, 1H), 7.31-7.50 (m, 2H), 2.50 (s, 3H). MS(M+1): 415.

### Compound 4-9

### N-(7-methyl-2-(3,4,5-trifluorophenyl)thieno[3,2-d]pyrimidin-4-yl)-5-nitrothiophene-2-carboxamide

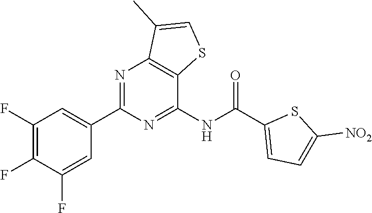 [SMILES: Cc1csc2c(NC(=O)c3ccc([N+](=O)[O-])s3)nc(-c3cc(F)c(F)c(F)c3)nc12]

¹H NMR (400 MHz, DMSO-d₆): δ 11.94 (br. s., 1H), 8.28-8.36 (m, 3H), 8.25-8.27 (m, 1H), 8.14 (d, J=1.0 Hz, 1H), 2.50 (s, 3H). MS(M+1): 451.

### Compound 4-10

### N-(2-(4-(dimethylamino)phenyl)-7-methylthieno[3,2-d]pyrimidin-4-yl)-5-nitrothiophene-2-carboxamide

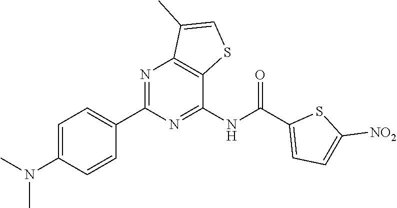 [SMILES: Cc1csc2c(NC(=O)c3ccc([N+](=O)[O-])s3)nc(-c3ccc(N(C)C)cc3)nc12]

¹H NMR (400 MHz, DMSO-d₆): δ 11.85 (br. s., 1H), 8.33-8.51 (m, 2H), 8.30 (br. s., 1H), 8.24 (d, J=3.9 Hz, 1H), 8.01 (br. s., 1H), 6.84 (d, J=8.8 Hz, 2H), 3.02 (s, 6H), 2.47 (d, J=1.0 Hz, 3H). MS(M+1): 440.

### Compound 4-11

### N-(2-(3,3-difluoropyrrolidin-1-yl)-7-methylthieno[3,2-d]pyrimidin-4-yl)-5-nitrothiophene-2-carboxamide

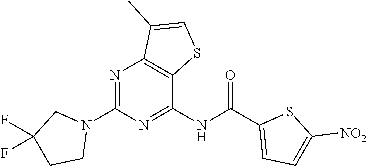 [SMILES: Cc1csc2c(NC(=O)c3ccc([N+](=O)[O-])s3)nc(N3CCC(F)(F)C3)nc12]

¹H NMR (400 MHz, DMSO-d₆): δ 11.59 (br. s., 1H), 8.16-8.30 (m, 2H), 7.87 (d, J=1.0 Hz, 1H), 3.89-4.07 (m, 2H), 3.84 (t, J=7.3 Hz, 2H), 2.52-2.67 (m, 3H), 2.30 (d, J=1.0 Hz, 3H), MS(M+1): 426.

### Compound 4-12

### N-(2-(4-(tert-butyl)phenoxy)-7-methylthieno[3,2-d]pyrimidin-4-yl)-5-nitrothiophene-2-carboxamide

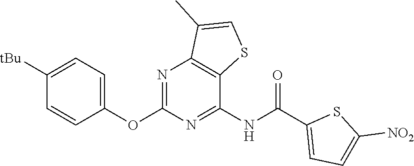 [SMILES: Cc1csc2c(NC(=O)c3ccc([N+](=O)[O-])s3)nc(Oc3ccc(C(C)(C)C)cc3)nc12]

¹H NMR (400 MHz, DMSO-d₆): δ 12.07 (s, 1H), 8.21-8.25 (m, 1H), 8.18-8.21 (m, 1H), 8.05 (d, J=1.0 Hz, 1H), 7.34-7.55 (m, 2H), 7.10-7.25 (m, 2H), 2.26 (s, 3H), 1.31 (s, 9H). MS(M+1): 469.

### Compound 4-13

### N-(2-(2-fluoro-5-(trifluoromethyl)phenyl)-7-methylthieno[3,2-d]pyrimidin-4-yl)-5-nitrothiophene-2-carboxamide

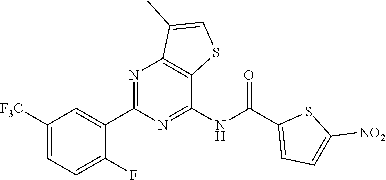 [SMILES: Cc1csc2c(NC(=O)c3ccc([N+](=O)[O-])s3)nc(-c3cc(C(F)(F)F)ccc3F)nc12]

¹H NMR (400 MHz, DMSO-d₆): δ 12.12 (br. s., 1H), 8.48 (dd, J=6.8, 2.4 Hz, 1H), 8.27 (d, J=4.4 Hz, 1H), 8.17-8.25 (m, 1H), 8.16 (d, J=4.4 Hz, 1H), 7.88-8.05 (m, 1H), 7.65 (t, J=9.5 Hz, 1H), 2.47 (d, J=1.0 Hz, 3H). MS(M+1): 483. Yellow solid

### Compound 4-14

### N-(2-(2-fluoro-4-(trifluoromethyl)phenyl)-7-methylthieno[3,2-d]pyrimidin-4-yl)-5-nitrothiophene-2-carboxamide

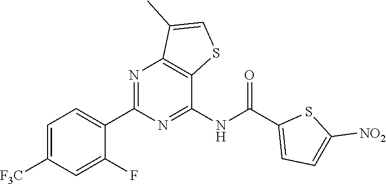 [SMILES: Cc1csc2c(NC(=O)c3ccc([N+](=O)[O-])s3)nc(-c3ccc(C(F)(F)F)cc3F)nc12]

¹H NMR (400 MHz, DMSO-d₆): δ 12.18 (br. s., 1H), 8.32 (t, J=7.8 Hz, 1H), 8.23-8.28 (m, 1H), 8.18-8.23 (m, 1H), 8.15 (d, J=1.0 Hz, 1H), 7.86 (d, J=10.3 Hz, 1H), 7.79 (d, J=8.3 Hz, 1H), 2.46 (d, J=1.0 Hz, 3H). MS(M+1): 483. Yellow solid.

### Compound 4-15

### N-(2-(4-fluorophenyl)-7-phenylthieno[3,2-d]pyrimidin-4-yl)-5-nitrothiophene-2-carboxamide

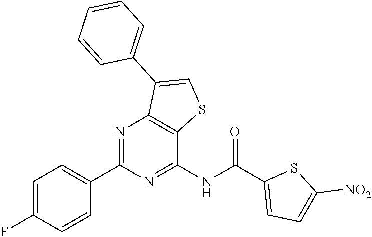 [SMILES: O=C(Nc1nc(-c2ccc(F)cc2)nc2c(-c3ccccc3)csc12)c1ccc([N+](=O)[O-])s1]

¹H NMR (400 MHz, DMSO-d₆): δ 12.08 (s, 1H), 8.69 (s, 1H), 8.56 (dd, J=8.8, 5.4 Hz, 2H), 8.34 (d, J=4.4 Hz, 1H), 8.27 (d, J=4.4 Hz, 1H), 8.09-8.21 (m, 2H), 7.51-7.62 (m, 2H), 7.36-7.49 (m, 3H). MS(M+1): 477. Yellow solid.

### Compound 4-16

### N-(2-(4-chloro-3-fluorophenyl)-7-phenylthieno[3,2-d]pyrimidin-4-yl)-5-nitrothiophene-2-carboxamide

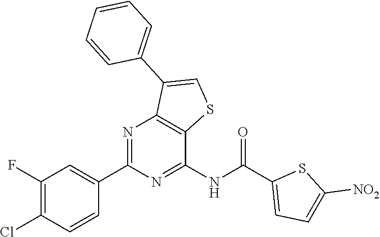 [SMILES: O=C(Nc1nc(-c2ccc(Cl)c(F)c2)nc2c(-c3ccccc3)csc12)c1ccc([N+](=O)[O-])s1]

¹H NMR (400 MHz DMSO-d₆): δ 12.09 (s, 1H), 8.69-8.74 (m, 1H), 8.31-8.40 (m, 3H), 8.23-8.30 (m, 1H), 8.06-8.18 (m, 2H), 7.76-7.88 (m, 1H), 7.51-7.64 (m, 2H), 7.40-7.50 (m, 1H). MS(M+1): 511. Yellow solid.

### Compound 4-17

### N-(2-(2-fluoro-5-(trifluoromethyl)phenyl)-7-phenylthieno[3,2-d]pyrimidin-4-yl)-5-nitrothiophene-2-carboxamide

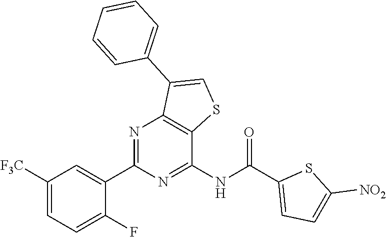 [SMILES: O=C(Nc1nc(-c2cc(C(F)(F)F)ccc2F)nc2c(-c3ccccc3)csc12)c1ccc([N+](=O)[O-])s1]

¹H NMR (400 MHz, DMSO-d₆): δ 12.21 (br. s., 1H), 8.77 (s, 1H), 8.58 (dd, J=6.8, 2.4 Hz, 1H), 8.32 (d, J=4.4 Hz, 1H), 8.25 (d, J=4.4 Hz, 1H), 8.14-8.20 (m, 2H), 8.00 (dt, J=8.2, 3.5 Hz, 1H), 7.63-7.73 (m, 1H), 7.47-7.56 (m, 2H), 7.38-7.46 (m, 1H) MS(M+1): 545. Ashy solid.

### Compound 4-18

### N-(7-(3-fluorophenyl)-2-(4-fluorophenyl)thieno[3,2-d]pyrimidin-4-yl)-5-nitrothiophene-2-carboxamide

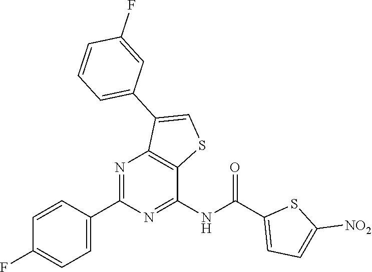 [SMILES: O=C(Nc1nc(-c2ccc(F)cc2)nc2c(-c3cccc(F)c3)csc12)c1ccc([N+](=O)[O-])s1]

¹H NMR (400 MHz, DMSO-d₆): δ 12.09 (s, 1H), 8.81 (s, 1H), 8.55 (dd, J=8.8, 5.9 Hz, 2H), 8.34 (d, J=4.4 Hz, 1H), 8.26 (d, J=4.4 Hz, 1H), 8.04-8.09 (m, 1H), 8.02 (d, J=7.8 Hz, 1H), 7.60 (td, J=7.9, 6.6 Hz, 1H), 7.38-7.49 (m, 2H), 7.17-7.33 (m, 1H)

MS(M+1): 495. Orange solid

### Compound 4-19

### N-(7-(4-(tert-butyl)phenyl)-2-(4-fluorophenyl)thieno[3,2-d]pyrimidin-4-yl)-5-nitrothiophene-2-carboxamide

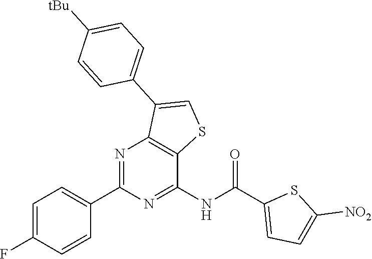 [SMILES: CC(C)(C)c1ccc(-c2csc3c(NC(=O)c4ccc([N+](=O)[O-])s4)nc(-c4ccc(F)cc4)nc23)cc1]

¹H NMR (400 MHz, DMSO-d₆): δ 12.09 (br. s., 1H), 8.63 (s, 1H), 8.57 (dd, J=8.8, 5.9 Hz, 2H), 8.28-8.35 (m, 1H), 8.19-8.28 (m, 1H), 8.04-8.17 (m, 2H), 7.52-7.65 (m, 2H), 7.43 (t, J=8.8 Hz, 2H), 1.37 (s, 9H). MS(M+1): 533. Yellow solid.

### Compound 4-20

### tert-butyl 4-(2-(4-fluorophenyl)-6-(5-nitrothiophene-2-carboxamide)-9H-purin-9-yl) piperidine-1-carboxylate

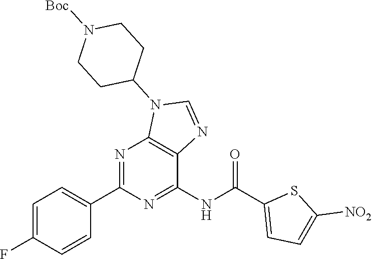 [SMILES: CC(C)(C)OC(=O)N1CCC(n2cnc3c(NC(=O)c4ccc([N+](=O)[O-])s4)nc(-c4ccc(F)cc4)nc32)CC1]

¹H NMR (400 MHz, DMSO-d₆): δ 11.76 (br. s., 1H), 8.65 (s, 1H), 8.43-8.58 (m, 2H), 8.15-8.28 (m, 2H), 7.27-7.46 (m, 2H), 4.73-4.89 (m, 1H), 4.16 (d, J=11.7 Hz, 2H), 3.0) (br. s., 2H), 2.06-2.25 (m, 4H). MS(M+1): 568. Yellow solid.

### Compound 4-21

### N-(9-cyclohexyl-2-(6-fluoropyridin-3-yl)-9H-purin-6-yl)-5-nitrothiophene-2-carboxamide

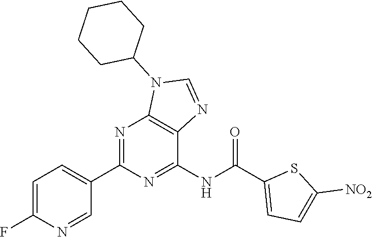 [SMILES: O=C(Nc1nc(-c2ccc(F)nc2)nc2c1ncn2C1CCCCC1)c1ccc([N+](=O)[O-])s1]

¹H NMR (400 MHz, DMSO-d₆): δ 11.82 (s, 1H), 9.22 (d, J=2.4 Hz, 1H), 8.89 (td, J=8.3, 2.4 Hz, 1H), 8.68 (s, 1H), 8.23 (q, J=4.4 Hz, 2H), 7.38 (dd, J=8.8, 2.4 Hz, 1H), 4.56-4.69 (m, 1H), 1.99-2.18 (m, 4H), 1.82-1.97 (m, 2H), 1.75 (d, J=13.2 Hz, 1H), 1.41-1.61 (m, 2H), 1.25-1.40 (m, 1H). MS(M+1) 468. Khaki solid.

### Compound 4-22

### N-(9-cyclohexyl-2-(6-ethoxypyridin-3-yl)-9H-purin-6-yl)-5-nitrothiophene-2-carboxamide

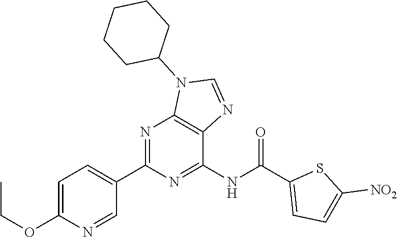 [SMILES: CCOc1ccc(-c2nc(NC(=O)c3ccc([N+](=O)[O-])s3)c3ncn(C4CCCCC4)c3n2)cn1]

¹H NMR (400 MHz, DMSO-d₆): δ 11.76 (br. s., 1H), 9.18 (d, J=2.4 Hz, 1H), 8.55-8.68 (m, 2H), 8.16-8.27 (m, 2H), 6.90-7.02 (m, 1H), 4.52-4.68 (m, 1H), 4.33-4.46 (m, 2H), 1.97-2.20 (m, 4H), 1.91 (t, J=6.6 Hz, 2H), 1.75 (d, J=12.2 Hz, 1H), 1.52 (q, J=12.9 Hz, 2H), 1.30-1.42 (m, 4H). MS(M+1): 494. Khaki solid.

TABLE 5

| | 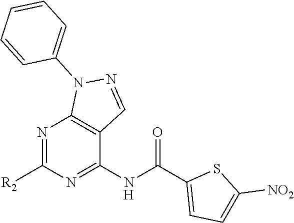 | |
|---|---|---|
| | | Hep3B |
| | | LC50 |
| Compound | R₂ | (uM) |
| 5-1 | piperidin-4-yl | >5.0 |
| 5-2 | 1-acryloylpiperidin-4-yl | 2.47 |
| 5-3 | pyrrolidin-1-yl | 4.91 |
| 5-4 | (S)-pyrrolidin-3-o1-yl | 1.01 |
| 5-5 | (R)-3-cyanopyrrolidin-1-yl | 0.50 |
| 5-6 | 3,3-difluoropyrrolidin-1-yl | 0.53 |
| 5-7 | morpholinyl | 1.46 |
| 5-8 | (2S, 6R)-2,6-dimethylmorpholinyl | 1.71 |
| 5-9 | 4-(chlorophenyl)sulfonamido | >6.0 |
| 5-10 | 2-(2-ethoxyethoxy)ethoxy | >5.0 |
| 5-11 | 2-(dimethylamino)ethoxy | 4.13 |
| 5-12 | 2-morpholinoethoxy | >3.0 |
| 5-13 | phenoxy | >5.0 |
| 5-14 | 4-fluorophenoxy | >5.0 |
| 5-15 | phenylthio | >5.0 |
| 5-16 | 4-(chlorophenyl)thio | 0.36 |
| 5-17 | furan-2-yl | 0.40 |
| 5-18 | thiophen-2-yl | 0.58 |
| 5-19 | 5-chlorothiophen-2-yl | 0.17 |
| 5-20 | thiophen-3-yl | 1.11 |
| 5-21 | 3,5-dimethylisoxazol-4-yl | 0.76 |
| 5-22 | 3-(trifluoromethyl)-1H-pyrazol-1-yl | 0.38 |
| 5-23 | phenyl | 0.43 |
| 5-24 | pyrimidin-5-yl | 0.96 |
| 5-25 | 2-fluorophenyl | 0.46 |
| 5-26 | 3-fluorophenyl | 0.41 |
| 5-27 | 4-fluorophenyl | 0.27 |
| 5-28 | 6-fluoropyridin-3-yl | 0.13 |
| 5-29 | 3-chlorophenyl | 0.21 |
| 5-30 | 4-chlorophenyl | 0.35 |
| 5-31 | 6-chloropyridin-3-yl | 0.10 |
| 5-32 | 4-cyanophenyl | 0.11 |
| 5-33 | 6-cyanopyridin-3-yl | 0.30 |
| 5-34 | 6-methylpyridin-3-yl | 0.85 |
| 5-35 | 6-methoxypyridin-3-yl | 0.18 |
| 5-36 | 4-methoxyphenyl | 0.60 |
| 5-37 | 4-(tert-butyl)phenyl | 0.78 |
| 5-38 | 3-(trifluoromethyl)phenyl | 0.45 |
| 5-39 | 4-(trifluoromethyl)phenyl | 0.32 |
| 5-40 | 4-(trifluoromethoxy)phenyl | 0.43 |
| 5-41 | 3-(dimethylamino)phenyl | 0.45 |
| 5-42 | 6-(6-(piperidin-1-yl)pyridin-3-yl | 0.51 |
| 5-43 | 6-morpholinopyridin-3-yl | 0.57 |
| 5-44 | 3,4-difluorophenyl | 0.25 |
| 5-45 | 2,4-difluorophenyl | 0.44 |
| 5-46 | 4-chloro-3-fluorophenyl | 0.24 |
| 5-47 | 4-chloro-2-fluorophenyl | 0.13 |
| 5-48 | 3,4-dichlorophenyl | 0.41 |
| 5-49 | 2,4-dichlorophenyl | 0.27 |
| 5-50 | 2-fluoro-5-(trifluorometbyl)phenyl | 0.35 |
| 5-51 | 2-fluoro-4-(trifluoromethyl)phenyl | 0.37 |
| 5-52 | 3-fluoro-4-(trifluoromethyl)phenyl | >1.25 |
| 5-53 | 3,4,5-trifluorophenyl | >1.25 |
| 5-54 | 6-(2-methoxyethoxy)pyridin-3-yl | 0.34 |
| 5-55 | 6-(2-ethoxyethoxy)pyridin-3-yl | 0.49 |
| 5-56 | 6-(2,2,3, 3-tetrafluoropropoxy)pyridin-3-yl | 0.40 |
| 5-57 | 6-(2-(2-ethoxyethoxy)ethoxy)pyridin-3-yl | 0.79 |

### Compound 5-1

### 5-nitro-N-(1-phenyl-6-(piperidin-4-yl)-1H-pyrazolo[3,4-d]pyrimidin-4-yl)thiophene-2-carboxamide

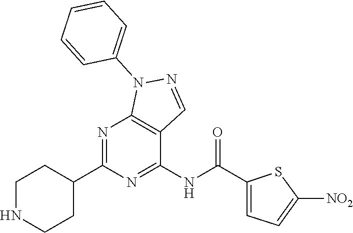 [SMILES: O=C(Nc1nc(C2CCNCC2)nc2c1cnn2-c1ccccc1)c1ccc([N+](=O)[O-])s1]

¹H NMR (400 MHz, DMSO-d₆): δ 8.36 (s, 1H), 8.29-8.24 (m, 2H), 8.07 (d, J=4.4 Hz, 1H), 7.66 (d, J=3.9 Hz, 1H), 7.54 (t, J=7.9 Hz, 2H), 7.31 (t, J=7.3 Hz, 1H), 3.38-3.36 (m, 2H), 3.07-3.02 (m, 3H), 2.15-2.02 (m, 4H). MS(M+1): 450. Yellow solid.

### Compound 5-2

### N-(6-(1-acryloylpiperidin-4-yl)-1-phenyl-1H-pyrazolo[3,4-d]pyrimidin-4-yl)-5-nitrothiophene-2-carboxamide

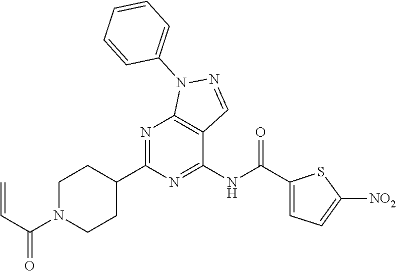 [SMILES: C=CC(=O)N1CCC(c2nc(NC(=O)c3ccc([N+](=O)[O-])s3)c3cnn(-c4ccccc4)c3n2)CC1]

¹H NMR (400 MHz, DMSO-d₆): δ 12.11 (s, 1H), 8.58 (s, 1H), 8.30 (brs, 1H), 8.23-8.20 (m, 3H), 7.60 (t, J=7.8 Hz, 2H), 7.40 (t, J=7.4 Hz, 1H), 6.87 (dd, J=16.6, 10.3 Hz, 1H), 6.12 (dd, J=16.6, 2.5 Hz, 1H), 5.69 (dd, J=10.3, 2.5 Hz, 1H), 4.55-4.52 (m, 1H), 4.21-4.17 (m, 1H), 3.27-3.21 (m, 2H), 2.91-2.84 (m, 1H), 2.12-2.08 (m, 2H), 1.83-1.78 (m, 2H) MS(M+1): 504. Yellow solid.

### Compound 5-3

### 5-nitro-N-(1-phenyl-6-(pyrrolidin-1-yl)-1H-pyrazolo[3,4-d]pyrimidin-4-yl)thiophene-2-carboxamide

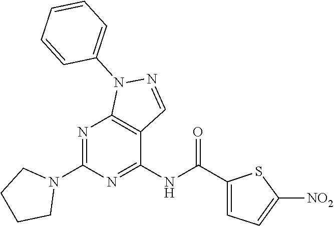 [SMILES: O=C(Nc1nc(N2CCCC2)nc2c1cnn2-c1ccccc1)c1ccc([N+](=O)[O-])s1]

¹H NMR (400 MHz, DMSO-d₆): δ 8.35 (dd, J=8.8, 1.0 Hz, 2H), 8.05 (t, J=2.2 Hz, 2H), 7.64 (d, J=4.4 Hz, 1H), 7.44-7.52 (m, 2H), 7.21 (t, J=7.3 Hz, 1H), 3.54-3.62 (m, 4H), 1.90-1.97 (m, 4H). MS(M+1): 436.

### Compound 5-4

### (S)—N-(6-(3-hydroxypyrrolidin-1-yl)-1-phenyl-1H-pyrazolo[3,4-d]pyrimidin-4-yl)-5-nitrothiophene-2-carboxamide

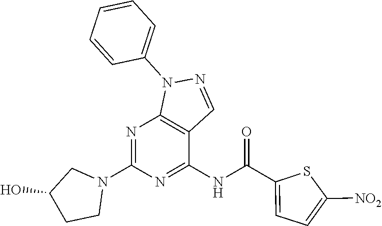 [SMILES: O=C(Nc1nc(N2CC[C@H](O)C2)nc2c1cnn2-c1ccccc1)c1ccc([N+](=O)[O-])s1]

¹H NMR (400 MHz, DMSO-d₆): δ 11.56 (s, 1H), 8.30-8.20 (m, 5H), 7.53 (t, J=7.8 Hz, 2H), 7.29 (t, J=7.3 Hz, 1H), 5.00 (d, J=3.5 Hz, 1H), 4.42 (s, 1H), 3.70-3.60 (m, 4H), 2.09-1.99 (m, 1H), 1.94 (brm, 1H). MS(M+1): 452. Yellow solid.

### Compound 5-5

### (R)—N-(6-(3-cyanopyrrolidin-1-yl)-1-phenyl-1H-pyrazolo[3,4-d]pyrimidin-4-yl)-5-nitrothiophene-2-carboxamide

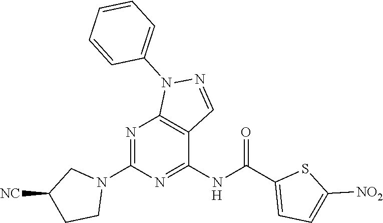

¹H-NMR (DMSO-d₆, 400 MHz): δ 11.62 (s, 1H), 8.31-8.22 (m, 5H), 7.55 (t, J=7.8 Hz, 2H), 7.32 (t, J=7.4 Hz, 1H), 4.00-3.59 (m, 5H), 2.52-2.38 (m, 1H), 2.33-1.91 (m, 2H). MS(M+1): 461. Yellow solid.

### Compound 5-6

### N-(6-(3,3-difluoropyrrolidin-1-yl)-1-phenyl-1H-pyrazolo[3,4-d]pyrimidin-4-yl)-5-nitrothiophene-2-carboxamide

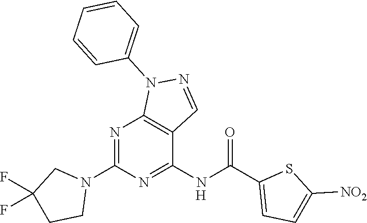

¹H NMR (400 MHz, DMSO-d₆): δ 11.63 (br. s., 1H), 8.33 (s, 1H), 8.30 (d, J=4.4 Hz, 1H), 8.25 (d, J=7.3 Hz, 2H), 8.22 (d, J=4.4 Hz, 1H), 7.55 (dd, J=8.3, 7.3 Hz, 2H), 7.29-7.35 (m, 1H), 4.05 (t, J=13.2 Hz, 2H), 3.88 (t, J=7.1 Hz, 2H), 2.59 (tt, J=14.2, 7.3 Hz, 2H). MS(M+1): 472.

### Compound 5-7

### N-(6-morpholino-1-phenyl-1H-pyrazolo[3,4-d]pyrimidin-4-yl)-5-nitrothiophene-2-carboxamide

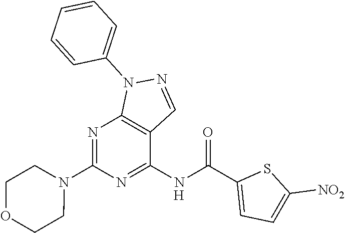

¹H NMR (400 MHz, DMSO-d₆): δ 11.52 (br. s., 1H), 8.33 (s, 1H), 8.29 (d, J=4.4 Hz, 1H), 8.23 (d, J=4.4 Hz, 1H), 8.19 (dd, J=8.8, 1.0 Hz, 2H), 7.50-7.58 (m, 2H), 7.28-7.35 (m, 1H), 3.82-3.90 (m, 4H), 3.68-3.76 (m, 4H). MS (M+1): 452.

### Compound 5-8

### N-(6-((2S,6R)-2,6-dimethylmorpholino)-1-phenyl-1H-pyrazolo[3,4-d]pyrimidin-4-yl)-5-nitrothiophene-2-carboxamide

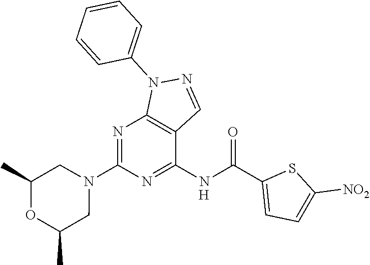

¹H NMR (400 MHz, DMSO-d₆): δ 11.46 (s, 1H), 8.30 (s, 1H), 8.28 (d, J=4.4 Hz, 1H), 8.20 (d, J=4.4 Hz, 1H), 8.17 (d, J=7.3 Hz, 2H), 7.54 (t, J=8.1 Hz, 2H), 7.31 (t, J=7.6 Hz, 1H), 4.62 (d, J=12.2 Hz, 2H), 3.55-3.67 (m, 2H), 2.64 (dd, J=13.2, 10.8 Hz, 2H), 1.19 (s, 3H), 1.18 (s, 3H). MS(M+1): 480. Orange solid.

### Compound 5-9

### N-(6-((4-chlorophenyl)sulfonamido)-1-phenyl-1H-pyrazolo[3,4-d]pyrimidin-4-yl)-5-nitrothiophene-2-carboxamide

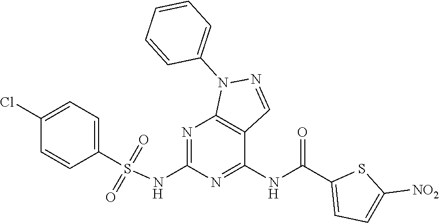

¹H NMR (400 MHz, DMSO-d₆): δ 12.06 (br. s., 1H), 11.95 (br. s., 1H), 8.42 (s, 1H), 8.29 (br. s., 1H), 8.23 (d, J=4.4 Hz, 1H), 8.04 (d, J=8.3 Hz, 2H), 7.96 (d, J=7.8 Hz, 2H), 7.52-7.66 (m, 4H), 7.35-7.46 (m, 1H). MS(M+1): 590. Cream solid.

### Compound 5-10

### N-(6-(2-(2-ethoxyethoxy)ethoxy)-1-phenyl-1H-pyrazolo[3,4-d]pyrimidin-4-yl)-5-nitrothiophene-2-carboxamide

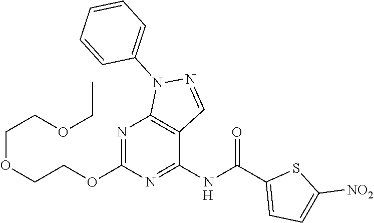

¹H NMR (400 MHz, DMSO-d₆): δ 12.12 (br. s., 1H), 8.52 (s, 1H), 8.32 (d, J=4.4 Hz, 1H), 8.21 (d, J=4.4 Hz, 1H), 8.17 (dd, J=8.8, 1.0 Hz, 2H), 7.53-7.61 (m, 2H), 7.33-7.41 (m, 1H), 4.50-4.59 (m, 2H), 3.77-3.85 (m, 2H), 3.59 (dd, J=5.9, 3.9 Hz, 2H), 3.45-3.50 (m, 2H), 3.40 (q, J=6.8 Hz, 2H), 1.03-1.10 (m, 3H). MS(M+1): 499.

Yellow solid.

### Compound 5-11

### N-(6-(2-(dimethylamino)ethoxy)-1-phenyl-1H-pyrazolo[3,4-d]pyrimidin-4-yl)-5-nitrothiophene-2-carboxamide

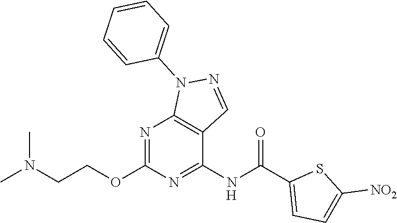

¹H NMR (400 MHz, DMSO-d₆): δ 8.57 (s, 1H), 8.32 (d, J=4.9 Hz, 1H), 8.24 (d, J=4.4 Hz, 1H), 8.16 (d, J=7.8 Hz, 2H), 7.60 (t, J=7.8 Hz, 2H), 7.38-7.45 (m, 1H), 4.74-4.83 (m, 2H), 3.57-3.65 (m, 2H), 2.88 (s, 6H). MS (M+1): 454.

### Compound 5-12

### N-(6-(2-morpholinoethoxy)-1-phenyl-1H-pyrazolo[3,4-d]pyrimidin-4-yl)-5-nitrothiophene-2-carboxamide

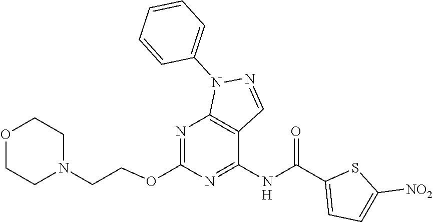

¹H NMR (400 MHz, DMSO-d₆): δ 8.54 (s, 1H), 8.34 (d, J=4.4 Hz, 1H), 8.23 (d, J=4.4 Hz, 1H), 8.17 (dd, J=8.8, 1.0 Hz, 2H), 7.54-7.62 (m, 2H), 7.39 (t, J=7.3 Hz, 1H), 4.58 (t, J=5.4 Hz, 2H), 3.56 (br s., 4H), 2.82 (br. s., 2H). MS(M+1): 496.

### Compound 5-13

### 5-nitro-N-(6-phenoxy-1-phenyl-1H-pyrazolo[3,4-d]pyrimidin-4-yl)thiophene-2-carboxamide

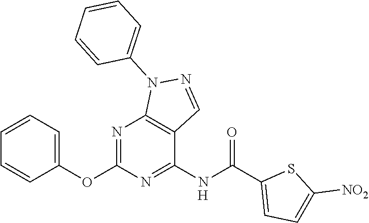

¹H NMR (400 MHz, DMSO-d₆): δ 12.29 (s, 1H), 8.56 (s, 1H), 8.33 (d, J=4.4 Hz, 1H), 8.21 (d, J=4.4 Hz, 1H), 7.98 (dd, J=8.6, 1.2 Hz, 2H), 7.45-7.55 (m, 2H), 7.38-7.45 (m, 2H), 7.25-7.38 (m, 4H). MS(M+1) 459. gray solid.

### Compound 5-14

### N-(6-(4-fluorophenoxy)-1-phenyl-1H-pyrazolo[3,4-d]pyrimidin-4-yl)-5-nitrothiophene-2-carboxamide

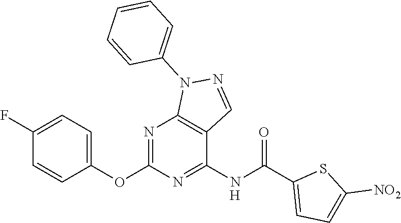

¹H NMR (400 MHz, DMSO-d₆): δ 12.29 (br. s., 1H), 8.56 (s, 1H), 8.33 (d, J=4.4 Hz, 1H), 8.22 (d, J=4.4 Hz, 1H), 7.96-8.00 (m, 2H), 7.37-7.48 (m, 4H), 7.28-7.37 (m, 3H). MS(M+1) 477. gray solid.

### Compound 5-15

### 5-nitro-N-(1-phenyl-6-(phenylthio)-1H-pyrazolo[3,4-d]pyrimidin-4-yl)thiophene-2-carboxamide

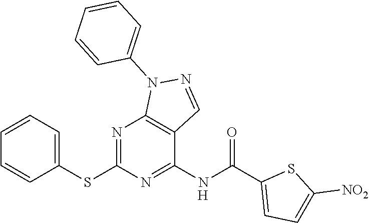

¹H NMR (400 MHz, DMSO-d₆): δ 12.27 (s, 1H), 8.5) (s, 1H), 8.33 (d, J=4.4 Hz, 1H), 8.19 (d, J=4.4 Hz, 1H), 7.81-7.91 (m, 2H), 7.69-7.81 (m, 2H), 7.53-7.69 (m, 3H), 7.18-7.39 (m, 3H).

MS(M+1): 475. Yellow-brown solid.

### Compound 5-16

### N-(6-((4-chlorophenyl)thio)-1-phenyl-1H-pyrazolo[3,4-d]pyridin-4-yl)-5-nitrothiophene-2-carboxamide

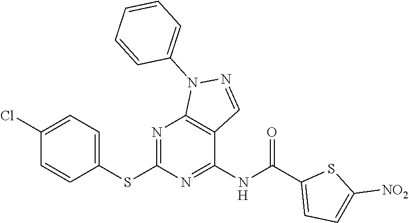

¹H NMR (400 MHz, DMSO-d₆): δ 12.44 (s, 1H), 8.47 (s, 1H), 8.04-8.21 (m, 2H), 7.82-7.99 (m, 2H), 7.68-7.81 (m, 2H), 7.56-7.68 (m, 2H), 7.22-7.43 (m, 3H)

MS(M+1): 509. Yellow solid.

### Compound 5-17

### N-(6-(furan-2-yl)-1-phenyl-1H-pyrazolo[3,4-d]pyrimidin-4-yl)-5-nitrothiophene-2-carboxamide

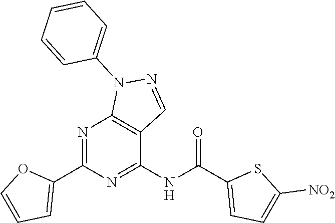

¹H NMR (400 MHz, DMSO-d₆): δ 12.20 (br. s., 1H), 8.57 (s, 1H), 8.38 (d, J=4.4 Hz, 1H), 8.19-8.32 (m, 3H), 8.01 (d, J=1.0 Hz, 1H), 7.58-7.68 (m, 2H), 7.47 (d, J=2.9 Hz, 1H), 7.39-7.46 (m, 1H), 6.77 (dd, J=3.4, 2.0 Hz, 1H). MS(M+1): 433. Light khaki solid.

### Compound 5-18

### 5-nitro-N-(1-phenyl-6-(thiophen-2-yl)-1H-pyrazolo[3,4-d]pyrimidin-4-yl)thiophene-2-carboxamide

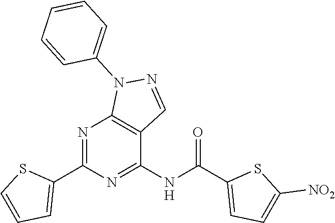

¹H NMR (400 MHz, DMSO-d₆): δ 12.07 (br. s., 1H), 8.57 (s, 1H), 8.38 (d, J=4.4 Hz, 1H), 8.25-8.30 (m, 2H), 8.24 (d, J=4.4 Hz, 1H), 8.11 (dd, J=3.7, 1.2 Hz, 1H), 7.84 (dd, J=4.9, 1.5 Hz, 1H), 7.59-7.67 (m, 2H), 7.39-7.45 (m, 1H), 7.27 (dd, J=5.1, 3.7 Hz, 1H). MS(M+1): 449. Light khaki solid.

### Compound 5-19

### N-(6-(5-chlorothiophen-2-yl)-1-phenyl-1H-pyrazolo[3,4-d]pyrimidin-4-yl)-5-nitrothiophene-2-carboxamide

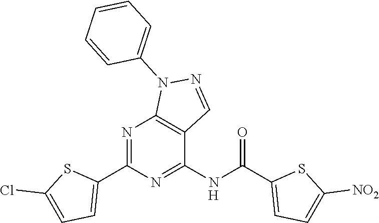

¹H NMR (400 MHz, DMSO-d₆): δ 12.07 (br. s., 1H), 8.58 (s, 1H), 8.36 (d, J=4.4 Hz, 1H), 8.22-8.28 (m, 3H), 7.93 (d, J=3.9 Hz, 1H), 7.60-7.66 (m, 2H), 7.38-7.45 (m, 1H), 7.29 (d, J=4.4 Hz, 1H). MS(M+1): 483. Yellow solid.

### Compound 5-20

### 5-nitro-N-(1-phenyl-6-(thiophen-3-yl)-1H-pyrazolo[3,4-d]pyrimidin-4-yl)thiophene-2-carboxamide

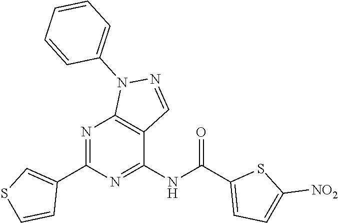 [SMILES: O=C(Nc1nc(-c2ccsc2)nc2c1cnn2-c1ccccc1)c1ccc([N+](=O)[O-])s1]

¹H NMR (400 MHz, DMSO-d₆): δ 12.01 (br. s., 1H), 8.57 (s, 1H), 8.51 (dd, J=2.9, 1.0 Hz, 1H), 8.37 (d, J=4.4 Hz, 1H), 8.28-8.33 (m, 2H), 8.23 (d, J=4.4 Hz, 1H), 7.94 (dd, J=4.9, 1.0 Hz. 1H), 7.71 (dd, J=5.1, 3.2 Hz, 1H), 7.59-7.67 (m, 2H), 7.37-7.45 (m, 1H). MS(M+1) 449. Yellow-brown solid.

### Compound 5-21

### N-(6-(3,5-dimethylisoxazol-4-yl)-1-phenyl-1H-pyrazolo[3,4-d]pyrimidin-4-yl)-5-nitrothiophene-2-carboxamide

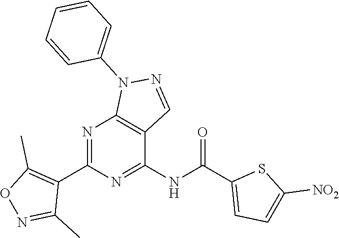 [SMILES: Cc1noc(C)c1-c1nc(NC(=O)c2ccc([N+](=O)[O-])s2)c2cnn(-c3ccccc3)c2n1]

¹H NMR (400 MHz, DMSO-d₆): δ 11.78 (s, 1H), 8.61 (s, 1H), 8.29-8.33 (m, 1H), 8.25-8.29 (m, 1H), 8.18 (d, J=8.3 Hz, 2H), 7.61 (1, J=7.8 Hz, 2H), 7.38-7.47 (m, 1H), 2.90 (s, 3H), 2.63 (s, 3H). MS(M+1) 462. Brown solid.

### Compound 5-22

### 5-nitro-N-(1-phenyl-6-(3-(trifluoromethyl)-1H-pyrazol-1-yl)-1H-pyrazolo[3,4-d]pyrimidin-4-yl)thiophene-2-carboxamide

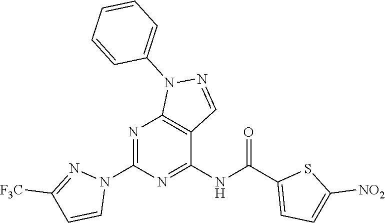 [SMILES: O=C(Nc1nc(-n2ccc(C(F)(F)F)n2)nc2c1cnn2-c1ccccc1)c1ccc([N+](=O)[O-])s1]

¹H NMR (400 MHz, DMSO-d₆): δ 12.40 (s, 1H), 8.90-8.94 (m, 1H), 8.61 (s, 1H), 8.37 (d, J=4.4 Hz, 1H), 8.21 (d, J=4.4 Hz, 1H), 8.18 (dd, J=8.6, 1.2 Hz, 2H), 7.57-7.65 (m, 2H), 7.39-7.46 (m, 1H), 7.12 (d, J=2.9 Hz, 1H). MS(M+1): 501. Light Yellow solid.

### Compound 5-23

### N-(1,6-diphenyl-1H-pyrazolo[3,4-d]pyrimidin-4-yl)-5-nitrothiophene-2-carboxamide

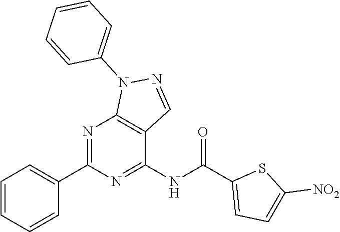 [SMILES: O=C(Nc1nc(-c2ccccc2)nc2c1cnn2-c1ccccc1)c1ccc([N+](=O)[O-])s1]

¹H NMR (400 MHz, DMSO-d₆): δ 12.00 (br s., 1H), 8.61 (s, 1H), 8.50-8.57 (m, 2H), 8.37 (d, J=4.4 Hz, 1H), 8.29 (dd, J=8.8, 1.0 Hz, 2H), 8.23 (d, J=4.4 Hz, 1H), 7.61-7.67 (m, 2H), 7.54-7.61 (m, 3H), 7.38-7.45 (m, 1H). MS(M+1): 443. Yellow solid.

### Compound 5-24

### 5-nitro-N-(1-phenyl-6-(pyrimidin-5-yl)-1H-pyrazolo[3,4-d]pyrimidin-4-yl)thiophene-2-carboxamide

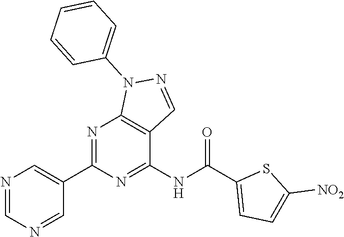 [SMILES: O=C(Nc1nc(-c2cncnc2)nc2c1cnn2-c1ccccc1)c1ccc([N+](=O)[O-])s1]

¹H NMR (400 MHz, DMSO-d₆): δ 12.10 (br. s., 1H), 9.72 (s, 2H), 9.37 (s, 1H), 8.67 (s, 1H), 8.36 (d, J=4.4 Hz, 1H), 8.22-8.33 (m, 3H), 7.57-7.72 (m, 2H), 7.36-7.50 (m, 1H). MS(M+1): 445. Light khaki solid.

### Compound 5-25

### N-(6-(2-fluorophenyl)-1-phenyl-1H-pyrazolo[3,4-d]pyrimidin-4-yl)-5-nitrothiophene-2-carboxamide

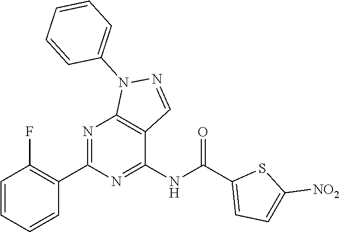 [SMILES: O=C(Nc1nc(-c2ccccc2F)nc2c1cnn2-c1ccccc1)c1ccc([N+](=O)[O-])s1]

¹H NMR (400 MHz, DMSO-d₆): δ 12.21 (br. s., 1H), 8.66 (s, 1H), 8.38 (d, J=3.9 Hz, 1H), 8.30 (d, J=7.8 Hz, 2H), 8.22-8.25 (m, 1H), 8.18-8.22 (m, 1H), 7.6 (t, J=8.1 Hz, 3H), 7.40 (t, J=7.6 Hz, 3H). MS(M+1): 461.

### Compound 5-26

### N-(6-(3-fluorophenyl)-1-phenyl-1H-pyrazolo[3,4-d]pyrimidin-4-yl)-5-nitrothiophene-2-carboxamide

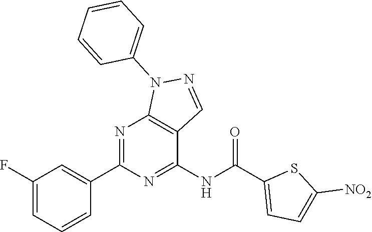 [SMILES: O=C(Nc1nc(-c2cccc(F)c2)nc2c1cnn2-c1ccccc1)c1ccc([N+](=O)[O-])s1]

¹H NMR (400 MHz, DMSO-d₆): δ 11.99 (s, 1H), 8.64 (s, 1H), 8.34-8.40 (m, 2H), 8.21-8.30 (m, 4H), 7.59-7.68 (m, 3H), 7.39-7.46 (m, 2H). MS(M+1): 461.

### Compound 5-27

### N-(6-(4-fluorophenyl)-1-phenyl-1H-pyrazolo[3,4-d]pyridin-4-yl)-5-nitrothiophene-2-carboxamide

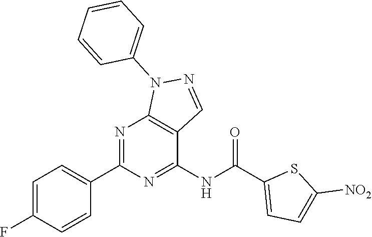 [SMILES: O=C(Nc1nc(-c2ccc(F)cc2)nc2c1cnn2-c1ccccc1)c1ccc([N+](=O)[O-])s1]

¹H NMR (400 MHz, DMSO-d₆): δ 11.95 (s, 1H), 8.59 (s, 1H), 8.55 (dd, J=8.8, 5.9 Hz, 2H), 8.36 (d, J=4.4 Hz, 1H), 8.20-8.28 (m, 3H), 7.62 (t, J=7.8 Hz, 2H), 7.35-7.45 (m, 3H), MS(M+1): 461.

### Compound 5-28

### N-(6-(6-fluoropyridin-3-yl)-1-phenyl-1H-pyrazolo[3,4-d]pyrimidin-4-yl)-5-nitrothiophene-2-carboxamide

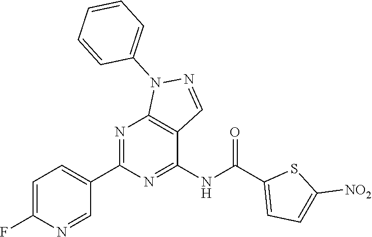 [SMILES: O=C(Nc1nc(-c2ccc(F)nc2)nc2c1cnn2-c1ccccc1)c1ccc([N+](=O)[O-])s1]

¹H NMR (400 MHz, DMSO-d₆): δ 12.06 (br. s., 1H), 9.30 (d, J=2.4 Hz, 1H), 8.93 (td, J=8.3, 2.4 Hz, 1H), 8.65 (s, 1H), 8.36 (d, J=4.4 Hz, 1H), 8.21-8.34 (m, 3H), 7.55-7.70 (m, 2H), 7.35-7.48 (m, 2H). MS(M+1): 462. Yellow solid.

### Compound 5-29

### N-(6-(3-chlorophenyl)-1-phenyl-1H-pyrazolo[3,4-d]pyrimidin-4-yl)-5-nitrothiophene-2-carboxamide

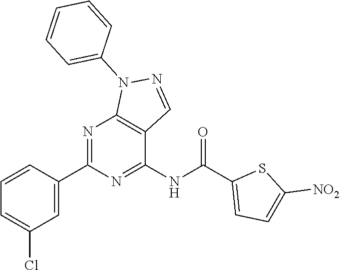 [SMILES: O=C(Nc1nc(-c2cccc(Cl)c2)nc2c1cnn2-c1ccccc1)c1ccc([N+](=O)[O-])s1]

¹H NMR (400 MHz, DMSO-d₆): δ 12.04 (s, 1H), 8.66 (s, 1H), 8.50-8.56 (m, 1H), 8.48 (dt, J=7.0, 1.9 Hz, 1H), 8.39 (d, J=4.4 Hz, 1H), 8.21-8.32 (m, 3H), 7.55-7.75 (m, 4H), 7.32-7.51 (m, 1H), MS(M+1): 477. Yellow solid.

### Compound 5-30

### N-(6-(4-chlorophenyl)-1-phenyl-1H-pyrazolo[3,4-d]pyrimidin-4-yl)-5-nitrothiophene-2-carboxamide

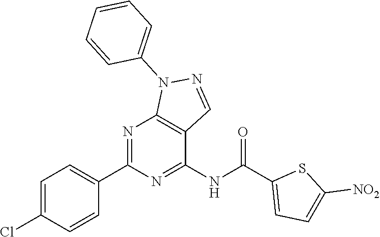 [SMILES: O=C(Nc1nc(-c2ccc(Cl)cc2)nc2c1cnn2-c1ccccc1)c1ccc([N+](=O)[O-])s1]

¹H NMR (400 MHz, DMSO-d₆): δ 12.06 (br. s, 1H), 8.64 (s, 1H), 8.51-8.58 (m, 2H), 8.37 (d, J=4.4 Hz, 1H), 8.25-8.31 (m, 3H), 7.61-7.71 (m, 4H), 7.38-7.49 (m, 1H)

MS(M+1): 474.

### Compound 5-31

### N-(6-(6-chloropyridin-3-yl)-1-phenyl-1H-pyrazolo[3,4-d]pyrimidin-4-yl)-5-nitrothiophene-2-carboxamide

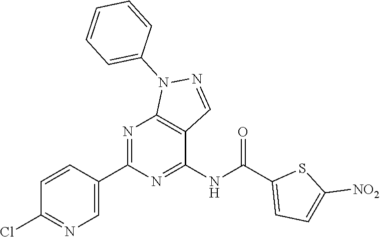 [SMILES: O=C(Nc1nc(-c2ccc(Cl)nc2)nc2c1cnn2-c1ccccc1)c1ccc([N+](=O)[O-])s1]

¹H NMR (400 MHz, DMSO-d₆): δ 12.13 (s, 1H), 9.49 (d, J=2.4 Hz, 1H), 8.83 (dd, J=8.3, 2.4 Hz, 1H), 8.70 (s, 1H), 8.40 (d, J=4.4 Hz, 1H), 8.25-8.32 (m, 3H), 7.79 (d, J=8.3 Hz, 1H), 7.64-7.69 (m, 2H), 7.42-7.48 (m, 1H). MS(M+1): 478 Orange solid.

### Compound 5-32

### N-(6-(4-cyanophenyl)-1-phenyl-1H-pyrazolo[3,4-d]pyrimidin-4-yl)-5-nitrothiophene-2-carboxamide

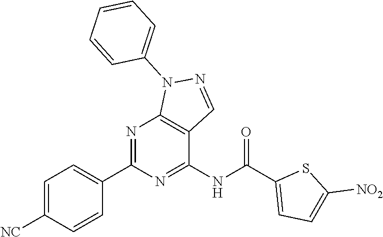 [SMILES: N#Cc1ccc(-c2nc(NC(=O)c3ccc([N+](=O)[O-])s3)c3cnn(-c4ccccc4)c3n2)cc1]

¹H NMR (400 MHz, DMSO-d₆): δ 12.07 (br. s., 1H), 8.62-8.69 (m, 3H), 8.36 (d, J=4.4 Hz, 1H), 8.21-8.28 (m, 3H), 8.01-8.09 (m, 2H), 7.61-7.68 (m, 2H), 7.41-7.48 (m, 1H). MS(M+1): 468. Light khaki solid.

### Compound 5-33

### N-(6-(6-cyanopyridin-3-yl)-1-phenyl-1H-pyrazolo[3,4-d]pyrimidin-4-yl)-5-nitrothiophene-2-carboxamide

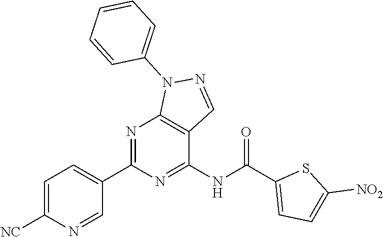 [SMILES: N#Cc1ccc(-c2nc(NC(=O)c3ccc([N+](=O)[O-])s3)c3cnn(-c4ccccc4)c3n2)cn1]

¹H NMR (400 MHz, DMSO-d₆): δ 12.0) (br. s., 1H), 9.66 (d, J=1.5 Hz, 1H), 8.83-8.91 (m, 1H), 8.61 (s, 1H), 8.33 (d, J=4.4 Hz, 1H), 8.17-8.25 (m, 4H), 7.55-7.64 (m, 2H), 7.36-7.45 (m, 1H). MS(M+1): 469. Red solid.

### Compound 5-34

### N-(6-(6-methylpyridin-3-yl)-1-phenyl-1H-pyrazolo[3,4-d]pyrimidin-4-yl)-5-nitrothiophene-2-carboxamide

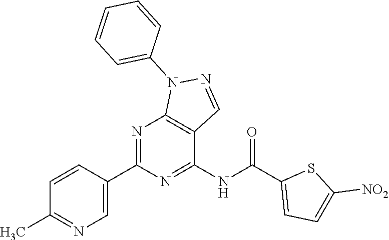 [SMILES: Cc1ccc(-c2nc(NC(=O)c3ccc([N+](=O)[O-])s3)c3cnn(-c4ccccc4)c3n2)cn1]

¹H NMR (400 MHz, DMSO-d₆): δ 12.06 (br. s., 1H), 9.54 (d, J=2.0 Hz, 1H), 8.59-8.70 (m, 2H), 8.37 (d, J=4.4 Hz, 1H), 8.16-8.34 (m, 3H), 7.64 (t, J=8.1 Hz, 2H), 7.30-7.54 (m, 2H), 2.52-2.61 (m, 3H). MS(M+1): 458. Dark orange solid.

### Compound 5-35

### N-(6-(6-methoxypyridin-3-yl)-1-phenyl-1H-pyrazolo[3,4-d]pyrimidin-4-yl)-5-nitrothiophene-2-carboxamide

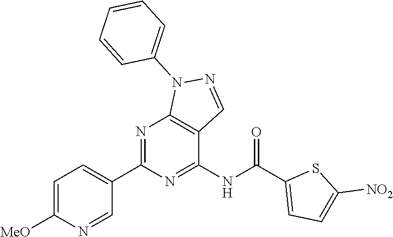 [SMILES: COc1ccc(-c2nc(NC(=O)c3ccc([N+](=O)[O-])s3)c3cnn(-c4ccccc4)c3n2)cn1]

¹H NMR (400 MHz, DMSO-d₆): δ 11.87 (s, 1H), 9.22 (d, J=2.4 Hz, 1H), 8.54-8.64 (m, 2H), 8.26-8.20 (m, 4H), 7.60 (t, J=7.8 Hz, 2H), 7.39 (t, J=7.8 Hz, 1H), 6.96 (d, J=8.8 Hz, 1H), 3.94 (s, 3H). MS(M+1): 474. Yellow solid.

### Compound 5-36

### N-(6-(4-methoxyphenyl)-1-phenyl-1H-pyrazolo[3,4-d]pyrimidin-4-yl)-5-nitrothiophene-2-carboxamide

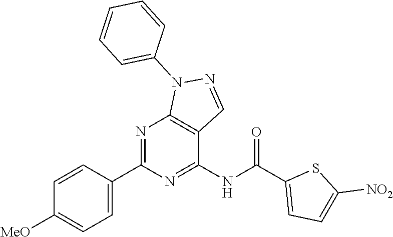 [SMILES: COc1ccc(-c2nc(NC(=O)c3ccc([N+](=O)[O-])s3)c3cnn(-c4ccccc4)c3n2)cc1]

¹H NMR (400 MHz, DMSO-d₆): δ 11.98 (br. s., 1H), 8.60 (s, 1H), 8.40-8.58 (m, J=8.8 Hz, 2H), 8.38 (d, J=4.4 Hz, 1H), 8.30 (dd, J=8.8, 1.0 Hz, 2H), 8.26 (d, J=4.4 Hz, 1H), 7.58-7.70 (m, 2H), 7.38-7.48 (m, 1H), 7.08-7.20 (m, 2H), 3.87 (s, 3H)

MS(M+1): 473. Yellow solid.

### Compound 5-37

### N-(6-(4-(tert-butyl)phenyl)-1-phenyl-1H-pyrazolo[3,4-d]pyrimidin-4-yl)-5-nitrothiophene-2-carboxamide

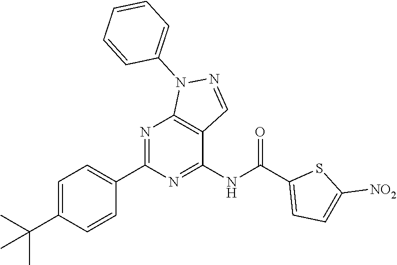 [SMILES: CC(C)(C)c1ccc(-c2nc(NC(=O)c3ccc([N+](=O)[O-])s3)c3cnn(-c4ccccc4)c3n2)cc1]

¹H NMR (400 MHz, DMSO-d₆): δ 12.04 (br. s., 1H), 8.62 (s, 1H), 8.48 (d, J=8.8 Hz, 2H), 8.40 (d, J=4.4 Hz, 1H), 8.32 (d, J=7.8 Hz, 2H), 8.26 (d, J=4.4 Hz, 1H), 7.58-7.68 (m, 4H), 7.40-7.47 (m, 1H), 1.35 (s, 9H). MS (M+1): 499. Yellow solid.

### Compound 5-38

### 5-nitro-N-(1-phenyl-6-(3-(trifluoromethyl)phenyl)-1H-pyrazolo[3,4-d]pyrimidin-4-yl)thiophene-2-carboxamide

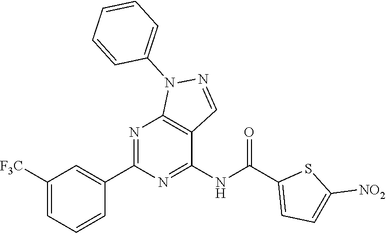 [SMILES: O=C(Nc1nc(-c2cccc(C(F)(F)F)c2)nc2c1cnn2-c1ccccc1)c1ccc([N+](=O)[O-])s1]

¹H NMR (400 MHz, DMSO-d₆): δ 12.07 (br. s., 1H), 8.77-8.85 (m, 2H), 8.66 (s, 1H), 8.38 (d, J=4.4 Hz, 1H), 8.23-8.31 (m, 3H), 7.% (d, J=7.8 Hz, 1H), 7.85 (t, J=7.8 Hz, 1H), 7.65 (t, J=8.1 Hz, 2H), 7.41-7.48 (m, 1H). MS(M+1): 511. Yellow solid.

### Compound 5-39

### 5-nitro-N-(1-phenyl-6-(4-(trifluoromethyl)phenyl)-1H-pyrazolo[3,4-d]pyrimidin-4-yl)thiophene-2-carboxamide

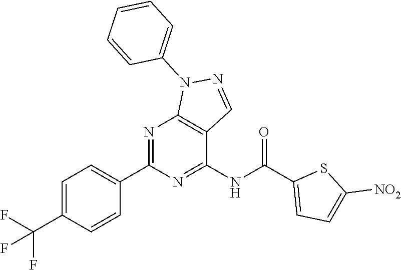 [SMILES: O=C(Nc1nc(-c2ccc(C(F)(F)F)cc2)nc2c1cnn2-c1ccccc1)c1ccc([N+](=O)[O-])s1]

¹H NMR (400 MHz, DMSO-d₆): δ 12.05 (s, 1H), 8.64-8.70 (m, J=7.8 Hz, 2H), 8.61 (s, 1H), 8.37 (d, J=4.4 Hz, 1H), 8.21-8.27 (m, 3H), 7.90-7.96 (m, J=8.3 Hz, 2H), 7.58-7.66 (m, 2H), 7.40-7.46 (m, 1H). MS (M+1): 511. Pale yellow solid.

### Compound 5-40

### 5-nitro-N-(1-phenyl-6-(4-(trifluoromethoxy)phenyl)-1H-pyrazolo[3,4-d]pyrimidin-4-yl)thiophene-2-carboxamide

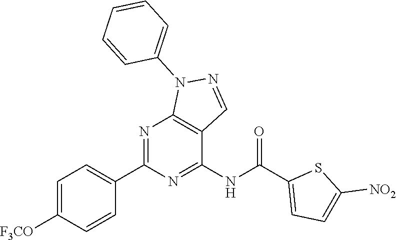 [SMILES: O=C(Nc1nc(-c2ccc(OC(F)(F)F)cc2)nc2c1cnn2-c1ccccc1)c1ccc([N+](=O)[O-])s1]

¹H NMR (400 MHz, DMSO-d₆): δ 12.08 (s, 1H), 8.60-8.68 (m, 3H), 8.38 (d, J=4.4 Hz, 1H), 8.23-8.32 (m, 3H), 7.61-7.68 (m, 2H), 7.59 (d, J=8.3 Hz, 2H), 7.40-7.48 (m, 1H), MS (M+1): 527. Pale yellow solid.

### Compound 5-41

### N-(6-(3-(dimethylamino)phenyl)-1-phenyl-1H-pyrazolo[3,4-d]pyrimidin-4-yl)-5-nitrothiophene-2-carboxamide

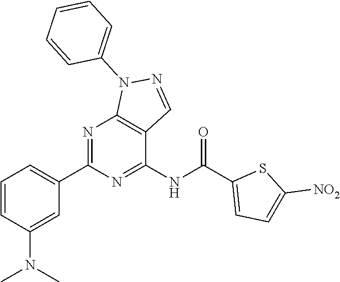 [SMILES: CN(C)c1cccc(-c2nc(NC(=O)c3ccc([N+](=O)[O-])s3)c3cnn(-c4ccccc4)c3n2)c1]

¹H NMR (400 MHz, DMSO-d₆): δ 11.95 (br. s., 1H), 8.60 (br. s., 1H), 8.27-8.48 (m, 3H), 8.23 (br. s., 1H), 7.94 (br. s., 1H), 7.86 (d, J=7.3 Hz, 1H), 7.63 (t, J=7.3 Hz, 2H), 7.30-7.53 (m, 2H), 6.94 (d, J=8.3 Hz, 1H), 3.02 (s, 6H). MS(M+1): 486. Brick red solid.

### Compound 5-42

### 5-nitro-N-(1-phenyl-6-(6-(piperidin-1-yl)pyridin-3-yl)-1H-pyrazolo[3,4-d]pyrimidin-4-yl)thiophene-2-carboxamide

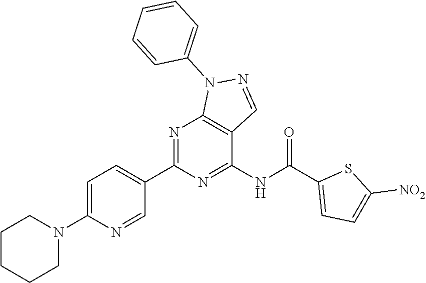 [SMILES: O=C(Nc1nc(-c2ccc(N3CCCCC3)nc2)nc2c1cnn2-c1ccccc1)c1ccc([N+](=O)[O-])s1]

¹H NMR (400 MHz, DMSO-d₆): δ 11.92 (br. s., 1H), 9.23 (d, J=2.0 Hz, 1H), 8.55 (s, 1H), 8.49 (dd, J=9.3, 2.4 Hz, 1H), 8.35 (d, J=4.4 Hz, 1H), 8.31 (d, J=7.3 Hz, 2H), 8.25 (d, J=4.4 Hz, 1H), 7.59-7.67 (m, 2H), 7.36-7.44 (m, 1H), 6.97 (d, J=8.8 Hz, 1H), 3.61-3.72 (m, 4H), 1.65 (d, J=4.9 Hz, 2H), 1.58 (d, J=3.9 Hz, 4H). MS(M+1): 527. Yellow solid.

### Compound 5-43

### N-(6-(6-morpholinopyridin-3-yl)-1-phenyl-1H-pyrazolo[3,4-d]pyrimidin-4-yl)-5-nitrothiophene-2-carboxamide

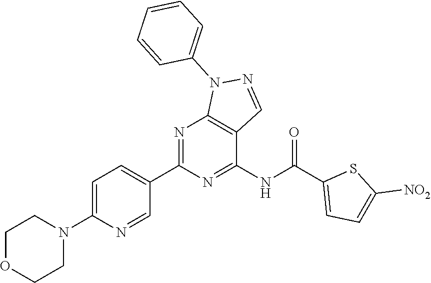 [SMILES: O=C(Nc1nc(-c2ccc(N3CCOCC3)nc2)nc2c1cnn2-c1ccccc1)c1ccc([N+](=O)[O-])s1]

¹H NMR (400 MHz, DMSO-d₆): δ 11.82 (br. s., 1H), 9.22 (d, J=2.0 Hz, 1H), 8.52 (s, 1H), 8.50 (dd, J=9.3, 2.4 Hz, 1H), 8.34 (d, J=4.4 Hz, 1H), 8.27 (dd, J=8.6, 1.2 Hz, 2H), 8.21 (d, J=4.4 Hz, 1H), 7.57-7.64 (m, 2H), 7.35-7.42 (m, 1H), 6.95 (d, J=9.3 Hz, 1H), 3.68-3.75 (m, 4H), 3.55-3.64 (m, 4H). MS(M+1): 529. Yellow green solid.

### Compound 5-44

### N-(6-(3,4-difluorophenyl)-1-phenyl-1H-pyrazolo[3,4-d]pyrimidin-4-yl)-5-nitrothiophene-2-carboxamide

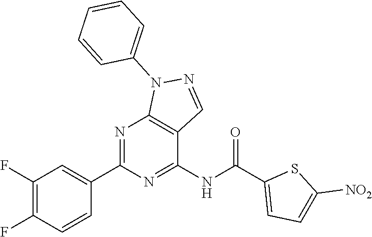 [SMILES: O=C(Nc1nc(-c2ccc(F)c(F)c2)nc2c1cnn2-c1ccccc1)c1ccc([N+](=O)[O-])s1]

¹H NMR (400 MHz, DMSO-d₆): δ 11.96 (br. s., 1H), 8.63 (s, 1H), 8.41 (ddd, J=12.0, 8.1, 2.0 Hz, 1H), 8.32-8.38 (m, 2H), 8.21-8.30 (m, 3H), 7.60-7.69 (m, 3H), 7.39-7.46 (m, 1H) MS(M+1): 479. Yellow solid.

### Compound 5-45

### N-(6-(2,4-difluorophenyl)-1-phenyl-1H-pyrazolo[3,4-d]pyrimidin-4-yl)-5-nitrothiophene-2-carboxamide

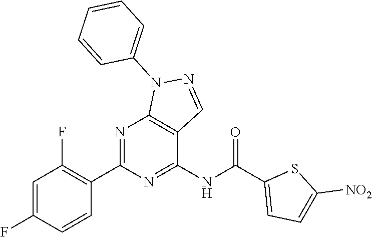 [SMILES: O=C(Nc1nc(-c2ccc(F)cc2F)nc2c1cnn2-c1ccccc1)c1ccc([N+](=O)[O-])s1]

¹H NMR (400 MHz, DMSO-d₆): δ 12.13 (br. s., 1H), 8.64 (s, 1H), 8.33-8.42 (m, 1H), 8.24-8.33 (m, 3H), 8.22 (d, J=4.4 Hz, 1H), 7.54-7.66 (m, 2H), 7.36-7.49 (m, 2H), 7.23-7.36 (m, 1H). MS(M+1): 479. Yellow solid.

### Compound 5-46

### N-(6-(4-chloro-3-fluorophenyl)-1-phenyl-1H-pyrazolo[3,4-d]pyrimidin-4-yl)-5-nitrothiophene-2-carboxamide

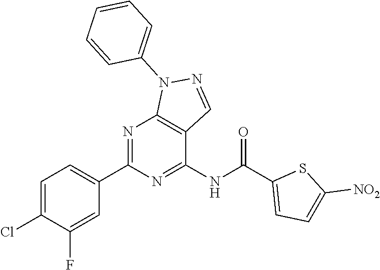 [SMILES: O=C(Nc1nc(-c2ccc(Cl)c(F)c2)nc2c1cnn2-c1ccccc1)c1ccc([N+](=O)[O-])s1]

¹H NMR (400 MHz, DMSO-d₆): δ 11.97 (br. s., 1H), 8.63 (s, 1H), 8.28-8.42 (m, 4H), 8.19-8.28 (m, 3H), 7.80 (t, J=7.8 Hz, 1H), 7.57-7.69 (m, 2H), 7.37-7.48 (m, 1H).

MS(M+1) 495. Yellow solid.

### Compound 5-47

### N-(6-(4-chloro-2-fluorophenyl)-1-phenyl-1H-pyrazolo[3,4-d]pyrimidin-4-yl)-5-nitrothiophene-2-carboxamide

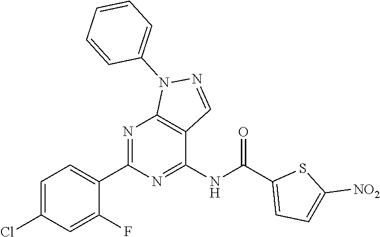 [SMILES: O=C(Nc1nc(-c2ccc(Cl)cc2F)nc2c1cnn2-c1ccccc1)c1ccc([N+](=O)[O-])s1]

¹H NMR (DMSO-d₆): δ 12.11 (br. s., 1H), 8.62 (d, J=1.0 Hz, 1H), 8.34 (d, J=4.4 Hz, 1H), 8.23-8.31 (m, 3H), 8.21 (d, J=4.4 Hz, 1H), 7.55-7.65 (m, 3H), 7.46-7.52 (m, 1H), 7.36-7.43 (m, 1H). MS(M+1): 495. Yellow-brown solid.

### Compound 5-48

### N-(6-(3,4-dichlorophenyl)-1-phenyl-1H-pyrazolo[3,4,d]pyrimidin-4-yl)-5-nitrothiophene-2-carboxamide

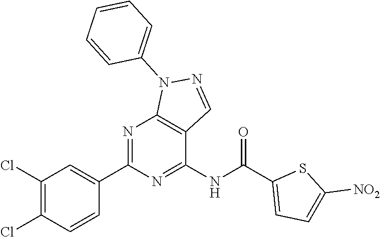 [SMILES: O=C(Nc1nc(-c2ccc(Cl)c(Cl)c2)nc2c1cnn2-c1ccccc1)c1ccc([N+](=O)[O-])s1]

¹H NMR (400 MHz, DMSO-d₆): δ 11.96 (br. s., 1H), 8.60 (d, J=2.0 Hz, 1H), 8.59 (s, 1H), 8.36-8.39 (m, 1H), 8.35 (d, J=4.4 Hz, 1H), 8.23 (d, J=4.4 Hz, 1H), 8.16-8.21 (m, 2H), 7.80 (d, J=8.8 Hz, 1H), 7.57-7.64 (m, 2H), 7.38-7.44 (m, 1H) MS(M+1): 511. Brown solid.

### Compound 5-49

### N-(6-(2-(4-dichlorophenyl)-1-phenyl-1H-pyrazolo[3,4-d]pyrimidin-4-yl)-5-nitrothiophene-2-carboxamide

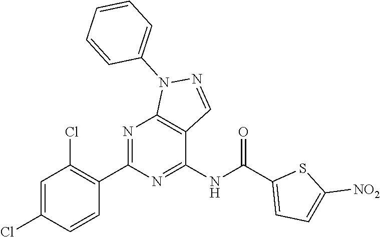 [SMILES: O=C(Nc1nc(-c2ccc(Cl)cc2Cl)nc2c1cnn2-c1ccccc1)c1ccc([N+](=O)[O-])s1]

¹H NMR (400 MHz, DMSO-d₆): δ 12.32 (br. s., 1H), 8.69 (s, 1H), 8.36 (d, J=4.4 Hz, 1H), 8.19-8.26 (m, 3H), 7.94 (d, J=8.3 Hz, 1H), 7.82 (d, J=2.0 Hz, 1H), 7.55-7.66 (m, 3H), 7.37-7.44 (m, 1H). MS(M+1): 512. Yellow solid.

### Compound 5-50

### N-(6-(2-fluoro-5-(trifluoromethyl)phenyl)-1-phenyl-1H-pyrazolo[3,4-d]pyrimidin-4-yl)-5-nitrothiophene-2-carboxamide

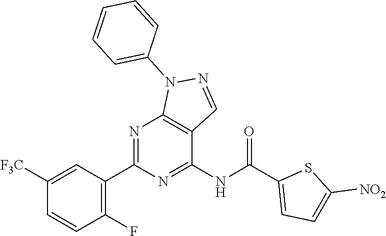 [SMILES: O=C(Nc1nc(-c2cc(C(F)(F)F)ccc2F)nc2c1cnn2-c1ccccc1)c1ccc([N+](=O)[O-])s1]

¹H NMR (400 MHz, DMSO-d₆): δ 12.20 (br. s., 1H), 8.69 (s, 1H), 8.62 (dd, J=6.8, 2.4 Hz, 1H), 8.36 (d, J=4.4 Hz, 1H), 8.30 (dd, J=8.6, 1.2 Hz, 2H), 8.25 (d, J=4.4 Hz, 1H), 7.98-8.08 (m, 1H), 7.65-7.73 (m, 1H), 7.58-7.65 (m, 2H), 7.39-7.45 (m, 1H)

MS(M+1): 529. Pale yellow solid.

### Compound 5-51

### N-(6-(2-fluoro-4-(trifluoromethyl)phenyl)-1-phenyl-1H-pyrazolo[3,4-d]pyrimidin-4-yl)-5-nitrothiophene-2-carboxamide

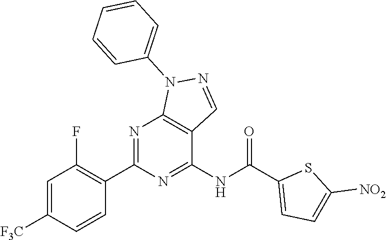 [SMILES: O=C(Nc1nc(-c2ccc(C(F)(F)F)cc2F)nc2c1cnn2-c1ccccc1)c1ccc([N+](=O)[O-])s1]

¹H NMR (400 MHz, DMSO-d₆): δ 12.16 (br. s., 1H), 8.60 (s, 1H), 8.28-8.46 (m, 2H), 8.13-8.28 (m, 3H), 7.85 (d, J=10.8 Hz, 1H), 7.76 (d, J=8.3 Hz, 1H), 7.50-7.63 (m, 2H), 7.32-7.43 (m, 1H). MS(M+1): 529. Light khaki solid.

### Compound 5-52

### N-(6-(3-fluoro-4-(trifluoromethyl)phenyl)-1-phenyl-1H-pyrazolo[3,4-d]pyrimidin-4-yl)-5-nitrothiophene-2-carboxamide

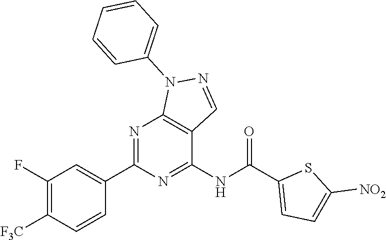 [SMILES: O=C(Nc1nc(-c2ccc(C(F)(F)F)c(F)c2)nc2c1cnn2-c1ccccc1)c1ccc([N+](=O)[O-])s1]

¹H NMR (400 MHz, DMSO-d₆): δ 12.10 (br. s., 1H), 8.69 (s, 1H), 8.41-8.55 (m, 2H), 8.35 (d, J=4.4 Hz, 1H), 8.21-8.31 (m, 3H), 8.04 (t, J=8.1 Hz, 1H), 7.61-7.72 (m, 2H), 7.41-7.51 (m, 1H). MS(M+1): 529. Yellow solid.

### Compound 5-53

### 5-nitro-N-(1-phenyl-6-(3,4,5-trifluorophenyl)-1H-pyrazolo[3,4-d]pyrimidin-4-yl)thiophene-2-carboxamide

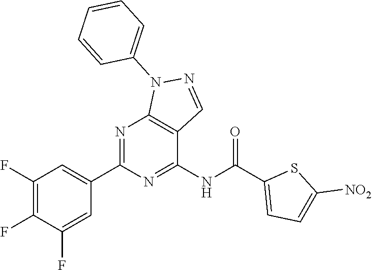 [SMILES: O=C(Nc1nc(-c2cc(F)c(F)c(F)c2)nc2c1cnn2-c1ccccc1)c1ccc([N+](=O)[O-])s1]

¹H NMR (DMSO-d₆): δ 11.79 (br. s., 1H), 8.56 (s, 1H), 8.30 (d, J=4.4 Hz, 1H), 8.22 (d, J=4.4 Hz, 1H), 8.10-8.21 (m, 4H), 7.59 (t, J=7.8 Hz, 2H), 7.35-7.43 (m, 1H)

MS(M+1): 497. Yellow-brown solid.

### Compound 5-54

### N-(6-(6(2-methoxyethoxy)pyridin-3-yl)-1-phenyl-1H-pyrazolo[3,4-d]pyrimidin-4-yl)-5-nitrothiophene-2-carboxamide

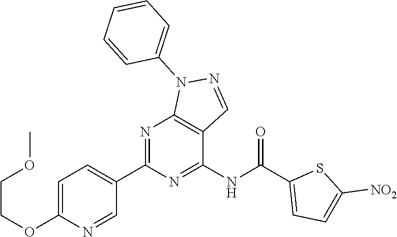 [SMILES: COCCOc1ccc(-c2nc(NC(=O)c3ccc([N+](=O)[O-])s3)c3cnn(-c4ccccc4)c3n2)cn1]

¹H NMR (400 MHz, DMSO-d₆): δ 11.96 (br. s., 1H), 9.26 (d, J=2.4 Hz, 1H), 8.68 (dd, J=8.8, 2.4 Hz, 1H), 8.60 (s, 1H), 8.36 (d, J=4.4 Hz, 1H), 8.28 (d, J=7.3 Hz, 2H), 8.25 (d, J=4.4 Hz, 1H), 7.59-7.68 (m, 2H), 7.37-7.46 (m, 1H), 7.02 (d, J=8.8 Hz, 1H), 4.43-4.52 (m, 2H), 3.70 (dd, J=5.4, 3.9 Hz, 2H). MS(M+1): 518. Yellow solid.

### Compound 5-55

### N-(6-(6-(2-ethoxyethoxy)pyridin-3-yl)-1-phenyl-1H-pyrazolo[3,4-d]pyrimidin-4-yl)-5-nitrothiophene-2-carboxamide

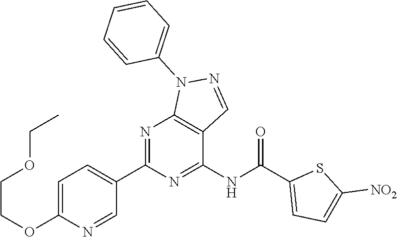 [SMILES: CCOCCOc1ccc(-c2nc(NC(=O)c3ccc([N+](=O)[O-])s3)c3cnn(-c4ccccc4)c3n2)cn1]

¹H NMR (400 MHz, DMSO-d₆): δ 11.84 (br. s., 1H), 9.20 (d, J=2.4 Hz, 1H), 8.62 (dd, J=8.3, 2.4 Hz, 1H), 8.54 (s, 1H), 8.32 (d, J=4.4 Hz, 1H), 8.24 (dd, J=8.8, 1.0 Hz, 2H), 8.20 (d, J=4.4 Hz, 1H), 7.55-7.64 (m, 2H), 7.34-7.42 (m, 1H), 6.97 (d, J=7.8 Hz, 1H), 4.44 (dd, J=5.6, 4.4 Hz, 2H), 3.68-3.77 (m, 2H), 3.51 (q, J=7.0 Hz, 2H), 1.14 (t, J=7.0 Hz, 3H). MS(M+1): 532. Yellow solid.

### Compound 5-56

### 5-nitro-N-(1-phenyl-6-(6-(2,2,3,3-tetrafluoropropoxy)pyridin-3-yl)-1H-pyrazolo[3,4-d]pyrimidin-4-yl)thiophene-2-carboxamide

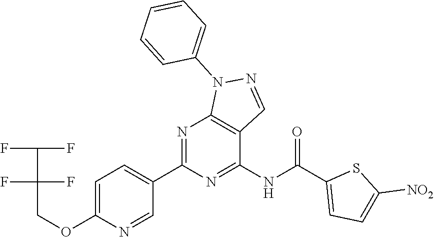 [SMILES: O=C(Nc1nc(-c2ccc(OCC(F)(F)C(F)F)nc2)nc2c1cnn2-c1ccccc1)c1ccc([N+](=O)[O-])s1]

¹H NMR (400 MHz, DMSO-d₆): δ 12.03 (br. s., 1H), 9.31 (d, J=2.4 Hz, 1H), 8.77-8.82 (m, 1H), 8.65 (s, 1H), 8.37 (d, J=4.4 Hz, 1H), 8.28-8.34 (m, 2H), 8.25-8.28 (m, 1H), 7.62-7.70 (m, 2H), 7.40-7.48 (m, 1H), 7.18 (d, J=9.3 Hz, 1H), 6.71 (t, J=5.4 Hz, 1H), 4.98 (t, J=13.9 Hz, 2H). MS(M+1) 574. Yellow solid.

### Compound 5-57

### N-(6-(6-(2-(2-ethoxyethoxy)ethoxy)pyridin-3-yl)-1-phenyl-1H-pyrazolo[3,4-d]pyrimidin-4-yl)-5-nitrothiophene-2-carboxamide

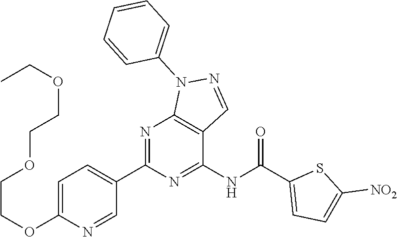 [SMILES: CCOCCOCCOc1ccc(-c2nc(NC(=O)c3ccc([N+](=O)[O-])s3)c3cnn(-c4ccccc4)c3n2)cn1]

¹H NMR (400 MHz, DMSO-d₆): δ 11.88 (br. s., 1H), 9.22 (d, J=2.0 Hz, 1H), 8.64 (dd, J=8.8, 2.4 Hz, 1H), 8.56 (s, 1H), 8.33 (d, J=4.4 Hz, 1H), 8.25 (dd, J=8.8, 1.0 Hz, 2H), 8.21 (d, J=4.4 Hz, 1H), 7.57-7.65 (m, 2H), 7.35-7.43 (m, 1H), 6.99 (d, J=9.3 Hz, 1H), 4.46 (dd, J=5.4, 3.9 Hz, 2H), 3.78 (dd, J=5.4, 3.9 Hz, 2H), 3.57-3.63 (m, 2H), 3.48-3.53 (m, 2H), 3.44 (q, J=6.8 Hz, 2H), 1.05-1.13 (m, 3H). MS(M+1): 576. Beige solid.

TABLE 6

| | 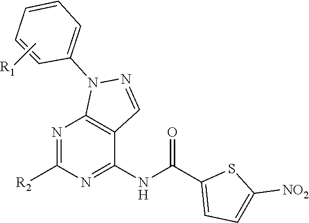 | | |
|---|---|---|---|
| | | | Hep3B |
| | | | LC₅₀ |
| Compound | R₁ | R₂ | (uM) |
| 6-1 | 2-Me | 2-(hydroxymethyl)pyrrolidin-1-yl | >3 |
| 6-2 | 2-Me | 6-fluoropyridin-3-yl | >3 |
| 6-3 | 4-Me | 2-(hydroxymethyl)pyrrolidin-1-yl | 0.87 |
| 6-4 | 4-Me | 4-fluorophenyl | 0.28 |
| 6-5 | 4-Me | 6-fluoropyridin-3-yl | 0.11 |
| 6-6 | 4-Me | 6-methoxypyridin-3-yl | 0.40 |
| 6-7 | 4-Me | 4-chlorophenyl | 0.29 |
| 6-8 | 4-Me | 4-(trifluoromethyl)phenyl | 0.35 |
| 6-9 | 4-Me | benzo[d][1,3]dioxo1-5-yl | 0.40 |
| 6-10 | 4-Me | 2,3-dihydrobenzo[b][1,4]dioxin-6-yl | 0.62 |
| 6-11 | 4-Me | 2,4-difluorophenyl | 0.28 |
| 6-12 | 4-Me | 6-(2-methoxyethoxy)pyridin-3-yl | 0.26 |
| 6-13 | 4-Me | 6-(2-ethoxyethoxy)pyridin-3-yl | 0.51 |
| 6-14 | 4-Me | 6-(2-(2-ethoxyethoxy)ethoxy )pyridin-3-yl | 0.22 |
| 6-15 | 4-OMe | cyclohexylamino | >1.25 |
| 6-16 | 4-OMe | cyclohexyloxy | 1.36 |
| 6-17 | 4-OMe | 6-methylpyridin-3-yl | 0.19 |
| 6-18 | 4-OMe | 4-fluorophenyl | 0.18 |
| 6-19 | 4-OMe | 6-fluoropyridin-3-yl | 0.06 |
| 6-20 | 4-OMe | 4-chloropbenyl | 0.32 |
| 6-21 | 4-OMe | 6-chloropyridin-3-yl | 0.12 |
| 6-22 | 4-OMe | 4-chloro-2-fluorophenyl | 0.38 |
| 6-23 | 4-OMe | 3,4-difluorophenyl | 0.17 |
| 6-24 | 4-OMe | benzo[d][1,3]dioxol-5-yl | 0.19 |
| 6-25 | 4-OMe | 6-propoxypyridin-3-yl | 0.21 |
| 6-26 | 4-OMe | 6-(2-methoxyethoxy)pyridin-3-yl | 0.22 |
| 6-27 | 4-OMe | 6-(2-ethoxyethoxy)pyridin-3-yl | 0.21 |
| 6-28 | 4-OMe | 6-(2-(2-ethoxyethoxy)ethoxy)pyridin-3-yl | 0.19 |
| 6-29 | 4-OMe | 4,4-dimethylcyclohex-1-en-1-yl | 0.82 |
| 6-30 | 4-OMe | 4,4-dimethylcyclohexyl | 1.32 |
| 6-31 | 3-Cl | 2-(hydroxymethyl)pyrrolidin-1-yl | >1.25 |
| 6-32 | 3-Cl | methyl L-prolinate | 0.56 |
| 6-33 | 3-Cl | thiophen-3-yl | 0.29 |
| 6-34 | 3-Cl | 3-fluorophenyl | 0.19 |
| 6-35 | 3-Cl | 4-fluorophenyl | 0.11 |
| 6-36 | 3-Cl | 6-fluoropyridin-3-yl | 0.09 |
| 6-37 | 3-Cl | 3-chloropbenyl | 0.23 |
| 6-38 | 3-Cl | 4-chlorophenyl | 0.38 |
| 6-39 | 3-Cl | benzo[d][1,3]dioxol-5-yl | 0.27 |
| 6-40 | 3-Cl | 3-(trifluoromethoxy)phenyl | 0.48 |
| 6-41 | 3-Cl | 2-fluoro-4-(trifluoromethyl)phenyl | 0.32 |
| 6-42 | 3-Cl | 2,4-bis(trifluoromethyl)phenyl | 0.76 |
| 6-43 | 3-Cl | 6-(2-methoxyethoxy)pyridin-3-yl | 0.09 |
| 6-44 | 3-Cl | 6-(2-ethoxyethoxy)pyridin-3-yl | 0.45 |
| 6-45 | 3-Cl | 6-(2-(2-ethoxyethoxy)ethoxy )pyridin-3-yl | 0.22 |
| 6-46 | 4-Cl | 3-methyl-1H-pyrazol-1-yl | 0.16 |
| 6-47 | 4-Cl | piperidin-1-yl | 0.71 |
| 6-48 | 4-Cl | 4-fluorophenyl | 0.18 |
| 6-49 | 4-Cl | 4-chlorophenyl | 0.17 |
| 6-50 | 4-Cl | benzo[d][1,3]dioxol-5-yl | 0.16 |
| 6-51 | 4-Cl | 6-(2-methoxyethoxy)pyridin-3-yl | 0.05 |
| 6-52 | 4-Cl | 6-(2-ethoxyethoxy)pyridin-3-yl | 0.22 |
| 6-53 | 4-Cl | 6-(2-(2-ethoxyethoxy)ethoxy)pyridin-3-yl | 0.13 |
| 6-54 | 4-CF₃ | 6-fluoropyridin-3-yl | 0.13 |
| 6-55 | 4-CF₃ | 4-chlorophenyl | 0.78 |
| 6-56 | 4-CF₃ | 6-(2-(2-ethoxyethoxy)ethoxy )pyridin-3-yl | 0.20 |
| 6-57 | 4-^{t}Bu | methoxy | 0.70 |
| 6-58 | 4-^{t}Bu | 2-(dimethylamino)ethoxy | 0.69 |
| 6-59 | 4-^{t}Bu | 2,2,3,3-tetrafluoropropoxy | 0.50 |
| 6-60 | 4-^{t}Bu | 2-morpholinoethoxy | 0.89 |
| 6-61 | 4-^{t}Bu | 2-(2-ethoxyethoxy)ethoxy | 0.54 |
| 6-62 | 4-^{t}Bu | pyrrolidin-1-yl | 0.71 |
| 6-63 | 4-^{t}Bu | piperidin-1-yl | 1.92 |
| 6-64 | 4-^{t}Bu | 3-methylpiperidin-1-yl | 1.07 |
| 6-65 | 4-^{t}Bu | 3,3-difluoroazetidin-1-yl | 0.32 |
| 6-66 | 4-^{t}Bu | 3,3-difluoropyrrolidin-1-yl | 0.13 |
| 6-67 | 4-^{t}Bu | 4,4-difluoropiperidin-1-yl | 0.73 |
| 6-68 | 4-^{t}Bu | 2-methylenepyrrolidin-1-yl | 1.44 |
| 6-69 | 4-^{t}Bu | methyl-L-prolinate | 0.44 |
| 6-70 | 4-^{t}Bu | Z-prolinyl | >1.25 |
| 6-71 | 4-^{t}Bu | 2-(hydroxymethyl)pyrrolidin-1-yl | 0.28 |
| 6-72 | 4-^{t}Bu | 2-((2-(2-ethoxyethoxy)ethoxy) | 0.67 |
| | | methyl)pyrrolidin-1-yl | |
| 6-73 | 4-^{t}Bu | 4-(tert-butyl)piperazin-1-yl | 0.65 |
| 6-74 | 4-^{t}Bu | 1H-imidazol-1-yl | 0.33 |
| 6-75 | 4-^{t}Bu | 1H-pyrazol-1-yl | 0.24 |
| 6-76 | 4-^{t}Bu | 5-(tert-butyl)-1,3,4-oxadiazol-2-yl | 0.31 |
| 6-77 | 4-^{t}Bu | 3-methyl-1H-pyrazol-1-yl | 0.25 |
| 6-78 | 4-^{t}Bu | 4-(trifluoromethyl)-1H-pyrazol-1-yl | 0.57 |
| 6-79 | 4-^{t}Bu | 2-fluorophenyl | 0.69 |
| 6-80 | 4-^{t}Bu | 3-fluorophenyl | 0.78 |
| 6-81 | 4-^{t}Bu | 4-fluorophenyl | 0.94 |
| 6-82 | 4-^{t}Bu | 6-methoxypyridin-3-yl | 0.38 |
| 6-83 | 4-^{t}Bu | 6-(2-(2-ethoxyethoxy)ethoxy)pyridin-3-yl | 0.41 |
| 6-84 | 4-OCF₃ | TBSO-azetidin-1-yl | 1.13 |
| 6-85 | 4-OCF₃ | 3,3-difluoropyrrolidin-1-yl | 0.20 |
| 6-86 | 4-OCF₃ | 4-(tert-butyl)phenyl | 3.46 |
| 6-87 | 4-OCF₃ | 4-(tert-pentyl)phenyl | >2 |
| 6-88 | 4-OCF₃ | 4-(trifluoromethyl)phenyl | 0.42 |
| 6-89 | 3-F | 4-fluorophenyl | 0.12 |
| 6-90 | 3-F | 6-fluoropyridin-3-yl | 0.14 |
| 6-91 | 3-F | 4-chlorophenyl | 0.18 |
| 6-92 | 3-F | benzo[d][1,3]dioxol-5-yl | 0.45 |
| 6-93 | 3-F | 6-((2-methoxyethyl)(methyl) | 0.40 |
| | | amino)pyridin-3-yl | |
| 6-94 | 3-F | 6-(2-methoxyethoxy)pyridin-3-yl | 0.28 |
| 6-95 | 3-F | 6-(2-ethoxyethoxy)pyridin-3-yl | 0.29 |
| 6-96 | 3-F | 6-(2-(2-ethoxyethoxy)ethoxy)pyridin-3-yl | 0.35 |
| 6-97 | 4-F | 3,3-difluoropyrrolidin-1-yl | 0.57 |
| 6-98 | 4-F | 4,4-difluoropiperidin-1-yl | 0.43 |
| 6-99 | 4-F | (2S,6R)-2,6-dimethylmorpbolinyl | 0.61 |
| 6-100 | 4-F | Methyl L-prolinate | 0.74 |
| 6-101 | 4-F | Ethyl L-prolinate | >1.25 |
| 6-102 | 4-F | (S)-2-(hydroxymethyl)pyrrolidin-1-yl | 1.30 |
| 6-103 | 4-F | (S)- 2-(methoxymethyl)pyrrolidin-1-yl | 0.78 |
| 6-104 | 4-F | (S)-2-(ethoxymethyl)pyrrolidin-1-yl | 0.38 |
| 6-105 | | 2-(propoxymethyl)pyrrolidin-1-yl | 0.42 |
| 6-106 | 4-F | (S)-2-(phenoxymethyl)pyrrolidin-1-yl | 0.43 |
| 6-107 | | thiophen-3-yl | 0.37 |
| 6-108 | 4-F | 3-methyl-1H-pyrazol-1-yl | 0.09 |
| 6-109 | 4-F | 3-(trifluoromethyl)-1H-pyrazol-1-yl | 0.38 |
| 6-110 | 4-F | phenyl | 0.23 |
| 6-111 | 4-F | pyridin-3-yl | 0.09 |
| 6-112 | 4-F | pyrimidin-5-yl | 0.22 |
| 6-113 | 4-F | 2-fluorophenyl | 0.40 |
| 6-114 | 4-F | 3-fluorophenyl | 0.20 |
| 6-115 | 4-F | 4-fluorophenyl | 0.22 |
| 6-116 | 4-F | 6-fluoropyridin-3-yl | 0.09 |
| 6-117 | | 3-chlorophenyl | 0.15 |
| 6-118 | 4-F | 4-chlorophenyl | 0.25 |
| 6-119 | 4-F | 6-chloropyridin-3-yl | 0.08 |
| 6-120 | 4-F | 6-methylpyridin-3-yl | 0.25 |
| 6-121 | 4-F | 6-methoxypyridin-3-yl | 0.07 |
| 6-122 | 4-F | 4-(trifluoromethyl)phenyl | 0.28 |
| 6-123 | 4-F | 4-(trifluoromethoxy)phenyl | 0.34 |
| 6-124 | 4-F | benzo[d][1,3]dioxol-5-yl | 0.55 |
| 6-125 | 4-F | 3,4-difluorophenyl | 0.10 |
| 6-126 | 4-F | 2,4-difluorophenyl | 0.35 |
| 6-127 | 4-F | 4-chloro-3-fluorophenyl | 0.15 |
| 6-128 | 4-F | 4-chloro-2-fluorophenyl | 0.07 |
| 6-129 | 4-F | 4-chloro-2-ethoxyphenyl | >3 |
| 6-130 | 4-F | 6-(2-(dimethylamino)ethoxy)pyridin-3-yl | 0.26 |
| 6-131 | 4-F | 6-((2-methoxyetbyl)(methyl) | 0.37 |
| | | amino)pyridin-3-yl | |
| 6-132 | 4-F | 4-propoxyphenyl | 0.43 |
| 6-133 | 4-F | 6-propoxypyridin-3-yl | 0.16 |
| 6-134 | 4-F | 6-(2-hydroxyethoxy)pyridin-3-yl | 0.22 |
| 6-135 | 4-F | 6-(2-methoxyethoxy)pyridin-3-yl | 0.22 |
| 6-136 | 4-F | 6-(2-ethoxyethoxy)pyridin-3-yl | 0.26 |
| 6-137 | 4-F | 6-(2-(2-ethoxyethoxy)ethoxy)pyridin-3-yl | 0.18 |
| 6-138 | 4-F | 2-fluoro-4-(2-methoxyethoxy)phenyl | >1.25 |
| 6-139 | 4-F | 6-(2-(2-ethoxyethoxy )ethoxy)- | 0.21 |
| | | 4-methylpyridin-3-yl | |
| 6-140 | 4-F | 4-chloro-2-(2-(2-ethoxyethoxy) | 4.83 |
| | | etboxy)phenyl | |
| 6-141 | 4-F | 6-morpholinopyridin-3-yl | 0.42 |
| 6-142 | 4-F | 4,4-dimethylcyclohex-1-en-1-yl | 0.74 |
| 6-143 | 4-F | 4-methylcyclobexyl | 1.02 |
| 6-144 | 4-F | 4,4-dimethylcyclohex-1-en-1-yl | 0.55 |
| 6-145 | 4-F | 4,4-dimethylcyclohexyl | 0.89 |

### Compound 6-1

### (S)—N-(6-(2-(hydroxymethyl)pyrrolidin-1-yl)-1-(o-tolyl)-1H-pyrazolo[3,4-d]pyrimidin-4-yl)-5-nitrothiophene-2-carboxamide

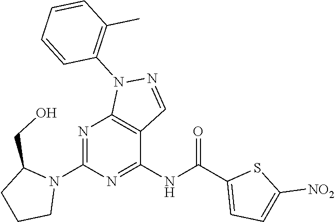 [SMILES: Cc1ccccc1-n1ncc2c(NC(=O)c3ccc([N+](=O)[O-])s3)nc(N3CCC[C@H]3CO)nc21]

¹H NMR (400 MHz, DMSO-d₆): δ 11.46 (br. s., 1H), 8.29 (br. s., 1H), 8.23 (br. s., 2H), 7.30-7.47 (m, 4H), 4.62 (br. s., 1H), 4.23 (br. s., 1H), 4.01 (br. s., 1H), 3.50 (br. s., 4H), 2.17 (s, 3H), 1.85-2.07 (m, 3H), 1.76-1.85 (m, 1H). MS(M+1): 480. Yellow solid.

### Compound 6-2

### N-(6-(6-fluoropyridin-3-yl)-1-(o-tolyl)-1H-pyrazolo[3,4-d]pyridin-4-yl)-5-nitrothiophene-2-carboxamide

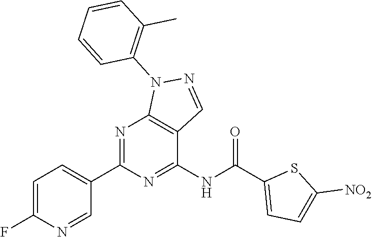 [SMILES: Cc1ccccc1-n1ncc2c(NC(=O)c3ccc([N+](=O)[O-])s3)nc(-c3ccc(F)nc3)nc21]

¹H NMR (400 MHz, DMSO-d₆): δ 12.11 (br. s., 1H), 9.17 (d, J=2.4 Hz, 1H), 8.76 (td, J=8.3, 2.4 Hz, 1H), 8.66 (s, 1H), 8.38 (d, J=4.4 Hz, 1H), 8.27 (d, J=4.4 Hz, 1H), 7.49-7.59 (m, 3H), 7.41-7.49 (m, 1H), 7.35 (dd, J=8.8, 2.4 Hz, 1H), 2.16 (s, 3H)

MS(M+1): 476. Light yellow solid.

### Compound 6-3

### (S)—N-(6-(2-(hydroxymethyl)pyrrolidin-1-yl)-1-(p-tolyl)-1H-pyrazolo[3,4-d]pyrimidin-4-yl)-5-nitrothiophene-2-carboxamide

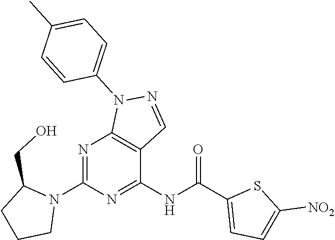 [SMILES: Cc1ccc(-n2ncc3c(NC(=O)c4ccc([N+](=O)[O-])s4)nc(N4CCC[C@H]4CO)nc32)cc1]

¹H NMR (400 MHz, DMSO-d₆): δ 11.46 (br. s., 1H), 8.26 (br. s., 1H), 8.19-8.24 (m, 2H), 8.08-8.15 (m, J=8.3 Hz, 2H), 7.24-7.38 (m, J=8.3 Hz, 2H), 4.73 (br s., 1H), 4.25 (br. s., 1H), 3.49-3.74 (m, 4H), 2.32-2.43 (m, 3H), 1.93-2.13 (m, 3H), 1.91 (br. s., 1H). MS(M+1): 480. Yellow solid.

### Compound 6.4

### N-(6-(4-fluorophenyl)-1-(p-tolyl)-1H-pyrazolo[3,4-d]pyrimidin-4-yl)-5-nitrothiophene-2-carboxamide

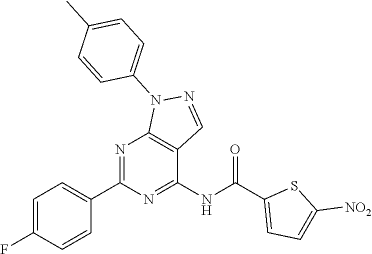 [SMILES: Cc1ccc(-n2ncc3c(NC(=O)c4ccc([N+](=O)[O-])s4)nc(-c4ccc(F)cc4)nc32)cc1]

¹H NMR (400 MHz, DMSO-d₆): δ 12.03 (br. s., 1H), 8.51-8.66 (m, 3H), 8.36 (d, J=4.4 Hz, 1H), 8.26 (d, J=4.4 Hz, 1H), 8.06-8.19 (m, 2H), 7.35-7.52 (m, 4H), 2.41 (s, 3H). MS(M+1): 475. Yellow solid.

### Compound 6-5

### N-(6-(6-fluoropyridin-3-yl)-1-(p-tolyl)-1H-pyrazolo[3,4-d]pyrimidin-4-yl)-5-nitrothiophene-2-carboxamide

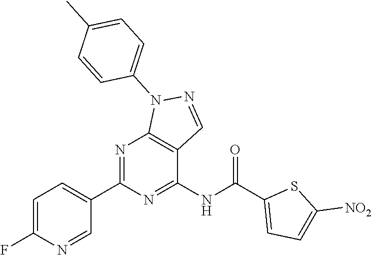 [SMILES: Cc1ccc(-n2ncc3c(NC(=O)c4ccc([N+](=O)[O-])s4)nc(-c4ccc(F)nc4)nc32)cc1]

¹H NMR (400 MHz, DMSO-d₆): δ 12.11 (br. s., 1H), 9.30 (d, J=2.4 Hz, 1H), 8.94 (td, J=8.3, 2.4 Hz, 1H), 8.63 (s, 1H), 8.31 (d, J=4.4 Hz, 1H), 8.25 (d, J=4.4 Hz, 1H), 8.10-8.20 (m, 2H), 7.39-7.49 (m, 3H), 2.41 (s, 3H). MS(M+1): 476. Light khaki solid.

### Compound 6-6

### N-(6-(6-methoxypyridin-3-yl)-1-(p-tolyl)-1H-pyrazolo[3,4-d]pyrimidin-4-yl)-5-nitrothiophene-2-carboxamide

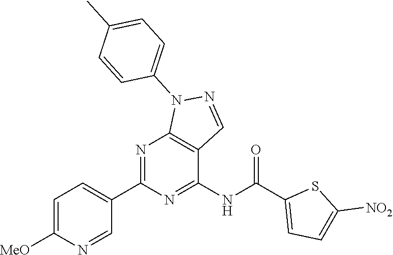 [SMILES: COc1ccc(-c2nc(NC(=O)c3ccc([N+](=O)[O-])s3)c3cnn(-c4ccc(C)cc4)c3n2)cn1]

¹H NMR (400 MHz, DMSO-d₆): δ 11.97 (br. s., 1H), 9.27 (d, J=2.4 Hz, 1H), 8.67 (dd, J=8.6, 2.2 Hz, 1H), 8.57 (s, 1H), 8.35 (d, J=4.4 Hz, 1H), 8.25 (d, J=4.9 Hz, 1H), 8.09-8.18 (m, J=8.8 Hz, 2H), 7.38-7.47 (m, J=8.3 Hz, 2H), 7.01 (d, J=8.3 Hz, 1H), 3.96 (s, 3H), 2.40 (s, 3H). MS(M+1): 488. Yellow solid.

### Compound 6-7

### N-(6-(4-chlorophenyl)-1-(p-tolyl)-1H-pyrazolo[3,4,d]pyridin-4-yl)-5-nitrothiophene-2-carboxamide

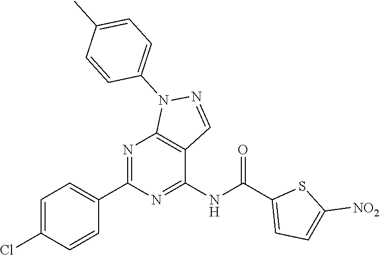 [SMILES: Cc1ccc(-n2ncc3c(NC(=O)c4ccc([N+](=O)[O-])s4)nc(-c4ccc(Cl)cc4)nc32)cc1]

¹H NMR (400 MHz, DMSO-d₆): δ 12.05 (br. s., 1H), 8.61 (s, 1H), 8.49-8.57 (m, 2H), 8.34 (d, J=4.4 Hz, 1H), 8.26 (d, J=4.4 Hz, 1H), 8.08-8.16 (m, 2H), 7.63-7.71 (m, 2H), 7.45 (d, J=7.8 Hz, 2H), 2.42 (s, 3H), MS(M+1): 491. Silver solid.

### Compound 6-8

### 5-nitro-N-(1-(p-tolyl)-6-(4-(trifluoromethyl)phenyl)-1H-pyrazolo[3,4-d]pyrimidin-4-yl)thiophene-2-carboxamide

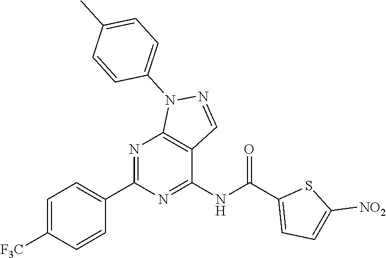 [SMILES: Cc1ccc(-n2ncc3c(NC(=O)c4ccc([N+](=O)[O-])s4)nc(-c4ccc(C(F)(F)F)cc4)nc32)cc1]

¹H NMR (400 MHz, DMSO-d₆): δ 12.10 (br. s., 1H), 8.70 (d, J=8.3 Hz, 2H), 8.63 (s, 1H), 8.37 (d, J=4.4 Hz, 1H), 8.26 (d, J=4.4 Hz, 1H), 8.08-8.17 (m, 2H), 7.91-7.99 (m, J=8.3 Hz, 2H), 7.44 (d, J=8.3 Hz, 2H), 2.42 (s, 3H). MS(M+1): 525. Silver solid.

### Compound 6-9

### N-(6-(benzo[d][1,3]dioxol-5-yl)-1-(p-tolyl)-1H-pyrazolo[3,4-d]pyridin-4-yl)-5-nitrothiophene-2-carboxamide

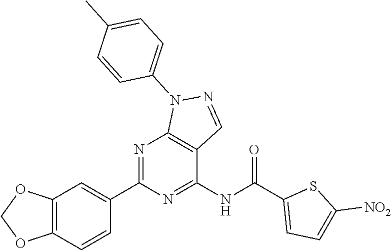 [SMILES: Cc1ccc(-n2ncc3c(NC(=O)c4ccc([N+](=O)[O-])s4)nc(-c4ccc5c(c4)OCO5)nc32)cc1]

¹H NMR (400 MHz, DMSO-d₆): δ 11.84 (br. s., 1H), 8.54 (s, 1H), 8.34 (d, J=4.4 Hz, 1H), 8.24 (d, J=4.4 Hz, 1H), 8.06-8.16 (m, 3H), 7.96 (d, J=1.5 Hz, 1H), 7.42 (d, J=8.3 Hz, 2H), 7.09 (d, J=8.3 Hz, 1H), 6.14 (s, 2H), 2.40 (s, 3H). MS(M+1): 501. Yellow solid.

### Compound 6-10

### N-(6-(2,3-dihydrobenzo[b][1,4]dioxin-6-yl)-1-(p-tolyl)-1H-pyrazolo[3,4-d]pyrimidin-4-yl)-5-nitrothiophene-2-carboxamide

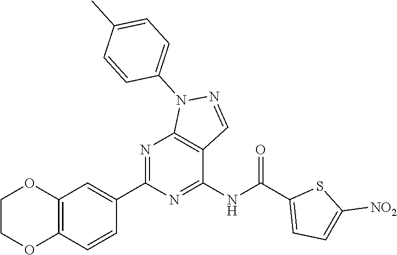 [SMILES: Cc1ccc(-n2ncc3c(NC(=O)c4ccc([N+](=O)[O-])s4)nc(-c4ccc5c(c4)OCCO5)nc32)cc1]

¹H NMR (400 MHz, DMSO-d₆): δ 11.91 (br. s., 1H), 8.55 (s, 1H), 8.35 (d, J=4.4 Hz, 1H), 8.25 (d, J=4.4 Hz, 1H), 8.06-8.15 (m, J=8.3 Hz, 2H), 7.98-8.06 (m, 2H), 7.40-7.49 (m, J=8.3 Hz, 2H), 7.03 (d, J=8.3 Hz, 1H), 4.24-4.39 (m, 4H), 2.40 (s, 3H). MS(M+1): 515. Khaki solid.

### Compound 6-11

### N-(6-(2-(4-difluorophenyl)-1-(p-tolyl)-1H-pyrazolo[3,4-d]pyrimidin-4-yl)-5-nitrothiophene-2-carboxamide

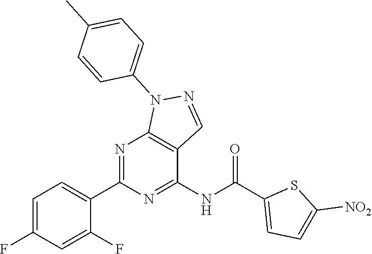 [SMILES: Cc1ccc(-n2ncc3c(NC(=O)c4ccc([N+](=O)[O-])s4)nc(-c4ccc(F)cc4F)nc32)cc1]

¹H NMR (400 MHz, DMSO-d₆): δ 12.14 (br. s., 1H), 8.61 (s, 1H), 8.34 (d, J=4.4 Hz, 1H), 8.29 (td, J=8.8, 6.8 Hz, 1H), 8.23 (d, J=4.4 Hz, 1H), 8.06-8.17 (m, 2H), 7.44 (ddd, J=11.4, 9.2, 2.4 Hz, 1H), 7.35-7.41 (m, J=8.3 Hz, 2H), 7.22-7.34 (m, 1H), 2.38 (s, 4H). MS(M+1): 493. Light yellow solid.

### Compound 6-12

### N-(6-(6-(2-methoxyethoxy)pyridin-3-yl)-1-(p-tolyl)-1H-pyrazolo[3,4-d]pyrimidin-4-yl)-5-nitrothiophene-2-carboxamide

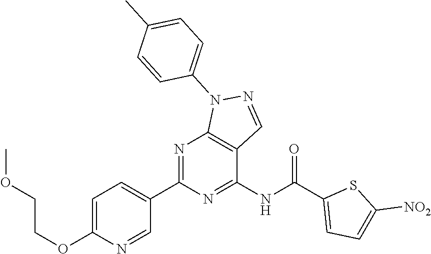 [SMILES: COCCOc1ccc(-c2nc(NC(=O)c3ccc([N+](=O)[O-])s3)c3cnn(-c4ccc(C)cc4)c3n2)cn1]

¹H NMR (400 MHz, DMSO-d₆): δ11.91 (br. s., 1H), 9.23 (d, J=2.4 Hz, 1H), 8.64 (dd, J=8.8, 2.4 Hz, 1H), 8.55 (s, 1H), 8.33 (d, J=4.9 Hz, 1H), 8.23 (d, J=4.4 Hz, 1H), 8.04-8.15 (m, J=8.3 Hz, 2H), 7.36-7.46 (m, J=8.8 Hz, 2H), 7.00 (d, J=8.3 Hz, 1H), 4.33-4.55 (m, 2H), 3.63-3.77 (m, 2H), 3.32 (s, 3H), 2.39 (s, 3H). MS(M+1): 532. Khaki solid.

### Compound 6-13

### N-(6-(6-(2-ethoxyethoxy)pyridin-3-yl)-1-(p-tolyl)-1H-pyrazolo[3,4-d]pyrimidin-4-yl)-5-nitrothiophene-2-carboxamide

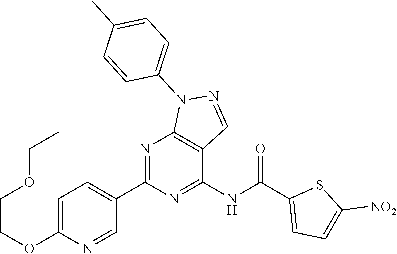 [SMILES: CCOCCOc1ccc(-c2nc(NC(=O)c3ccc([N+](=O)[O-])s3)c3cnn(-c4ccc(C)cc4)c3n2)cn1]

¹H NMR (400 MHz, DMSO-d₆): δ 11.89 (br. s., 1H), 9.22 (d, J=2.4 Hz, 1H), 8.63 (dd, J=8.6, 2.2 Hz, 1H), 8.54 (s, 1H), 8.34 (d, J=4.4 Hz, 1H), 8.23 (d, J=4.4 Hz, 1H), 8.05-8.13 (m, J=8.3 Hz, 2H), 7.36-7.47 (m, J=8.3 Hz, 2H), 7.00 (d, J=8.8 Hz, 1H), 4.46 (dd, J=5.6, 4.2 Hz, 2H), 3.70-3.78 (m, 2H), 3.51 (q, J=6.8 Hz, 2H), 2.39 (s, 3H), 1.14 (t, J=6.8 Hz, 3H). MS(M+1): 546. Yellow solid.

### Compound 6-14

### N-(6-(6(2-(2-ethoxyethoxy)pyridin-3-yl)-1-(p-tolyl)-1H-pyrazolo[3,4-d]pyrimidin-4-yl)-5-nitrothiophene-2-carboxamide

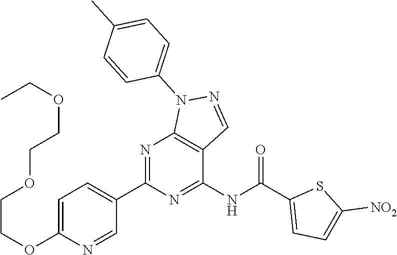 [SMILES: CCOCCOCCOc1ccc(-c2nc(NC(=O)c3ccc([N+](=O)[O-])s3)c3cnn(-c4ccc(C)cc4)c3n2)cn1]

¹H NMR (400 MHz, DMSO-d₆): δ 11.87 (br. s., 1H), 9.21 (d, J=2.0 Hz, 1H), 8.62 (dd, J=8.8, 2.4 Hz, 1H), 8.53 (s, 1H), 8.33 (d, J=4.4 Hz, 1H), 8.22 (d, J=4.4 Hz, 1H), 8.03-8.15 (m, 2H), 7.36-7.44 (m, J=8.3 Hz, 2H), 6.98 (d, J=9.3 Hz, 1H), 4.46 (dd, J=5.4, 3.9 Hz, 2H), 3.78 (dd, J=5.4, 3.9 Hz, 2H), 3.58-3.64 (m, 2H), 3.47-3.55 (m, 2H), 3.44 (q, J=6.8 Hz, 2H), 2.38 (s, 3H), 1.10 (t, J=6.8 Hz, 3H). MS(M+1): 590. Yellow solid.

### Compound 6-15

### N-(6-(cyclohexylamino)-1-(4-methoxyphenyl)-1H-pyrazolo[3,4-d]pyrimidin-4-yl)-5-nitrothiophene-2-carboxamide

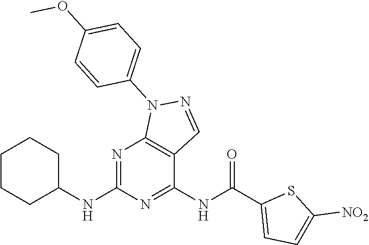 [SMILES: COc1ccc(-n2ncc3c(NC(=O)c4ccc([N+](=O)[O-])s4)nc(NC4CCCCC4)nc32)cc1]

¹H NMR (400 MHz, DMSO-d₆): δ 11.68 (br., 1H), 8.12-8.29 (m, 2H), 8.05 (d, J=8.3 Hz, 3H), 7.29 (br., 1H), 7.08 (d, J=8.8 Hz, 2H), 3.82 (s, 4H), 1.93 (d, J=18.6 Hz, 2H), 1.68-1.83 (m, 2H), 1.62 (d, J=11.7 Hz, 1H), 1.34 (br. s., 4H), 1.23 (br. s., 1H). MS(M+1): 494. Yellow solid.

### Compound 6-16

### N-(6-(cyclohexyloxy)-1-(4-methoxyphenyl)-1H-pyrazolo[3,4-d]pyrimidin-4-yl)-5-nitrothiophene-2-carboxamide

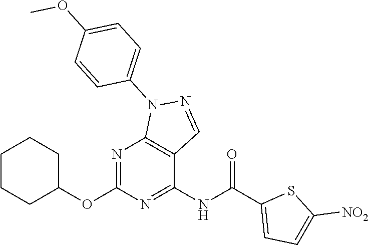 [SMILES: COc1ccc(-n2ncc3c(NC(=O)c4ccc([N+](=O)[O-])s4)nc(OC4CCCCC4)nc32)cc1]

¹H NMR (400 MHz, DMSO-d₆): δ 12.08 (br. s., 1H), 8.48 (s, 1H), 8.32 (d, J=4.4 Hz, 1H), 8.23 (d, J=4.4 Hz, 1H), 7.98-8.07 (m, J=9.3 Hz, 2H), 7.09-7.18 (m, J=9.3 Hz, 2H), 5.02-5.15 (m, 1H), 3.83 (s, 3H), 2.00 (d, J=3.9 Hz, 2H), 1.75 (d, J=5.9 Hz, 2H), 1.50-1.67 (m, 3H), 1.34-1.50 (m, 3H). MS(M+1): 495. Yellow solid.

### Compound 6-17

### N-(1-(4-methoxyphenyl)-6-(6-methylpyridin-3-yl)-1H-pyrazolo[3,4,d]pyrimidin-4-yl)-5-nitrothiophene-2-carboxamide

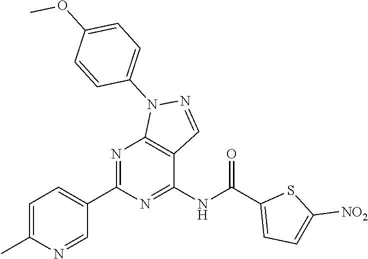 [SMILES: COc1ccc(-n2ncc3c(NC(=O)c4ccc([N+](=O)[O-])s4)nc(-c4ccc(C)nc4)nc32)cc1]

¹H NMR (400 MHz, DMSO-d₆): δ 11.91 (s, 1H), 9.47 (d, J=2.4 Hz, 1H), 8.59 (dd, J=8.1, 2.2 Hz, 1H), 8.52 (s, 1H), 8.34 (d, J=4.4 Hz, 1H), 8.21 (d, J=4.4 Hz, 1H), 8.09 (d, J=9.3 Hz, 2H), 7.42 (d, J=8.3 Hz, 1H), 7.15 (d, J=8.8 Hz, 2H), 3.84 (s, 3H), 2.55 (s, 3H). MS(M+1): 488. Orange solid.

### Compound 6-18

### N-(6-(4-fluorophenyl)-1-(4-methoxyphenyl)-1H-pyrazolo[3,4-d]pyrimidin-4-yl)-5-nitrothiophene-2-carboxamide

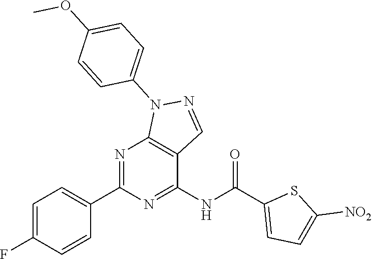 [SMILES: COc1ccc(-n2ncc3c(NC(=O)c4ccc([N+](=O)[O-])s4)nc(-c4ccc(F)cc4)nc32)cc1]

¹H NMR (400 MHz, DMSO-d₆): δ 12.04 (br. s., 1H), 8.54-8.62 (m, 3H), 8.36 (d, J=4.4 Hz, 1H), 8.26 (d, J=4.4 Hz, 1H), 8.09-8.17 (m, 2H), 7.37-7.47 (m, 2H), 7.17-7.24 (m, 2H), 3.86 (s, 3H). MS(M+1): 491. Yellow solid.

### Compound 6-19

### N-(6-(6-fluoropyridin-3-yl)-1-(4-methoxyphenyl)-1H-pyrazolo[3,4-d]pyrimidin-4-yl)-5-nitrothiophene-2-carboxamide

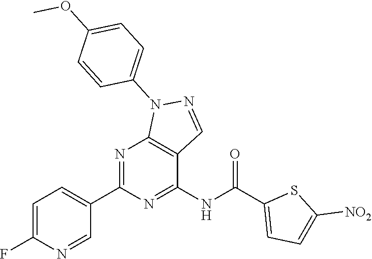 [SMILES: COc1ccc(-n2ncc3c(NC(=O)c4ccc([N+](=O)[O-])s4)nc(-c4ccc(F)nc4)nc32)cc1]

¹H NMR (400 MHz, DMSO-d₆): δ 12.06 (br s., 1H), 9.30 (d, J=2.0 Hz, 1H), 8.93 (td, J=8.2, 2.2 Hz, 1H), 8.63 (s, 1H), 8.37 (d, J=4.4 Hz, 1H), 8.26 (d, J=4.4 Hz, 1H), 8.09-8.18 (m, J=9.3 Hz, 2H), 7.42 (dd, J=8.8, 2.4 Hz, 1H), 7.15-7.24 (m, J=9.3 Hz, 2H), 3.86 (s, 3H). MS(M+1): 492. Yellow solid.

### Compound 6-20

### N-(6-(4-chlorophenyl)-1-(4-methoxyphenyl)-1H-pyrazolo[3,4-d]pyrimidin-4-yl)-5-nitrothiophene-2-carboxamide

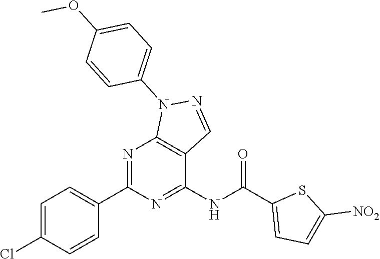 [SMILES: COc1ccc(-n2ncc3c(NC(=O)c4ccc([N+](=O)[O-])s4)nc(-c4ccc(Cl)cc4)nc32)cc1]

¹H NMR (400 MHz, DMSO-d₆): δ 11.98 (br. s., 1H), 8.56 (s, 1H), 8.46-8.52 (m, J=8.3 Hz, 2H), 8.37 (d, J=4.4 Hz, 1H), 8.24 (d, J=4.4 Hz, 1H), 8.06-8.12 (m, J=8.8 Hz, 2H), 7.63 (d, J=8.3 Hz, 2H), 7.17 (d, J=9.3 Hz, 2H), 3.85 (s, 3H). MS(M+1): 507. Yellow solid.

### Compound 6-21

### N-(6-(6-chloropyridin-3-yl)-1-(4-methoxyphenyl)-1H-pyrazolo[3,4,d]pyrimidin-4-yl)-5-nitrothiophene-2-carboxamide

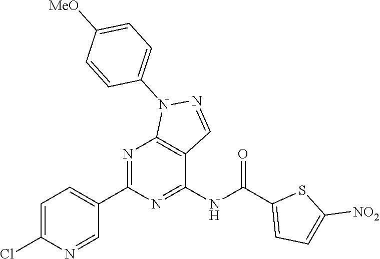 [SMILES: COc1ccc(-n2ncc3c(NC(=O)c4ccc([N+](=O)[O-])s4)nc(-c4ccc(Cl)nc4)nc32)cc1]

¹H NMR (400 MHz, DMSO-d₆): δ 12.07 (br. s., 1H), 9.44 (d, J=2.4 Hz, 1H), 8.79 (dd, J=8.3, 2.4 Hz, 1H), 8.63 (s, 1H), 8.38 (d, J=4.4 Hz, 1H), 8.08-8.17 (m, 2H), 7.76 (d, J=8.8 Hz, 1H), 7.15-7.23 (m, 2H), 3.79-3.90 (m, 3H). MS(M+1): 508. Black solid.

### Compound 6-22

### N-(6-(4-chloro-2-fluorophenyl)-1-(4-methoxyphenyl)-1H-pyrazolo[3,4-d]pyrimidin-4-yl)-5-nitrothiophene-2-carboxamide

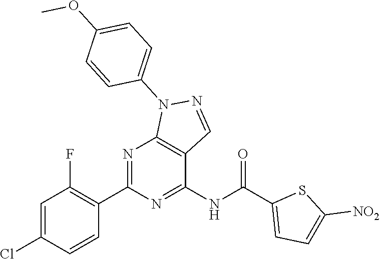 [SMILES: COc1ccc(-n2ncc3c(NC(=O)c4ccc([N+](=O)[O-])s4)nc(-c4ccc(Cl)cc4F)nc32)cc1]

¹H NMR (400 MHz, DMSO-d₆): δ 12.12 (br. s., 1H), 8.59 (s, 1H), 8.35 (d, J=4.4 Hz, 1H), 8.19-8.27 (m, 2H), 8.11 (d, J=8.8 Hz, 2H), 7.62 (dd, J=10.8, 2.0 Hz, 1H), 7.49 (dd, J=8.3, 2.0 Hz, 1H), 7.13 (d, J=9.3 Hz, 2H), 3.83 (s, 3H). MS(M+1): 525. Yellow solid.

### Compound 6-23

### N-(6-(3,4-difluorophenyl)-1-(4-methoxyphenyl)-1H-pyrazolo[3,4-d]pyrimidin-4-yl)-5-nitrothiophene-2-carboxamide

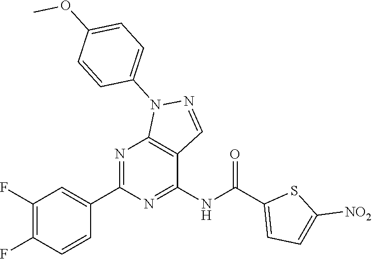 [SMILES: COc1ccc(-n2ncc3c(NC(=O)c4ccc([N+](=O)[O-])s4)nc(-c4ccc(F)c(F)c4)nc32)cc1]

¹H NMR (400 MHz, DMSO-d₆): δ 11.89 (br. s., 1H), 8.56 (s, 1H), 8.28-8.41 (m, 3H), 8.24 (d, J=4.4 Hz, 1H), 8.04-8.11 (m, 2H), 7.62 (dt, J=10.3, 8.6 Hz, 1H), 7.11-7.21 (m, 2H), 3.85 (s, 3H). MS(M+1) 509. Orange solid.

### Compound 6-24

### N-(6-(benzo[d][1,3]dioxol-5-yl)-1-(4-methoxyphenyl)-1H-pyrazolo[3,4-d]pyrimidin-4-yl)-5-nitrothiophene-2-carboxamide

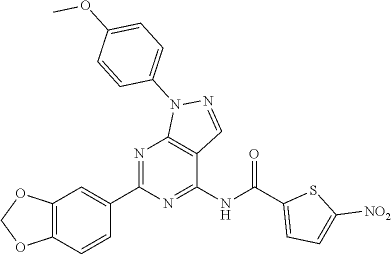 [SMILES: COc1ccc(-n2ncc3c(NC(=O)c4ccc([N+](=O)[O-])s4)nc(-c4ccc5c(c4)OCO5)nc32)cc1]

¹H NMR (400 MHz, DMSO-d₆): δ 11.87 (s, 1H), 8.56 (s, 1H), 8.37 (d, J=4.4 Hz, 1H), 8.26 (d, J=4.4 Hz, 1H), 8.08-8.18 (m, 3H), 7.98 (d, J=1.5 Hz, 1H), 7.19 (d, J=9.3 Hz, 2H), 7.11 (d, J=8.3 Hz, 1H), 6.15 (s, 2H), 3.86 (s, 3H). MS(M+1): 517. Orange solid

### Compound 6-25

### N-(1-(4-m)ethoxyphenyl)-6-(6-propoxypyridin-3-yl)-1H-pyrazolo[3,4-d]pyrimidin-4-yl)-5-nitrothiophene-2-carboxamide

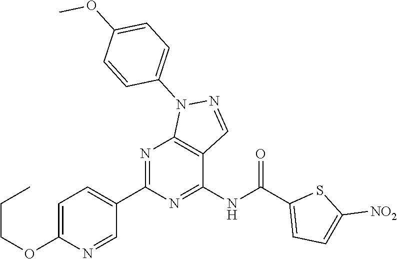 [SMILES: CCCOc1ccc(-c2nc(NC(=O)c3ccc([N+](=O)[O-])s3)c3cnn(-c4ccc(OC)cc4)c3n2)cn1]

¹H NMR (400 MHz, DMSO-d₆): δ 11.90 (br. s., 1H), 9.23 (d, J=2.0 Hz, 1H), 8.64 (dd, J=8.8, 2.4 Hz, 1H), 8.54 (s, 1H), 8.35 (d, J=4.4 Hz, 1H), 8.24 (d, J=4.4 Hz, 1H), 8.09-8.15 (m, 2H), 7.13-7.21 (m, 2H), 6.96 (d, J=8.8 Hz, 1H), 4.30 (t, J=6.6 Hz, 2H), 3.85 (s, 3H), 1.77 (sxt, J=7.1 Hz, 2H), 0.99 (t, J=7.6 Hz, 3H). MS(M+1): 532 Orange solid.

### Compound 6-26

### N-(6-(6-(2-methoxyethoxy)pyridin-3-yl)-1-(4-methoxyphenyl)-1H-pyrazolo[3,4,d]pyrimidin-4-yl)-5-nitrothiophene-2-carboxamide

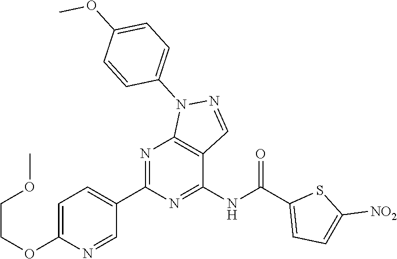 [SMILES: COCCOc1ccc(-c2nc(NC(=O)c3ccc([N+](=O)[O-])s3)c3cnn(-c4ccc(OC)cc4)c3n2)cn1]

¹H NMR (400 MHz, DMSO-d₆): δ 11.90 (br. s., 1H), 9.23 (d, J=2.0 Hz, 1H), 8.65 (dd, J=8.8, 2.4 Hz, 1H), 8.54 (s, 1H), 8.35 (d, J=4.4 Hz, 1H), 8.24 (d, J=4.4 Hz, 1H), 8.08-8.15 (m, 2H), 7.13-7.20 (m, 2H), 7.00 (d, J=8.8 Hz, 1H), 4.42-4.52 (m, 2H), 3.85 (s, 3H), 3.66-3.72 (m, 2H) MS(M+1): 548 Orange solid.

### Compound 6-27

### N-(6-(6-(2-ethoxyethoxy)pyridin-3-yl)-1-(4-methoxyphenyl)-1H-pyrazolo[3,4-d]pyrimidin-4-yl)-5-nitrothiophene-2-carboxamide

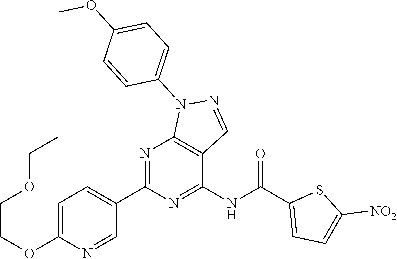 [SMILES: CCOCCOc1ccc(-c2nc(NC(=O)c3ccc([N+](=O)[O-])s3)c3cnn(-c4ccc(OC)cc4)c3n2)cn1]

¹H NMR (400 MHz, DMSO-d₆): δ 11.85 (br. s., 1H), 9.21 (d, J=2.0 Hz, 1H), 8.62 (dd, J=8.6, 2.2 Hz, 1H), 8.51 (s, 1H), 8.33 (d, J=4.4 Hz, 1H), 8.22 (d, J=4.4 Hz, 1H), 8.05-8.14 (m, 2H), 7.10-7.19 (m, 2H), 6.98 (d, J=8.8 Hz, 1H), 4.45 (dd, J=5.4, 3.9 Hz, 2H), 3.84 (s, 3H), 3.73 (dd, J=5.4, 3.9 Hz, 2H), 3.51 (q. J=6.8 Hz, 2H), 1.09-1.17 (m, 3H). MS(M+1): 562. Yellow solid.

### Compound 6-28

### N-(6-(6-(2-(2-ethoxyethoxy)ethoxy)pyridin-3-yl)-1-(4-methoxyphenyl)-1H-pyrazolo[3,4-d]pyrimidin-4-yl)-5-nitrothiophene-2-carboxamide

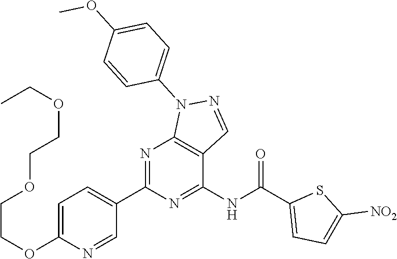 [SMILES: CCOCCOCCOc1ccc(-c2nc(NC(=O)c3ccc([N+](=O)[O-])s3)c3cnn(-c4ccc(OC)cc4)c3n2)cn1]

¹H NMR (400 MHz, DMSO-d₆): δ 11.79 (br. s., 1H), 9.17 (d, J=2.0 Hz, 1H), 8.59 (dd, J=8.8, 2.4 Hz, 1H), 8.48 (s, 1H), 8.31 (d, J=4.4 Hz, 1H), 8.20 (d, J=4.4 Hz, 1H), 8.04-8.11 (m, J=8.8 Hz, 2H), 7.09-7.16 (m, J=9.3 Hz, 2H), 6.95 (d, J=8.3 Hz, 1H), 4.44 (dd, J=5.6, 4.2 Hz, 2H), 3.83 (s, 3H), 3.75-3.80 (m, 2H), 3.57-3.62 (m, 2H), 3.48-3.53 (m, 2H), 3.43 (q, J=6.8 Hz, 2H), 1.10 (1, J=7.1 Hz, 3H). MS(M+1): 606. Yellow solid.

### Compound 6-29

### N-(6-(4,4-dimethylcyclohex-1-en-1-yl)-1-(4-methoxyphenyl)-1H-pyrazolo[3,4-d]pyrimidin-4-yl)-5-nitrothiophene-2-carboxamide

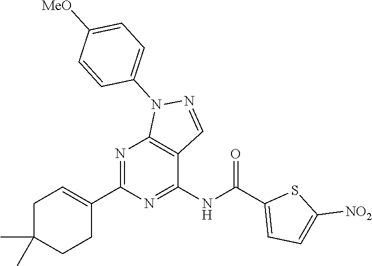 [SMILES: COc1ccc(-n2ncc3c(NC(=O)c4ccc([N+](=O)[O-])s4)nc(C4=CCC(C)(C)CC4)nc32)cc1]

¹H NMR (400 MHz, DMSO-d₆): δ 11.78 (br. s., 1H), 8.49 (s, 1H), 8.32 (br. s., 1H), 8.22 (d, J=4.4 Hz, 1H), 8.02-8.18 (m, J=8.8 Hz, 2H), 7.37 (br. s., 1H), 7.07-7.21 (m, 2H), 3.83 (s, 3H), 2.63 (br. s., 2H), 2.02-2.19 (m, 2H), 1.52 (t, J=6.4 Hz, 2H), 0.96 (s, 6H). MS(M+1): 505. Yellow solid.

### Compound 6-30

### N-(6-(4,4-dimethylcyclohexyl)-1-(4-methoxyphenyl)-1H-pyrazolo[3,4-d]pyrimidin-4-yl)-5-nitrothiophene-2-carboxamide

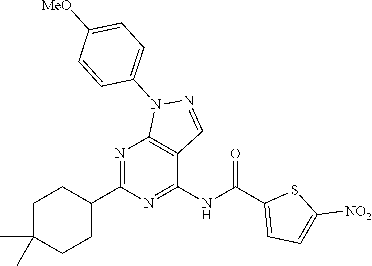 [SMILES: COc1ccc(-n2ncc3c(NC(=O)c4ccc([N+](=O)[O-])s4)nc(C4CCC(C)(C)CC4)nc32)cc1]

¹H NMR (400 MHz, DMSO-d₆): δ 12.07 (br. s., 1H), 8.52 (s, 1H), 8.28 (br. s., 1H), 8.21 (d, J=4.4 Hz, 1H), 7.96-8.11 (m, J=8.8 Hz, 2H), 7.06-7.20 (m, 2H), 3.84 (s, 3H), 2.76-2.85 (m, 1H), 1.77-1.94 (m, 4H), 1.50 (d, J=12.7 Hz, 2H), 1.34 (td, J=12.6, 5.1 Hz, 2H), 0.97 (d, J=3.4 Hz, 6H). MS(M+1): 507. Yellow solid.

### Compound 6-31

### (S)—N-(1-(3-chlorophenyl)-6(2-(hydroxymethyl)pyrrolidin-1-yl)-1H-pyrazolo[3,4-d]pyrmidin-4-yl)-5-nitrothiophene-2-carboxamide

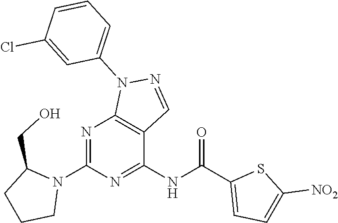 [SMILES: O=C(Nc1nc(N2CCC[C@H]2CO)nc2c1cnn2-c1cccc(Cl)c1)c1ccc([N+](=O)[O-])s1]

¹H NMR (400 MHz, DMSO-d₆): δ 11.53 (br s., 1H), 8.44 (br. s., 1H), 8.25-8.36 (m, 3H), 8.18-8.25 (m, 1H), 7.56 (t, J=8.3 Hz, 1H), 7.30-7.38 (m, 1H), 4.73 (br. s., 1H), 4.26 (br. s., 1H), 3.66 (br. s., 4H), 2.06 (d, J=13.2 Hz, 3H), 1.92 (d, J=6.8 Hz, 1H).

MS(M+1) 500. Orange solid.

### Compound 6-32

### methyl (1-(3-chlorophenyl)-4-(5-nitrothiophene-2-carboxamido)-1H-pyrazolo[3,4-d] pyrimidin-6-yl)-L-prolinate

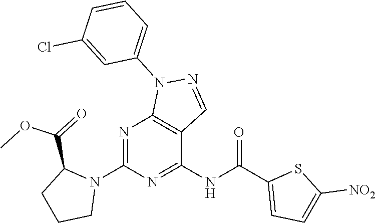 [SMILES: COC(=O)[C@@H]1CCCN1c1nc(NC(=O)c2ccc([N+](=O)[O-])s2)c2cnn(-c3cccc(Cl)c3)c2n1]

¹H NMR (400 MHz, DMSO-d₆): δ 11.66 (br. s., 1H), 8.24-8.35 (m, 3H), 8.16-8.24 (m, 2H), 7.53 (t, J=8.1 Hz, 1H), 7.36 (d, J=7.8 Hz, 1H), 4.57-4.64 (m, 1H), 3.71-3.88 (m, 2H), 3.57-3.71 (m, 3H), 2.41 (dt, J=8.2, 4.0 Hz, 1H), 1.93-2.15 (m, 3H).

MS(M+1): 528. Yellow solid.

### Compound 6-33

### N-(1-(3-chlorophenyl)-6-(thiophen-3-yl)-1H-pyrazolo[3,4-d]pyrimidin-4-yl)-5-nitrothiophene-2-carboxamide

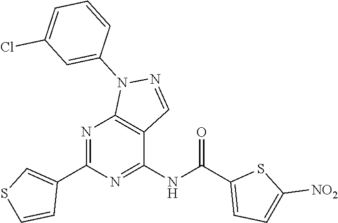 [SMILES: O=C(Nc1nc(-c2ccsc2)nc2c1cnn2-c1cccc(Cl)c1)c1ccc([N+](=O)[O-])s1]

¹H NMR (400 MHz, DMSO-d₆): δ 12.10 (s, 1H), 8.63 (s, 1H), 8.53 (dd, J=2.9, 1.0 Hz, 1H), 8.37-8.43 (m, 3H), 8.27 (d, J=4.4 Hz, 1H), 7.91-7.97 (m, 1H), 7.73-7.78 (m, 1H), 7.68 (t, J=8.1 Hz, 1H), 7.49 (dd, J=7.8, 2.4 Hz, 1H). MS(M+1): 483. Pale green solid.

### Compound 6-34

### N-(1-(3-chlorophenyl)-6-(3-fluorophenyl)-1H-pyrazolo[3,4-d]pyrimidin-4-yl)-5-nitrothiophene-2-carboxamide

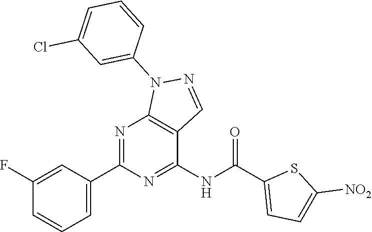 [SMILES: O=C(Nc1nc(-c2cccc(F)c2)nc2c1cnn2-c1cccc(Cl)c1)c1ccc([N+](=O)[O-])s1]

¹H NMR (400 MHz, DMSO-d₆): δ 12.06 (br. s., 1H), 8.67 (s, 1H), 8.39 (t, J=2.0 Hz, 1H), 8.33-8.38 (m, 3H), 8.22-8.28 (m, 2H), 7.62-7.72 (m, 2H), 7.48-7.52 (m, 1H), 7.45 (td, J=8.6, 2.4 Hz, 1H). MS(M+1): 495. Yellow solid.

### Compound 6-35

### N-(1-(3-chlorophenyl)-6-(4-fluorophenyl)-1H-pyrazolo[3,4-d]pyrimidin-4-yl)-5-nitrothiophene-2-carboxamide

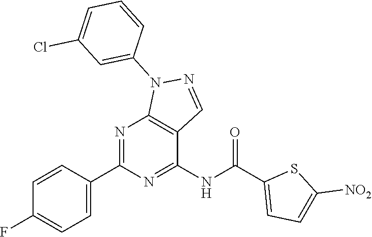 [SMILES: O=C(Nc1nc(-c2ccc(F)cc2)nc2c1cnn2-c1cccc(Cl)c1)c1ccc([N+](=O)[O-])s1]

¹H NMR (400 MHz, DMSO-d₆): δ 12.01 (br. s., 1H), 8.60 (s, 1H), 8.48-8.57 (m, 2H), 8.30-8.37 (m, 3H), 8.24 (d, J=4.4 Hz, 1H), 7.60-7.68 (m, 1H), 7.44-7.49 (m, 1H), 7.37-7.44 (m, 2H). MS(M+1): 495. Yellow solid.

### Compound 6-36

### N-(1-(3-chlorophenyl)-6-(6-fluoropyridin-3-yl)-1H-pyrazolo[3,4-d]pyrimidin-4-yl)-5-nitrothiophene-2-carboxamide

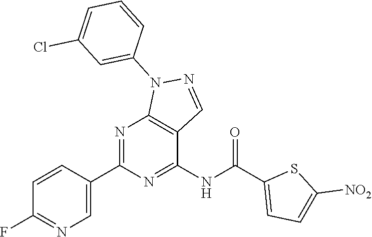 [SMILES: O=C(Nc1nc(-c2ccc(F)nc2)nc2c1cnn2-c1cccc(Cl)c1)c1ccc([N+](=O)[O-])s1]

¹H NMR (400 MHz, DMSO-d₆): δ 12.06 (br. s., 1H), 9.27 (d, J=2.4 Hz, 1H), 8.89 (td, J=8.1, 2.4 Hz, 1H), 8.64 (s, 1H), 8.28-8.40 (m, 3H), 8.25 (d, J=4.4 Hz, 1H), 7.66 (t, J=8.1 Hz, 1H), 7.45-7.49 (m, 1H), 7.43 (dd, J=8.6, 2.7 Hz, 1H). MS(M+1): 496. Pale yellow solid.

### Compound 6-37

### N-(1,6-bis(3-chlorophenyl)-1H-pyrazolo[3,4-d]pyrimidin-4-yl)-5-nitrothiophene-2-carboxamide

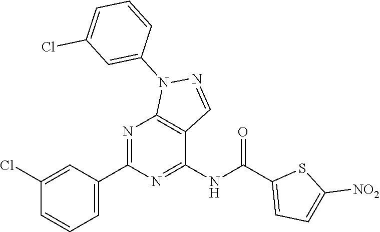 [SMILES: O=C(Nc1nc(-c2cccc(Cl)c2)nc2c1cnn2-c1cccc(Cl)c1)c1ccc([N+](=O)[O-])s1]

¹H NMR (400 MHz, DMSO-d₆): δ 12.07 (br. s., 1H), 8.67 (s, 1H), 8.51-8.55 (m, 1H), 8.45 (dt, J=7.0, 1.9 Hz, 1H), 8.39 (d, J=2.2 Hz, 1H), 8.37 (d, J=4.4 Hz, 1H), 8.32 (ddd, J=8.3, 2.0, 1.0 Hz, 1H), 8.26 (d, J=4.4 Hz, 1H), 7.61-7.71 (m, 3H), 7.47-7.52 (m, 1H). MS(M+1): 511. Pale yellow solid.

### Compound 6-38

### N-(1-(3-chlorophenyl)-6-(4-chlorophenyl)-1H-pyrazolo[3,4-d]pyrimidin-4-yl)-5-nitrothiophene-2-carboxamide

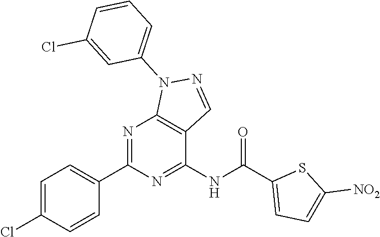 [SMILES: O=C(Nc1nc(-c2ccc(Cl)cc2)nc2c1cnn2-c1cccc(Cl)c1)c1ccc([N+](=O)[O-])s1]

¹H NMR (4007 MHz, DMSO-d₆): δ 12.02 (br. s., 1H), 8.62 (s, 1H), 8.44-8.54 (m, 2H), 8.37 (d, J=4.4 Hz, 1H), 8.29-8.35 (m, 2H), 8.25 (d, J=4.4 Hz, 1H), 7.60-7.71 (m, 3H), 7.43-7.50 (m, 1H). MS(M+1): 511. Pale yellow solid.

### Compound 6-39

### N-(6-(benzo[d][1,3]dioxol-5-yl)-1-(3-chlorophenyl)-1H-pyrazolo[3,4-d]pyrimidin-4-yl)-5-nitrothiophene-2-carboxamide

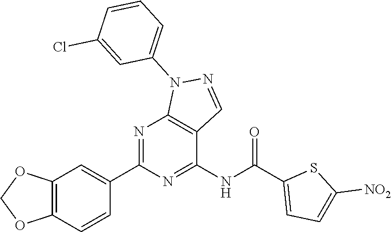 [SMILES: O=C(Nc1nc(-c2ccc3c(c2)OCO3)nc2c1cnn2-c1cccc(Cl)c1)c1ccc([N+](=O)[O-])s1]

¹H NMR (400 MHz, DMSO-d₆): δ 11.93 (br. s., 1H), 8.59 (s, 1H), 8.39 (t, J=2.0 Hz, 1H), 8.29-8.35 (m, 2H), 8.25 (d, J=4.4 Hz, 1H), 8.13 (dd, J=8.3, 1.5 Hz, 1H), 7.96 (d, J=1.5 Hz, 1H), 7.66 (t, J=8.3 Hz, 1H), 7.41-7.50 (m, 1H), 7.12 (d, J=8.3 Hz, 1H), 6.15 (s, 2H). MS(M+1): 521. Orange solid.

### Compound 6-40

### N-(1-(3-chlorophenyl)-6-(3-(trifluoromethoxy)phenyl)-1H-pyrazolo[3,4-d]pyrimidin-4-yl)-5-nitrothiophene-2-carboxamide

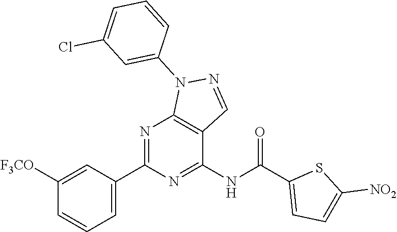 [SMILES: O=C(Nc1nc(-c2cccc(OC(F)(F)F)c2)nc2c1cnn2-c1cccc(Cl)c1)c1ccc([N+](=O)[O-])s1]

¹H NMR (400 MHz, DMSO-d₆): δ 12.02 (s, 1H), 8.64 (s, 1H), 8.51 (dt, J=7.8, 1.2 Hz, 1H), 8.38-8.45 (m, 2H), 8.36 (d, J=4.4 Hz, 1H), 8.17-8.31 (m, 2H), 7.74 (t, J=8.1 Hz, 1H), 7.65 (t, J=8.1 Hz, 1H), 7.55-7.62 (m, 1H), 7.40-7.52 (m, 1H). MS(M+1): 561. Yellow solid.

### Compound 6-41

### N-(1-(3-chlorophenyl)-6-(2-fluoro-4-(trifluoromethyl)phenyl)-1H-pyrazolo[3,4-d]pyrimidin-4-yl)-5-nitrothiophene-2-carboxamide

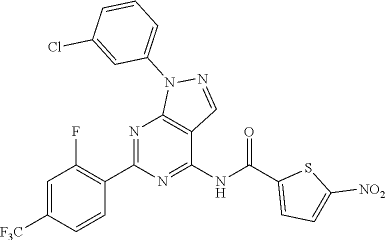 [SMILES: O=C(Nc1nc(-c2ccc(C(F)(F)F)cc2F)nc2c1cnn2-c1cccc(Cl)c1)c1ccc([N+](=O)[O-])s1]

¹H NMR (400 MHz, DMSO-d₆): δ 12.27 (br. s., 1H), 8.69 (s, 1H), 8.43-8.50 (m, 2H), 8.36 (d, J=4.4 Hz, 1H), 8.29 (dd, J=8.3, 1.5 Hz, 1H), 8.24 (d, J=4.4 Hz, 1H), 7.92 (d, J=10.8 Hz, 1H), 7.82 (d, J=8.3 Hz, 1H), 7.63 (t, J=8.1 Hz, 1H), 7.44-7.49 (m, 1H). MS(M+1): 563. Ashy solid.

### Compound 6-42

### N-(6-(2-(4-bis(trifluoromethyl)phenyl)-1-(3-chlorophenyl)-1H-pyrazolo[3,4-d]pyrimidin-4-yl)-5-nitrothiophene-2-carboxamide

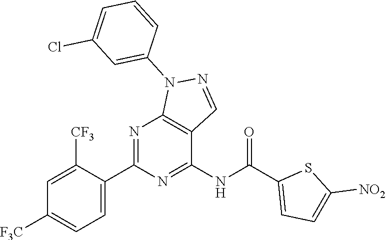 [SMILES: O=C(Nc1nc(-c2ccc(C(F)(F)F)cc2C(F)(F)F)nc2c1cnn2-c1cccc(Cl)c1)c1ccc([N+](=O)[O-])s1]

¹H NMR (400 MHz DMSO-d₆): δ 12.43 (s, 1H), 8.77 (s, 1H), 8.32-8.40 (m, 2H), 8.27-8.32 (m, 2H), 8.23 (d, J=4.4 Hz, 1H), 8.14-8.22 (m, 2H), 7.58-7.64 (m, 1H), 7.42-7.51 (m, 1H). MS(M+1): δ 13. White solid.

### Compound 6-43

### N-(1-(3-chlorophenyl)-6-(6-(2-methoxyethoxy)pyridin-3-yl)-1H-pyrazolo[3,4-d]pyrimidin-4-yl)-5-nitrothiophene-2-carboxamide

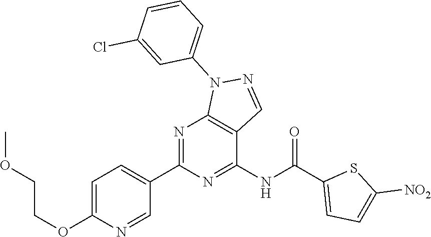 [SMILES: COCCOc1ccc(-c2nc(NC(=O)c3ccc([N+](=O)[O-])s3)c3cnn(-c4cccc(Cl)c4)c3n2)cn1]

¹H NMR (400 MHz, DMSO-d₆): δ 11.88 (br. s., 1H), 9.18 (d, J=2.0 Hz, 1H), 8.60 (dd, J=8.8, 2.4 Hz, 1H), 8.56 (s, 1H), 8.26-8.38 (m, 3H), 8.23 (d, J=4.4 Hz, 1H), 7.63 (t, J=8.3 Hz, 1H), 7.38-7.47 (m, 1H), 6.99 (d, J=8.8 Hz, 1H), 4.43-4.53 (m, 2H), 3.65-3.78 (m, 2H), 3.34 (s, 3H). MS(M+1): 552. Light yellow solid.

### Compound 6-44

### N-(1-(3-chlorophenyl)-6-(6-(2-ethoxyethoxy)pyridin-3-yl)-1H-pyrazolo[3,4-d]pyrimidin-4-yl)-5-nitrothiophene-2-carboxamide

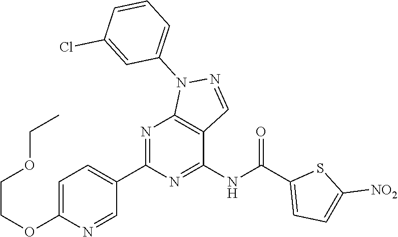 [SMILES: CCOCCOc1ccc(-c2nc(NC(=O)c3ccc([N+](=O)[O-])s3)c3cnn(-c4cccc(Cl)c4)c3n2)cn1]

¹H NMR (400 MHz, DMSO-d₆): δ 11.91 (br. s., 1H), 9.19 (d, J=2.4 Hz, 1H), 8.61 (dd, J=8.8, 2.4 Hz, 1H), 8.56 (s, 1H), 8.28-8.37 (m, 3H), 8.23 (d, J=4.4 Hz, 1H), 7.63 (t, J=8.3 Hz, 1H), 7.41-7.49 (m, 1H), 7.00 (d, J=8.8 Hz, 1H), 4.46 (dd, J=5.6, 4.2 Hz, 2H), 3.70-3.79 (m, 2H), 3.52 (q, J=6.8 Hz, 2H), 1.14 (t, J=7.1 Hz, 3H). MS(M+1): 566.

Light yellow solid.

### Compound 6-45

### N-(1-(3-chlorophenyl)-6-(6-(2-(2-ethoxyethoxy)ethoxy)pyridin-3-yl)-1H-pyrazolo[3,4-d]pyrimidin-4-yl)-5-nitrothiophene-2-carboxamide

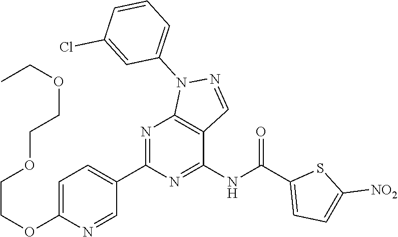 [SMILES: CCOCCOCCOc1ccc(-c2nc(NC(=O)c3ccc([N+](=O)[O-])s3)c3cnn(-c4cccc(Cl)c4)c3n2)cn1]

¹H NMR (400 MHz, DMSO-d₆): δ 11.80 (br. s., 1H), 9.12 (d, J=2.4 Hz, 1H), 8.40-8.59 (m, 2H), 8.13-8.40 (m, 4H), 7.51-7.69 (m, 1H), 7.33-7.49 (m, 1H), 6.95 (d, J=8.8 Hz, 1H), 4.38-4.50 (m, 2H), 3.74-3.83 (m, 2H), 3.57-3.63 (m, 2H), 3.48-3.54 (m, 2H), 3.44 (q, J=6.8 Hz, 2H), 1.10 (t, J=7.1 Hz, 3H). MS(M+1): 610. Brown solid.

### Compound 6-46

### N-(1-(4-chlorophenyl)-6-(3-methyl-1H-pyrazol-1-yl)-1H-pyrazolo[3,4-d]pyrimidin-4-yl)-5-nitrothiophene-2-carboxamide

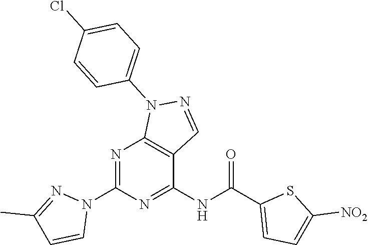 [SMILES: Cc1ccn(-c2nc(NC(=O)c3ccc([N+](=O)[O-])s3)c3cnn(-c4ccc(Cl)cc4)c3n2)n1]

¹H NMR (400 MHz, DMSO-d₆): δ 12.34 (br. s., 1H), 8.56-8.73 (m, 2H), 8.20-8.41 (m, 4H), 7.65-7.73 (m, 2H), 6.44-6.52 (m, 1H), 2.32-2.36 (m, 3H). MS(M+1): 481. Yellow solid.

### Compound 6-47

### N-(1-(4-chlorophenyl)-6-(piperidin-1-yl)-1H-pyrazolo[3,4,d]pyrimidin-4-yl)-5-nitrothiophene-2-carboxamide

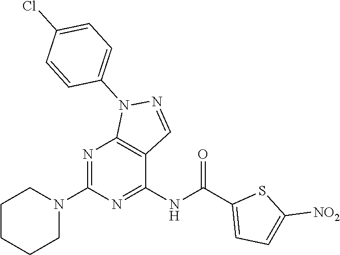 [SMILES: O=C(Nc1nc(N2CCCCC2)nc2c1cnn2-c1ccc(Cl)cc1)c1ccc([N+](=O)[O-])s1]

¹H NMR (400 MHz, DMSO-d₆): δ 11.49 (br. s., 1H), 8.27-8.34 (m, 2H), 8.19-8.27 (m, 3H), 7.58-7.65 (m, 2H), 3.89 (d, J=5.4 Hz, 4H), 1.64-1.72 (m, 2H), 1.53-1.64 (m, 4H) MS(M+1): 484. Orange solid.

### Compound 6-48

### N-(1-(4-chlorophenyl)-6-(4-fluorophenyl)-1H-pyrazolo[3,4-d]pyrimidin-4-yl)-5-nitrothiophene-2-carboxamide

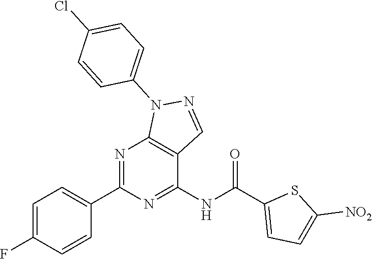 [SMILES: O=C(Nc1nc(-c2ccc(F)cc2)nc2c1cnn2-c1ccc(Cl)cc1)c1ccc([N+](=O)[O-])s1]

¹H NMR (400 MHz, DMSO-d₆): δ 8.64 (s, 1H), 8.54-8.63 (m, 2H), 8.29-8.41 (m, 3H), 8.26 (d, J=4.4 Hz, 1H), 7.71 (d, J=8.8 Hz, 2H), 7.43 (t, J=8.8 Hz, 2H). MS(M+1): 495. Yellow orange solid.

### Compound 6-49

### N-(1,6-bis(4-chlorophenyl)-1H-pyrazolo[3,4-d]pyrimidin-4-yl)-5-nitrothiophene-2-carboxamide

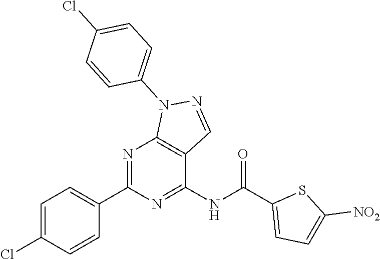 [SMILES: O=C(Nc1nc(-c2ccc(Cl)cc2)nc2c1cnn2-c1ccc(Cl)cc1)c1ccc([N+](=O)[O-])s1]

¹H NMR (400 MHz, DMSO-d₆): δ 12.14 (br. s., 1H), 8.65 (s, 1H), 8.55 (d, J=8.3 Hz, 2H), 8.34 (d, J=8.8 Hz, 3H), 8.25 (d, J=4.4 Hz, 1H), 7.71 (d, J=8.8 Hz, 2H), 7.66 (d, J=8.3 Hz, 2H), MS(M+1): 511, Khaki solid.

### Compound 6-50

### N-(6-(benzo[d][1,3]dioxol-5-yl)-1(4-chlorophenyl)-1H-pyrazolo[3,4-d]pyrimidin-4-yl)-5-nitrothiophene-2-carboxamide

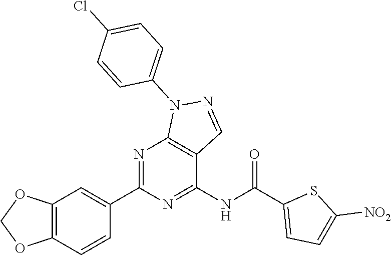 [SMILES: O=C(Nc1nc(-c2ccc3c(c2)OCO3)nc2c1cnn2-c1ccc(Cl)cc1)c1ccc([N+](=O)[O-])s1]

¹H NMR (400 MHz, DMSO-d₆): δ 11.94 (br. s., 1H), 8.62 (s, 1H), 8.33-8.38 (m, 3H), 8.23 (d, J=4.4 Hz, 1H), 8.18 (dd, J=8.3, 2.0 Hz, 1H), 8.02 (d, J=2.0 Hz, 1H), 7.65-7.75 (m, 2H), 7.09 (d, J=8.3 Hz, 1H), 6.15 (s, 2H). MS(M+1): 521. Light orange solid.

### Compound 6-51

### N-(1-(4-chlorophenyl)-6-(6-(2-methoxyethoxy)pyridin-3-yl)-1H-pyrazolo[3,4-d]pyrimidin-4-yl)-5-nitrothiophene-2-carboxamide

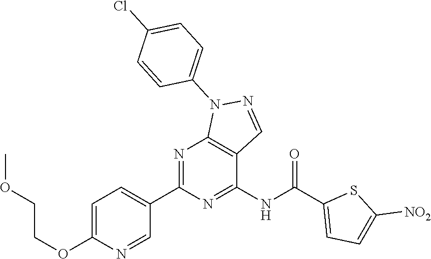 [SMILES: COCCOc1ccc(-c2nc(NC(=O)c3ccc([N+](=O)[O-])s3)c3cnn(-c4ccc(Cl)cc4)c3n2)cn1]

¹H NMR (400 MHz, DMSO-d₆): δ 11.97 (br. s., 1H), 9.27 (d, J=2.0 Hz, 1H), 8.69 (dd, J=8.8, 2.4 Hz, 1H), 8.60 (s, 1H), 8.29-8.39 (m, 3H), 8.25 (d, J=4.4 Hz, 1H), 7.66-7.75 (m, 2H), 7.01 (d, J=8.3 Hz, 1H), 4.48 (dd, J=5.6, 3.7 Hz, 2H), 3.66-3.77 (m, 2H), 3.32 (s, 3H). MS(M+1) 552. Khaki solid.

### Compound 6-52

### N-(1-(4-chlorophenyl)-6-(6-(2-ethoxyethoxy)pyridin-3-yl)-1H-pyrazolo[3,4-d]pyrimidin-4-yl)-5-nitrothiophene-2-carboxamide

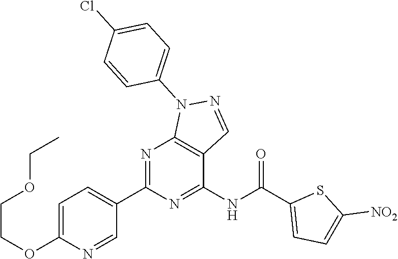 [SMILES: CCOCCOc1ccc(-c2nc(NC(=O)c3ccc([N+](=O)[O-])s3)c3cnn(-c4ccc(Cl)cc4)c3n2)cn1]

¹H NMR (400 Hz, DMSO-d₆): δ 11.92 (br. s., 1H), 9.23 (d, J=2.4 Hz, 1H), 8.65 (dd, J=8.8, 2.4 Hz, 1H), 8.57 (s, 1H), 8.28-8.37 (m, 3H), 8.24 (d, J=4.4 Hz, 1H), 7.61-7.72 (m, 2H), 6.99 (d, J=8.8 Hz, 1H), 4.37-4.50 (m, 2H), 3.69-3.80 (m, 2H), 3.52 (q, J=7.2 Hz, 2H), 1.08-1.19 (m, 3H). MS(M+1) 566. Khaki solid.

### Compound 6-53

### N-(1-(4-chlorophenyl)-6-(6-(2-(2-ethoxyethoxy)ethoxy)pyridin-3-yl)-1H-pyrazolo[3,4-d]pyrimidin-4-yl)-5-nitrothiophene-2-carboxamide

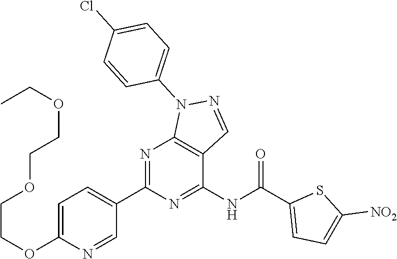 [SMILES: CCOCCOCCOc1ccc(-c2nc(NC(=O)c3ccc([N+](=O)[O-])s3)c3cnn(-c4ccc(Cl)cc4)c3n2)cn1]

¹H NMR (400 MHz, DMSO-d₆): δ 11.91 (br. s., 1H), 9.23 (d, J=2.0 Hz, 1H), 8.64 (dd, J=8.3, 2.4 Hz, 1H), 8.56 (s, 1H), 8.26-8.39 (m, 3H), 8.23 (d, J=4.4 Hz, 1H), 7.60-7.71 (m, 2H), 6.98 (d, J=8.3 Hz, 1H), 4.42-4.52 (m, 2H), 3.74-3.84 (m, 2H), 3.58-3.66 (m, 2H), 3.49-3.55 (m, 2H), 3.44 (q, J=6.8 Hz, 2H), 1.07-1.13 (m, 3H). MS (M+1): 610. Brown solid.

### Compound 6-54

### N-(6-(6-fluoropyridin-3-yl)-1-(4-(trifluoromethyl)phenyl)-1H-pyrazolo[3,4-d]pyrimidin-4-yl)-5-nitrothiophene-2-carboxamide

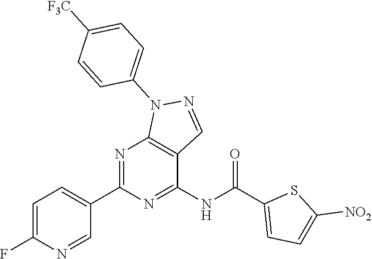 [SMILES: O=C(Nc1nc(-c2ccc(F)nc2)nc2c1cnn2-c1ccc(C(F)(F)F)cc1)c1ccc([N+](=O)[O-])s1]

¹H NMR (400 MHz, DMSO-d₆): δ 12.08 (s, 1H), 9.38 (s, 1H), 8.94-9.07 (m, 1H), 8.73 (s, 1H), 8.57-8.66 (m, J=8.3 Hz, 2H), 8.40 (d, J=4.4 Hz, 1H), 8.20-8.17 (d, J=4.4 Hz, 1H), 7.88-7.99 (m, J=8.3 Hz, 2H), 7.33 (d, J=7.3 Hz, 1H). MS(M+1): 530.

White brown solid.

### Compound 6-55

### N-(6-(4-chlorophenyl)-1(4-(trifluoromethyl)phenyl)-1H-pyrazolo[3,4-d]pyrimidin-4-yl)-5-nitrothiophene-2-carboxamide

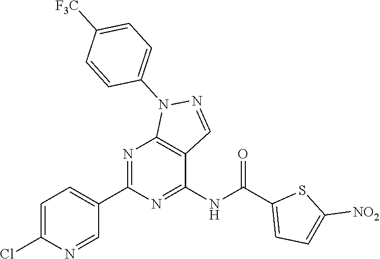 [SMILES: O=C(Nc1nc(-c2ccc(Cl)nc2)nc2c1cnn2-c1ccc(C(F)(F)F)cc1)c1ccc([N+](=O)[O-])s1]

¹H NMR (400 MHz, DMSO-d₆): δ 12.12 (br. s., 1H), 8.70 (s, 2H), 8.59 (t, J=7.8 Hz, 3H), 8.37 (br. s., 1H), 8.27 (br. s., 1H), 8.02 (d, J=7.8 Hz, 2H), 7.67 (d, J=8.3 Hz, 2H). MS(M+1): 545. Lavender solid.

### Compound 6-56

### N-(6(6-(2-(2-ethoxyethoxy)ethoxy)pyridin-3-yl)-1-(4-(trifluoromethyl)phenyl)-1H-pyrazolo[3,4-d]pyrimidin-4-yl)-5-nitrothiophene-2-carboxamide

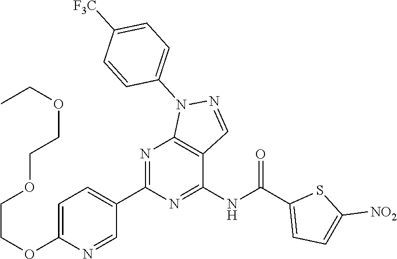 [SMILES: CCOCCOCCOc1ccc(-c2nc(NC(=O)c3ccc([N+](=O)[O-])s3)c3cnn(-c4ccc(C(F)(F)F)cc4)c3n2)cn1]

¹H NMR (400 MHz, DMSO-d₆): δ 11.98 (br. s., 1H), 9.29 (br. s., 1H), 8.71 (d, J=7.8 MHz, 1H), 8.64 (s, 1H), 8.54-8.63 (m, J=7.8 Hz, 2H), 8.35 (d, J=3.9 Hz, 1H), 8.25 (d, J=3.9 Hz, 1H), 7.93-8.06 (m, J=7.8 Hz, 2H), 7.01 (d, J=8.8 Hz, 1H), 4.48 (br. s., 2H), 3.79 (br. s., 2H), 3.57-3.62 (m, 2H), 3.50-3.54 (m, 2H), 3.44 (d, J=6.8 Hz, 2H), 1.10 (t, J=6.8 Hz, 3H). MS(M+1) 644. Yellow solid.

### Compound 6-57

### N-(1-(4-(tert-butyl)phenyl)-6-methoxy-1H-pyrazolo[3,4-d]pyrimidin-4-yl)-5-nitrothiophene-2-carboxamide

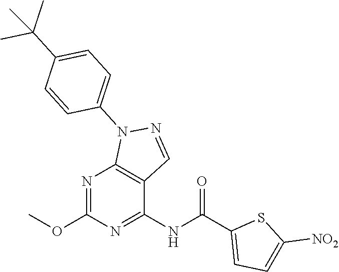 [SMILES: COc1nc(NC(=O)c2ccc([N+](=O)[O-])s2)c2cnn(-c3ccc(C(C)(C)C)cc3)c2n1]

¹H NMR (400 MHz, DMSO-d₆): δ 12.14 (br. s., 1H), 8.50 (s, 1H), 8.33 (d, J=4.4 Hz, 1H), 8.21 (d, J=4.4 Hz, 1H), 8.08-8.14 (m, J=8.8 Hz, 2H), 7.56-7.63 (m, J=8.8 Hz, 2H), 4.05 (s, 3H), 1.34 (s, 9H). MS (M+1): 453. Light yellow solid.

### Compound 6-58

### N-(1-(4-(tert-butyl)phenyl)-6-(2-(dimethylamino)ethoxy)-1H-pyrazolo[3,4-d]pyrimidin-4-yl)-5-nitrothiophene-2-carboxamide

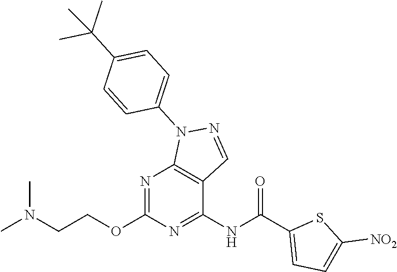 [SMILES: CN(C)CCOc1nc(NC(=O)c2ccc([N+](=O)[O-])s2)c2cnn(-c3ccc(C(C)(C)C)cc3)c2n1]

¹H NMR (400 MHz, DMSO-d₆): δ 12.14 (br. s., 1H), 9.58 (br. s., 1H), 8.55 (s, 1H), 8.32 (d, J=4.4 Hz, 1H), 8.24 (d, J=4.4 Hz, 1H), 8.03-8.10 (m, J=8.8 Hz, 2H), 7.57-7.63 (m, J=8.8 Hz, 2H), 4.78 (dd, J=5.6, 4.2 Hz, 2H), 3.58-3.66 (m, 2H), 2.89 (s, 6H), 1.35 (s, 9H). MS (M+1) 510. Yellow solid.

### Compound 6-59

### N-(1-(4-(tert-butyl)phenyl)-6-(2,2,3,3-tetrafluoropropoxy)-1H-pyrazolo[3,4-d]pyrimidin-4-yl)-5-nitrothiophene-2-carboxamide

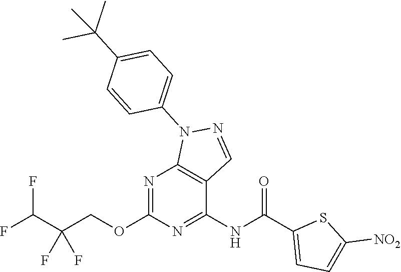 [SMILES: CC(C)(C)c1ccc(-n2ncc3c(NC(=O)c4ccc([N+](=O)[O-])s4)nc(OCC(F)(F)C(F)F)nc32)cc1]

¹H NMR (400 MHz, DMSO-d₆): δ 12.24 (s, 1H), 8.58 (s, 1H), 8.34 (d, J=4.4 Hz, 1H), 8.23 (d, J=4.4 Hz, 1H), 8.03-8.09 (m, J=8.3 Hz, 2H), 7.57-7.63 (m, J=8.8 Hz, 2H), 6.68 (t, J=5.4 Hz, 1H), 5.03 (t, J=13.9 Hz, 2H), 1.35 (s, 9H). MS(M+1): 553.

### Compound 6-60

### N-(1-(4-(tert-butyl)phenyl)-6-(2-morpholinoethoxy)-1H-pyrazolo[3,4-d]pyrimidin-4-yl)-5-nitrothiophene-2-carboxamide

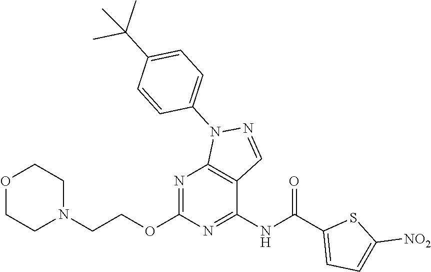 [SMILES: CC(C)(C)c1ccc(-n2ncc3c(NC(=O)c4ccc([N+](=O)[O-])s4)nc(OCCN4CCOCC4)nc32)cc1]

¹H NMR (400 MHz, DMSO-d₆): δ 8.51 (s, 1H), 8.32 (d, J=4.4 Hz, 1H), 8.22 (d, J=4.4 Hz, 1H), 8.04-8.11 (m, J=8.8 Hz, 2H), 7.56-7.62 (m, J=8.8 Hz, 2H), 4.55 (t, J=5.6 Hz, 2H), 3.52-3.58 (m, 4H), 2.76 (t, J=5.6 Hz, 2H), 1.34 (s, 9H). MS(M+1): 552.

### Compound 6-61

### N-(1-(4-tert-butyl)phenyl)-6-(2-(2-ethoxyethoxy)ethoxy)-1H-pyrazolo[3,4-d]pyrimidin-4-yl)-5-nitrothiophene-2-carboxamide

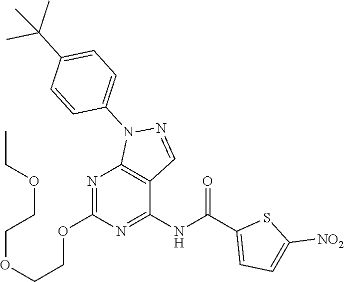 [SMILES: CCOCCOCCOc1nc(NC(=O)c2ccc([N+](=O)[O-])s2)c2cnn(-c3ccc(C(C)(C)C)cc3)c2n1]

¹H NMR (400 MHz, DMSO-d₆): δ 12.11 (br. s., 1H), 8.51 (s, 1H), 8.32 (d, J=4.9 Hz, 1H), 8.21 (d, J=4.4 Hz, 1H), 8.05-8.10 (m, J=8.8 Hz, 2H), 7.56-7.61 (m, J=8.8 Hz, 2H), 4.52-4.59 (m, 2H), 3.79-3.85 (m, 2H), 3.57-3.63 (m, 2H), 3.46-3.51 (m, 2H), 3.41 (q, J=7.2 Hz, 2H), 1.34 (s, 9H), 1.04-1.10 (m, 3H). MS(M+1): 555. Light green solid

### Compound 6-62

### N-(1-(4-(tert-butyl)phenyl)-6-(pyrrolidin-1-yl)-1H-pyrazolo[3,4-d]pyrimidin-4-yl)-5-nitrothiophene-2-carboxamide

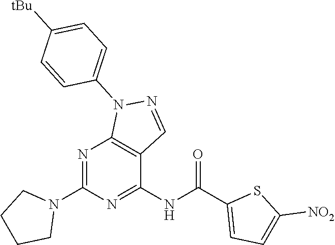 [SMILES: CC(C)(C)c1ccc(-n2ncc3c(NC(=O)c4ccc([N+](=O)[O-])s4)nc(N4CCCC4)nc32)cc1]

¹H NMR (400 MHz, DMSO-d₆): δ 8.20-8.28 (m, J=8.8 Hz, 2H), 8.04 (d, J=4.4 Hz, 1H), 8.02 (s, 1H), 7.61 (d, J=3.9 Hz, 1H), 7.46-7.52 (m, J=8.8 Hz, 2H), 3.58 (t, J=6.6 Hz, 4H), 1.89-1.99 (m, 4H), 1.32 (s, 9H). MS(M+1): 492.

### Compound 6-63

### N-(1-(4-(tert-butyl)phenyl)-6-(piperidin-1-yl)-1H-pyrazolo[3,4-d]pyrimidin-4-yl)-5-nitrothiophene-2-carboxamide

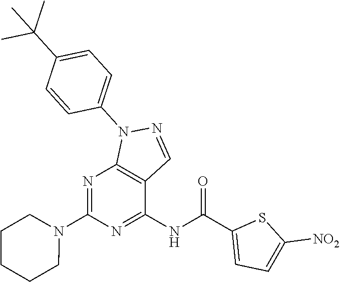 [SMILES: CC(C)(C)c1ccc(-n2ncc3c(NC(=O)c4ccc([N+](=O)[O-])s4)nc(N4CCCCC4)nc32)cc1]

¹H NMR (400 MHz, DMSO-d₆): δ 11.46 (br. s., 1H), 8.29 (d, J=4.4 Hz, 1H), 8.26 (s, 1H), 8.23 (d, J=4.4 Hz, 1H), 8.06-8.15 (m, 2H), 7.49-7.60 (m, 2H), 3.89 (d, J=4.9 Hz, 4H), 1.66 (br. s., 2H), 1.53-1.64 (m, 4H), 1.33 (s, 9H). MS(M+1): 506.

### Compound 6-64

### N-(1-(4-(tert-butyl)phenyl)-6-(3-methylpiperidin-1-yl)-1H-pyrazolo[3,4-d]pyrimidin-4-yl)-5-nitrothiophene-2-carboxamide

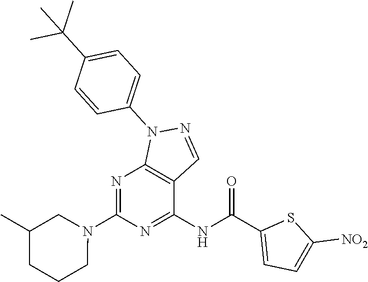 [SMILES: CC1CCCN(c2nc(NC(=O)c3ccc([N+](=O)[O-])s3)c3cnn(-c4ccc(C(C)(C)C)cc4)c3n2)C1]

¹H NMR (400 MHz, DMSO-d₆): δ 11.45 (s, 1H), 8.20-8.32 (m, 3H), 8.06-8.16 (m, 2H), 7.49-7.62 (m, 2H), 4.68 (d, J=12.7 Hz, 2H), 3.01 (t, J=11.7 Hz, 1H), 2.64-2.79 (m, 1H), 1.82 (d, J=12.2 Hz, 1H), 1.67-1.79 (m, 1H), 1.61 (dd, J=10.5.3.7 MHz, 1H), 1.47 (q, J=12.2 Hz, 1H), 1.14-1.29 (m, 1H), 0.96 (d, J=6.4 Hz, 3H). MS(M+1): 520.

### Compound 6-65

### N-(1-(4-(tert-butyl)phenyl)-6-(3,3-difluoroazetidin-1-yl)-1H-pyrazolo[3,4-d]pyrimidin-4-yl)-5-nitrothiophene-2-carboxamide

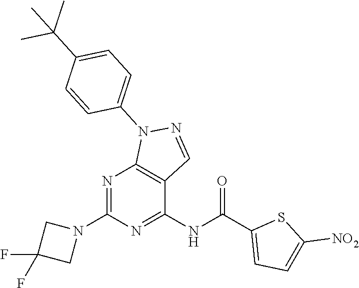 [SMILES: CC(C)(C)c1ccc(-n2ncc3c(NC(=O)c4ccc([N+](=O)[O-])s4)nc(N4CC(F)(F)C4)nc32)cc1]

¹H NMR (400 MHz, DMSO-d₆): δ 11.90 (s, 1H), 8.34 (s, 1H), 8.31 (d, J=4.4 Hz, 1H), 8.20 (d, J=4.9 Hz, 1H), 8.06-8.12 (m, J=8.8 Hz, 2H), 7.51-7.58 (m, J=8.8 Hz, 2H), 4.60 (t, J=12.5 Hz, 4H), 1.33 (s, 9H). MS(M+1): 514.

### Compound 6-66

### N-(1-(4-(tert-butyl)phenyl)-6-(3,3-difluoropyrrolidin-1-yl)-1H-pyrazolo[3,4,d]pyrimidin-4-yl)-5-nitrothiophene-2-carboxamide

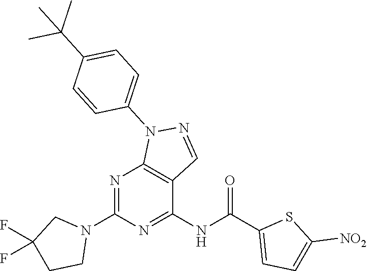 [SMILES: CC(C)(C)c1ccc(-n2ncc3c(NC(=O)c4ccc([N+](=O)[O-])s4)nc(N4CCC(F)(F)C4)nc32)cc1]

¹H NMR (400 MHz, DMSO-d₆): δ 11.62 (br. s., 1H), 8.28-8.33 (m, 2H), 8.23 (d, J=4.9 Hz, 1H), 8.13-8.19 (m, J=8.8 Hz, 2H), 7.53-7.60 (m, J=8.8 Hz, 2H), 4.06 (t, J=13.0 Hz, 2H), 3.88 (t, J=7.3 Hz, 2H), 2.59 (tt, J=14.3, 7.2 Hz, 2H), 1.34 (s, 9H).

MS (M+1): 528. Yellow solid.

### Compound 6-67

### N-(1(4-(tert-butyl)phenyl)-6-(4,4-difluoropiperidin-1-yl)-1H-pyrazolo[3,4-d]pyrimidin-4-yl)-5-nitrothiophene-2-carboxamide

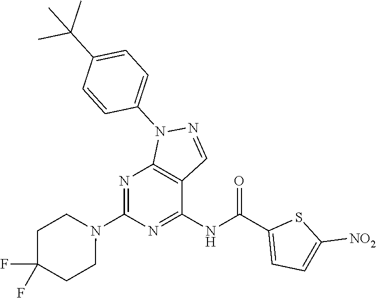 [SMILES: CC(C)(C)c1ccc(-n2ncc3c(NC(=O)c4ccc([N+](=O)[O-])s4)nc(N4CCC(F)(F)CC4)nc32)cc1]

¹H NMR (400 MHz, DMSO-d₆): δ 11.56 (br. s., 1H), 8.32 (s, 1H), 8.30 (d, J=4.4 Hz, 1H), 8.23 (d, J=4.4 Hz, 1H), 8.09 (d, J=88 Hz, 2H), 7.57 (d, J=8.8 Hz, 2H), 4.03 (t, J=5.4 Hz, 4H), 1.99-2.17 (m, 4H), 1.34 (s, 9H). MS(M+1): 542. Yellow solid.

### Compound 6-68

### N-(1-(4-(tert-butyl)phenyl)-6-(2-methylenepyrimidin-1-yl)-1H-pyrazolo[3,4-d]pyrimidin-4-yl)-5-nitrothiophene-2-carboxamide

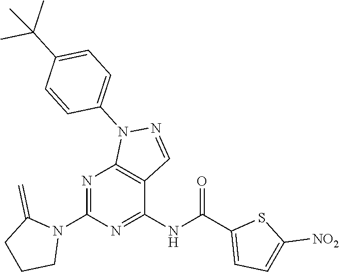 [SMILES: C=C1CCCN1c1nc(NC(=O)c2ccc([N+](=O)[O-])s2)c2cnn(-c3ccc(C(C)(C)C)cc3)c2n1]

¹H NMR (400 MHz, DMSO-d₆): δ 8.55 (s, 1H), 8.12 (d, J=4.4 Hz, 1H), 7.87-8.00 (m, J=8.3 Hz, 2H), 7.78 (d, J=4.4 Hz, 1H), 7.53-7.61 (m, J=8.8 Hz, 2H), 4.39-4.52 (m, 1H), 4.27-4.37 (m, 1H), 4.14-4.26 (m, 1H), 3.61-3.75 (m, 1H), 2.06-2.19 (m, 1H), 2.02 (br. s., 1H), 1.85-1.99 (m, 1H), 1.57-1.74 (m, 1H), 1.33 (s, 9H). MS(M+1): 504.

Yellow solid.

### Compound 6-69

### methyl(1-(4-(tert-butyl)phenyl)4-(5-nitrothiophene-2-carboxamido)-1H-pyrazolo[3,4-d]pyrimidin-6-yl)-L-prolinate

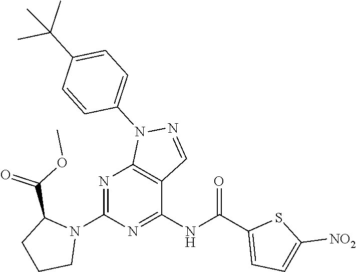 [SMILES: COC(=O)[C@@H]1CCCN1c1nc(NC(=O)c2ccc([N+](=O)[O-])s2)c2cnn(-c3ccc(C(C)(C)C)cc3)c2n1]

¹H NMR (400 MHz, DMSO-d₆): δ 11.64 (br. s., 1H), 8.21-8.35 (m, 3H), 8.03-8.13 (m, J=8.8 Hz, 2H), 7.46-7.60 (m, 2H), 4.57 (dd, J=8.6, 3.7 Hz, 1H), 3.82 (br. s., 2H), 3.61-3.68 (m, 3H), 2.40 (br. s., 1H), 2.03 (d, J=3.4 Hz, 3H), 1.34 (s, 9H).

MS(M+1) 550. Yellow solid.

### Compound 6-70

### (1-(4-(tert-butyl)phenyl)-4-(5-nitrothiophene-2-carboxamido)-1H-pyrazolo[3,4-d]pyrimidin-6-yl)-L-proline

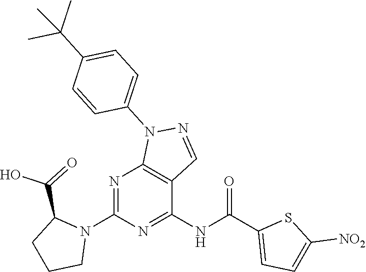 [SMILES: CC(C)(C)c1ccc(-n2ncc3c(NC(=O)c4ccc([N+](=O)[O-])s4)nc(N4CCC[C@H]4C(=O)O)nc32)cc1]

¹H NMR (400 MHz, DMSO-d₆): δ 12.63 (br. s., 1H), 11.63 (br. s., 1H), 8.12-8.36 (m, 5H), 7.39-7.62 (m, 2H), 4.48 (dd, J=8.6, 3.7 Hz, 1H), 3.80 (t, J=5.4 Hz, 2H), 3.61-3.74 (m, 1H), 2.25-2.45 (m, 2H), 1.87-2.16 (m, 3H), 1.33 (s, 9H). MS(M+1): 536

Yellow solid.

### Compound 6-71

### (S)—N-(1-(4-(tert-butyl)phenyl)-6-(2-(hydroxymethyl)pyrrolidin-1-yl)-1H-pyrazolo[3,4-d]pyrimidin-4-yl)-5-nitrothiophene-2-carboxamide

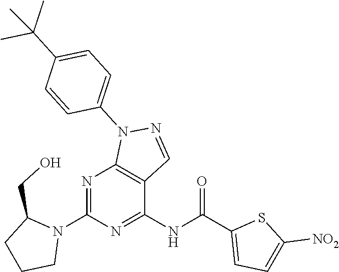 [SMILES: CC(C)(C)c1ccc(-n2ncc3c(NC(=O)c4ccc([N+](=O)[O-])s4)nc(N4CCC[C@H]4CO)nc32)cc1]

¹H NMR (400 MHz, DMSO-d₆): δ 11.48 (br. s., 1H), 8.15-8.38 (m, 5H), 7.54 (d, J=9.3 Hz, 2H), 4.77 (br. s., 1H), 4.26 (br. s., 1H), 3.53-3.83 (m, 4H), 1.84-2.14 (m, 4H), 1.33 (s, 9H). MS(M+1): 522. Yellow solid.

### Compound 6-72

### (S)—N-(1-(4-(tert-butyl)phenyl)-6-(2-((2-(2-ethoxyethoxy)ethoxy)methyl)pyrrolidin-1-yl)-1H-pyrazolo[3,4-d]pyrimidin-4-yl)-5-nitrothiophene-2-carboxamide

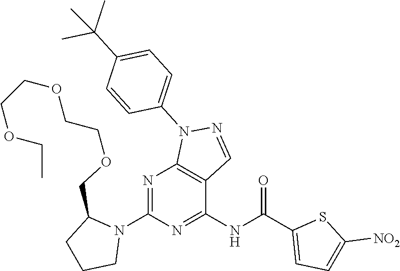 [SMILES: CCOCCOCCOC[C@@H]1CCCN1c1nc(NC(=O)c2ccc([N+](=O)[O-])s2)c2cnn(-c3ccc(C(C)(C)C)cc3)c2n1]

¹H NMR (400 MHz, DMSO-d₆): δ 11.51 (br. s., 1H), 8.28 (br. s., 1H), 8.24 (s, 1H), 8.21 (d, J=3.9 Hz, 1H), 8.12-8.19 (m, J=8.3 Hz, 2H), 7.46-7.57 (m, J=8.3 Hz, 2H), 4.36 (br. s., 1H), 3.78 (br. s., 1H), 3.63 (br. s., 3H), 3.53 (br. s., 3H), 3.44-3.50 (m, 3H), 3.42 (d, J=4.9 Hz, 2H), 3.37 (q, J=7.0 Hz, 2H), 2.03 (br. s., 3H), 1.92 (br. s., 1H), 1.33 (s, 9H), 1.05 (t, J=7.1 Hz, 3H). MS(M+1): δ 38. Orange solid.

### Compound 6-73

### N-(1-(4-(tert-butyl)phenyl)-6-(4-tert-butyl)piperazin-1-yl)-1H-pyrazolo[3,4-d]pyrimidin-4-yl)-5-nitrothiophene-2-carboxamide

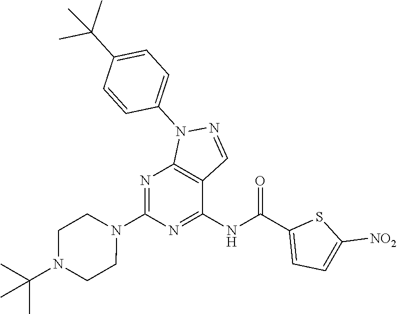 [SMILES: CC(C)(C)c1ccc(-n2ncc3c(NC(=O)c4ccc([N+](=O)[O-])s4)nc(N4CCN(C(C)(C)C)CC4)nc32)cc1]

¹H NMR (400 MHz, DMSO-d₆): δ 11.68 (br. s, 1H), 8.31-8.37 (m, 2H), 8.23 (d, J=4.4 Hz, 1H), 8.06-8.12 (m, 2H), 7.49-7.60 (m, 2H), 4.91 (d, J=11.7 Hz, 2H), 3.65 (br. s., 4H), 3.06 (d, J=8.8 Hz, 2H), 1.38 (br s., 9H), 1.34 (s, 9H). MS(M+1): 563. Yellow solid.

### Compound 6-74

### N-(1-(4-(tert-butyl)phenyl)-6-(1H-imidazol-1-yl)-1H-pyrazolo[3,4-d]pyrimidin-4-yl)-5-nitrothiophene-2-carboxamide

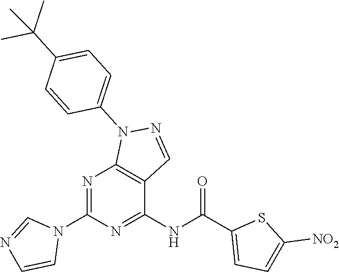 [SMILES: CC(C)(C)c1ccc(-n2ncc3c(NC(=O)c4ccc([N+](=O)[O-])s4)nc(-n4ccnc4)nc32)cc1]

¹H NMR (400 MHz, DMSO-d₆): δ 12.16 (br. s., 1H), 8.69 (s, 1H), 8.64 (s, 1H), 8.34 (d, J=4.4 Hz, 1H), 8.26 (d, J=4.4 Hz, 1H), 8.11-8.18 (m, J=8.8 Hz, 2H), 8.05 (t, J=1.2 Hz, 1H), 7.61-7.68 (m, J=8.8 Hz, 2H), 7.19 (s, 1H), 1.36 (s, 9H). MS(M+1): 489

Yellow solid.

### Compound 6-75

### N-(1-(4-tert-butyl)phenyl)-6-(4-(1H-pyrazol-1-yl)-1H-pyrazolo[3,4-d]pyrimidin-4-yl)-5-nitrothiophene-2-carboxamide

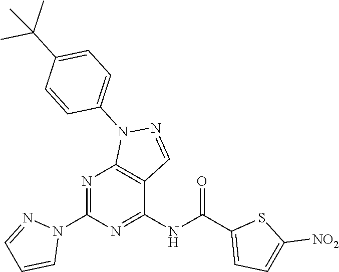 [SMILES: CC(C)(C)c1ccc(-n2ncc3c(NC(=O)c4ccc([N+](=O)[O-])s4)nc(-n4cccn4)nc32)cc1]

¹H NMR (400 MHz, DMSO-d₆): δ 8.81 (br. s., 1H), 8.51 (br. s., 1H), 8.14-8.24 (m, J=8.8 Hz, 2H), 8.09 (br s., 1H), 7.74 (br. s., 2H), 7.55-7.67 (m, J=8.3 Hz, 2H), 6.65 (br s., 1H), 1.36 (s, 9H). MS(M+1): 489. Yellow solid.

### Compound 6-76

### N-(6-(5-(tert-butyl)-1,3,4-oxadiazol-2-yl)-1-(4-(tert-butyl)phenyl)-1H-pyrazolo[3,4-d]pyrimidin-4-yl)-5-nitrothiophene-2-carboxamide

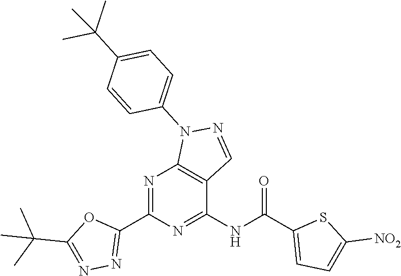 [SMILES: CC(C)(C)c1ccc(-n2ncc3c(NC(=O)c4ccc([N+](=O)[O-])s4)nc(-c4nnc(C(C)(C)C)o4)nc32)cc1]

¹H NMR (400 MHz, DMSO-d₆): δ 12.52 (s, 1H), 8.68 (s, 1H), 8.36 (br. s., 1H), 8.23 (d, J=4.4 Hz, 1H), 8.01-8.19 (m, J=8.8 Hz, 2H), 7.55-7.77 (m, J=8.3 Hz, 2H), 1.48 (s, 9H), 1.36 (s, 9H). MS(M+1): 547. White solid.

### Compound 6-77

### N-(1-(4-(tert-butyl)phenyl)-6-(3-methyl-1H-pyrazol-1-yl)-1H-pyrazolo[3,4-d]pyrimidin-4-yl)-5-nitrothiophene-2-carboxamide

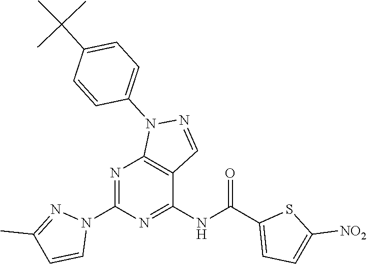 [SMILES: Cc1ccn(-c2nc(NC(=O)c3ccc([N+](=O)[O-])s3)c3cnn(-c4ccc(C(C)(C)C)cc4)c3n2)n1]

¹H NMR (400 MHz, DMSO-d₆): δ 12.31 (br. s., 1H), 8.65 (d, J=2.9 Hz, 1H), 8.56 (s, 1H), 8.35 (d, J=4.4 Hz, 1H), 8.23 (d, J=4.4 Hz, 1H), 7.90-8.15 (m, 2H), 7.44-7.69 (m, 2H), 6.47 (d, J=2.4 Hz, 1H), 2.33 (s, 3H), 1.36 (s, 9H). MS(M+1): 503. Yellow solid.

### Compound 6-78

### N-(1-(4-(ter-butyl)phenyl)-6-(4-(trifluoromethyl)-1H-pyrazol-1-yl)-1H-pyrazolo[3,4-d]pyrimidin-4-yl)-5-nitrothiophene-2-carboxamide

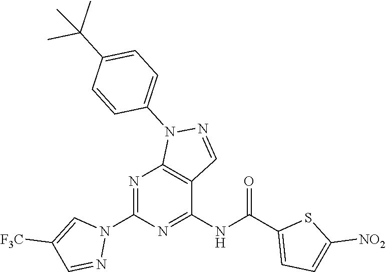 [SMILES: CC(C)(C)c1ccc(-n2ncc3c(NC(=O)c4ccc([N+](=O)[O-])s4)nc(-n4cc(C(F)(F)F)cn4)nc32)cc1]

¹H NMR (400 MHz, DMSO-d₆): δ 12.33 (br. s., 1H), 9.25 (s, 1H), 8.66 (s, 1H), 8.37 (s, 1H), 8.21-8.31 (m, 2H), 8.11-8.19 (m, 2H), 7.61-7.69 (m, 2H), 1.37 (s, 9H). MS(M+1): 557.

### Compound 6-79

### N-(1-(4-(tert-butyl)phenyl)-6-(2-fluorophenyl)-1H-pyrazolo[3,4-d]pyrimidin-4-yl)-5-nitrothiophene-2-carboxamide

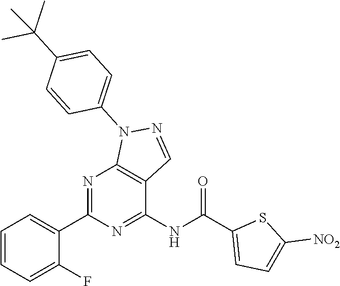 [SMILES: CC(C)(C)c1ccc(-n2ncc3c(NC(=O)c4ccc([N+](=O)[O-])s4)nc(-c4ccccc4F)nc32)cc1]

¹H NMR (400 MHz, DMSO-d₆): δ 12.17 (br. s., 1H), 8.64 (s, 1H), 8.36 (d, J=4.4 Hz, 1H), 8.13-8.31 (m, 4H), 7.55-7.68 (m, 3H), 7.35-7.47 (m, 2H), 1.35 (s, 9H). MS(M+1): 517.

### Compound 6-80

### N-(1-(4-(tert-butyl)phenyl)-6-(3-fluorophenyl)-1H-pyrazolo[3,4-d]pyrimidin-4-yl)-5-nitrothiophene-2-carboxamide

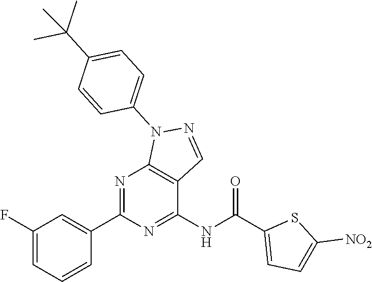 [SMILES: CC(C)(C)c1ccc(-n2ncc3c(NC(=O)c4ccc([N+](=O)[O-])s4)nc(-c4cccc(F)c4)nc32)cc1]

¹H NMR (400 MHz, DMSO-d₆): δ 12.03 (br. s., 1H), 8.53-8.66 (m, 3H), 8.36 (d, J=4.4 Hz, 1H), 8.26 (d, J=4.4 Hz, 1H), 8.12-8.23 (m, 2H), 7.60-7.71 (m, 2H), 7.39-7.49 (m, 2H), 1.37 (s, 9H). MS(M+1): 517.

### Compound 6-81

### N-(1-(4-(tert-butyl)phenyl)-6-(4-fluorophenyl)-1H-pyrazolo[3,4-d]pyrimidin-4-yl)-5-nitrothiophene-2-carboxamide

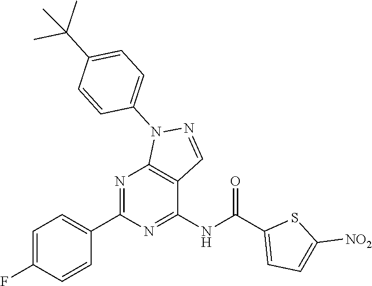 [SMILES: CC(C)(C)c1ccc(-n2ncc3c(NC(=O)c4ccc([N+](=O)[O-])s4)nc(-c4ccc(F)cc4)nc32)cc1]

¹H NMR (400 MHz, DMSO-d₆): δ 12.00 (br. s., 1H), 8.63 (s, 1H), 8.33-8.42 (m, 2H), 8.21-8.29 (m, 2H), 8.14-8.21 (m, 2H), 7.60-7.69 (m, 3H), 7.37-7.47 (m, 1H), 1.37 (s, 9H). MS(M+1): 517.

### Compound 6-82

### N-(1-(4-(tert-butyl)phenyl)-6-(6-methoxypyridin-3-yl)-1H-pyrazolo[3,4-d]pyrimidin-4-yl)-5-nitrothiophene-2-carboxamide

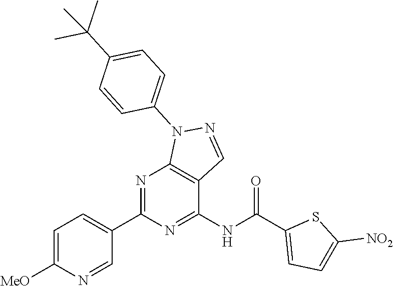 [SMILES: COc1ccc(-c2nc(NC(=O)c3ccc([N+](=O)[O-])s3)c3cnn(-c4ccc(C(C)(C)C)cc4)c3n2)cn1]

¹H NMR (400 MHz, DMSO-d₆): δ 11.90 (d, J=6.3 Hz, 1H), 9.52-9.24 (m, 1H), 8.66-8.53 (m, 2H), 8.34-8.14 (m, 4H), 7.61 (d, J=6.8 Hz, 2H), 6.99-6.96 (m, 1H), 3.94 (s, 3H), 1.35 (s, 9H). MS(M+1): 530. Yellow-brown solid.

### Compound 6-83

### N-(1-(4-(ter-butyl)phenyl)-6-(6-(2-(2-ethoxyethoxy)ethoxy)pyridin-3-yl)-1H-pyrazolo[3,4-d]pyrimidin-4-yl)-5-nitrothiophene-2-carboxamide

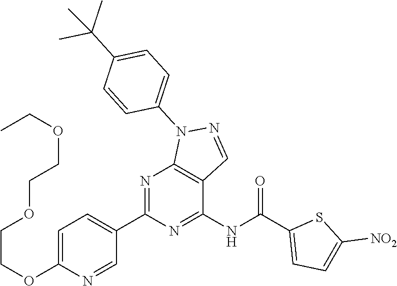 [SMILES: CCOCCOCCOc1ccc(-c2nc(NC(=O)c3ccc([N+](=O)[O-])s3)c3cnn(-c4ccc(C(C)(C)C)cc4)c3n2)cn1]

¹H NMR (400 MHz, DMSO-d₆): δ 11.92 (br. s., 1H), 9.25 (d, J=2.0 Hz, 1H), 8.67 (dd, J=8.8, 2.4 Hz, 1H), 8.56 (s, 1H), 8.34 (d, J=4.4 Hz, 1H), 8.23 (d, J=4.4 Hz, 1H), 8.08-8.20 (m, 2H), 7.57-7.69 (m, 2H), 7.01 (d, J=8.8 Hz, 1H), 4.47 (dd, J=5.6, 4.2 Hz, 2H), 3.78 (dd, J=5.4, 3.9 Hz, 2H), 3.56-3.64 (m, 2H), 3.47-3.53 (m, 2H), 3.43 (q, J=6.8 Hz, 2H), 1.36 (s, 9H), 1.10 (t, J=7.1 Hz, 3H). MS(M+1): δ 32. White solid.

### Compound 6-84

### N-(6-(3-((tert-butyldimethylsilyl)oxy)azetidin-1-yl)-1-(4-(trifluoroethoxy)phenyl)-1H-pyrazolo[3,4-d]pyrimidin-4-yl)-5-nitrothiophene-2-carboxamide

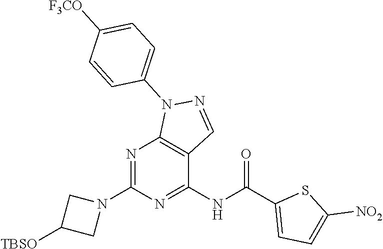 [SMILES: CC(C)(C)[Si](C)(C)OC1CN(c2nc(NC(=O)c3ccc([N+](=O)[O-])s3)c3cnn(-c4ccc(OC(F)(F)F)cc4)c3n2)C1]

¹H NMR (400 MHz, DMSO-d₆): δ 11.75 (br. s., 1H), 8.33-8.42 (m, 2H), 8.27-8.33 (m, 2H), 8.21 (d, J=4.4 Hz, 1H), 7.55 (d, J=8.3 Hz, 2H), 4.74-4.87 (m, 1H), 4.44 (dd, J=9.8, 6.4 Hz, 2H), 3.93 (dd, J=9.8, 4.4 Hz, 2H), 0.82-0.96 (m, 9H), 0.05-0.13 (m, 6H). MS(M+1): δ 36.

### Compound 6-85

### N-(6-(3,3-difluoropyrrolidin-1-yl)-1-(4-(trifluoromethoxy)phenyl)-1H-pyrazolo[3,4-d]pyrimidin-4-yl)-5-nitrothiophene-2-carboxamide

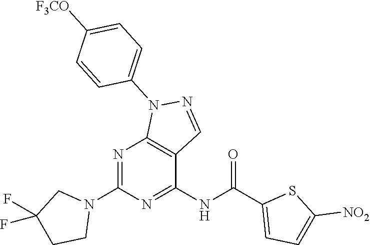 [SMILES: O=C(Nc1nc(N2CCC(F)(F)C2)nc2c1cnn2-c1ccc(OC(F)(F)F)cc1)c1ccc([N+](=O)[O-])s1]

¹H NMR (400 MHz, DMSO-d₆): δ 11.65 (br. s., 1H), 8.40 (d, J=9.3 Hz, 2H), 8.35 (s, 1H), 8.31 (d, J=4.4 Hz, 1H), 8.22 (d, J=4.4 Hz, 1H), 7.55 (d, J=8.8 Hz, 2H), 4.06 (t, J=13.2 Hz, 2H), 3.88 (t, J=7.3 Hz, 2H), 2.52-2.68 (m, 2H). MS(M+1): 556.

### Compound 6-86

### N-(6-(4-(tert-butyl)phenyl)-1-(4-(trifluoromethoxy)phenyl)-1H-pyrazolo[3,4-d]pyrimidin-4-yl)-5-nitrothiophene-2-carboxamide

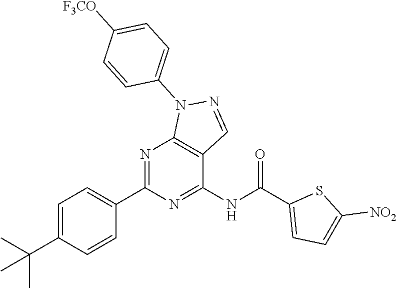 [SMILES: CC(C)(C)c1ccc(-c2nc(NC(=O)c3ccc([N+](=O)[O-])s3)c3cnn(-c4ccc(OC(F)(F)F)cc4)c3n2)cc1]

¹H NMR (400 MHz, DMSO-d₆): δ 12.06 (br. s., 1H), 8.62 (s, 1H), 8.43-8.55 (m, 4H), 8.38 (d, J=4.4 Hz, 1H), 8.25 (d, J=4.4 Hz, 1H), 7.54-7.73 (m, 4H), 1.35 (s, 9H).

MS(M+1): 583.

### Compound 6-87

### 5-nitro-N-(6-(4-(tert-pentyl)phenyl)-1-(4-(trifluoroethoxy)phenyl)-1H-pyrazolo[3,4-d]pyrimidin-4-yl)thiophene-2-carboxamide

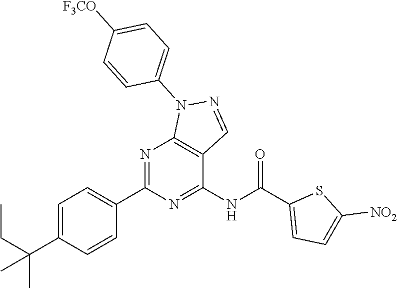 [SMILES: CCC(C)(C)c1ccc(-c2nc(NC(=O)c3ccc([N+](=O)[O-])s3)c3cnn(-c4ccc(OC(F)(F)F)cc4)c3n2)cc1]

¹H NMR (400 MHz, DMSO-d₆): δ 12.04 (br. s., 1H), 8.63 (s, 1H), 8.42-8.51 (m, 4H), 8.38 (d, J=4.4 Hz, 1H), 8.25 (d, J=4.4 Hz, 1H), 7.65 (d, J=8.3 Hz, 2H), 7.55 (d, J=8.3 Hz, 2H), 1.69 (q, J=7.3 Hz, 2H), 1.32 (s, 6H), 0.67 (t, J=7.3 Hz, 3H). MS(M+1): 597.

### Compound 6-88

### 5-nitro-N-(1-(4-(trifluoromethoxy)phenyl)-6-(4-(trifluormethyl)phenyl)-1H-pyrazolo[3,4-d]pyrimidin-4-yl)thiophene-2-carboxamide

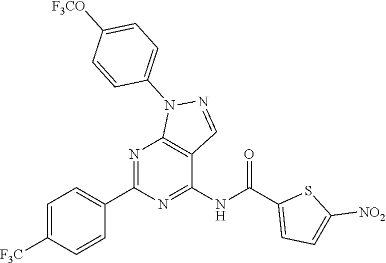 [SMILES: O=C(Nc1nc(-c2ccc(C(F)(F)F)cc2)nc2c1cnn2-c1ccc(OC(F)(F)F)cc1)c1ccc([N+](=O)[O-])s1]

¹H NMR (400 MHz, DMSO-d₆): δ 12.15 (br. s., 1H), 8.70-8.78 (m, J=7.8 Hz, 2H), 8.68 (s, 1H), 8.40-8.48 (m, 2H), 8.38 (d, J=4.4 Hz, 1H), 8.26 (d, J=4.4 Hz, 1H), 7.91-8.00 (m, J=8.3 Hz, 2H), 7.65 (d, J=8.3 Hz, 2H). MS(M+1): 595. Light yellow solid.

### Compound 6-89

### N-(1-(3-fluorophenyl)-6-(4-fluorophenyl)-1H-pyrazolo[3,4-d]pyrimidin-4-yl)-5-nitrothiophene-2-carboxamide

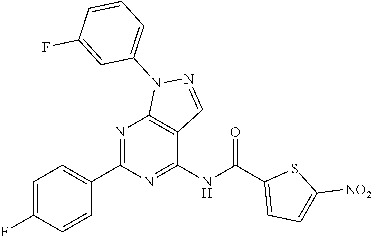 [SMILES: O=C(Nc1nc(-c2ccc(F)cc2)nc2c1cnn2-c1cccc(F)c1)c1ccc([N+](=O)[O-])s1]

¹H NMR (400 MHz, DMSO-d₆): δ 1.99 (br. s., 1H), 8.43-8.64 (m, 3H), 8.35 (d, J=4.4 Hz, 1H), 8.07-8.29 (m, 3H), 7.58-7.77 (m, 1H), 7.41 (t, J=8.8 Hz, 2H), 7.12-7.35 (m, 1H) MS(M+1): 479. Light yellow solid.

### Compound 6-90

### N-(1-(3-fluorophenyl)-6-(6-fluoropyridin-3-yl)-1H-pyrazolo[3,4-d]pyrimidin-4-yl)-5-nitrothiophene-2-carboxamide

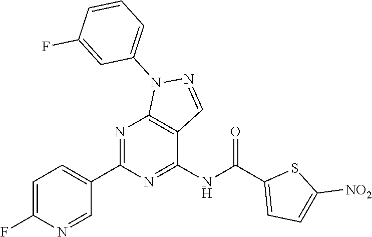 [SMILES: O=C(Nc1nc(-c2ccc(F)nc2)nc2c1cnn2-c1cccc(F)c1)c1ccc([N+](=O)[O-])s1]

¹H NMR (400 MHz, DMSO-d₆): δ 11.97 (br. s., 1H), 9.21 (d, J=2.4 Hz, 1H), 8.84 (td, J=8.1, 2.4 Hz, 1H), 8.54-8.64 (m, 1H), 8.28 (d, J=4.4 Hz, 1H), 8.19-8.24 (m, 1H), 8.17 (dd, J=8.3, 1.5 Hz, 1H), 8.06 (dt, J=11.0, 2.1 Hz, 1H), 7.63 (td, J=8.3, 6.4 Hz, 1H), 7.38 (dd, J=8.6, 2.7 Hz, 1H), 7.22 (td, J=8.6, 2.4 Hz, 1H). MS(M+1): 480. Light yellow solid.

### Compound 6-91

### N-(6-(4-chlorophenyl)-1-(3-fluorophenyl)-1H-pyrazolo[3,4-d]pyrimidin-4-yl)-5-nitrothiophene-2-carboxamide

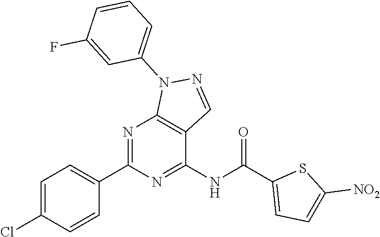 [SMILES: O=C(Nc1nc(-c2ccc(Cl)cc2)nc2c1cnn2-c1cccc(F)c1)c1ccc([N+](=O)[O-])s1]

¹H NMR (400 MHz, DMSO-d₆): δ 11.99 (br. s., 1H), 8.61 (s, 1H), 8.45-8.51 (m, 2H), 8.36 (d, J=4.4 Hz, 1H), 8.24 (d, J=4.4 Hz, 1H), 8.18 (dd, J=8.3, 1.5 Hz, 1H), 8.11 (dt, J=10.8, 2.4 Hz, 1H), 7.60-7.70 (m, 3H), 7.19-7.29 (m, 1H). MS(M+1): 495. Yellow-green solid

### Compound 6-92

### N-(6-(benzo[d][1,3]dioxol-5-yl)-1-(3-fluorophenyl)-1H-pyrazolo[3,4-d]pyrimidin-4-yl)-5-nitrothiophene-2-carboxamide

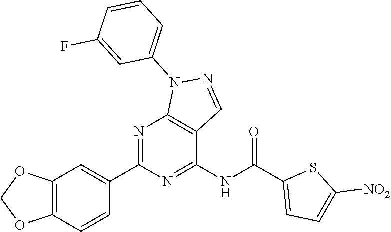 [SMILES: O=C(Nc1nc(-c2ccc3c(c2)OCO3)nc2c1cnn2-c1cccc(F)c1)c1ccc([N+](=O)[O-])s1]

¹H NMR (400 MHz, DMSO-d₆): δ 11.84 (s, 1H), 8.56 (s, 1H), 8.34 (d, J=4.4 Hz, 1H), 8.23 (d, J=4.4 Hz, 1H), 8.16-8.21 (m, 1H), 8.07-8.15 (m, 2H), 7.93 (d, J=1.5 Hz, 1H), 7.65 (td, J=8.3, 6.8 Hz, 1H), 7.23 (td, J=8.1, 2.4 Hz, 1H), 7.09 (d, J=8.3 Hz, 1H), 6.14 (s, 2H). MS(M+1): 505. Yellow solid.

### Compound 6-93

### N-(1-(3-fluorophenyl)-6-(6-((2-methoxyethyl)(ethyl)amino)pyridin-3-yl)-1H-pyrazolo[3,4-d]pyrimidin-4-yl)-5-nitrothiophene-2-carboxamide

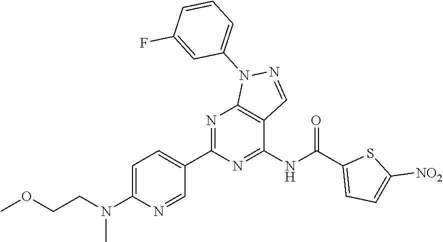 [SMILES: COCCN(C)c1ccc(-c2nc(NC(=O)c3ccc([N+](=O)[O-])s3)c3cnn(-c4cccc(F)c4)c3n2)cn1]

¹H NMR (400 MHz, DMSO-d₆): δ 11.86 (br. s., 1H), 9.20 (d, J=2.4 Hz, 1H), 8.54 (s, 1H), 8.45 (dd, J=9.0, 2.2 Hz, 1H), 8.35 (d, J=4.4 Hz, 1H), 8.20-8.26 (m, 2H), 8.16 (dt, J=10.9, 2.4 Hz, 1H), 7.66 (td, J=8.3, 6.8 Hz, 1H), 7.18-7.26 (m, 1H), 6.79 (d, J=9.3 Hz, 1H), 3.80 (t, J=5.6 Hz, 2H), 3.56 (t, J=5.6 Hz, 2H), 3.28 (s, 3H), 3.13 (s, 3H). MS(M+1): 549. Orange solid.

### Compound 6-94

### N-(1-(3-fluorophenyl)-6-(6-(2-methoxyethoxy)pyridin-3-yl)-1H-pyrazolo[3,4-d]pyrimidin-4-yl)-5-nitrothiophene-2-carboxamide

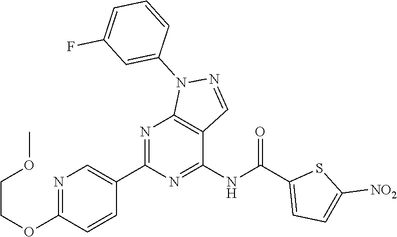 [SMILES: COCCOc1ccc(-c2nc(NC(=O)c3ccc([N+](=O)[O-])s3)c3cnn(-c4cccc(F)c4)c3n2)cn1]

¹H NMR (400 MHz, DMSO-d₆): δ 12.00 (br. s., 1H), 9.25 (d, J=1.5 Hz, 1H), 8.66 (dd, J=8.8, 2.4 Hz, 1H), 8.61 (s, 1H), 8.36 (d, J=4.4 Hz, 1H), 8.20-8.26 (m, 2H), 8.13 (dt, J=10.9, 2.4 Hz, 1H), 7.67 (td, J=8.3, 6.8 Hz, 1H), 7.20-7.29 (m, 1H), 7.03 (d, J=8.8 Hz, 1H), 4.43-4.52 (m, 2H), 3.65-3.73 (m, 2H), 3.33 (s, 3H). MS(M+1): 536. Bright yellow solid.

### Compound 6-95

### N-(6-(6-(2-ethoxyethoxy)pyridin-3-yl)-1-(3-fluorophenyl)-1H-pyrazolo[3,4-d]pyrimidin-4-yl)-5-nitrothiophene-2-carboxamide

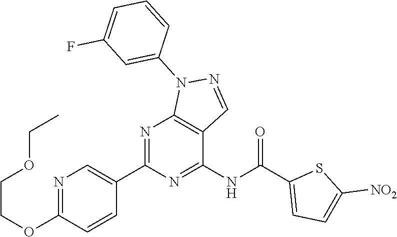 [SMILES: CCOCCOc1ccc(-c2nc(NC(=O)c3ccc([N+](=O)[O-])s3)c3cnn(-c4cccc(F)c4)c3n2)cn1]

¹H NMR (400 MHz, DMSO-d₆): δ 11.96 (br. s., 1H), 9.23 (d, J=2.4 Hz, 1H), 8.65 (dd, J=8.8, 2.4 Hz, 1H), 8.59 (s, 1H), 8.33 (d, J=4.4 Hz, 1H), 8.18-4.26 (m, 2H), 8.12 (dt, J=10.9, 2.4 Hz, 1H), 7.66 (td, J=8.3, 6.4 Hz, 1H), 7.24 (td, J=8.2, 2.2 Hz, 1H), 7.03 (d, J=8.8 Hz, 1H), 4.47 (dd, J=5.4, 3.9 Hz, 2H), 3.74 (dd, J=5.4, 3.9 Hz, 2H), 3.52 (q, J=7.2 Hz, 2H), 1.10-1.19 (m, 3H). MS(M+1): 550. Yellow solid.

### Compound 6-96

### N-(6-(6-(2-(2-ethoxyethoxy)ethoxy)pyridin-3-yl)-1-(3-fluorophenyl)-1H-pyrazolo[3,4-d]pyrimidin-4-yl)-5-nitrothiophene-2-carboxamide

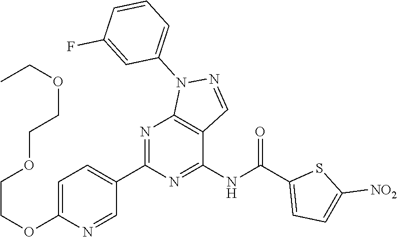 [SMILES: CCOCCOCCOc1ccc(-c2nc(NC(=O)c3ccc([N+](=O)[O-])s3)c3cnn(-c4cccc(F)c4)c3n2)cn1]

¹H NMR (400 MHz, DMSO-d₆): δ 11.80 (br. s., 1H), 9.12 (d, J=2.4 Hz, 1H), 8.55 (dd, J=8.8, 2.4 Hz, 1H), 8.49 (s, 1H), 8.11-8.35 (m, 4H), 7.49-7.64 (m, 1H), 7.32-7.41 (m, 1H), 6.95 (d, J=8.8 Hz, 1H), 4.37-4.50 (m, 2H), 3.69-3.85 (m, 2H), 3.56-3.64 (m, 2H), 3.48-3.54 (m, 2H), 3.44 (q, J=6.8 Hz, 2H), 1.10 (t, J=7.1 Hz, 3H). MS(M+1) 594. Yellow solid.

### Compound 6-97

### N-(6-(3,3-difluoropyrrolidin-1-yl)-1-(4-fluorophenyl)-1H-pyrazolo[3,4-d]pyrimidin-4-yl)-5-nitrothiophene-2-carboxamide

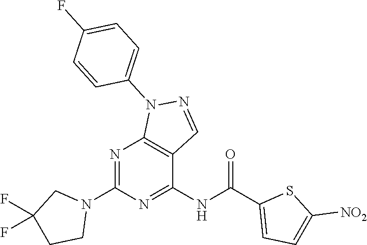 [SMILES: O=C(Nc1nc(N2CCC(F)(F)C2)nc2c1cnn2-c1ccc(F)cc1)c1ccc([N+](=O)[O-])s1]

¹H NMR (400 MHz, DMSO-d₆): δ 11.65 (br. s., 1H), 8.15-8.41 (m, 5H), 7.27-7.46 (m, 2H), 4.05 (t, J=13.0 Hz, 2H), 3.88 (t, J=7.3 Hz, 2H), 2.54-2.68 (m, 2H).

MS(M+1): 490

### Compound 6-98

### N-(6-(4,4-difluoropiperidin-1-yl)-1-(4-fluorophenyl)-1H-pyrazolo[3,4-d]pyrimidin-4-yl)-5-nitrothiophene-2-carboxamide

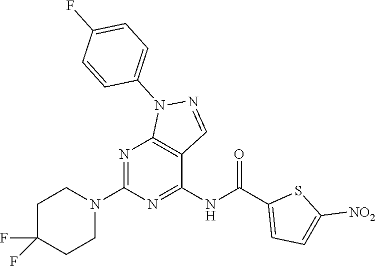 [SMILES: O=C(Nc1nc(N2CCC(F)(F)CC2)nc2c1cnn2-c1ccc(F)cc1)c1ccc([N+](=O)[O-])s1]

¹H NMR (400 MHz, DMSO-d₆): δ 11.57 (s, 1H), 8.27-8.37 (m, 2H), 8.12-8.27 (m, 3H), 7.29-7.45 (m, 2H), 4.03 (t, J=5.6 Hz, 4H), 1.95-2.16 (m, 4H). MS(M+1): 504.

### Compound 6-99

### N-(6-((2S,6R)-2,6-dimethylmorpholino)-1-(4-fluorophenyl)-1H-pyrazolo[3,4-d]pyrimidin-4-yl)-5-nitrothiophene-2-carboxamide

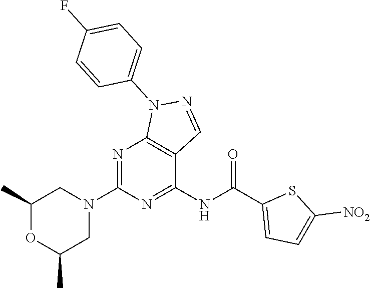 [SMILES: C[C@@H]1CN(c2nc(NC(=O)c3ccc([N+](=O)[O-])s3)c3cnn(-c4ccc(F)cc4)c3n2)C[C@H](C)O1]

¹H NMR (4007 MHz, DMSO-d₆): δ 11.50 (s, 1H), 8.26-8.37 (m, 2H), 8.10-826 (m, 3H), 7.23-7.53 (m, 2H), 4.63 (d, J=10.8 Hz, 2H), 3.61 (ddd, J=10.6, 6.2, 2.7 Hz, 2H), 2.65 (dd, J=13.2, 10.8 Hz, 2H), 1.09-1.28 (m, 6H). MS(M+1): 498. Dark yellow solid.

### Compound 6-100

### methyl (1-(4-fluorophenyl)-4-(5-nitrothiophene-2-carboxamido)-1H-pyrazolo [3,4-d]pyrimidin-6-yl)-L-prolinate

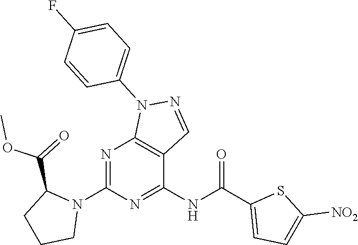 [SMILES: COC(=O)[C@@H]1CCCN1c1nc(NC(=O)c2ccc([N+](=O)[O-])s2)c2cnn(-c3ccc(F)cc3)c2n1]

¹H NMR (400 MHz, DMSO-d₆): δ 11.65 (br. s., 1H), 8.15-8.34 (m, 5H), 7.30-7.44 (m, 2H), 4.56 (dd, J=8.6, 3.7 Hz, 1H), 3.81 (t, J=6.1 Hz, 2H), 3.64 (s, 3H), 2.28-2.46 (m, 1H), 1.88-2.14 (m, 3H). MS(M+1): 512. yellow solid.

### Compound 6-10

### ethyl (1-(4-fluorophenyl)-4-(5-nitrothiophene-2-carboxamido)-1H-pyrazolo[3,4-d] pyrimidin-6-yl)-L-prolinate

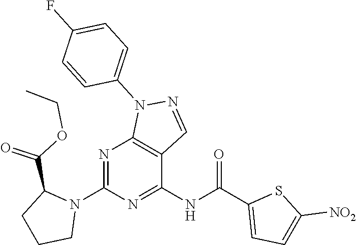 [SMILES: CCOC(=O)[C@@H]1CCCN1c1nc(NC(=O)c2ccc([N+](=O)[O-])s2)c2cnn(-c3ccc(F)cc3)c2n1]

¹H NMR (400 MHz, DMSO-d₆): δ 11.60 (br. s., 1H), 8.13-8.36 (m, 5H), 7.26-7.44 (m, 2H), 4.58 (dd, J=8.3, 3.4 Hz, 1H), 3.94-4.19 (m, 2H), 3.65-3.90 (m, 2H), 2.29-2.47 (m, 1H), 2.02 (d, J=3.4 Hz, 3H), 1.04-1.22 (m, 3H). MS(M+1): 526. Orange solid.

### Compound 6-102

### (S)—N-(1-(4-fluorophenyl)-6-(2-(hydroxymethyl)pyrrolidin-1-yl)-1H-pyrazolo[3,4-d]pyrimidin-4-yl)-5-nitrothiophene-2-carboxamide

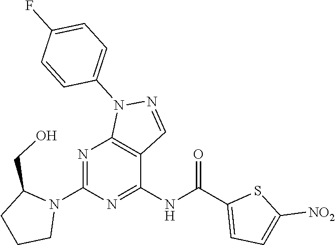 [SMILES: O=C(Nc1nc(N2CCC[C@H]2CO)nc2c1cnn2-c1ccc(F)cc1)c1ccc([N+](=O)[O-])s1]

¹H NMR (400 MHz, DMSO-d₆): δ 11.54 (br. s., 1H), 8.24-8.35 (m, 4H), 8.22 (d, J=4.4 Hz, 1H), 7.37 (t, J=8.8 Hz, 2H), 4.26 (br. s., 1H), 3.51-3.80 (m, 5H), 1.85-2.15 (m, 4H). MS(M+1): 484. Yellow solid.

### Compound 6-103

### (S)—N-(1-(4-fluorophenyl)-6-(2-(methoxymethyl)pyrrolidin-1-yl)-1H-pyrazolo[3,4-d]pyrimidin-4-yl)-5-nitrothiophene-2-carboxamide

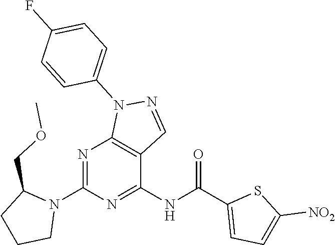 [SMILES: COC[C@@H]1CCCN1c1nc(NC(=O)c2ccc([N+](=O)[O-])s2)c2cnn(-c3ccc(F)cc3)c2n1]

¹H NMR (400 MHz, DMSO-d₆): δ 11.51 (br. s., 1H), 8.23-8.33 (m, 4H), 8.20 (d, J=4.4 Hz, 1H), 7.35 (t, J=8.8 Hz, 2H), 4.35 (br. s., 1H), 3.52-3.76 (m, 3H), 3.39-3.49 (m, 1H), 2.02 (br. s., 3H), 1.78-1.97 (m, 1H). MS(M+1): 498. Yellow solid.

### Compound 6-104

### (S)—N-(6-(2-(ethoxymethyl)pyrrolidin-1-yl)-1-(4-fluorophenyl)-1H-pyrazolo[3,4-d]pyridin-4-yl)-5-nitrothiophene-2-carboxamide

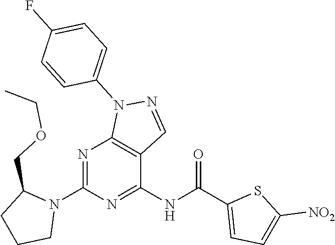 [SMILES: CCOC[C@@H]1CCCN1c1nc(NC(=O)c2ccc([N+](=O)[O-])s2)c2cnn(-c3ccc(F)cc3)c2n1]

¹H NMR (400 MHz, DMSO-d₆): δ 11.52 (br. s., 1H), 8.23-8.38 (m, 4H), 8.19-8.23 (m, 1H), 7.35 (t, J=8.1 Hz, 2H), 4.36 (br. s., 1H), 3.72 (d, J=7.8 Hz, 1H), 3.63 (br. s., 2H), 3.38-3.56 (m, 3H), 2.03 (br. s., 3H), 1.92 (d, J=6.4 Hz, 1H), 1.11 (t, J=7.1 Hz, 3H). MS(M+1): 512. Yellow solid.

### Compound 6-105

### (S)—N-(1-(4-fluorophenyl)-6-(2-(propoxymethyl)pyrrolidin-1-yl)-1H-pyrazolo[3,4-d]pyrimidin-4-yl)-5-nitrothiophene-2-carboxamide

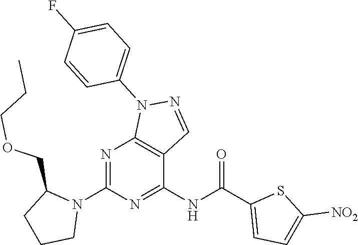 [SMILES: CCCOC[C@@H]1CCCN1c1nc(NC(=O)c2ccc([N+](=O)[O-])s2)c2cnn(-c3ccc(F)cc3)c2n1]

¹H NMR (400 MHz, DMSO-d₆): δ 11.51 (br. s., 1H), 8.16-8.36 (m, 5H), 7.25-7.43 (m, 2H), 4.25-4.46 (m, 1H), 3.71 (dd, J=9.0, 2.7 Hz, 1H), 3.63 (br. s., 2H), 3.33-3.51 (m, 3H), 1.96-2.15 (m, 3H), 1.92 (br. s., 1H), 1.43-1.56 (m, 2H), 0.85 (t, J=7.3 Hz, 3H).

MS(M+1): 526. Orange solid.

### Compound 6-106

### (S)—N-(1-(4-fluorophenyl)-6-(2-(phenoxymethyl)pyrrolidin-1-yl)-1H-pyrazolo[3,4-d]pyrimidin-4-yl)-5-nitrothiophene-2-carboxamide

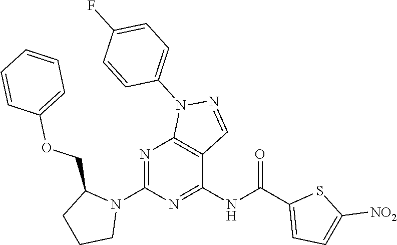 [SMILES: O=C(Nc1nc(N2CCC[C@H]2COc2ccccc2)nc2c1cnn2-c1ccc(F)cc1)c1ccc([N+](=O)[O-])s1]

¹H NMR (400 MHz, DMSO-d₆): δ 11.51 (br. s., 1H), 8.27 (s, 2H), 8.20 (d, J=3.9 Hz, 2H), 7.37 (br. s., 1H), 7.20-7.29 (m, 2H), 7.06 (br. s., 1H), 6.97 (d, J=7.8 Hz, 2H), 6.91 (t, J=6.8 Hz, 1H), 4.41 (br. s., 1H), 4.32 (d, J=4.9 Hz, 1H), 4.06 (t, J=8.6 Hz, 1H), 3.71 (br. s., 2H), 2.14 (br. s., 3H), 1.99 (br. s., 1H) MS(M+1): 560. Yellow solid.

### Compound 6-107

### N-1-(4-fluorophenyl)-6-(thiophen-3-yl)-1H-pyrazolo[3,4-d]pyrimidin-4-yl)-5-nitrothiophene-2-carboxamide

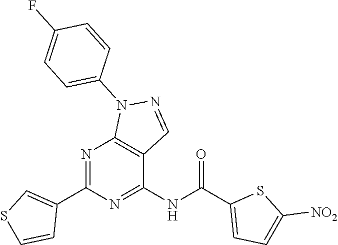 [SMILES: O=C(Nc1nc(-c2ccsc2)nc2c1cnn2-c1ccc(F)cc1)c1ccc([N+](=O)[O-])s1]

¹H NMR (400 MHz, DMSO-d₆): δ 12.03 (s, 1H), 8.57 (s, 1H), 8.52-8.51 (m, 1H), 8.36 (d, J=7.8 Hz, 1H), 8.35-8.29 (m, 2H), 8.24 (d, J=8.8 Hz, 1H), 7.96-7.94 (m, 1H), 7.73-7.71 (m, 1H), 7.48-7.43 (m, 2H). MS(M+1): 467. Yellow solid.

### Compound 6-108

### N-(1-(4-fluorophenyl)-6-(4-(3-methyl-1H-pyrazol-1-yl)-1H-pyrazolo[3,4-d]pyrimidin-4-yl)-5-nitrothiophene-2-carboxamide

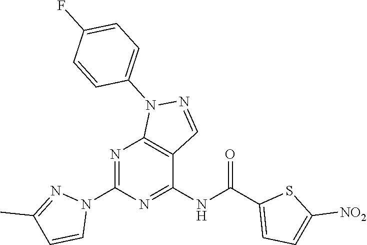 [SMILES: Cc1ccn(-c2nc(NC(=O)c3ccc([N+](=O)[O-])s3)c3cnn(-c4ccc(F)cc4)c3n2)n1]

¹H NMR (400 MHz, DMSO-d₆): δ 12.33 (br. s., 1H), 8.66 (d, J=2.4 Hz, 1H), 8.59 (s, 1H), 8.36 (d, J=4.4 Hz, 1H), 8.15-8.33 (m, 3H), 7.34-7.58 (m, 2H), 6.48 (d, J=2.4 Hz, 1H), 2.33 (s, 3H). MS(M+1): 465. Yellow solid.

### Compound 6-109

### N-(1-(4-fluorophenyl)-6-(3-(trifluoromethyl)-1H-pyrazol-1-yl)-1H-pyrazolo[3,4-d]pyrimidin-4-yl)-5-nitrothiophene-2-carboxamide

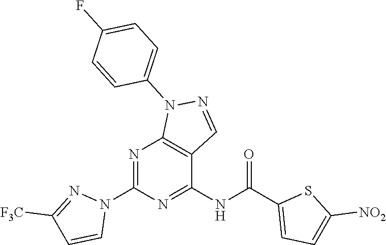 [SMILES: O=C(Nc1nc(-n2ccc(C(F)(F)F)n2)nc2c1cnn2-c1ccc(F)cc1)c1ccc([N+](=O)[O-])s1]

¹H NMR (400 MHz, DMSO-d₆): δ 12.47 (s, 1H), 8.91-9.06 (m, 1H), 8.67 (s, 1H), 8.39 (d, J=4.4 Hz, 1H), 8.14-8.29 (m, 3H), 7.37-7.55 (m, 2H), 7.15 (d, J=2.9 Hz, 1H).

MS(M+1) 519. Light yellow solid.

### Compound 6-110

### N-(1-(4-fluorophenyl)-6-phenyl-1H-pyrazolo[3,4-d]pyrimidin-4-yl)-5-nitrothiophene-2-carboxamide

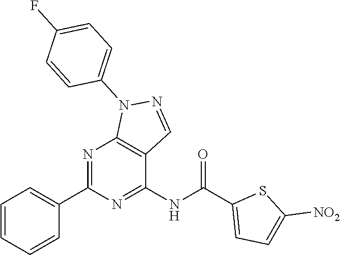 [SMILES: O=C(Nc1nc(-c2ccccc2)nc2c1cnn2-c1ccc(F)cc1)c1ccc([N+](=O)[O-])s1]

¹H NMR (400 MHz, DMSO-d₆): δ 12.02 (br. s., 1H), 8.62 (s, 1H), 8.44-8.59 (m, 2H), 8.16-8.44 (m, 4H), 7.55-7.69 (m, 3H), 7.40-7.55 (m, 2H). MS(M+1): 461.

### Compound 6-111

### N-(1-(4-fluorophenyl)-6-(pyridin-3-yl)-1H-pyrazolo[3,4-d]pyrimidin-4-yl)-5-nitrothiophene-2-carboxamide

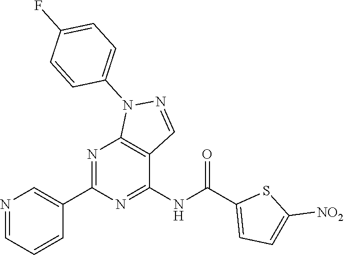 [SMILES: O=C(Nc1nc(-c2cccnc2)nc2c1cnn2-c1ccc(F)cc1)c1ccc([N+](=O)[O-])s1]

¹H NMR (400 MHz, DMSO-d₆): δ 12.08 (br. s., 1H), 9.65-9.69 (m, 1H), 8.77-8.81 (m, 1H), 8.74-8.77 (m, 1H), 8.65 (s, 1H), 8.37 (d, J=4.4 Hz, 1H), 8.28-8.34 (m, 2H), 8.26 (d, J=4.4 Hz, 1H), 7.59-7.65 (m, 1H), 7.45-7.53 (m, 2H). MS(M+1): 462. Yellow solid

### Compound 6-112

### N-(1-(4-fluorophenyl)-6-(pyrimidin-5-yl)-1H-pyrazolo[3,4-d]pyrimidin-4-yl)-5-nitrothiophene-2-carboxamide

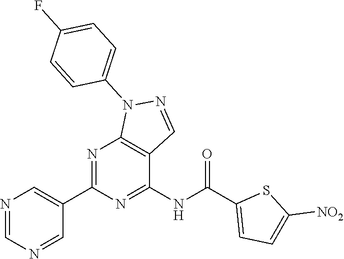 [SMILES: O=C(Nc1nc(-c2cncnc2)nc2c1cnn2-c1ccc(F)cc1)c1ccc([N+](=O)[O-])s1]

¹H NMR (400 MHz, DMSO-d₆): δ 12.12 (br. s., 1H), 9.74 (s, 2H), 9.38 (s, 1H), 8.68 (s, 1H), 8.18-8.45 (m, 4H), 7.38-7.59 (m, 2H). MS(M+1): 463. Khaki solid.

### Compound 6-113

### N-(6-(2-fluorophenyl)-1-(4-fluorophenyl)-1H-pyrazolo[3,4-d]pyrimidin-4-yl)-5-nitrothiophene-2-carboxamide

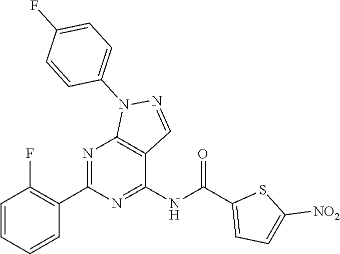 [SMILES: O=C(Nc1nc(-c2ccccc2F)nc2c1cnn2-c1ccc(F)cc1)c1ccc([N+](=O)[O-])s1]

¹H NMR (400 MHz, DMSO-d₆): δ 12.19 (br. s., 1H), 8.65 (s, 1H), 8.13-8.41 (m, 51H), 7.54-7.67 (m, 1H), 7.33-7.54 (m, 4H). MS(M+1): 479. White solid.

### Compound 6-114

### N-(6-(3-fluorophenyl)-1-(4-fluorophenyl)-1H-pyrazolo[3,4-d]pyrimidin-4-yl)-5-nitrothiophene-2-carboxamide

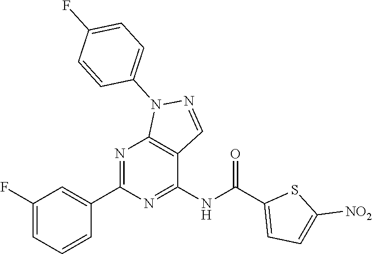 [SMILES: O=C(Nc1nc(-c2cccc(F)c2)nc2c1cnn2-c1ccc(F)cc1)c1ccc([N+](=O)[O-])s1]

¹H NMR (400 MHz, DMSO-d₆): δ 11.97 (br. s., 1H), 8.61 (s, 1H), 8.14-8.39 (m, 6H), 7.62 (td, J=8.1, 5.9 Hz, 1H), 7.36-7.55 (m, 3H). MS(M+1): 479. Khaki solid.

### Compound 6-115

### N-(1,6-bis(4-fluorophenyl)-1H-pyrazolo[3,4-d]pyrimidin-4-yl)-5-nitrothiophene-2-carboxamide

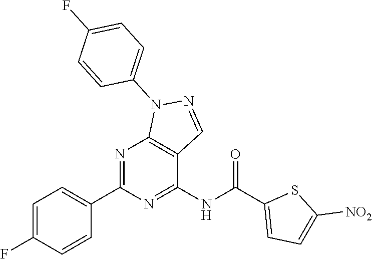 [SMILES: O=C(Nc1nc(-c2ccc(F)cc2)nc2c1cnn2-c1ccc(F)cc1)c1ccc([N+](=O)[O-])s1]

¹H NMR (400 MHz, DMSO-d₆): δ 12.02 (br. s., 1H), 8.52-8.66 (m, 3H), 8.37 (d, J=4.4 Hz, 1H), 8.17-8.33 (m, 3H), 7.31-7.55 (m, 4H). MS(M+1): 479. Yellow solid.

### Compound 6-116

### N-(1-(4-fluorophenyl)-6-(6-fluoropyridin-3-yl)-1H-pyrazolo[3,4-d]pyrimidin-4-yl)-5-nitrothiophene-2-carboxamide

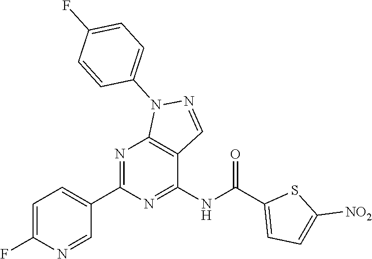 [SMILES: O=C(Nc1nc(-c2ccc(F)nc2)nc2c1cnn2-c1ccc(F)cc1)c1ccc([N+](=O)[O-])s1]

¹H NMR (400 MHz, DMSO-d₆): δ 12.05 (s, 1H), 9.28 (s, 1H), 8.94-8.90 (m, 1H), 8.83 (d, J=7.8 Hz, 1H), 8.63 (s, 1H), 8.39 (d, J=8.8 Hz, 1H), 8.30-8.25 (m, 3H), 7.91-7.88 (m, 1H), 7.42-7.39 (m, 1H). MS(M+1): 480. Yellow solid.

### Compound 6-117

### N-(6-(3-chlorophenyl)-1-(4-fluorophenyl)-1H-pyrazolo[3,4-d]pyrimidin-4-yl)-5-nitrothiophene-2-carboxamide

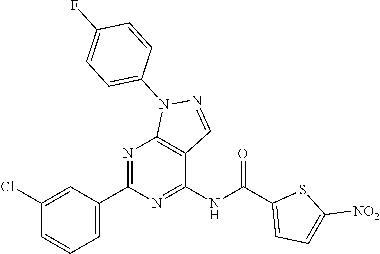 [SMILES: O=C(Nc1nc(-c2cccc(Cl)c2)nc2c1cnn2-c1ccc(F)cc1)c1ccc([N+](=O)[O-])s1]

¹H NMR (400 MHz, DMSO-d₆): δ 12.01 (s, 1H), 8.64 (s, 1H), 8.53-8.52 (m, 1H), 8.47-8.45 (m, 1H), 8.38 (d, J=7.8 Hz, 1H), 8.29-8.26 (m, 3H), 7.67-7.60 (m, 2H), 7.51-7.47 (m, 2H). MS(M+1): 495. Yellow solid.

### Compound 6-1.18

### N-(6-(chlorophenyl)-1-(4-fluorophenyl)-1H-pyrazolo[3,4-d]pyrimidin-4-yl)-5-nitrothiophene-2-carboxamide

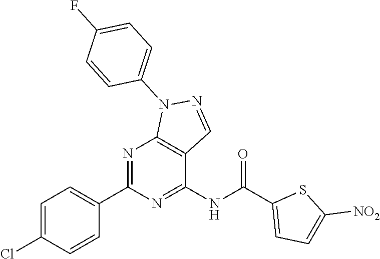 [SMILES: O=C(Nc1nc(-c2ccc(Cl)cc2)nc2c1cnn2-c1ccc(F)cc1)c1ccc([N+](=O)[O-])s1]

¹H NMR (400 MHz, DMSO-d₆): δ 12.00 (br. s., 1H), 8.60 (s, 1H), 8.47-8.53 (m, 2H), 8.37 (d, J=4.4 Hz, 1H), 8.23-8.29 (m, 3H), 7.60-7.67 (m, 2H), 7.43-7.50 (m, 2H). MS(M+1): 495. Yellow solid.

### Compound 6-119

### N-(6-(6-chloropyridin-3-yl)-1-(4-fluorophenyl)-1H-pyrazolo[3,4-d]pyrimidin-4-yl)-5-nitrothiophene-2-carboxamide

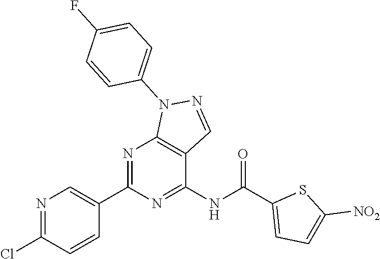 [SMILES: O=C(Nc1nc(-c2ccc(Cl)nc2)nc2c1cnn2-c1ccc(F)cc1)c1ccc([N+](=O)[O-])s1]

¹H NMR (400 MHz, DMSO-d₆): δ 12.06 (br. s., 1H), 9.43 (d, J=2.4 Hz, 1H), 8.77 (dd, J=8.3, 2.4 Hz, 1H), 8.63 (s, 1H), 8.36 (d, J=4.4 Hz, 1H), 8.23-8.30 (m, 3H), 7.73 (d, J=8.3 Hz, 1H), 7.46 (t, J=8.8 Hz, 2H). MS(M+1): 496. yellow solid.

### Compound 6-120

### N-(1-(4-fluorophenyl)-6-(6-methylpyridin-3-yl)-1H-pyrazolo[ 3,4-d]pyrimidin-4-yl)-5-nitrothiophene-2-carboxamide

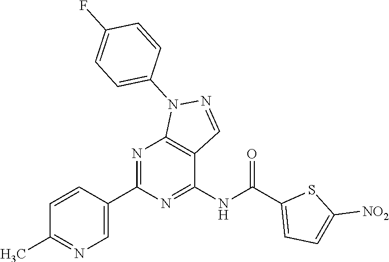 [SMILES: Cc1ccc(-c2nc(NC(=O)c3ccc([N+](=O)[O-])s3)c3cnn(-c4ccc(F)cc4)c3n2)cn1]

¹H NMR (400 Hz, DMSO-d₆): δ 12.05 (br. s., 1H), 9.54 (d, J=2.0 Hz, 1H), 8.66 (dd, J=8.1, 2.2 Hz, 1H), 8.63 (s, 1H), 8.38 (d, J=4.4 Hz, 1H), 8.27-8.34 (m, 2H), 8.26 (d, J=4.4 Hz, 1H), 7.43-7.54 (m, 3H), 2.58 (s, 3H). MS(M+1): 476. Amber solid.

### Compound 6-121

### N-(1-(4-fluorophenyl)-6-(6-methoxypyridin-3-yl)-1H-pyrazolo[3,4-d]pyrimidin-4-yl)-5-nitrothiophene-2-carboxamide

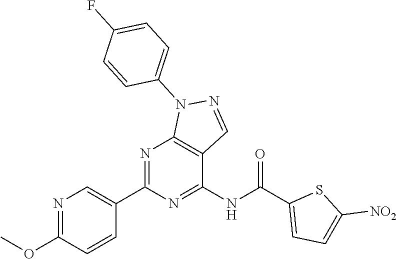 [SMILES: COc1ccc(-c2nc(NC(=O)c3ccc([N+](=O)[O-])s3)c3cnn(-c4ccc(F)cc4)c3n2)cn1]

¹H NMR (400 MHz, DMSO-d₆): δ 11.90 (s, 1H), 9.24 (d, J=2.4 Hz, 1H), 8.63 (dd, J=8.8, 2.4 Hz, 1H), 8.55 (s, 1H), 8.34 (d, J=4.4 Hz, 1H), 8.28 (dd, J=9.3, 4.9 Hz, 2H), 8.23 (d, J=4.4 Hz, 1H), 7.44 (t, J=8.8 Hz, 2H), 6.97 (d, J=8.8 Hz, 1H), 3.94 (s, 3H). MS(M+1): 492. Yellow solid.

### Compound 6-122

### N-(1-(4-fluorophenyl)-6-(4-(trifluoromethyl)phenyl)-1H-pyrazolo[3,4-d]pyrimidin-4-yl)-5-nitrothiophene-2-carboxamide

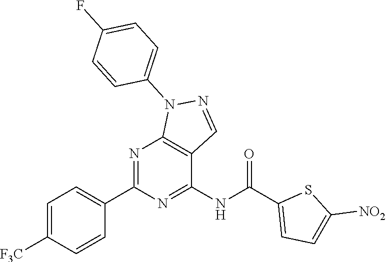 [SMILES: O=C(Nc1nc(-c2ccc(C(F)(F)F)cc2)nc2c1cnn2-c1ccc(F)cc1)c1ccc([N+](=O)[O-])s1]

¹H NMR (400 MHz, DMSO-d₆): δ 12.06 (s, 1H), 8.62-8.69 (m, J=8.3 Hz, 2H), 8.60 (s, 1H), 8.37 (d, J=4.4 Hz, 1H), 8.13-8.28 (m, 3H), 7.84-7.99 (m, J=8.3 Hz, 2H), 7.45 (t, J=8.8 Hz, 2H). MS(M+1): 529.

### Compound 6-123

### N-(1-(4-fluorophenyl)-6-(4-(trifluoromethoxy)phenyl)-1H-pyrazolo[3,4d]pyrimidin-4-yl)-5-nitrothiophene-2-carboxamide

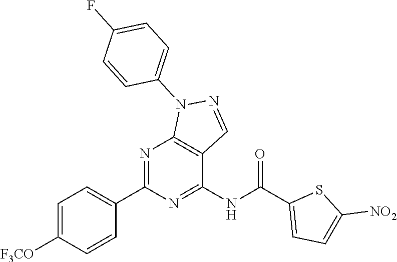 [SMILES: O=C(Nc1nc(-c2ccc(OC(F)(F)F)cc2)nc2c1cnn2-c1ccc(F)cc1)c1ccc([N+](=O)[O-])s1]

¹H NMR (400 MHz, DMSO-d₆): δ 12.11 (br. s., 1H), 8.52-8.74 (m, 3H), 8.22-8.42 (m, 4H), 7.58 (d, J=7.8 Hz, 2H), 7.49 (t, J=8.8 Hz, 2H). MS(M+1): 545. Yellow solid.

### Compound 6-124

### N-(6-(benzo[d][1,3]dioxol-5-yl)-1-(4-fluorophenyl)-1H-pyrazolo[3,4-d]pyrimidin-4-yl)-5-nitrothiophene-2-carboxamide

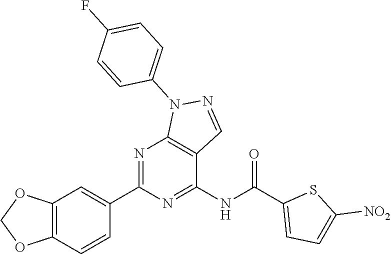 [SMILES: O=C(Nc1nc(-c2ccc3c(c2)OCO3)nc2c1cnn2-c1ccc(F)cc1)c1ccc([N+](=O)[O-])s1]

¹H NMR (400 MHz, DMSO-d₆): δ 11.90 (br. s., 1H), 8.58 (s, 1H), 8.35 (d, J=4.4 Hz, 1H), 8.23-8.34 (m, 3H), 8.14 (dd, J=8.3, 1.5 Hz, 1H), 7.98 (d, J=1.5 Hz, 1H), 7.43-7.53 (m, 2H), 7.10 (d, J=7.8 Hz, 1H), 6.15 (s, 2H). MS(M+1): 505. Light yellow solid.

### Compound 6-125

### N-(6-(3,4-difluorophenyl)-1-(4-fluorophenyl)-1H-pyrazolo[3,4-d]pyrimidin-4-yl)-5-nitrothiophen-2-carboxamide

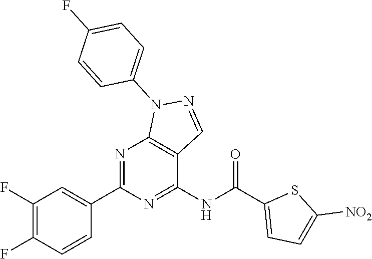 [SMILES: O=C(Nc1nc(-c2ccc(F)c(F)c2)nc2c1cnn2-c1ccc(F)cc1)c1ccc([N+](=O)[O-])s1]

¹H NMR (400 MHz, DMSO-d₆): δ 11.95 (br. s., 1H), 8.61 (s, 1H), 8.31-8.44 (m, 3H), 8.18-8.30 (m, 3H), 7.58-7.70 (m, 1H), 7.47 (t, J=8.8 Hz, 2H). MS(M+1): 497. Brown solid.

### Compound 6-126

### N-(6-(2-(4-difluorophenyl)-1-(4-fluorophenyl)-1H-pyrazolo[3,4-d]pyrimidin-4-yl)-5-nitrothiophene-2-carboxamide

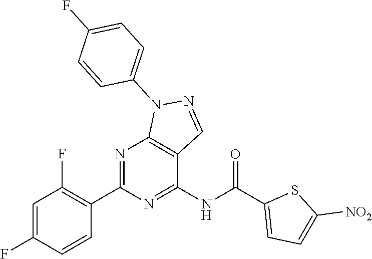 [SMILES: O=C(Nc1nc(-c2ccc(F)cc2F)nc2c1cnn2-c1ccc(F)cc1)c1ccc([N+](=O)[O-])s1]

¹H NMR (400 MHz, DMSO-d₆): δ 12.20 (br. s., 1H), 8.60 (s, 1H), 8.23-8.35 (m, 3H), 8.18-8.23 (m, 2H), 7.38-7.49 (m, 3H), 7.29 (td. J=8.3, 2.4 Hz, 1H). MS(M+1): 497.

ashy solid.

### Compound 6-127

### N-6-(4-chloro-3-fluorophenyl)-1-(4-fluorophenyl)-1H-pyrazolo[3,4-d]pyrimidin-4-yl)-5-nitrothiophene-2-carboxamide

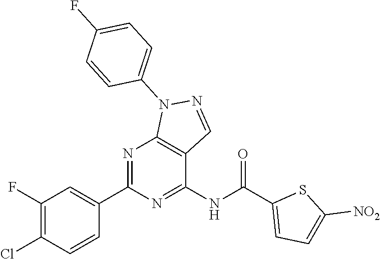 [SMILES: O=C(Nc1nc(-c2ccc(Cl)c(F)c2)nc2c1cnn2-c1ccc(F)cc1)c1ccc([N+](=O)[O-])s1]

¹H NMR (400 MHz, DMSO-d₆): δ 11.97 (br. s., 1H), 8.61 (s, 1H), 8.29-8.39 (m, 3H), 8.20-8.29 (m, 3H), 7.78 (t, J=8.1 Hz, 1H), 7.36-7.53 (m, 2H). MS(M+1): 513. Light khaki solid.

### Compound 6-128

### N-(6-(4-chloro-2-fluorophenyl)-1-(4-fluorophenyl)-1H-pyrazolo[3,4,d]pyrimidin-4-yl)-5-nitrothiophene-2-carboxamide

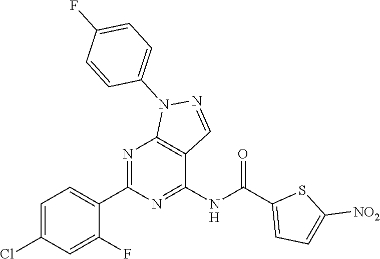

¹H NMR (400 MHz, DMSO-d₆): δ 12.15 (br. s., 1H), 8.63 (s, 1H), 8.36 (d, J=4.4 Hz, 1H), 8.20-8.32 (m, 4H), 7.63 (dd, J=11.0, 2.2 Hz, 1H), 7.50 (dd, J=8.3, 2.0 Hz, 1H), 7.39-7.48 (m, 2H). MS(M+1): 513. Yellow-brown solid.

### Compound 6-129

### N-(6-(4-chloro-2-ethoxyphenyl)-1-(4-fluorophenyl)-1H-pyrazolo[3,4-d]pyrimidin-4-yl)-5-nitrothiophene-2-carboxamide

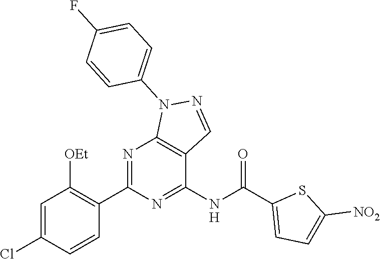

¹H NMR (400 MHz, DMSO-d₆): δ 8.53 (s, 1H), 8.33 (dd, J=9.0, 5.1 Hz, 2H), 7.82-8.20 (m, 3H), 7.41 (t, J=8.8 Hz, 2H), 7.27 (s, 1H), 7.14 (d, J=8.8 Hz, 1H), 4.19 (br. s., 2H), 1.30 (t, J=7.1 Hz, 3H). MS(M+1): 539. Light yellow solid.

### Compound 6-130

### N-(6-(6-(2-(dimethylamino)ethoxy)pyridin-3-yl)-1-(4-fluorophenyl)-1H-pyrazolo[3,4-d]pyrimidin-4-yl)-5-nitrothiophene-2-carboxamide

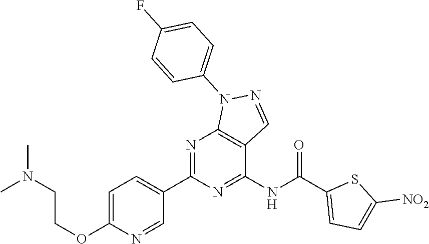

¹H NMR (400 MHz, D₂O+DMSO-d₆): δ 9.14 (d, J=2.1 Hz, 1H), 8.62 (dd, J=8.8, 2.1 Hz, 1H), 8.43 (s, 1H), 8.22 (d, J=4.4 Hz, 1H), 8.18-8.14 (m, 2H), 8.10 (d, J=4.4 Hz, 1H), 7.36 (4, J=8.8 Hz, 2H), 6.99 (d, J=8.8 Hz, 1H), 4.66-4.63 (m, 2H), 3.56-3.53 (m, 2H), 2.88 (s, 6H). MS-ESI (M+1): 549. Yellow solid.

### Compound 6-131

### N-(1-(4-fluorophenyl)-6-(6-((2-methoxyethyl)(methyl)amino)pyridin-3-yl)-1H-pyrazolo[3,4-d]pyrimidin-4-yl)-5-nitrothiophene-2-carboxamide

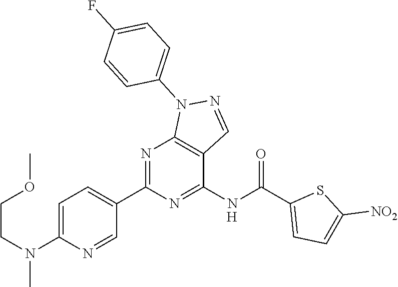

¹H NMR (400 MHz, DMSO-d₆): δ 11.82 (br. s., 1H), 9.18 (d, J=2.0 Hz, 1H), 8.50 (s, 1H), 8.44 (dd, J=9.3, 2.4 Hz, 1H), 8.24-8.37 (m, 3H), 8.22 (d, J=4.4 Hz, 1H), 7.44 (t, J=8.8 Hz, 2H), 6.75 (d, J=9.3 Hz, 1H), 3.78 (t, J=5.6 Hz, 2H), 3.55 (t, J=5.9 Hz, 2H), 3.27 (s, 3H), 3.12 (s, 3H). MS(M+1) 549. Orange solid.

### Compound 6-132

### N-(1-(4-fluorophenyl)-6-(4-propoxyphenyl)-1H-pyrrolo[3,4-d]pyrimidin-4-yl)-5-nitrothiophene-2-carboxamide

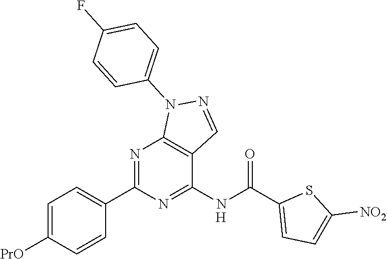

¹H NMR (400 MHz, DMSO-d₆): δ 11.99 (br. s., 1H), 8.58 (s, 1H), 8.39-8.54 (m, 2H), 8.29-8.39 (m, 3H), 8.26 (d, J=4.4 Hz, 1H), 7.38-7.65 (m, 2H), 6.99-7.23 (m, 2H), 4.04 (t, J=6.6 Hz, 2H), 1.79 (tt, J=7.0, 6.6 Hz, 2H), 1.02 (t, J=7.0 Hz, 3H). MS(M+1): 519. Yellow solid.

### Compound 6-133

### N-(1-(4-fluorophenyl)-6-(6-propoxypyridin-3-yl)-1H-pyrazolo[3,4-d]pyrimidin-4-yl)-5-nitrothiophene-2-carboxamide

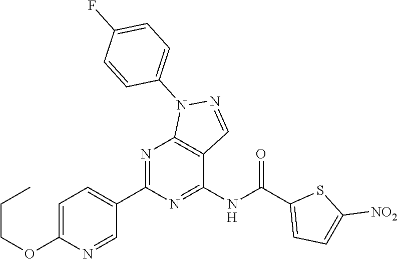

¹H NMR (400 MHz, DMSO-d₆): δ 11.84 (brs, 1H), 9.19 (d, J=2.1 Hz, 1H), 8.60 (dd, J=8.8, 2.1 Hz, 1H), 8.52 (s, 1H), 8.32 (d, J=4.4 Hz, 1H), 8.28-8.23 (m, 2H), 8.21 (d, J=4.4 Hz, 1H), 7.42 (t, J=8.8 Hz, 2H), 6.92 (d, J=8.8 Hz, 1H), 4.28 (t, J=6.8 Hz, 2H), 1.80-1.71 (m, 2H), 0.99 (t, J=6.8 Hz, 3H). MS(M+1): 520. Yellow solid.

### Compound 6-134

### N-(1-(4-fluorophenyl)-6-(6-(2-hydroxyethoxy)pyridin-3-yl)-1H-pyrazolo[3,4,d]pyrimidin-4-yl)-5-nitrothiophene-2-carboxamide

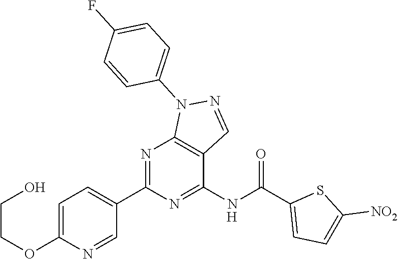

¹H NMR (400 MHz, DMSO-d₆): δ 9.21 (d, J=2.0 Hz, 1H), 8.62-8.76 (m, 1H), 8.37-8.43 (m, 2H), 8.33-8.37 (m, 1H), 8.06 (d, J=3.9 Hz, 1H), 7.58 (d, J=3.9 Hz, 1H), 7.37-7.48 (m, 2H), 6.93 (d, J=9.3 Hz, 1H), 4.88 (br. s., 1H), 4.30-4.44 (m, 2H), 3.76 (t, J=4.9 Hz, 2H). MS(M+1): 522. Orange solid.

### Compound 6-135

### N-(1-(4-fluorophenyl)-6-(6-(2-methoxyethoxy)pyridin-3-yl)-1H-pyrazolo[3,4-d]pyrimidin-4-yl)-5-nitrothiophene-2-carboxamide

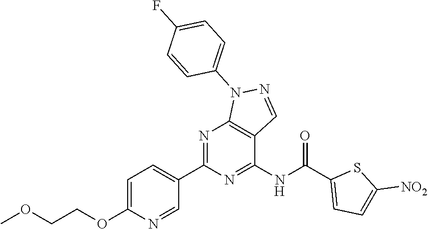

¹H NMR (400 MHz, DMSO-d₆): δ 11.62 (s, 1H), 9.04 (d, J=1.0 Hz, 1H), 8.45 (dd, J=8.3, 1.8 Hz, 1H), 8.37 (s, 1H), 8.24-8.12 (m, 4H), 7.32 (t, J=8.3 Hz, 2H), 6.85 (d, J=8.8 Hz, 1H), 4.40-4.38 (m, 2H), 3.69-3.66 (m, 2H), 3.34 (s, 3H). MS(M+1): 536. Yellow solid.

### Compound 6-136

### N-(6-(6-(2-ethoxyethoxy)pyridin-3-yl)-1-(4-fluorophenyl)-1H-pyrazolo[3,4-d]pyrimidin-4-yl)-5-nitrothiophene-2-carboxamide

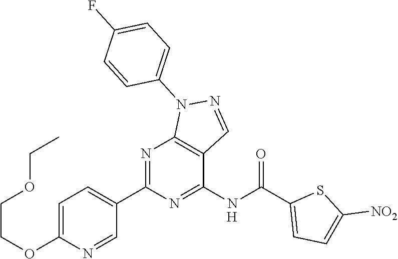

¹H NMR (400 MHz, DMSO-d₆): δ 11.89 (br. s., 1H), 9.21 (d, J=2.0 Hz, 1H), 8.63 (dd, J=8.8, 2.4 Hz, 1H), 8.54 (s, 1H), 8.18-8.38 (m, 4H), 7.34-7.52 (m, 2H), 6.98 (d, J=8.3 Hz, 1H), 4.45 (dd, J=5.6, 4.2 Hz, 2H), 3.73 (dd, J=5.6, 4.2 Hz, 2H), 3.52 (q, J=6.8 Hz, 2H), 1.14 (t, J=6.8 Hz, 3H). MS(M+1): 550. Yellow solid.

### Compound 6-137

### N-(6-(6-(2-(2-ethoxyethoxy)ethoxy)pyridin-3-yl)-1-(4-fluorophenyl)-1H-pyrazolo[3,4-d]pyrimidin-4-yl)-5-nitrothiophene-2-carboxamide

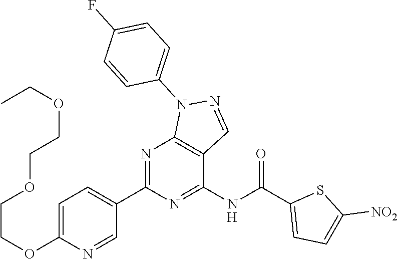

¹H NMR (400 MHz, DMSO-d₆): δ 11.95 (s, 1H), 9.23 (d, J=2.4 Hz, 1H), 8.66 (dd, J=8.8, 2.4 Hz, 1H), 8.56 (s, 1H), 8.33-8.22 (m, 4H), 7.45 (t, J=8.8 Hz, 2H), 6.99 (d, J=8.8 Hz, 1H), 4.47-4.45 (m, 2H), 3.79-3.77 (m, 2H), 3.60-3.58 (m, 2H), 3.51-3.49 (m, 2H), 3.43 (q, J=6.8 Hz, 2H), 1.09 (t, J=6.8 Hz, 3H). MS(M+1): 594. Yellow solid.

### Compound 6-138

### N-(6-(2-fluoro-4-(2-methoxyethoxy)phenyl)-1-(4-fluorophenyl)-1H-pyrazolo[3,4,d]pyrimidin-4-yl)-5-nitrothiophene-2-carboxamide

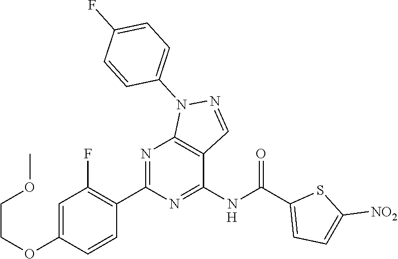

¹H NMR (400 Hz, DMSO-d₆): δ 12.14 (br. s., 1H), 8.61 (s, 1H), 8.18-8.42 (m, 5H), 7.37-7.53 (m, 2H), 6.92-7.08 (m, 2H), 4.22 (dd, J=5.1, 3.7 Hz, 2H), 3.62-3.76 (m, 2H), 3.33 (s, 3H). MS(M+1): 553. Bright yellow solid.

### Compound 6-139

### N-(6-(6-(2-(2-ethoxyethoxy)ethoxy)-4-methylpyridin-3-yl)-1-(4-fluorophenyl)-1H-pyrazolo[3,4-d]pyrimidin-4-yl)-5-nitrothiophene-2-carboxamide

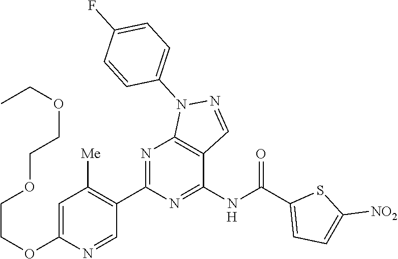

¹H NMR (400 MHz, DMSO-d₆): δ 11.93 (s, 1H), 8.11 (s, 1H), 8.55 (s, 1H), 8.30 (d, J=4.2 Hz, 1H), 8.19-8.15 (m, 1H), 7.42-7.38 (m, 2H), 6.79 (s, 1H), 4.43-4.41 (m, 2H), 3.77-3.74 (m, 2H), 3.60-3.57 (m, 2H), 3.51-3.49 (m, 2H), 3.43 (q, J=6.8 Hz, 2H), 2.63 (s, 3H), 1.10 (t, J=6.8 Hz, 3H). MS(M+1): 608. Yellow solid.

### Compound 6-140

### N-(6-(4-chloro-2-(2-(2-ethoxyethoxy)ethoxy)phenyl)-1-(4-fluorophenyl)-1H-pyrazolo[3,4-d]pyrimidin-4-yl)-5-nitrothiophene-2-carboxamide

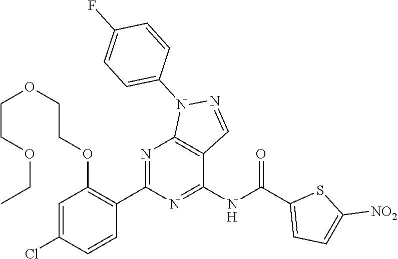

¹H NMR (400 MHz, DMSO-d₆): δ 12.26 (br. s., 1H), 8.65 (s, 1H), 8.13-8.41 (m, 4H), 7.83 (br s., 1H), 7.45 (t, J=8.8 Hz, 2H), 7.37 (br. s., 1H), 7.20 (d, J=7.3 Hz, 1H), 4.26 (br. s., 2H), 3.70 (br. s., 2H), 3.38 (br. s., 2H), 3.15-3.26 (m, 4H), 0.91 (t, J=6.8 Hz, 3H). MS(M+1): δ 28. Yellow solid.

### Compound 6-141

### N-(1-(4-fluorophenyl)-6-(6-morpholinopyridin-3-yl)-1H-pyrazolo[3,4-d]pyrimidin-4-yl)-5-nitrothiophene-2-carboxamide

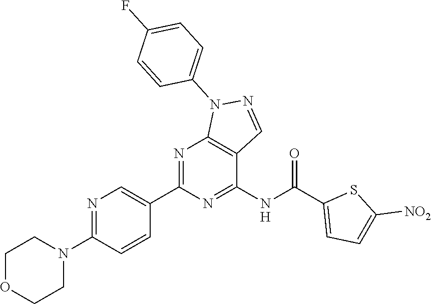

¹H NMR (400 MHz, DMSO-d₆): δ 11.95 (br. s., 1H), 9.27 (d, J=2.0 Hz, 1H), 8.53-8.58 (m, 2H), 8.30-8.37 (m, 3H), 8.25 (d, J=4.4 Hz, 1H), 7.44-7.52 (m, 2H), 7.00 (d, J=8.8 Hz, 1H), 3.69-3.78 (m, 4H), 3.58-3.67 (m, 4H). MS(M+1): 547. brown solid.

### Compound 6-142

### N-1-(4-fluorophenyl)4-(methylcyclohex-1-en-1-yl)-1H-pyrazolo[3,4-d]pyrimidin-4-yl)-5-nitrothiophene-2-carboxamide

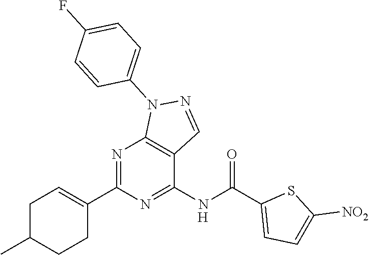

¹H NMR (400 MHz, DMSO-d₆): δ 11.85 (br. s., 1H), 8.52 (s, 1H), 8.15-8.41 (m, 4H), 7.32-7.53 (m, 3H), 2.82 (d, J=17.1 Hz, 1H), 2.36-2.48 (m, 2H), 1.81-2.03 (m, 2H), 1.74 (br. s., 1H), 1.23-1.42 (m, 1H), 1.02 (d, J=6.4 Hz, 3H). MS(M+1): 479. Pale yellow solid.

### Compound 6-143

### N-(1-(4-fluorophenyl)-6-(4-methylcyclohexyl)-1H-pyrazolo[3,4-d]pyrimidin-4-yl)-5-nitrothiophene-2-carboxamide

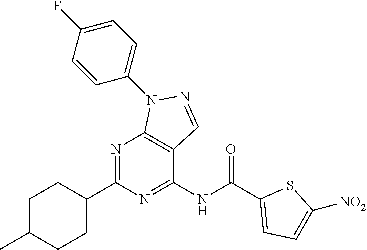 [SMILES: CC1CCC(c2nc(NC(=O)c3ccc([N+](=O)[O-])s3)c3cnn(-c4ccc(F)cc4)c3n2)CC1]

¹H NMR (400 MHz, DMSO-d₆)(cis and trans): δ 12.03 (br. s., 1H), 8.55 (s, 2H), 8.31 (br. s., 2H), 8.09-8.28 (m, 7H), 7.37-7.53 (m, 4H), 3.04 (tt, J=7.9, 4.0 Hz, 1H), 2.84 (tt, J=11.9, 3.2 Hz, 1H), 2.14 (br. s., 2H), 2.06 (d, J=11.2 Hz, 2H), 1.57-1.86 (m, 9H), 1.34-1.52 (m, 3H), 1.03-1.18 (m, 2H), 0.89-1.00 (m, 6H). MS(M+1): 481. Pale yellow solid.

### Compound 6-144

### N-(6-(4,4-dimethylcyclohex-1-en-1-yl)-1-(4-fluorophenyl)-1H-pyrazolo[3,4-d]pyrimidin-4-yl)-5-nitrothiophene-2-carboxamide

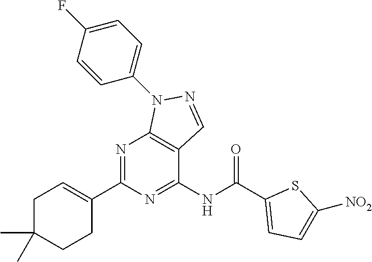 [SMILES: CC1(C)CC=C(c2nc(NC(=O)c3ccc([N+](=O)[O-])s3)c3cnn(-c4ccc(F)cc4)c3n2)CC1]

¹H NMR (400 MHz, DMSO-d₆): δ 11.90 (br. s., 1H), 8.54 (s, 1H), 8.17-8.42 (m, 4H), 7.34-7.57 (m, 3H), 2.66 (br. s., 2H), 2.13 (br. s., 2H), 1.54 (t, J=6.4 Hz, 2H), 0.98 (s, 6H). MS(M+1): 493. Pale yellow solid.

### Compound 6-145

### N-(6-(4,4-dimethylcyclohexyl)-1-(4-fluorophenyl)-1H-pyrazolo[3,4-d]pyrimidin-4-yl)-5-nitrothiophene-2-carboxamide

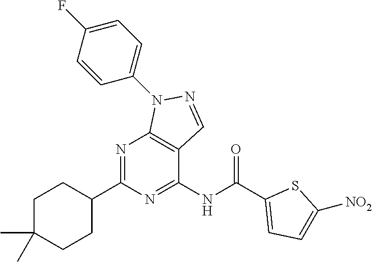 [SMILES: CC1(C)CCC(c2nc(NC(=O)c3ccc([N+](=O)[O-])s3)c3cnn(-c4ccc(F)cc4)c3n2)CC1]

¹H NMR (400 MHz, DMSO-d₆): δ 12.23 (br. s., 1H), 8.55 (s, 1H), 8.16-8.41 (m, 4H), 7.37-7.55 (m, 2H), 2.73-2.90 (m, 1H), 1.75-1.99 (m, 4H), 1.51 (d, J=12.7 Hz, 2H), 1.36 (td, J=12.5, 4.9 Hz, 2H), 0.98 (d, J=4.9 Hz, 6H). MS(M+1): 495. White solid.

TABLE 7

| | 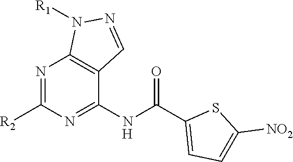 | | |
|---|---|---|---|
| | | | Hep3B |
| | | | LC₅₀ |
| Compound | R1 | R2 | (uM) |
| 7-1 | 5-(trifluoromethyl)pyridin-2-yl | 4-fluorophenyl | 0.26 |
| 7-2 | 5-(trifluoromethyl)pyridin-2-yl | 6-fluoropyridin-3-yl | 0.15 |
| 7-3 | 5-(trifluoromethyl)pyridin-2-yl | 4-chlorophenyl | 0.13 |
| 7-4 | 5-(trifluoromethyl)pyridin-2-yl | 4-(trifluoromethyl) | 0.22 |
| | | phenyl | |
| 7-5 | 5-(trifluoromethyl)pyridin-2-yl | 4-(trifluoromethoxy) | 0.31 |
| | | phenyl | |
| 7-6 | 5-(trifluoromethyl)pyridin-2-yl | 2,4-difluorophenyl | 0.15 |
| 7-7 | 5-(trifluoromethyl)pyridin-2-yl | 6-(2-(2- | 0.50 |
| | | etboxyethoxy)ethoxy) | |
| | | pyridin-3-yl | |
| 7-8 | pyridin-2-yl | 4-fluorophenyl | 0.58 |
| 7-9 | 2.4-dichlorophenyl | 4-fluorophenyl | 0.37 |
| 7-10 | 2,4-dichlorophenyl | 6-fluoropyridin-3-yl | >1.25 |
| 7-11 | 3,4-dichlorophenyl | (S)-2-(hydroxymethyl) | 0.51 |
| | | pyrrolidin-1- | |
| | | yl | |
| 7-12 | 2,4-difluorophenyl | 4-fluorophenyl | 0.43 |
| 7-13 | 2,4-difluorophenyl | 6-fluoropyridin-3-yl | 0.13 |
| 7-14 | 2,4-difluorophenyl | 4-chlorophenyl | 0.58 |
| 7-15 | benzyl | 6-chloropyridin-3-yl | 1.2 |
| 7-16 | benzyl | 4-chlorophenyl | >1.25 |
| 7-17 | benzyl | 6-methylpyridin-3-yl | >1.25 |
| 7-18 | 3-fluorobenzyl | 6-methoxypyridin-3-yl | >1.25 |
| 7-19 | 4-acrylamidophenyl | 4-fluorophenyl | 0.19 |

### Compound 7-1

### N-(6-(4-fluorophenyl)-1-(5-(trifluoromethyl)pyridin-2-yl)-1H-pyrazolo[3,4-d]pyrimidin-4-yl)-5-nitrothiophene-2-carboxamide

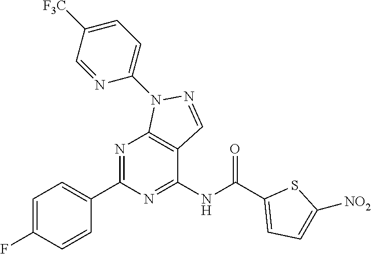 [SMILES: O=C(Nc1nc(-c2ccc(F)cc2)nc2c1cnn2-c1ccc(C(F)(F)F)cn1)c1ccc([N+](=O)[O-])s1]

¹H NMR (400 MHz, DMSO-d₆): δ 12.11 (s, 1H), 9.08 (s, 1H), 8.47-8.76 (m, 5H), 8.39 (d, J=4.4 Hz, 1H), 8.28 (d, J=4.4 Hz, 1H), 7.43 (t, J=8.6 Hz, 2H). MS(M+1): 530.

Light yellow solid.

### Compound 7-2

### N-(6-(6-fluoropyridin-3-yl)-1-(5-(trifluoromethyl)pyridin-2-yl)-1H-pyrazolo[3,4,d]pyrimidin-4-yl)-5-nitrothiophene-2-carboxamide

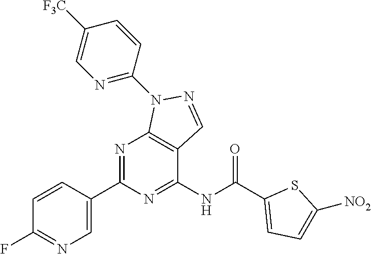 [SMILES: O=C(Nc1nc(-c2ccc(F)nc2)nc2c1cnn2-c1ccc(C(F)(F)F)cn1)c1ccc([N+](=O)[O-])s1]

¹H NMR (400 MHz, DMSO-d₆): δ 12.17 (brs, 1H), 9.36 (d, J=2.4 Hz, 1H), 9.09 (s, 1H), 9.02-8.97 (m, 1H), 8.76 (s, 1H), 8.68 (d, J=8.8 Hz, 1H), 8.57-8.55 (m, 1H), 8.37 (d, J=4.4 Hz, 1H), 8.27 (d, J=4.4 Hz, 2H), 7.45-7.42 (m, 1H). MS(M+1): 531. White solid.

### Compound 7-3

### N-(6-(4-chlorophenyl)-1-(5-(trifluoromethyl)pyridin-2-yl)-1H-pyrazolo[3,4-1]pyrimidin-4-yl)-5-nitrothiophene-2-carboxamide

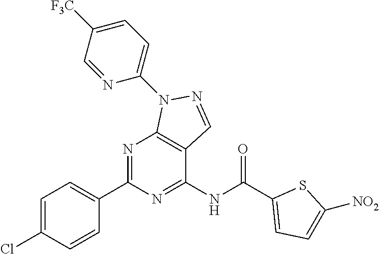 [SMILES: O=C(Nc1nc(-c2ccc(Cl)cc2)nc2c1cnn2-c1ccc(C(F)(F)F)cn1)c1ccc([N+](=O)[O-])s1]

¹H NMR (400 MHz, DMSO-d₆): δ 12.12 (s, 1H), 9.08 (s, 1H), 8.72 (s, 1H), 8.60-8.69 (m, 1H), 8.50-8.60 (m, 3H), 8.39 (d, J=4.4 Hz, 1H), 8.27 (d, J=4.4 Hz, 1H), 7.61-7.74 (m, 2H). MS(M+1): 546. Light yellow solid.

### Compound 7-4

### 5-nitro-N-(6-(4-(trifluoromethyl)phenyl)-1-(5-(trifluoromethyl)pyridin-2-yl)-1H-pyrazolo[3,4-d]pyrimidin-4-yl)thiophene-2-carboxamide

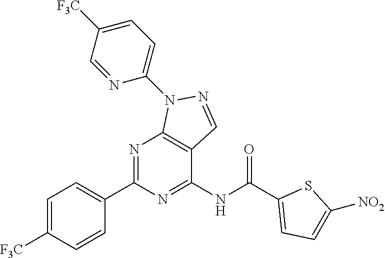 [SMILES: O=C(Nc1nc(-c2ccc(C(F)(F)F)cc2)nc2c1cnn2-c1ccc(C(F)(F)F)cn1)c1ccc([N+](=O)[O-])s1]

¹H NMR (400 MHz, DMSO-d₆): δ 12.21 (br. s., 1H), 9.08 (s, 1H), 8.74 (t, J=4.2 Hz, 3H), 8.64 (d, J=8.8 Hz, 1H), 8.51-8.60 (m, 1H), 8.38 (d, J=4.4 Hz, 1H), 8.27 (d, J=4.4 Hz, 1H), 7.96 (d, J=8.3 Hz, 2H). MS(M+1): 580. White milky solid.

### Compound 7-5

### 5-nitro-N-(6-(4-(trifluoromethoxy)phenyl)-1-(5-(trifluoromethyl)pyridin-2-yl)-1H-pyrazolo[3,4-d]pyrimidin-4-yl)thiophene-2-carboxamide

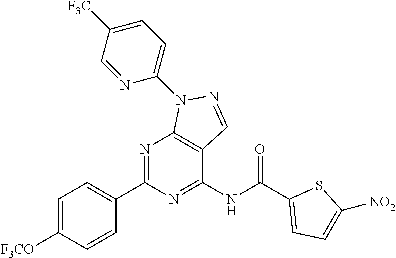 [SMILES: O=C(Nc1nc(-c2ccc(OC(F)(F)F)cc2)nc2c1cnn2-c1ccc(C(F)(F)F)cn1)c1ccc([N+](=O)[O-])s1]

¹H NMR (400 MHz, DMSO-d₆): δ 12.16 (br. s., 1H), 9.08 (s, 1H), 8.72 (s, 1H), 8.66 (dd, J=9.0, 2.2 Hz, 3H), 8.55 (dd, J=8.6, 2.2 Hz, 1H), 8.38 (d, J=4.4 Hz, 1H), 8.27 (d, J=4.4 Hz, 1H), 7.58 (d, J=7.8 Hz, 2H). MS(M+1): 596. White solid.

### Compound 7.6

### N-(6-(2-(4-difluorophenyl)-1-(5-(trifluoromethyl)pyridin-2-yl)-1H-pyrazolo[3,4-d]pyrimidin-4-yl)-5-nitrothiophene-2-carboxamide

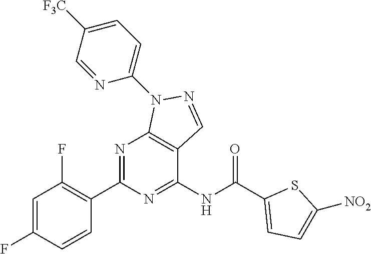 [SMILES: O=C(Nc1nc(-c2ccc(F)cc2F)nc2c1cnn2-c1ccc(C(F)(F)F)cn1)c1ccc([N+](=O)[O-])s1]

¹H NMR (400 MHz, DMSO-d₆): δ 12.25 (br. s., 1H), 9.05 (s, 1H), 8.73 (s, 1H), 8.67 (d, J=8.3 Hz, 1H), 8.54 (dd, J=8.8, 2.4 Hz, 1H), 8.37 (d, J=4.4 Hz, 1H), 8.17-8.34 (m, 2H), 7.46 (t, J=2.2 Hz, 1H), 7.32 (d, J=2.4 Hz, 1H). MS(M+1): 548. White solid.

### Compound 7-7

### N-(6-(6-(2-(2-ethoxyethoxy)ethoxy)pyridin-3-yl)-1-(5-(trifluoromethyl)pyridin-2-yl)-1H-pyrazolo[3,4-d]pyrimidin-4-yl)-5-nitrothiophene-2-carboxamide

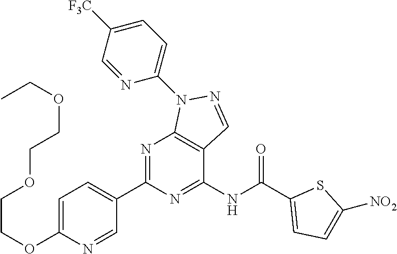 [SMILES: CCOCCOCCOc1ccc(-c2nc(NC(=O)c3ccc([N+](=O)[O-])s3)c3cnn(-c4ccc(C(F)(F)F)cn4)c3n2)cn1]

¹H NMR (400 MHz, DMSO-d₆): δ 11.96 (s, 1H), 9.25 (d, J=2.4 Hz, 1H), 9.02 (s, 1H), 8.68-8.64 (m, 3H), 8.53-8.50 (m, 1H), 8.34 (d, J=8.8 Hz, 1H), 8.23 (d, J=8.8 Hz, 1H), 6.98 (d, J=8.8 Hz, 1H), 4.47-4.45 (m, 2H), 3.79-3.77 (m, 2H), 3.60-3.58 (m, 2H), 3.52-3.49 (m, 2H), 3.43 (q, J=6.8 Hz, 2H), 1.10 (t, J=6.8 Hz, 3H). MS(M+1): δ 45. Yellow solid.

### Compound 7-8

### N-(6-(4-fluorophenyl)-1-(pyridin-2-yl)-1H-pyrazolo[3,4-d]pyrimidin-4-yl)-5-nitrothiophene-2-carboxamide

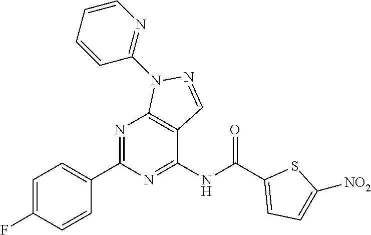 [SMILES: O=C(Nc1nc(-c2ccc(F)cc2)nc2c1cnn2-c1ccccn1)c1ccc([N+](=O)[O-])s1]

¹H NMR (400 MHz, DMSO-d₆): δ 12.04 (br. s., 1H), 8.67-8.71 (m, 1H), 8.65 (s, 1H), 8.53-8.60 (m, 2H), 8.39 (d, J=4.4 Hz, 1H), 8.23-8.30 (m, 2H), 8.11-8.18 (m, 1H), 7.52 (ddd, J=7.3, 4.9, 1.0 Hz, 1H), 7.35-7.45 (m, 2H). MS(M+1): 462. Yellow solid.

### Compound 7-9

### N-(1-(2-(4-dichlorophenyl)-6-(4-fluorophenyl)-1H-pyrazolo[3,4,d]pyrimidin-4-yl)-5-nitrothiophene-2-carboxamide

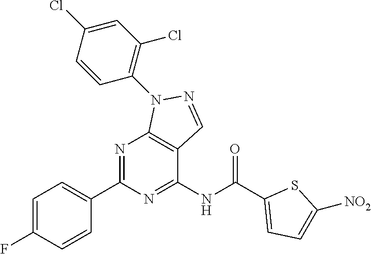 [SMILES: O=C(Nc1nc(-c2ccc(F)cc2)nc2c1cnn2-c1ccc(Cl)cc1Cl)c1ccc([N+](=O)[O-])s1]

¹H NMR (400 MHz, DMSO-d₆): δ 12.10 (br. s., 1H), 8.65 (s, 1H), 8.39-8.48 (m, 2H), 8.37 (d, J=4.4 Hz, 1H), 8.26 (d, J=4.4 Hz, 1H), 8.02 (d, J=2.4 Hz, 1H), 7.81 (d, J=8.3 Hz, 1H), 7.73 (dd, J=8.8, 2.4 Hz, 1H), 7.32-7.41 (m, 2H). MS(M+1): 530. Light khaki solid.

### Compound 7-10

### N-(1-(2-(4-dichlorophenyl)-6-(6-fluoropyridin-3-yl)-1H-pyrazolo[3,4-d]pyrimidin-4-yl)-5-nitrothiophene-2-carboxamide

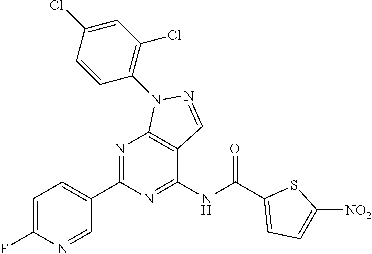 [SMILES: O=C(Nc1nc(-c2ccc(F)nc2)nc2c1cnn2-c1ccc(Cl)cc1Cl)c1ccc([N+](=O)[O-])s1]

¹H NMR (400 MHz, DMSO-d₆): δ 12.19 (br. s., 1H), 9.18 (d, J=2.4 Hz, 1H), 8.78 (td, J=8.3, 2.4 Hz, 1H), 8.68 (s, 1H), 8.31 (d, J=4.4 Hz, 1H), 8.26 (d, J=4.4 Hz, 1H), 8.02 (d, J=2.4 Hz, 1H), 7.81 (d, J=8.3 Hz, 1H), 768-7.78 (m, 1H), 7.35 (dd, J=8.8, 2.4 Hz, 1H). MS(M+1): 530. Light khaki solid.

### Compound 7-11

### (S)—N-(1-(3,4-dichlorophenyl)-6-(2-(hydroxymethyl)pyrrolidin-1-yl)-1H-pyrazolo[3,4-d]pyrimidin-4-yl)-5-nitrothiophene-2-carboxamide

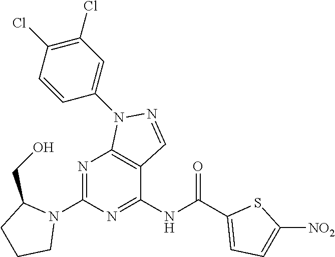 [SMILES: O=C(Nc1nc(N2CCC[C@H]2CO)nc2c1cnn2-c1ccc(Cl)c(Cl)c1)c1ccc([N+](=O)[O-])s1]

¹H NMR (400 MHz, DMSO-d₆): δ 11.54 (br. s., 1H), 8.63 (br. s., 1H), 8.25-8.34 (m, 2H), 8.18-8.25 (m, 1H), 7.77 (d, J=8.8 Hz, 1H), 4.73 (br. s, 1H), 4.25 (d, J=16.6 Hz, 1H), 3.65 (br. s., 4H), 1.98-2.18 (m, 3H), 1.91 (br. s., 1H). MS(M+1): 534. Khaki solid.

### Compound 7-12

### N-(1-(2-(4-difluorophenyl)-6-(4-fluorophenyl)-1H-pyrazolo[3,4-d]pyrimidin-4-yl)-5-nitrothiophene-2-carboxamide

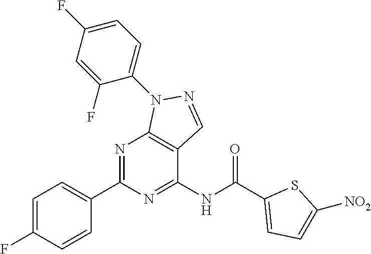 [SMILES: O=C(Nc1nc(-c2ccc(F)cc2)nc2c1cnn2-c1ccc(F)cc1F)c1ccc([N+](=O)[O-])s1]

¹H NMR (400 MHz, DMSO-d₆): δ 12.08 (br. s., 1H), 8.65 (s, 1H), 8.42-8.51 (m, 2H), 8.37 (d, J=4.4 Hz, 1H), 8.26 (d, J=4.4 Hz, 1H), 7.88 (td, J=8.8, 5.9 Hz, 1H), 7.63-7.73 (m, 1H), 7.32-7.44 (m, 3H). MS(M+1): 497. Yellow solid.

### Compound 7-13

### N-(1-(2-(4-difluorophenyl)-6-(6-fluoropyridin-3-yl)-1H-pyrazolo[3,4-d]pyrimidin-4-yl)-5-nitrothiophene-2-carboxamide

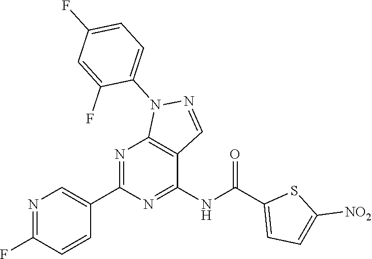 [SMILES: O=C(Nc1nc(-c2ccc(F)nc2)nc2c1cnn2-c1ccc(F)cc1F)c1ccc([N+](=O)[O-])s1]

¹H NMR (400 MHz, DMSO-d₆): δ 12.14 (br. s., 1H), 9.21 (d, J=2.4 Hz, 1H), 8.80 (td, J=8.4, 2.4 Hz, 1H), 8.69 (s, 1H), 8.38 (d, J=4.4 Hz, 1H), 8.27 (d, J=4.4 Hz, 1H), 7.90 (td, J=8.4, 6.0 Hz, 1H), 7.60-7.76 (m, 1H), 7.31-7.47 (m, 2H). MS(M+1): 498.

White solid.

### Compound 7-14

### N-(6-(4-chlorophenyl)-1-(2-(4-difluorophenyl)-1H-pyrazolo[3,4-d]pyrimidin-4-yl)-5-nitrothiophene-2-carboxamide

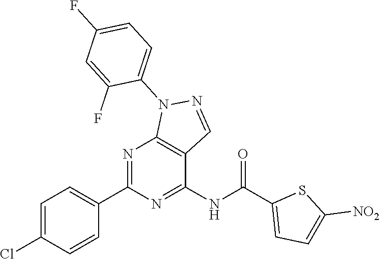 [SMILES: O=C(Nc1nc(-c2ccc(Cl)cc2)nc2c1cnn2-c1ccc(F)cc1F)c1ccc([N+](=O)[O-])s1]

¹H NMR (400 MHz, DMSO-d₆): δ 12.09 (br. s., 1H), 8.65 (s, 1H), 8.31-8.50 (m, 3H), 8.25 (d, J=4.4 Hz, 1H), 7.88 (td, J=8.8, 5.9 Hz, 1H), 7355-7.73 (m, 3H), 7.31-7.45 (m, 1H). MS(M+1): 513. Yellow solid.

### Compound 7-15

### N-(1-benzyl-6-(6-chloropyridin-3-yl)-1H-pyrazolo[3,4-d]pyrimidin-4-yl)-5-nitrothiophene-2-carboxamide

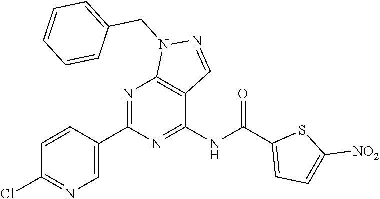 [SMILES: O=C(Nc1nc(-c2ccc(Cl)nc2)nc2c1cnn2Cc1ccccc1)c1ccc([N+](=O)[O-])s1]

¹H NMR (400 MHz, DMSO-d₆): δ12.03 (br. s., 1H), 9.48 (d, J=2.0 Hz, 1H), 8.85 (dd, J=8.3, 2.4 Hz, 1H), 8.46 (s, 1H), 832 (d, J=4.4 Hz, 1H), 8.24 (d, J=4.4 Hz, 1H), 7.75 (d, J=8.3 Hz, 1H), 7.19-7.46 (m, 5H), 5.74 (s, 2H). MS(M+1): 492. Lemon Chiffon solid.

### Compound 7-16

### N-(1-benzyl-6-(4-chlorophenyl)-1H-pyrazolo[3,4-d]pyrimidin-4-yl)-5-nitrothiophene-2-carboxamide

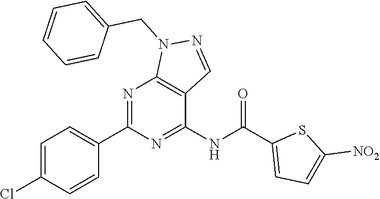 [SMILES: O=C(Nc1nc(-c2ccc(Cl)cc2)nc2c1cnn2Cc1ccccc1)c1ccc([N+](=O)[O-])s1]

¹H NMR (400 MHz, DMSO-d₆): δ 11.97 (br. s., 1H), 8.49-8.61 (m, 2H), 8.43 (s, 1H), 8.36 (d, J=4.4 Hz, 1H), 8.25 (d, J=4.4 Hz, 1H), 7.57-7.72 (m, 2H), 7.22-7.39 (m, 5H), 5.73 (s, 2H). MS(M+1): 491. Bright yellow solid.

### Compound 7-17

### N-(1-benzyl-6-(6-methylpyridin-3-yl)-1H-pyrazolo[3,4-d]pyrimidin-4-yl)-5-nitrothiophene-2-carboxamide

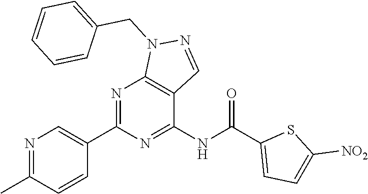 [SMILES: Cc1ccc(-c2nc(NC(=O)c3ccc([N+](=O)[O-])s3)c3cnn(Cc4ccccc4)c3n2)cn1]

¹H NMR (400 MHz, DMSO-d₆): δ 11.97 (s, 1H), 9.56 (d, J=2.0 Hz, 1H), 8.66-8.79 (m, 1H), 8.44 (s, 1H), 8.38 (d, J=4.4 Hz, 1H), 8.25 (d, J=4.4 Hz, 1H), 7.47 (d, J=7.8 Hz, 1H), 7.22-7.41 (m, 5H), 5.73 (s, 2H), 2.58 (s, 3H). MS(M+1): 472. Khaki solid.

### Compound 7-18

### N-1-(3-fluorobenzyl)-6-(6-methoxypyridin-3-yl)-1H-pyrazolo[3,4-d]pyrimidin-4-yl)-5-nitrothiophene-2-carboxamide

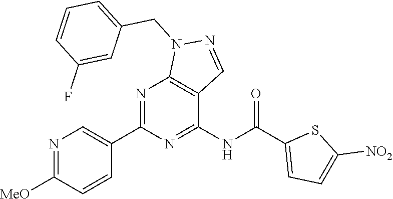 [SMILES: COc1ccc(-c2nc(NC(=O)c3ccc([N+](=O)[O-])s3)c3cnn(Cc4cccc(F)c4)c3n2)cn1]

¹H NMR (400 MHz, DMSO-d₆): δ 11.94 (br. s., 1H), 9.31 (d, J=2.4 Hz, 1H), 8.72 (dd, J=8.8, 2.4 Hz, 1H), 8.43 (s, 1H), 8.36 (d, J=4.4 Hz, 1H), 8.25 (d, J=4.4 Hz, 1H), 7.39 (td, J=7.9, 6.1 Hz, 1H), 7.06-7.23 (m, 3H), 7.01 (d, J=9.3 Hz, 1H), 5.74 (s, 2H), 3.96 (s, 3H). MS(M+1): 506. Bright yellow solid.

### Compound 7-19

### N-(1-(4-acrylamidophenyl)-6-(4-fluorophenyl)-1H-pyrazolo[3,4-d]pyrimidin-4-yl)-5-nitrothiophene-2-carboxamide

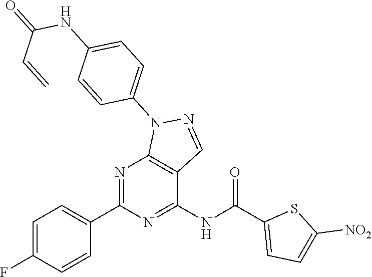 [SMILES: C=CC(=O)Nc1ccc(-n2ncc3c(NC(=O)c4ccc([N+](=O)[O-])s4)nc(-c4ccc(F)cc4)nc32)cc1]

¹H NMR (400 MHz, DMSO-d₆): δ 11.97 (br. s., 1H), 10.34 (s, 1H), 8.51-8.61 (m, 3H), 8.37 (d, J=4.4 Hz, 1H), 8.24 (d, J=4.4 Hz, 1H), 8.16-8.22 (m, J=8.8 Hz, 2H), 7.87-7.95 (m, J=8.8 Hz, 2H), 7.40 (t, J=9.0 Hz, 2H), 6.49 (dd, J=17.1, 10.3 Hz, 1H), 6.31 (dd, J=17.1, 2.0 Hz, 1H), 5.80 (dd, J=9.8, 2.0 Hz, 1H). MS(M+1): 530. Yellow solid.

TABLE 8

| | 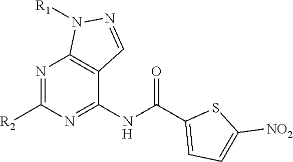 | | |
|---|---|---|---|
| | | | Hep3B |
| | | | LC₅₀ |
| Compound | R₁ | R₂ | (uM) |
| 8-1 | (S)-1-isopropylpyrrolidin-3-yl | 6-fluoropyridin-3-yl | >3 |
| 8-2 | (S)-N-1-acetylpyrrolidin-3-yl | 6-fluoropyridin-3-yl | >3 |
| 8-3 | (S)-N-1-acryloylpyrrolidin-3-yl | 6-fluoropyridin-3-yl | >3 |
| 8-4 | (S)-N-1-pivaloylpyrrolidin-3-yl | 6-fluoropyridin-3-yl | 1.49 |
| 8-5 | ethyl (S)-3-pyrrolidine- | 6-fluoropyridin-3-yl | >2 |
| | 1-carboxylate | | |
| 8-6 | ethyl (R)-3-pyrrolidine- | 6-fluoropyridin-3-yl | >2 |
| | 1-carboxylate | | |
| 8-7 | tert-butyl (S)-3-pyrrolidine- | 6-fluoropyridin-3-yl | 1.49 |
| | 1-carboxylate | | |
| 8-8 | cyclopentyl | 6-fluoropyridin-3-yl | 1.17 |
| 8-9 | cyclopentyl | 4-chlorophenyl | 0.94 |
| 8-10 | cycloheptyl | 4-fluorophenyl | 0.83 |
| 8-11 | cycloheptyl | 6-fluoropyridin-3-yl | NA |
| 8-12 | cycloheptyl | 4-methyl benzoate | 0.47 |
| 8-13 | (1R,5S)-3,3,5- | 4-fluorophenyl | 0.90 |
| | trimethylcyclohexyl | | |
| 8-14 | (1R,5S)-3,3,5- | 6-fluoropyridin-3-yl | 0.62 |
| | trimethylcyclohexyl | | |
| 8-15 | (1R,5S)-3,3,5- | 4-chlorophenyl | 1.27 |
| | trimethylcyclohexyl | | |
| 8-16 | (1R,5S)-3,3,5- | 6-chloropyridin-3-yl | 0.59 |
| | trimethylcyclohexyl | | |

### Compound 8-1

### (S)—N-(6-(6-fluoropyridin-3-yl)-1-(1-isopropylpyrrolidin-3-yl)-1H-pyrazolo[3,4-d]pyrimidin-4-yl)-5-nitrothiophene-2-carboxamide

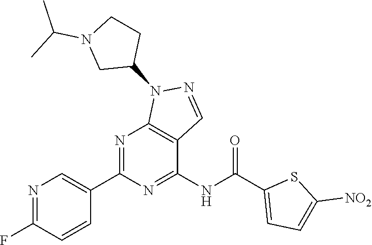

¹H NMR (400 MHz, DMSO-d₆): δ 11.84 (br. s., 1H), 9.30 (d, J=2.4 Hz, 1H), 8.96 (td, J=8.3, 2.4 Hz, 1H), 8.42 (s, 1H), 8.25-8.32 (m, 1H), 8.18-8.25 (m, 1H), 7.40 (dd, J=8.6, 2.7 Hz, 1H), 5.51-5.69 (m, 1H), 2.90 (d, J=19.1 Hz, 3H), 2.58 (br. s., 1H), 2.25-2.47 (m, 3H), 1.09 (d, J=6.4 Hz, 6H). MS(M+1): 497. Dark orange solid.

### Compound 8-2

### (S)—N-(1-(1-acetylpyrrolidin-3-yl)-6-(6-fluoropyridin-3-yl)-1H-pyrazolo[3,4-d]pyrimidin-4-yl)-5-nitrothiophene-2-carboxamide

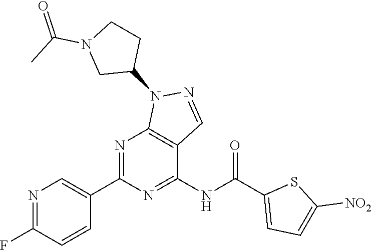

¹H NMR (400 MHz, DMSO-d₆): δ 12.01 (br. s., 1H), 9.32 (dd, J=5.4, 2.4 Hz, 1H), 8.89-9.05 (m, 1H), 8.45 (d, J=5.4 Hz, 1H), 8.33 (d, J=4.4 Hz, 1H), 8.25 (d, J=4.4 Hz, 1H), 7.42 (dd, J=8.6, 2.7 Hz, 1H), 3.57-4.11 (m, 5H), 2.38-2.60 (m, 2H), 1.99 (s, 3H). MS(M+1): 497. White solid.

### Compound 8-3

### (S)—N-(1-(1-acryloylpyrrolidin-3-yl)-6-(6-fluoropyridin-3-yl)-1H-pyrazolo[3,4-d]pyrimidin-4-yl)-5-nitrothiophene-2-carboxamide

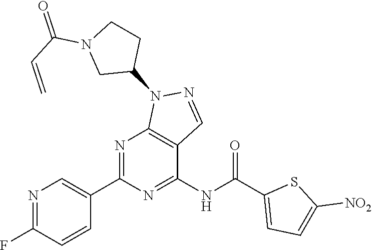

¹H NMR (400 MHz, DMSO-d₆): δ 12.01 (br. s., 1H), 9.21-9.43 (m, 1H), 8.86-9.06 (m, 1H), 8.45 (d, J=3.4 Hz, 1H), 8.34 (dd, J=4.4, 1.5 Hz, 1H), 8.25 (d, J=4.4 Hz, 1H), 7.42 (dd, J=8.6, 2.7 Hz, 1H), 6.51-6.75 (m, 1H), 6.18 (ddd, J=17.1, 7.8, 2.4 Hz, 1H), 5.62-5.83 (m, 2H), 3.59-4.23 (m, 4H), 2.25-2.64 (m, 2H). MS(M+1): 509. Rosy brown solid.

### Compound 8-4

### (S)—N-(6-(6-fluoropyridin-3-yl)-1-(1-pivaloylpyrrolidin-3-yl)-1H-pyrazolo[3,4-d]pyrimidin-4-yl)-5-nitrothiophene-2-carboxamide

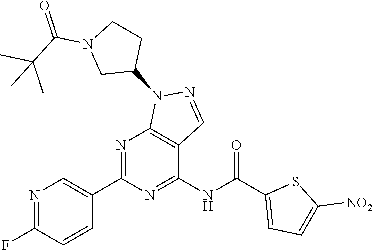

¹H NMR (400 MHz, DMSO-d₆): δ 11.93 (br. s., 1H), 9.29 (d, J=2.4 Hz, 1H), 8.94 (td, J=8.3, 2.4 Hz, 1H), 8.41 (s, 1H), 8.34 (d, J=4.4 Hz, 1H), 8.23 (d, J=4.4 Hz, 1H), 7.40 (dd, J=8.6, 2.7 Hz, 1H), 5.65 (br s., 1H), 4.00 (br. s., 2H), 3.83 (br. s., 2H), 2.44 (br. s., 2H), 1.19 (s, 9H). MS(M+1): 539. White solid.

### Compound 8-5

### ethyl (S)-3-(6-(6-fluoropyridin-3-yl)-4-(5-nitrothiophene-2-carboxamide)-1H-pyrazolo[3,4-d]pyrimidin-1-yl)pyrrolidine-1-carboxylate

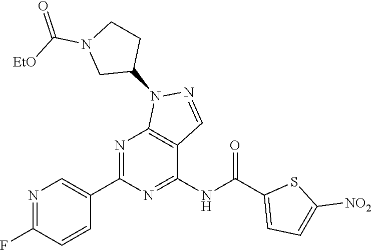

¹H NMR (400 MHz, DMSO-d₆): δ 11.94 (s, 1H), 9.28 (d, J=2.4 Hz, 1H), 8.93 (td, J=8.3, 2.4 Hz, 1H), 8.41 (s, 1H), 8.34 (d, J=4.4 Hz, 1H), 8.23 (d, J=4.4 Hz, 1H), 7.40 (dd, J=8.6, 2.7 Hz, 1H), 5.68 (br. s., 1H), 4.06 (quin, J=7.1 Hz, 2H), 3.86-3.95 (m, 1H), 3.75 (dd, J=11.2, 4.4 Hz, 1H), 3.48-3.71 (m, 2H), 2.30-2.48 (m, 2H), 1.19 (dt, J=16.6, 7.3 Hz, 3H). MS(M+1): 527. Burly wood solid.

### Compound 8-6

### ethyl (R)-3-(6-(6-fluoropyridin-3-yl)-4-(5-nitrothiophene-2-carboxamido)-1H-pyrazolo[3,4-d]pyrimidin-1-yl)pyrrolidine-1-carboxylate

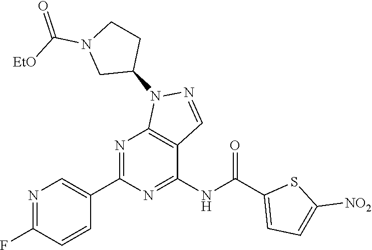 [SMILES: CCOC(=O)N1CC[C@@H](n2ncc3c(NC(=O)c4ccc([N+](=O)[O-])s4)nc(-c4ccc(F)nc4)nc32)C1]

¹H NMR (400 MHz, DMSO-d₆): δ 11.99 (br. s., 1H), 9.31 (d, J=2.4 Hz, 1H), 8.95 (td, J=8.3, 2.4 Hz, 1H), 8.44 (s, 1H), 8.34 (d, J=4.4 Hz, 1H), 8.25 (d, J=4.4 Hz, 1H), 7.41 (dd, J=8.6, 2.7 Hz, 1H), 5.70 (br s., 1H), 4.01-4.15 (m, 2H), 3.85-3.97 (m, 1H), 3.71-3.78 (m, 1H), 3.64 (d, J=6.8 Hz, 1H), 3.57 (d, J=6.4 Hz, 1H), 2.26-2.48 (m, 2H), 1.19 (dt, J=16.9, 7.0 Hz, 3H). MS(M+1): 527. Light yellow solid.

### Compound 8-7

### tert-butyl (S)-3-(6-(6-fluoropyridin-3-yl)4-(5-nitrothiophene-2-carboxamido)-1H-pyrazolo[3,4-d]pyrimidin-1-yl)pyrrolidine-1-carboxylate

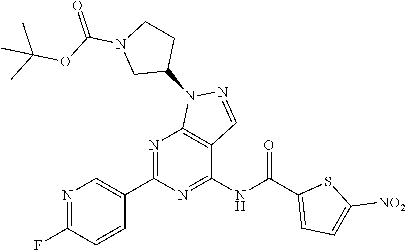

¹H NMR (400 MHz, DMSO-d₆): δ 12.01 (br. s., 1H), 9.32 (d, J=2.4 Hz, 1H), 8.96 (td, J=8.3, 2.4 Hz, 1H), 8.44 (s, 1H), 8.32 (d, J=4.4 Hz, 1H), 8.25 (d, J=4.4 Hz, 1H), 7.41 (dd, J=8.6, 2.7 Hz, 1H), 5.68 (br. s., 1H), 3.82-3.91 (m, 1H), 3.56-3.72 (m, 2H), 3.50 (br. s., 1H), 2.34-2.48 (m, 2H), 1.38-1.48 (m, 9H). MS(M+1): 555. Pale yellow solid.

### Compound 8-8

### N-(1-cyclopentyl-6-(6-fluoropyridin-3-yl)-1H-pyrazolo[3,4-d]pyrimidin-4-yl)-5-nitrothiophene-2-carboxamide

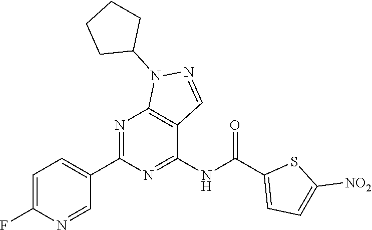 [SMILES: O=C(Nc1nc(-c2ccc(F)nc2)nc2c1cnn2C1CCCC1)c1ccc([N+](=O)[O-])s1]

¹H NMR (400 MHz, DMSO-d₆): δ 11.91 (br. s., 1H), 9.30 (d, J=2.4 Hz, 1H), 8.95 (id, J=8.3, 2.4 Hz, 1H), 8.41 (s, 1H), 8.31-839 (m, 1H), 8.24 (d, J=4.4 Hz, 1H), 7.40 (dd, J=8.6, 2.7 Hz, 1H), 5.46 (quin, J=7.3 Hz, 1H), 2.13-2.27 (m, 2H), 2.00-2.13 (m, 2H), 1.83-2.00 (m, 2H), 1.68-1.83 (m, 2H). MS(M+1): 454. Khaki solid.

### Compound 8-9

### N-(6-(4-chlorophenyl)-1-cyclopentyl-1H-pyrazolo[3,4-d]pyrimidin-4-yl)-5-nitrothiophene-2-carboxamide

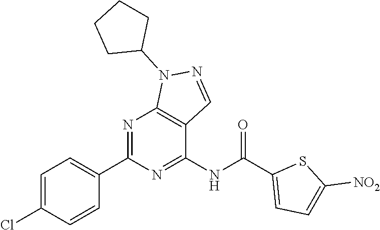 [SMILES: O=C(Nc1nc(-c2ccc(Cl)cc2)nc2c1cnn2C1CCCC1)c1ccc([N+](=O)[O-])s1]

¹H NMR (4004 Hz, DMSO-d₆): δ 11.87 (br. s., 1H), 8.45-8.62 (m, 2H), 8.30-8.45 (m, 2H), 8.21 (d, J=4.4 Hz, 1H), 7.56-7.71 (m, 2H), 5.43 (quin, J=7.2 Hz, 1H), 2.12-2.26 (m, 2H), 1.99-2.12 (m, 2H), 1.82-1.99 (m, 2H), 1.63-1.82 (m, 2H). MS(M+1): 469. Light yellow solid.

### Compound 8-10

### N-(1-cycloheptyl-6-(4-fluorophenyl)-1H-pyrazolo[3,4-d]pyrimidin-4-yl)-5-nitrothiophene-2-carboxamide

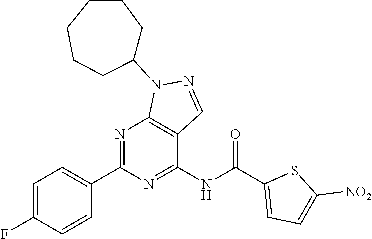 [SMILES: O=C(Nc1nc(-c2ccc(F)cc2)nc2c1cnn2C1CCCCCC1)c1ccc([N+](=O)[O-])s1]

¹H NMR (400 MHz, DMSO-d₆): δ 11.91 (br. s., 1H), 8.54-8.62 (m, 2H), 8.37 (s, 1H), 8.33 (d, J=4.4 Hz, 1H), 8.24 (d, J=4.4 Hz, 1H), 7.34-7.46 (m, 2H), 5.12 (tt, J=9.6, 4.8 Hz, 1H), 2.10-2.24 (m, 2H), 2.00-2.10 (m, 2H), 1.79-1.95 (m, 2H), 1.56-1.79 (m, 6H). MS(M+1): 481. Khaki solid.

### Compound 8-11

### N-(1-cycloheptyl-6-(6-fluoropyridin-3-yl)-1H-pyrazolo[3,4-d]pyrimidin-4-yl)-5-nitrothiophene-2-carboxamide

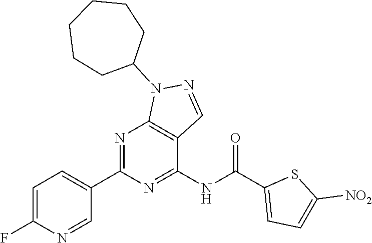 [SMILES: O=C(Nc1nc(-c2ccc(F)nc2)nc2c1cnn2C1CCCCCC1)c1ccc([N+](=O)[O-])s1]

¹H NMR (400 MHz, DMSO-d₆): δ 11.92 (br. s., 1H), 9.31 (d, J=2.4 Hz, 1H), 8.95 (td, J=8.3, 2.4 Hz, 1H), 840 (s, 1H), 8.35 (d, J=4.4 Hz, 1H), 8.24 (d, J=4.4 Hz, 1H), 7.40 (dd, J=8.6.2.7 Hz, 1H), 5.13 (it, J=9.6, 4.8 Hz, 1H), 2.10-2.23 (m, 2H), 1.98-2.10 (m, 2H), 1.78-1.91 (m, 2H), 1.55-1.78 (m, 6H). MS(M+1): 482. Khaki solid.

### Compound 8-12

### methyl 4-(1-cycloheptyl-4-(5-nitrothiophene-2-carboxamido)-1H-pyrazolo[3,4-d] pyrimidin-6-yl)benzoate

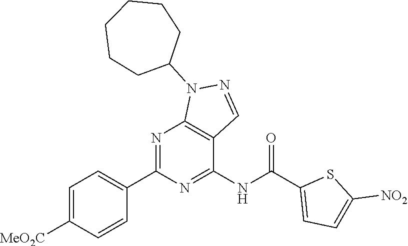 [SMILES: COC(=O)c1ccc(-c2nc(NC(=O)c3ccc([N+](=O)[O-])s3)c3cnn(C4CCCCCC4)c3n2)cc1]

¹H NMR (400 MHz, DMSO-d₆): δ 11.97 (br. s., 1H), 8.64-8.71 (m, J=8.8 Hz, 2H), 8.39 (s, 1H), 8.36 (d, J=4.4 Hz, 1H), 8.24 (d, J=4.4 Hz, 1H), 8.09-8.20 (m, J=8.3 Hz, 2H), 5.15 (tt, J=9.5, 4.9 Hz, 1H), 3.91 (s, 3H), 2.10-2.22 (m, 2H), 1.99-2.10 (m, 2H), 1.78-1.92 (m, 2H), 1.55-1.78 (m, 6H). MS(M+1): 521. Gray solid.

### Compound 8-13

### N-(6-(4-fluorophenyl)-1-((1R,5S)-3,3,5-trimethylcyclohexyl)-1H-pyrazolo[3,4-d]pyrimidin-4-yl)-5-nitrothiophene-2-carboxamide

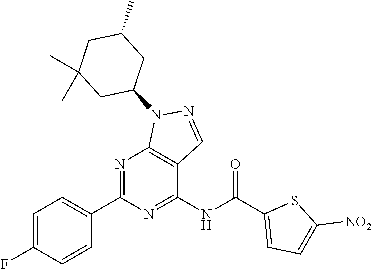 [SMILES: C[C@@H]1C[C@@H](n2ncc3c(NC(=O)c4ccc([N+](=O)[O-])s4)nc(-c4ccc(F)cc4)nc32)CC(C)(C)C1]

¹H NMR (400 MHz, DMSO-d₆) δ 11.88 (br. s., 1H), 8.57 (dd, J=8.8, 5.9 Hz, 2H), 8.32-8.42 (m, 2H), 8.25 (d, J=4.4 Hz, 1H), 7.35-7.48 (m, 2H), 5.04-5.17 (m, 1H), 2.37-2.47 (m, 2H), 2.32 (dd, J=14.2, 5.9 Hz, 1H), 1.67 (dd, J=13.7, 4.4 Hz, 1H), 1.53-1.62 (m, 1H), 1.47 (dd, J=12.7, 3.9 Hz, 1H), 1.10-1.16 (m, 1H), 1.08 (d, J=6.8 Hz, 3H), 0.99 (s, 3H), 0.59 (s, 3H). MS(M+1): 509. Yellow solid.

### Compound 8-14

### N-(6-(6-fluoropyridin-3-yl)-1-((1R,5S)-3,3,5-trimethylcyclohexyl)-1H-pyrazolo[3,4-d]pyrimidin-4-yl)-5-nitrothiophene-2-carboxamide

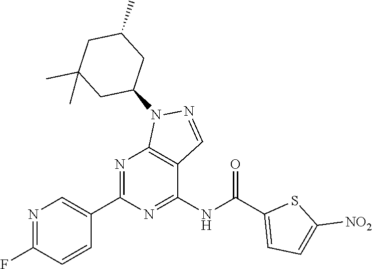 [SMILES: C[C@@H]1C[C@@H](n2ncc3c(NC(=O)c4ccc([N+](=O)[O-])s4)nc(-c4ccc(F)nc4)nc32)CC(C)(C)C1]

¹H NMR (400 MHz, DMSO-d₆): δ 11.94 (br. s., 1H), 9.29 (d, J=2.4 Hz, 1H), 8.93 (td, J=8.3, 2.4 Hz, 1H), 8.41 (s, 1H), 8.33 (d, J=4.4 Hz, 1H), 8.24 (d, J=4.4 Hz, 1H), 7.42 (dd, J=8.8, 2.4 Hz, 1H), 5.09-5.18 (m, 1H), 2.36-2.45 (m, 2H), 2.29 (dd, J=13.9, 6.1 Hz, 1H), 1.68 (dd, J=13.7, 3.9 Hz, 1H), 1.54-1.63 (m, 1H), 1.47 (d, J=9.3 Hz, 1H), 1.11-1.18 (m, 1H), 1.08 (d, J=6.8 Hz, 3H), 0.99 (s, 3H), 0.60 (s, 3H). MS(M+1): 510. Light yellow solid.

### Compound 8-15

### N-(6-(4-chlorophenyl)-1-((1R,5S)-3,3,5-trimethylcyclohexyl)-1H-pyrazolo[3,4-d]pyrimidin-4-yl)-5-nitrothiophene-2-carboxamide

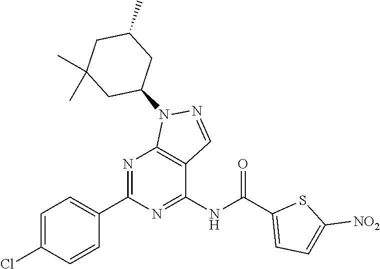 [SMILES: C[C@@H]1C[C@@H](n2ncc3c(NC(=O)c4ccc([N+](=O)[O-])s4)nc(-c4ccc(Cl)cc4)nc32)CC(C)(C)C1]

¹H NMR (400 MHz, DMSO-d₆): δ 11.89 (s, 1H), 8.45-8.59 (m, J=8.8 Hz, 2H), 8.31-8.42 (m, 2H), 8.24 (d, J=4.4 Hz, 1H), 7.56-7.72 (m, 2H), 5.04-5.20 (m, 1H), 2.36-2.45 (m, 2H), 2.31 (dd, J=13.7, 5.9 Hz, 1H), 1.67 (dd, J=13.7, 4.4 Hz, 1H), 1.51-1.62 (m, 1H), 1.46 (dd, J=13.0, 3.2 Hz, 1H), 1.09-1.16 (m, 1H), 1.07 (d, J=6.8 Hz, 3H), 0.98 (s, 3H), 0.58 (s, 3H). MS(M+1): 525. Yellow solid.

### Compound 8-16

### N-(6-(6-chloropyridin-3-yl)-1-((1R,5S)-3,3,5-trimethylcyclohexyl)-1H-pyrazolo[3,4-d]pyrimidin-4-yl)-5-nitrothiophene-2-carboxamide

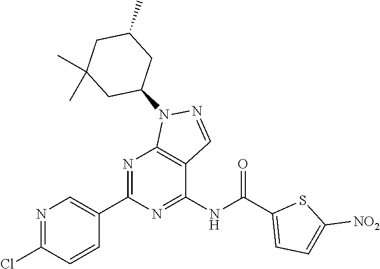 [SMILES: C[C@@H]1C[C@@H](n2ncc3c(NC(=O)c4ccc([N+](=O)[O-])s4)nc(-c4ccc(Cl)nc4)nc32)CC(C)(C)C1]

¹H NMR (400 MHz, DMSO-d₆): δ 11.96 (s, 1H), 9.45 (d, J=2.0 Hz, 1H), 8.80 (dd, J=8.3, 2.4 Hz, 1H), 8.43 (s, 1H), 8.36 (d, J=4.4 Hz, 1H), 8.25 (d, J=4.4 Hz, 1H), 7.77 (d, J=8.3 Hz, 1H), 5.04-5.22 (m, 1H), 2.35-2.45 (m, 2H), 2.29 (dd, J=13.9, 6.1 Hz, 1H), 1.68 (dd, J=13.9, 4.2 Hz, 1H), 1.59 (s, 1H), 1.43-1.50 (m, 1H), 1.05-1.17 (m, 4H), 0.99 (s, 3H), 0.60 (s, 3H). MS(M+1): 526. Yellow-green solid.

TABLE 9

| | 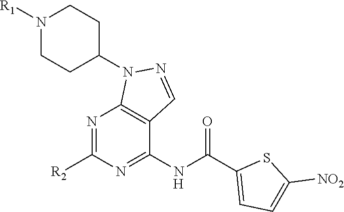 | | |
|---|---|---|---|
| | | | Hep3B |
| | | | LC₅₀ |
| Compound | R1 | R2 | (uM) |
| 9-1 | Isopropyl | 6-fluoropyridin-3-yl | 1.25 |
| 9-2 | ethyl formate | 6-fluoropyridin-3-yl | >5 |
| 9-3 | BOC | methyl 4-benzoate | 0.42 |
| 9-4 | BOC | 6-fluoropyridin-3-yl | 0.93 |
| 9-5 | BOC | 4-chlorophenyl | 0.45 |
| 9-6 | Acryloyl | 6-fluoropyridin-3-yl | 1.25 |
| 9-7 | Butyryl | 6-fluoropyridin-3-yl | >5 |
| 9-8 | Pivaloyl | 6-fluoropyridin-3-yl | 1.90 |
| 9-9 | 4,4,4-trifluorobutyl formate | 4-chlorophenyl | 1.07 |
| 9-10 | 2-(2-ethoxyethoxy)ethyl | thiophen-2-yl | >3 |
| | formate | | |
| 9-11 | 2-(2-ethoxyethoxy)ethyl | 4-chlorophenyl | >1.25 |
| | formate | | |
| 9-12 | 2-methoxyethyl formate | 4-chlorophenyl | 1.86 |
| 9-13 | 2-ethoxyethyl formate | 4-chlorophenyl | 1.62 |

### Compound 9-1

### N-(6-(6-fluoropyridin-3-yl)-1-(1-isopropylpiperidin-4-yl)-1H-pyrazolo[3,4-d]pyrimidin-4-yl))-5-nitrothiophene-2-carboxamide

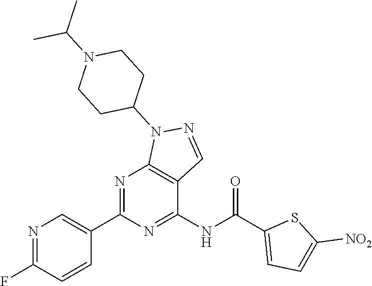 [SMILES: CC(C)N1CCC(n2ncc3c(NC(=O)c4ccc([N+](=O)[O-])s4)nc(-c4ccc(F)nc4)nc32)CC1]

¹H NMR (400 MHz, DMSO-d₆): δ 11.63 (br. s., 1H), 9.30 (d, J=2.0 Hz, 1H), 8.94 (td. J=8.3, 2.4 Hz, 1H), 8.38 (s, 1H), 8.14-8.31 (m, 2H), 7.39 (dd, J=8.6, 2.7 Hz, 1H), 4.92 (br. s., 1H), 3.05 (br. s., 2H), 2.93 (br. s., 1H), 2.45-2.65 (m, 2H), 2.17-2.29 (m, 2H), 1.94-2.07 (m, 2H), 1.08 (d, J=6.8 Hz, 6H). MS(M+1): 511. Light yellow solid.

### Compound 9-2

### ethyl 4-(6-(6-fluoropyridin-3-yl)-4-(5-nitrothiophene-2-carboxamide)-1H-pyrazolo [3,4-d]pyrimidin-1-yl)piperidine-1-carboxylate

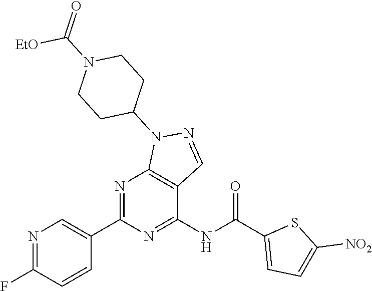 [SMILES: CCOC(=O)N1CCC(n2ncc3c(NC(=O)c4ccc([N+](=O)[O-])s4)nc(-c4ccc(F)nc4)nc32)CC1]

¹H NMR (400 MHz, DMSO-d₆): δ 12.05 (br. s., 1H), 9.31 (s, 1H), 8.96 (t, J=7.6 Hz, 1H), 8.40 (s, 1H), 8.23 (br. s., 2H), 7.41 (d, J=8.3 Hz, 1H), 5.15 (br. s., 1H), 3.98-4.26 (m, 4H), 3.13 (br. s., 2H), 2.08-2.03 (m, 4H), 1.22 (t, J=7.1 Hz, 3H). MS(M+1): 541. Yellow-green solid.

### Compound 9-3

### tert-butyl 4-(6-(4-(methoxycarbonyl)phenyl)-4-(5-nitrothiophene-2-carboxamido)-1H-pyrazolo[3,4-d]pyrmidin-1-yl)piperidine-1-carboxylate

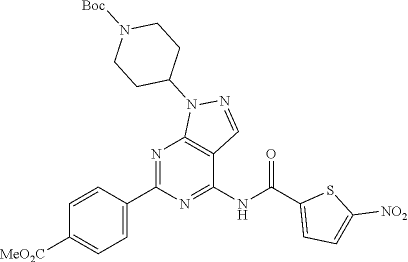 [SMILES: COC(=O)c1ccc(-c2nc(NC(=O)c3ccc([N+](=O)[O-])s3)c3cnn(C4CCN(C(=O)OC(C)(C)C)CC4)c3n2)cc1]

¹H NMR (DMSO-4, 400 MHz): δ 11.97 (br. s., 1H), 8.57-8.70 (m, 2H), 8.38 (s, 1H), 8.36 (d, J=4.4 Hz, 1H), 8.23 (d, J=4.4 Hz, 1H), 8.07-8.17 (m, 2H), 4.97-5.21 (m, 1H), 4.12 (d, J=12.2 Hz, 2H), 3.90 (s, 3H), 3.07 (brs., 2H), 1.94-2.14 (m, 4H), 1.45 (s, 9H). MS(M+1): δ 08. Yellow solid.

### Compound 94

### tert-butyl 4-(6-(6-fluoropyridin-3-yl)-4-(5-nitrothiophene-2-carboxamido)-1H-pyrazolo[3,4-d]pyrimidin-1-yl)piperidine-1-carboxylate

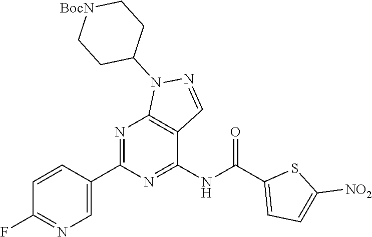 [SMILES: CC(C)(C)OC(=O)N1CCC(n2ncc3c(NC(=O)c4ccc([N+](=O)[O-])s4)nc(-c4ccc(F)nc4)nc32)CC1]

¹H NMR (400 MHz, DMSO-d₆): δ 11.90 (s, 1H), 9.28 (d, J=2.4 Hz, 1H), 8.92 (t, J=8.3, 1H), 8.38 (s, 1H), 8.32 (d, J=4.4 Hz, 1H), 8.21 (d, J=4.4 Hz, 1H), 7.38 (d, J=8.3, 2.7 Hz, 1H), 5.15-5.07 (m, 1H), 4.13-4.10 (brm, 2H), 3.06 (brs, 2H), 2.08-1.98 (m, 4H), 1.44 (s, 9H). MS(M+1): 569. Yellow solid.

### Compound 9-5

### tert-butyl 4-(6-(4-chlorophenyl)-4-(5-nitrothiophene-2-carboxamido)-1H-pyrazolo [3,4-d]pyrimidin-1-yl)piperidine-1-carboxylate

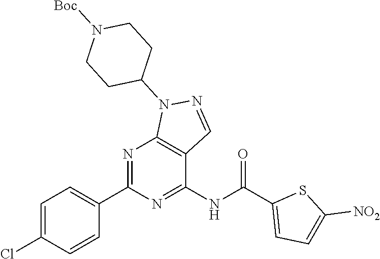 [SMILES: CC(C)(C)OC(=O)N1CCC(n2ncc3c(NC(=O)c4ccc([N+](=O)[O-])s4)nc(-c4ccc(Cl)cc4)nc32)CC1]

¹H NMR (400 MHz, DMSO-d₆): δ 11.96 (br. s., 1H), 8.48-8.61 (m, 2H), 8.40 (s, 1H), 8.34 (d, J=4.9 Hz, 1H), 8.24 (d, J=4.4 Hz, 1H), 7.61-7.69 (m, 2H), 5.02-5.19 (m, 1H), 4.12 (d, J=12.2 Hz, 2H), 3.07 (br. s., 2H), 1.94-2.13 (m, 4H), 1.45 (s, 9H). MS(M+1): 584. Light yellow solid.

### Compound 9-6

### N-(1-4 I-acryloylpiperidin-4-yl)-6-(6-fluoropyridin-3-yl)-1H-pyrazolo[3,4,d]pyrimidin-4-yl)-5-nitrothiophene-2-carboxamide

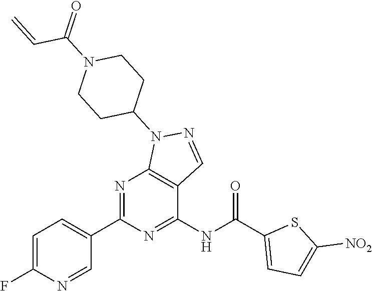 [SMILES: C=CC(=O)N1CCC(n2ncc3c(NC(=O)c4ccc([N+](=O)[O-])s4)nc(-c4ccc(F)nc4)nc32)CC1]

¹H NMR (400 MHz, DMSO-d₆): δ 11.98 (br. s., 1H), 9.32 (d, J=2.4 Hz, 1H), 8.96 (td, J=8.3, 2.4 Hz, 1H), 8.43 (s, 1H), 8.34 (d, J=4.4 Hz, 1H), 8.25 (d, J=4.4 Hz, 1H), 7.42 (dd, J=8.8, 2.4 Hz, 1H), 6.90 (dd, J=16.6, 10.8 Hz, 1H), 6.16 (dd, J=16.6, 2.4 Hz, 1H), 5.75 (s, 1H), 5.72 (dd, J=10.3, 2.4 Hz, 1H), 5.25 (t, J=7.1 Hz, 1H), 4.58 (d, J=13.7 Hz, 1H), 4.25 (d, J=13.2 Hz 1H), 3.43 (td, J=7.1, 4.9 Hz, 1H), 2.92-3.10 (m, 1H), 1.97-2.20 (m, 4H). MS(M+1): 523. Goldenrod powder.

### Compound 9-7

### N-(1-(1-butyrylpiperidin-4-yl)-6-(6-fluoropyridin-3-yl)-1H-pyrazolo[3,4-d]pyrimidin-4-yl)-5-nitrothiophene-2-carboxamide

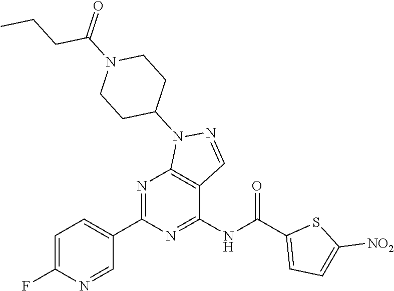 [SMILES: CCCC(=O)N1CCC(n2ncc3c(NC(=O)c4ccc([N+](=O)[O-])s4)nc(-c4ccc(F)nc4)nc32)CC1]

¹H NMR (400 MHz, DMSO-d₆): δ 11.95 (br. s., 1H), 9.31 (d, J=2.4 Hz, 1H), 8.95 (td, J=8.3, 2.4 Hz, 1H), 8.41 (s, 1H), 8.34 (d, J=4.4 Hz, 1H), 8.24 (d, J=4.4 Hz, 1H), 7.41 (dd, J=8.6, 2.7 Hz, 1H), 5.13-5.31 (m, 1H), 4.57 (d, J=13.2 Hz, 1H), 4.06 (d, J=13.7 Hz, 1H), 2.87 (t, J=11.5 Hz, 1H), 2.28-2.44 (m, 2H), 1.57 (sxt, J=7.3 Hz, 2H), 0.93 (t, J=7.3 Hz, 3H). MS(M+1): 539. Khaki solid.

### Compound 9-8

### N-(6-(6-fluoropyridin-3-yl)-1-(1-pivaloylpiperidin-4-yl)-1H-pyrazolo[3,4,d]pyrimidin-4-yl)-5-nitrothiophene-2-carboxamide

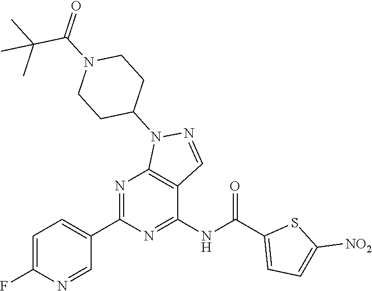 [SMILES: CC(C)(C)C(=O)N1CCC(n2ncc3c(NC(=O)c4ccc([N+](=O)[O-])s4)nc(-c4ccc(F)nc4)nc32)CC1]

¹H NMR (400 MHz, DMSO-d₆): δ 11.92 (br. s., 1H), 9.29 (d, J=2.4 Hz, 1H), 8.93 (td, J=8.3, 2.4 Hz, 1H), 8.39 (s, 1H), 8.34 (d, J=4.9 Hz, 1H), 8.23 (d, J=4.4 Hz, 1H), 7.40 (dd, J=8.6, 2.7 Hz, 1H), 5.13-5.26 (m, 1H), 4.45 (d, J=13.2 Hz, 2H), 3.05-3.18 (m, 2H), 1.99-2.13 (m, 4H), 1.25 (s, 9H). MS(M+1): 553. Light khaki solid.

### Compound 9-9

### 4,4,4-trifluorobutyl 4-(6-(chlorophenyl)-4-(5-nitrothiophene-2-carboxamido)-1H-pyrazolo[3,4-d]piperidin-1-yl)piperidine-1-carboxylate

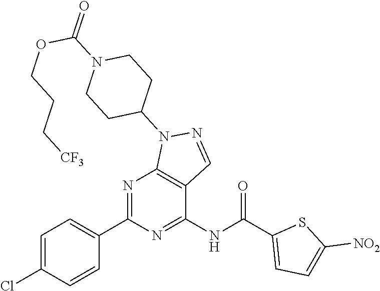 [SMILES: O=C(Nc1nc(-c2ccc(Cl)cc2)nc2c1cnn2C1CCN(C(=O)OCCCC(F)(F)F)CC1)c1ccc([N+](=O)[O-])s1]

¹H NMR (400 MHz, DMSO-d₆): δ 11.95 (br. s., 1H), 8.50-8.61 (m, 2H), 8.40 (s, 1H), 8.35 (d, J=4.4 Hz, 1H), 8.24 (d, J=4.4 Hz, 1H), 7.57-7.70 (m, 2H), 5.15 (dt, J=10.4, 5.3 Hz, 1H), 4.04-4.25 (m, 4H), 3.15 (br. s., 2H), 2.27-2.45 (m, 2H), 1.96-2.19 (m, 4H), 1.75-1.90 (m, 2H). MS(M+1): δ 38. Light yellow solid.

### Compound 9-10

### 2-(2-ethoxyethoxy)ethyl 4-(4-(5-nitrothiophene-2-carboxamido)-6-(thiophen-2-yl)-1H-pyrazolo[3,4-d]pyrimidin-1-yl)piperidine-1-carboxylate

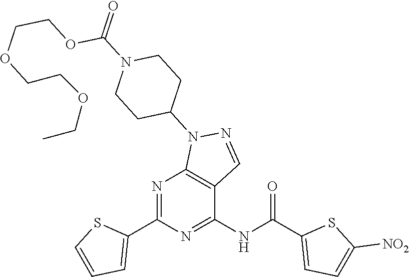 [SMILES: CCOCCOCCOC(=O)N1CCC(n2ncc3c(NC(=O)c4ccc([N+](=O)[O-])s4)nc(-c4cccs4)nc32)CC1]

¹H NMR (400 MHz, DMSO-d₆): δ 11.93 (s, 1H), 8.36 (d, J=4.4 Hz, 1H), 8.33 (s, 1H), 8.23 (d, J=4.4 Hz, 1H), 8.06-8.10 (m, 1H), 7.80 (dd, J=5.1, 1.2 Hz, 1H), 7.25 (dd, J=4.9, 3.4 Hz, 1H), 4.91-5.13 (m, 1H), 4.05-4.27 (m, 4H), 3.60-3.68 (m, 2H), 3.53-3.59 (m, 2H), 3.46-3.50 (m, 2H), 3.43 (q, J=7.2 Hz, 2H), 3.14 (br. s., 2H), 1.97-2.14 (m, 4H), 1.09 (t, J=7.1 Hz, 3H) MS(M+1): 616. Yellow solid.

### Compound 9-11

### 2-(2-ethoxyethoxy)ethyl 4-(6-(4-chlorophenyl)-4-(5-nitrothiophene-2-carboxamido)-1H-pyrazolo[3,4-d]pyrimidin-1-yl)piperidine-1-carboxylate

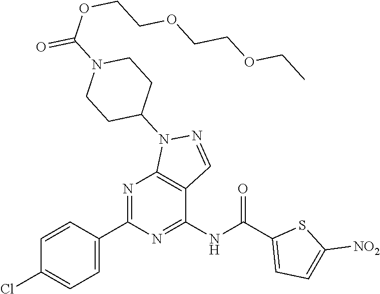 [SMILES: CCOCCOCCOC(=O)N1CCC(n2ncc3c(NC(=O)c4ccc([N+](=O)[O-])s4)nc(-c4ccc(Cl)cc4)nc32)CC1]

¹H NMR (400 MHz, DMSO-d₆): δ 11.95 (s, 1H), 8.55 (d, J=8.8 Hz, 2H), 8.4 (s, 1H), 8.34 (d, J=4.4 Hz, 1H), 8.24 (d, J=4.4 Hz, 1H), 7.65 (d, J=8.8 Hz, 2H), 5.19-5.12 (m, 1H), 4.18-4.14 (m, 4H), 3.65-3.4 (nm, 5H), 3.15 (br. s, 1H), 2.09-2.01 (m, 4H), 1.09 (t, J=6.9 Hz, 3H). MS(M+1): 644. Yellow-brown solid.

### Compound 9-12

### 2-methoxyethyl 4-(6-(4-chlorophenyl)4-(5-nitrothiophene-2-carboxamido)-1H-pyrazolo[3,4-d]pyrimidin-1-yl)piperidine-1-carboxylate

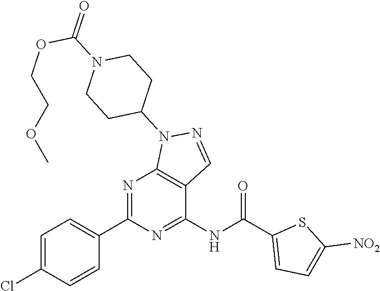 [SMILES: COCCOC(=O)N1CCC(n2ncc3c(NC(=O)c4ccc([N+](=O)[O-])s4)nc(-c4ccc(Cl)cc4)nc32)CC1]

¹H NMR (400 MHz, DMSO-d₆): δ 11.93 (br. s., 1H), 8.48-8.58 (m, 2H), 8.39 (s, 1H), 8.35 (d, J=4.4 Hz, 1H), 8.24 (d, J=4.4 Hz, 1H), 7.58-7.69 (m, 2H), 5.15 (dt, J=10.3, 5.1 Hz, 1H), 4.06-4.23 (m, 4H), 3.48-3.61 (m, 2H), 3.29 (s, 3H), 3.13 (d, J=16.1 Hz, 2H), 1.97-2.13 (m, 4H) MS(M+1): 586. Yellow solid.

### Compound 9-13

### 2-ethoxyethyl 4-(6-(4-chlorophenyl)-4-(5-nitrothiophene-2-carboxamido)-1H-pyrazolo [3,4-d]pyrimidin-1-yl)piperidine-1-carboxylate

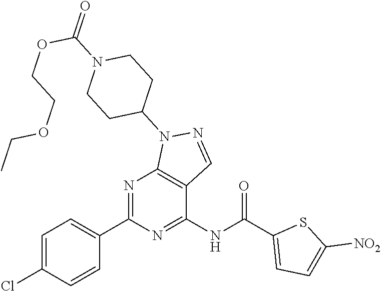 [SMILES: CCOCCOC(=O)N1CCC(n2ncc3c(NC(=O)c4ccc([N+](=O)[O-])s4)nc(-c4ccc(Cl)cc4)nc32)CC1]

¹H NMR (400 MHz, DMSO-d₆): & 11.93 (br. s., 1H), 8.47-8.59 (m, 2H), 8.30-8.44 (m, 2H), 8.23 (d, J=4.4 Hz, 1H), 7.59-7.70 (m, 2H), 5.06-5.22 (m, 1H), 4.05-4.28 (m, 4H), 3.52-3.67 (m, 2H), 3.48 (q, J=7.2 Hz, 2H), 3.15 (br. s., 2H), 1.96-2.14 (m, 4H), 1.03-1.16 (m, 3H). MS(M+1): δ 00. Light yellow solid.

TABLE 10

| | 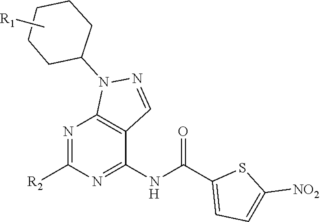 | | |
|---|---|---|---|
| | | | Hep3B |
| | | | LC₅₀ |
| Compound | R₁ | R₂ | (uM) |
| 10-1 | H | 3,3-difluoropyrrolidin-1-yl | 1.25 |
| 10-2 | H | 4,4-difluoropiperidin-1-yl | 3.12 |
| 10-3 | H | 2-(hydroxymethyl)pyrrolidin-1-yl | >5.0 |
| 10-4 | H | furan-2-yl | 1.19 |
| 10-5 | H | thiophen-2-yl | 0.75 |
| 10-6 | H | thiophen-3-yl | 1.03 |
| 10-7 | H | 5-methylthiophen-2-yl | 1.53 |
| 10-8 | H | 5-chlorothiophen-2-yl | 0.49 |
| 10-9 | H | 3,5-dimethylisoxazol-4-yl | 1.07 |
| 10-10 | H | pyridin-3-yl | 0.99 |
| 10-11 | H | 2-fluorophenyl | 1.94 |
| 10-12 | H | 3-fluorophenyl | 0.95 |
| 10-13 | H | 4-fluorophenyl | 0.70 |
| 10-14 | H | 6-fluoropyridin-3-yl | 0.89 |
| 10-15 | H | 2-fluoropyridin-4-yl | 1.17 |
| 10-16 | H | 3-chlorophenyl | 0.61 |
| 10-17 | H | 4-chlorophenyl | 0.53 |
| 10-18 | H | 6-chloropyridin-3-yl | 0.68 |
| 10-19 | H | 6-methylpyridin-3-yl | 1.13 |
| 10-20 | H | 4-cyanophenyl | 1.20 |
| 10-21 | H | 6-methoxypyridin-3-yl | 1.12 |
| 10-22 | H | 5-methoxypyridin-3-yl | 1.16 |
| 10-23 | H | 6-ethoxypyridin-3-yl | 0.57 |
| 10-24 | H | 4-(trifluoromethyl)phenyl | 0.64 |
| 10-25 | H | 6-(trifluoromethyl)pyridin-3-yl | |
| 10-26 | H | 4.(trifluoromethoxy)phenyl | 0.68 |
| 10-27 | H | 4-formylphenyl | 0.84 |
| 10-28 | H | 4-acetylphenyl | 1.93 |
| 10-29 | H | 4-((dimethylamino)methyl)phenyl | 1.51 |
| 10-30 | H | 4-(methoxymethyl)phenyl | 0.84 |
| 10-31 | H | methyl 3-benzoate | 0.58 |
| 10-32 | H | methyl 4-benzoate | 0.40 |
| 10-33 | H | ethyl 4-benzoate | 0.54 |
| 10-34 | H | 4-(methylsulfonyl)phenyl | 0.93 |
| 10-35 | H | 4-(tert-butyl)phenyl | 1.33 |
| 10-36 | H | benzo[d][1,3]dioxol-5-yl | 2.12 |
| 10-37 | H | 2,3-dihydrobenzo[b][1,4]dioxin-6-yl | 1.94 |
| 10-38 | H | 6-morpholinopyridin-3-yl | NA |
| 10-39 | H | 2,4-difluorophenyl | 1.24 |
| 10-40 | H | 3,4-difluorophenyl | 0.32 |
| 10-41 | H | 3,5-difluorophenyl | 0.79 |
| 10-42 | H | 2,4-dichlorophenyl | >1.25 |
| 10-43 | H | 3,4-dichlorophenyl | 0.77 |
| 10-44 | H | 3-chloro-4-ethoxyphenyl | >2.0 |
| 10-45 | H | 4-chloro-2-fluorophenyl | 0.87 |
| 10-46 | H | 2-chloro-4-fluorophenyl | 1.03 |
| 10-47 | H | 5-chloro-2-fluorophenyl | 0.63 |
| 10-48 | H | 3,4-dimethoxyphenyl | 0.97 |
| 10-49 | H | 6-(2,2,2-trifluoroethoxy)pyridin-3-yl | 0.63 |
| 10-50 | H | 4-(2-methoxyethoxy)phenyl | 2.64 |
| 10-51 | H | 6-(2-methoxyethoxy)pyridin-3-yl | 0.96 |
| 10-52 | H | 6-(2,2,3,3-tetrafluoropropoxy) | 0.42 |
| | | pyridin-3-yl | |
| 10-53 | H | 6-(2-(2-ethoxyethoxy)ethoxy) | 1.23 |
| | | pyridin-3-yl | |
| 10-54 | 4,4-difluoro | 4-fluorophenyl | 0.92 |
| 10-55 | 4,4-difluoro | 6-fluoropyridin-3-yl | 0.71 |
| 10-56 | 4,4-difluoro | 4-chlorophenyl | 0.77 |
| 10-57 | 4,4-difluoro | 6-chloropyridin-3-yl | 0.57 |
| 10-58 | 4,4-difluoro | 4-(trifluoromethyl)phenyl | 0.61 |
| 10-59 | 4,4-difluoro | 4-ethoxyphenyl | 1.28 |
| 10-60 | 4,4-difluoro | 4-propoxyphenyl | 0.83 |
| 10-61 | 4,4-dimethyl | 4-fluorophenyl | 0.55 |
| 10-62 | 4,4-dimethyl | 6-fluoropyridin-3-yl | 0.71 |
| 10-63 | 4,4-dimethyl | 4-chlorophenyl | 0.86 |
| 10-64 | 4,4-dimethyl | 6-chloropyridin-3-yl | 0.57 |
| 10-65 | 4,4-dimethyl | 4-chloro-2-fluorophenyl | 0.58 |
| 10-66 | 4,4-dimethyl | benzo[d][1,3]dioxol-5-yl | 0.61 |
| 10-67 | 3,5-dimethyl | 4-fluorophenyl | 0.65 |
| 10-68 | 3,5-dimethyl | 6-fluoropyridin-3-yl | 0.97 |
| 10-69 | 3,5-dimethyl | 4-chlorophenyl | 0.86 |
| 10-70 | 3,5-dimethyl | 6-chloropyridin-3-yl | 0.96 |
| 10-71 | 3,5-dimethyl | methyl 4-benzoate | 0.69 |
| 10-72 | 3,5-dimethyl | 4-chloro-2-fluorophenyl | 0.76 |

### Compound 10-1

### N-(1-cyclohexyl-6-(3,3-difluoropyrrolidin-1-yl)-1H-pyrazolo[3,4-d]pyrimidin-4-yl)-5-nitrothiophene-2-carboxamide

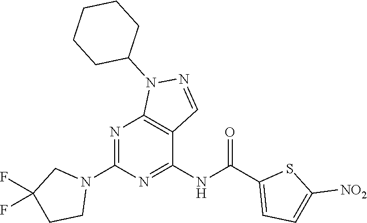 [SMILES: O=C(Nc1nc(N2CCC(F)(F)C2)nc2c1cnn2C1CCCCC1)c1ccc([N+](=O)[O-])s1]

¹H NMR (400 MHz, DMSO-d₆): δ 11.50 (br. s., 1H), 8.28 (d, J=4.4 Hz, 1H), 8.21 (d, J=4.4 Hz, 1H), 8.06 (s, 1H), 4.50-4.60 (m, 1H), 4.01 (t, J=13.2 Hz, 2H), 3.83 (t, J=7.3 Hz, 2H), 2.52-2.63 (m, 2H), 1.82-1.97 (m, 6H), 1.70 (d, J=12.7 Hz, 1H), 1.38-1.52 (m, 2H), 1.19-1.34 (m, 1H). MS(M+1): 478. Light yellow solid.

### Compound 10-2

### N-(1-cyclohexyl-6-(4,4-difluoropiperidin-1-yl)-1H-pyrazolo[3,4-d]pyrimidin-4-yl)-5-nitrothiophene-2-carboxamide

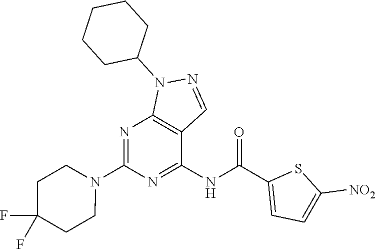 [SMILES: O=C(Nc1nc(N2CCC(F)(F)CC2)nc2c1cnn2C1CCCCC1)c1ccc([N+](=O)[O-])s1]

¹H NMR (400 MHz, DMSO-d₆): δ 11.44 (br. s., 1H), 8.24-8.30 (m, 1H), 8.22 (d, J=4.4 Hz, 1H), 8.07 (s, 1H), 4.54 (dt, J=9.8, 4.9 Hz, 1H), 4.00 (t, J=5.6 Hz, 4H), 1.96-2.14 (m, 4H), 1.80-1.96 (m, 6H), 1.66-1.76 (m, 1H), 1.37-1.51 (o, 2H), 1.18-1.31 (m, 1H). MS(M+1) 492. Light yellow solid.

### Compound 10-3

### (S)—N-(1-cyclohexyl-6-(2-(hydroxymethyl)pyrrolidin-1-yl)-1H-pyrazolo[3,4-d]pyrimidin-4-yl)-5-nitrothiophene-2-carboxamide

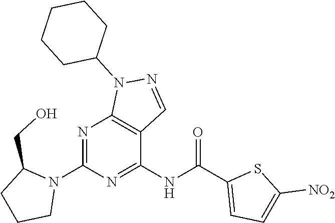 [SMILES: O=C(Nc1nc(N2CCC[C@H]2CO)nc2c1cnn2C1CCCCC1)c1ccc([N+](=O)[O-])s1]

¹H NMR (400 MHz, DMSO-d₆): δ 8.12-8.38 (m, 2H), 7.98 (s, 1H), 4.78 (br. s., 1H), 4.40-4.58 (m, 1H), 4.21 (br. s., 1H), 3.66 (br. s., 1H), 3.45-3.63 (m, 3H), 1.79-2.08 (m, 10H), 1.69 (d, J=13.2 Hz, 1H), 1.32-1.51 (m, 2H), 1.15-1.32 (m, 1H). MS(M+1): 472. Orange solid.

### Compound 10-4

### N-(1-cyclohexyl-6-(furan-2-yl)-1H-pyrazolo[3,4-d]pyrimidin-4-yl)-5-nitrothiophene-2-carboxamide

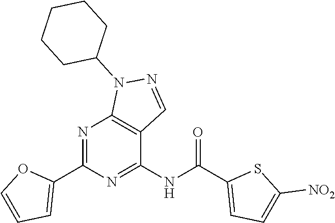 [SMILES: O=C(Nc1nc(-c2ccco2)nc2c1cnn2C1CCCCC1)c1ccc([N+](=O)[O-])s1]

¹H NMR (400 MHz, DMSO-d₆): δ 12.06 (br. s., 1H), 8.35 (d, J=4.4 Hz, 1H), 8.32 (s, 1H), 8.22 (d, J=4.4 Hz, 1H), 7.98 (s, 1H), 7.40 (d, J=2.9 Hz, 1H), 6.74 (dd, J=3.4, 2.0 Hz, 1H), 4.75-4.87 (m, 1H), 1.92-2.05 (m, 4H), 1.89 (d, J=13.2 Hz, 2H), 1.73 (d, J=12.7 Hz, 1H), 1.45-1.62 (m, 2H), 1.23-1.38 (m, 1H). MS(M+1): 439. Khaki solid.

### Compound 10-5

### N-(1-cyclohexyl-6-(thiophen-2-yl)-1H-pyrazolo[3,4-d]pyrimidin-4-yl)-5-nitrothiophene-2-carboxamide

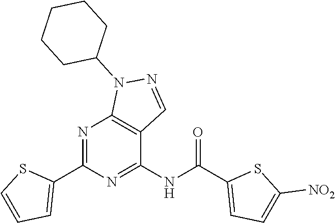 [SMILES: O=C(Nc1nc(-c2cccs2)nc2c1cnn2C1CCCCC1)c1ccc([N+](=O)[O-])s1]

¹H NMR (400 MHz, DMSO-d₆): δ 11.96 (br s., 1H), 8.33 (d, J=4.4 Hz, 1H), 8.30 (s, 1H), 8.23 (d, J=4.4 Hz, 1H), 8.07 (dd, J=3.9, 1.5 Hz, 1H), 7.79 (dd, J=4.9, 1.0 Hz, 1H), 7.20-7.28 (m, 1H), 4.77 (di, J=15.6, 7.8 Hz, 1H), 1.81-2.04 (m, 6H), 1.74 (d, J=12.7 Hz, 1H), 1.43-1.60 (m, 2H), 1.21-1.38 (m, 1H). MS(M+1): 455. Khaki solid.

### Compound 10-6

### N-(1-cyclohexyl-6-(thiophen-3-yl)-1H-pyrazolo[3,4-d]pyrimidin-4-yl)-5-nitrothiophene-2-carboxamide

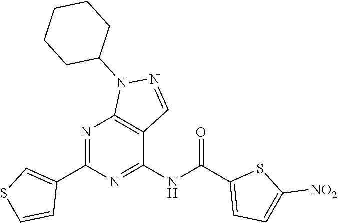 [SMILES: O=C(Nc1nc(-c2ccsc2)nc2c1cnn2C1CCCCC1)c1ccc([N+](=O)[O-])s1]

¹H NMR (400 MHz, DMSO-d₆): δ 11.93 (br. s., 1H), 8.46 (d, J=2.9 Hz, 1H), 8.29-8.37 (m, 2H), 8.24 (d, J=4.4 Hz, 1H), 7.94 (d, J=4.9 Hz, 1H), 7.69 (dd, J=5.1, 3.2 Hz, 1H), 4.78-4.91 (m, 1H), 1.94-2.06 (m, 4H), 1.89 (d, J=12.7 Hz, 2H), 1.74 (d, J=12.7 Hz, 1H), 1.45-1.60 (m, 2H), 1.23-1.39 (m, 1H). MS(M+1) 455. Khaki solid.

### Compound 10-7

### N-(1-cyclohexyl-6-(5-methylthiophen-2-yl)-1H-pyrazolo[3,4-d]pyrimidin-4-yl)-5-nitrothiophene-2-carboxamide

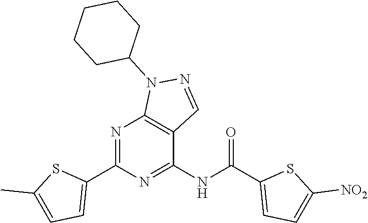 [SMILES: Cc1ccc(-c2nc(NC(=O)c3ccc([N+](=O)[O-])s3)c3cnn(C4CCCCC4)c3n2)s1]

¹H NMR (400 MHz, DMSO-d₆): δ 11.86 (br. s., 1H), 8.32-8.40 (m, 1H), 8.27 (s, 1H), 8.17-8.24 (m, 1H), 7.87 (d, J=3.4 Hz, 1H), 6.93 (dd, J=3.4, 1.0 Hz, 1H), 4.66-4.79 (m, 1H), 2.53 (s, 3H), 1.93-2.04 (m, 4H), 1.89 (d, J=13.2 Hz, 2H), 1.73 (d, J=12.7 Hz, 1H), 1.42-1.56 (m, 2H), 1.20-1.36 (m, 1H). MS(M+1): 469. Yellow solid.

### Compound 10-8

### N-(6-(5-chlorothiophen-2-yl)-1-cyclohexyl-1H-pyrazolo[3,4-d]pyrimidin-4-yl)-5-nitrothiophene-2-carboxamide

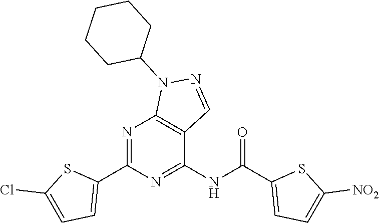 [SMILES: O=C(Nc1nc(-c2ccc(Cl)s2)nc2c1cnn2C1CCCCC1)c1ccc([N+](=O)[O-])s1]

¹H NMR (400 MHz, DMSO-d₆): δ 11.91 (br. s., 1H), 8.28-8.41 (m, 2H), 8.23 (d, J=4.4 Hz, 1H), 7.90 (d, J=3.9 Hz, 1H), 7.26 (d, J=3.9 Hz, 1H), 4.75 (t, J=7.3 Hz, 1H), 1.93-2.08 (m, 4H), 1.89 (d, J=13.2 Hz, 2H), 1.73 (d, J=12.7 Hz, 1H), 1.40-1.60 (m, 2H), 1.15-1.36 (m, 1H). MS(M+1): 489. Yellow solid.

### Compound 10-9

### N-(1-cyclohexyl-4-(3,5-dimethylisoxazol-4-yl)-1H-pyrazolo[3,4,d]pyrimidin-4-yl)-5-nitrothiophene-2-carboxamide

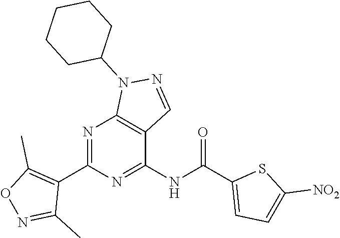 [SMILES: Cc1noc(C)c1-c1nc(NC(=O)c2ccc([N+](=O)[O-])s2)c2cnn(C3CCCCC3)c2n1]

¹H NMR (400 MHz, DMSO-d₆): δ 11.65 (br. s., 1H), 8.34 (s, 1H), 8.23-8.30 (m, 2H), 4.74 (dt, J=15.7, 7.8 Hz, 1H), 2.88 (s, 3H), 2.60-2.69 (m, 3H), 1.94-2.05 (m, 4H), 1.89 (d, J=13.2 Hz, 2H), 1.73 (d, J=13.2 Hz, 1H), 1.41-1.57 (m, 2H), 1.24-1.36 (m, 1H). MS(M+1): 468. Khaki solid.

### Compound 10-10

### N-(1-cyclohexyl-6-(pyridin-3-yl)-1H-pyrazolo[3,4,d]pyrimidin-4-yl)-5-nitrothiophene-2-carboxamide

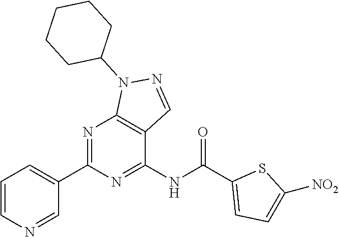 [SMILES: O=C(Nc1nc(-c2cccnc2)nc2c1cnn2C1CCCCC1)c1ccc([N+](=O)[O-])s1]

¹H NMR (400 MHz, DMSO-d₆): δ 11.95 (br. s., 1H), 9.67 (d, J=1.5 Hz, 1H), 8.69-8.86 (m, 2H), 8.41 (s, 1H), 8.36 (d, J=4.4 Hz, 1H), 8.25 (d, J=4.4 Hz, 1H), 7.55-7.67 (m, 1H), 4.80-5.01 (m, 1H), 1.94-2.08 (m, 4H), 1.83-1.94 (m, 2H), 1.74 (d, J=13.2 Hz, 1H), 1.48-1.66 (m, 2H), 1.23-1.39 (m, 1H). MS(M+1): 450. Khaki solid.

### Compound 10-11

### N-(1-cyclohexyl-6-(2-fluorophenyl)-1H-pyrazolo[3,4-d]pyrimidin-4-yl)-5-nitrothiophene-2-carboxamide

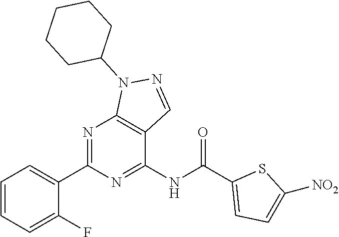 [SMILES: O=C(Nc1nc(-c2ccccc2F)nc2c1cnn2C1CCCCC1)c1ccc([N+](=O)[O-])s1]

¹H NMR (400 MHz, DMSO-d₆): δ 12.08 (br. s., 1H), 8.41 (s, 1H), 8.34 (br. s., 1H), 8.22 (d, J=4.4 Hz, 1H), 8.01-8.12 (m, 1H), 7.55-7.67 (m, 1H), 7.30-7.43 (m, 2H), 4.81 (dt, J=15.4, 7.9 Hz, 1H), 1.92-2.09 (m, 4H), 1.88 (d, J=13.2 Hz, 2H), 1.66-1.79 (m, 1H), 1.43-1.57 (m, 2H), 1.22-1.35 (m, 1H). MS(M+1): 467. Light khaki solid.

### Compound 10-12

### N-(1-cyclohexyl-6-(3-fluorophenyl)-1H-pyrazolo[3,4-d]pyrimidin-4-yl)-5-nitrothiophene-2-carboxamide

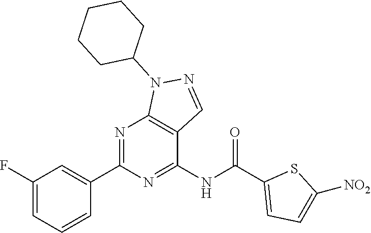 [SMILES: O=C(Nc1nc(-c2cccc(F)c2)nc2c1cnn2C1CCCCC1)c1ccc([N+](=O)[O-])s1]

¹H NMR (400 MHz, DMSO-d₆): δ 8.34-8.47 (m, 2H), 8.18-8.34 (m, 3H), 7.62 (td, J=7.9, 6.1 Hz, 1H), 7.40 (td, J=8.4, 2.2 Hz, 1H), 4.84-4.96 (m, 1H), 1.94-2.08 (m, 4H), 1.85-1.94 (m, 2H), 1.74 (d, J=12.7 Hz, 1H), 1.56 (d, J=8.3 Hz, 2H), 1.25-1.37 (m, 1H). MS(M+1): 467. Khaki solid.

### Compound 10-13

### N-(1-cyclohexyl-6-(4-fluorophenyl)-1H-pyrazolo[3,4-d]pyrimidin-4-yl)-5-nitrothiophene-2-carboxamide

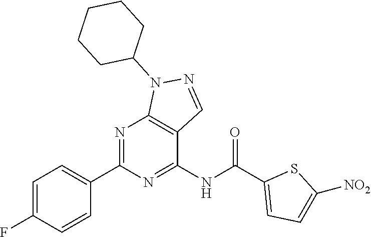 [SMILES: O=C(Nc1nc(-c2ccc(F)cc2)nc2c1cnn2C1CCCCC1)c1ccc([N+](=O)[O-])s1]

¹H NMR (400 MHz, DMSO-d₆): δ 11.88 (br. s., 1H), 8.53-8.64 (m, 2H), 8.32-8.42 (m, 2H), 8.25 (d, J=4.4 Hz, 1H), 7.34-7.47 (m, 2H), 4.83-4.96 (m, 1H), 1.95-2.10 (m, 4H), 1.91 (d, J=13.2 Hz, 2H), 1.69-1.81 (m, 1H), 1.46-1.64 (m, 2H), 1.24-1.39 (m, 1H). MS(M+1): 467.

### Compound 10-14

### N-(1-cyclohexyl-6-(6-fluoropyridin-3-yl)-1H-pyrazolo[3,4-d]pyrimidin-4-yl)-5-nitrothiophene-2-carboxamide

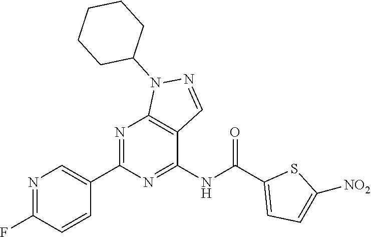 [SMILES: O=C(Nc1nc(-c2ccc(F)nc2)nc2c1cnn2C1CCCCC1)c1ccc([N+](=O)[O-])s1]

¹H NMR (400 MHz, DMSO-d₆): δ 11.95 (br. s., 1H), 9.31 (d, J=2.4 Hz, 1H), 8.96 (td, J=8.3, 2.4 Hz, 1H), 8.41 (s, 1H), 8.34 (d, J=4.4 Hz, 1H), 8.25 (d, J=4.4 Hz, 1H), 7.41 (dd, J=8.6, 2.7 Hz, 1H), 4.91 (t, J=6.8 Hz, 1H), 1.94-2.05 (m, 4H), 1.85-1.94 (m, 2H), 1.74 (d, J=12.7 Hz, 1H), 1.47-1.61 (m, 2H), 1.30 (d, J=12.7 Hz, 1H). MS(M+1): 468. Light yellow solid.

### Compound 10-15

### N-(1-cyclohexyl-6-(2-fluoropyridin-4-yl)-1H-pyrazolo[3,4-d]pyrimidin-4-yl)-5-nitrothiophene-2-carboxamide

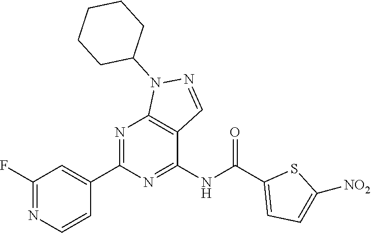 [SMILES: O=C(Nc1nc(-c2ccnc(F)c2)nc2c1cnn2C1CCCCC1)c1ccc([N+](=O)[O-])s1]

¹H NMR (400 MHz, DMSO-d₆): δ 12.00 (br s., 1H), 8.43-8.52 (m, 2H), 8.31-8.38 (m, 2H), 8.25 (d, J=4.4 Hz, 1H), 8.09 (s, 1H), 4.88-4.99 (m, 1H), 1.96-2.06 (m, 4H), 1.90 (d, J=12.7 Hz, 2H), 1.71-1.80 (m, 1H), 1.49-1.61 (m, 2H), 1.26-1.38 (m, 1H). MS (M+1): 468. Light yellow solid.

### Compound 10-16

### N-(6-(3-chlorophenyl)-1-cyclohexyl-1H-pyrazolo[3,4-d]pyrimidin-4-yl)-5-nitrothiophene-2-carboxamide

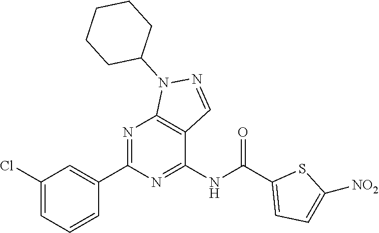 [SMILES: O=C(Nc1nc(-c2cccc(Cl)c2)nc2c1cnn2C1CCCCC1)c1ccc([N+](=O)[O-])s1]

¹H NMR (400 MHz, DMSO-d₆): δ 11.90 (br. s., 1H), 8.50-8.63 (m, 1H), 8.45-8.50 (m, 1H), 8.39 (s, 1H), 8.34 (d, J=4.4 Hz, 1H), 8.24 (d, J=4.4 Hz, 1H), 7.53-7.69 (m, 2H), 4.82-4.97 (m, 1H), 1.93-2.10 (m, 4H), 1.90 (d, J=13.2 Hz, 2H), 1.74 (d, J=13.2 Hz, 1H), 1.45-1.64 (m, 2H), 1.21-1.41 (m, 1H). MS(M+1): 483. White solid.

### Compound 10-17

### N-(6-(4-chlorophenyl)-1-cyclohexyl-1H-pyrazolo[3,4-d]pyrimidin-4-yl)-5-nitrothiophene-2-carboxamide

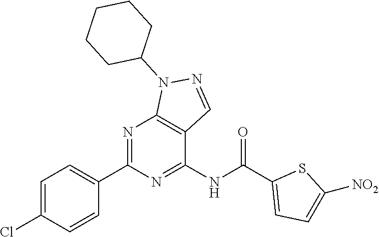 [SMILES: O=C(Nc1nc(-c2ccc(Cl)cc2)nc2c1cnn2C1CCCCC1)c1ccc([N+](=O)[O-])s1]

¹H NMR (400 MHz, DMSO-d₆): δ 11.88 (br. s., 1H), 8.49-8.55 (m, 2H), 8.30-8.41 (m, 2H), 8.22 (d, J=4.4 Hz, 1H), 7.58-7.69 (m, 2H), 4.82-4.92 (m, 1H), 1.92-2.03 (m, 4H), 1.89 (d, J=13.7 Hz, 2H), 1.69-1.81 (m, 1H), 1.44-1.62 (m, 2H), 1.22-1.37 (m, 1H). MS(M+1): 483. Yellow solid.

### Compound 10-18

### N-(6-(6-chloropyridin-3-yl)-1-cyclohexyl-1H-pyrazolo[3,4-d]pyrimidin-4-yl)-5-nitrothiophene-2-carboxamide

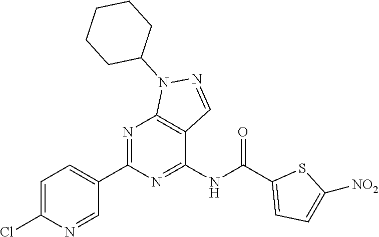 [SMILES: O=C(Nc1nc(-c2ccc(Cl)nc2)nc2c1cnn2C1CCCCC1)c1ccc([N+](=O)[O-])s1]

¹H NMR (400 MHz, DMSO-d₆): δ 11.97 (br. s., 1H), 9.46 (d, J=2.4 Hz, 1H), 8.82 (dd, J=8.3, 2.4 Hz, 1H), 8.42 (s, 1H), 8.35 (d, J=4.4 Hz, 1H), 8.25 (d, J=4.4 Hz, 1H), 7.75 (d, J=8.3 Hz, 1H), 4.77-5.01 (m, 1H), 1.94-2.07 (m, 4H), 1.83-1.94 (m, 2H), 1.74 (d, J=12.7 Hz, 1H), 1.44-1.64 (m, 2H), 1.20-1.39 (m, 1H). MS(M+1): 484. Light yellow solid.

### Compound 10-19

### N-(1-cyclohexyl-6-(6-methylpyrimidin-3-yl)-1H-pyrazolo[3,4-d]pyrimidin-4-yl)-5-nitrothiophene-2-carboxamide

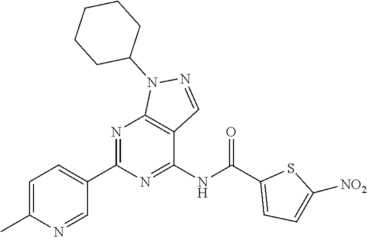 [SMILES: Cc1ccc(-c2nc(NC(=O)c3ccc([N+](=O)[O-])s3)c3cnn(C4CCCCC4)c3n2)cn1]

¹H NMR (400 MHz, DMSO-d₆): δ 11.95 (br. s., 1H), 9.54 (d, J=2.0 Hz, 1H), 8.68 (dd, J=8.1, 2.2 Hz, 1H), 8.38 (s, 1H), 8.34 (d, J=4.4 Hz, 1H), 8.24 (d, J=4.4 Hz, 1H), 7.46 (d, J=8.3 Hz, 1H), 4.81-4.98 (m, 1H), 2.58 (s, 3H), 1.95-2.05 (m, 4H), 1.90 (d, J=13.2 Hz, 2H), 1.74 (d, J=12.7 Hz, 1H), 1.46-1.62 (m, 2H), 1.21-1.36 (m, 1H). MS(M+1): 464. Yellow solid.

### Compound 10-20

### N-(6-(4-cyanophenyl)-1-cyclohexyl-1H-pyrazolo[3,4-d]pyrimidin-4-yl)-5-nitrothiophene-2-carboxamide

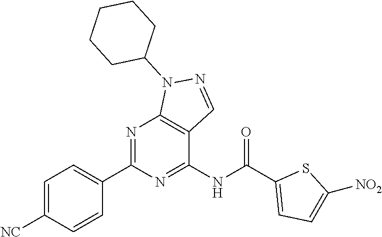 [SMILES: N#Cc1ccc(-c2nc(NC(=O)c3ccc([N+](=O)[O-])s3)c3cnn(C4CCCCC4)c3n2)cc1]

¹H NMR (400 MHz, DMSO-d₆): δ 11.96 (br. s., 1H), 8.65-8.73 (m, 2H), 8.41 (s, 1H), 8.35 (d, J=4.4 Hz, 1H), 8.24 (d, J=4.4 Hz, 1H), 8.01-8.09 (m, 2H), 4.84-4.96 (m, 1H), 1.95-2.07 (m, 4H), 1.90 (d, J=13.2 Hz, 2H), 1.70-1.81 (m, 1H), 1.48-1.62 (m, 2H), 1.25-1.40 (m, 1H). MS(M+1) 474. Light khaki solid.

### Compound 10-21

### N-(1-cyclohexyl-6-(6-methoxypyridin-3-yl)-1H-pyrazolo[3,4-d]pyrimidin-4-yl)-5-nitrothiophene-2-carboxamide

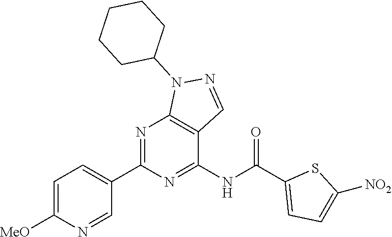 [SMILES: COc1ccc(-c2nc(NC(=O)c3ccc([N+](=O)[O-])s3)c3cnn(C4CCCCC4)c3n2)cn1]

¹H NMR (400 MHz, DMSO-d₆): δ 11.85 (br. s., 1H), 9.28 (d, J=2.0 Hz, 1H), 8.69 (dd, J=8.8, 2.4 Hz, 1H), 8.31-8.39 (m, 2H), 8.23 (d, J=4.4 Hz, 1H), 7.00 (d, J=8.8 Hz, 1H), 4.79-4.93 (m, 1H), 3.95 (s, 3H), 1.93-2.08 (m, 4H), 1.89 (d, J=13.2 Hz, 2H), 1.70-1.79 (m, 1H), 1.43-1.61 (m, 2H), 1.20-1.36 (m, 1H). MS(M+1): 480. Light khaki solid.

### Compound 10-22

### N-(1-cyclohexyl-6-(5-ethoxypyridin-3-yl)-1H-pyrazolo[3,4-d]pyrimidin-4-yl)-5-nitrothiophene-2-carboxamide

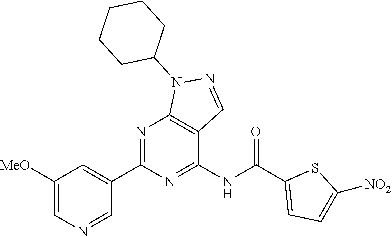 [SMILES: COc1cncc(-c2nc(NC(=O)c3ccc([N+](=O)[O-])s3)c3cnn(C4CCCCC4)c3n2)c1]

¹H NMR (400 MHz, DMSO-d₆): δ 11.92 (br. s., 1H), 9.29 (s, 1H), 8.47 (d, J=2.4 Hz, 1H), 8.41 (s, 1H), 8.36 (d, J=4.4 Hz, 1H), 8.27-8.34 (m, 1H), 8.25 (d, J=4.4 Hz, 1H), 4.92 (t, J=7.1 Hz, 1H), 3.97 (s, 3H), 1.95-2.03 (m, 4H), 1.90 (d, J=12.7 Hz, 2H), 1.74 (d, J=13.2 Hz, 1H), 1.48-1.61 (m, 2H), 1.24-1.36 (m, 1H). MS(M+1): 480. Goldenrod powder.

### Compound 10-23

### N-(1-cyclohexyl-6-(6-ethoxypyridin-3-yl)-1H-pyrazolo[3,4-d]pyrimidin-4-yl)-5-nitrothiophene-2-carboxamide

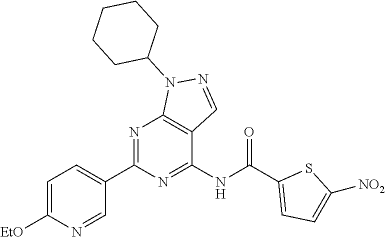 [SMILES: CCOc1ccc(-c2nc(NC(=O)c3ccc([N+](=O)[O-])s3)c3cnn(C4CCCCC4)c3n2)cn1]

¹H NMR (400 MHz, DMSO-d₆): δ 11.84 (br. s., 1H), 9.26 (d, J=2.0 Hz, 1H), 8.68 (dd, J=8.8, 2.4 Hz, 1H), 8.30-8.41 (m, 2H), 8.23 (d, J=4.4 Hz, 1H), 6.96 (d, J=8.8 Hz, 1H), 4.86 (t, J=6.8 Hz, 1H), 4.40 (q, J=7.3 Hz, 2H), 1.93-2.07 (m, 4H), 1.89 (d, J=13.7 Hz, 2H), 1.74 (d, J=12.7 Hz, 1H), 1.44-1.63 (m, 2H), 1.36 (t, J=6.8 Hz, 3H), 1.20-1.33 (m, 1H). MS(M+1): 494. Brown solid.

### Compound 10-24

### N-(1-cyclohexyl-6-(4-(trifluoromethyl)phenyl)-1H-pyrazolo[3,4-d]pyrimidin-4-yl)-5-nitrothiophene-2-carboxamide

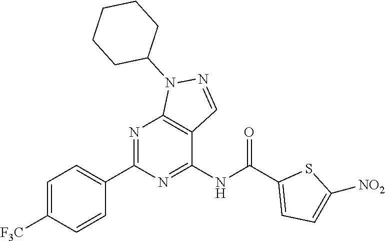 [SMILES: O=C(Nc1nc(-c2ccc(C(F)(F)F)cc2)nc2c1cnn2C1CCCCC1)c1ccc([N+](=O)[O-])s1]

¹H NMR (400 MHz, DMSO-d₆): δ 11.99 (br. s., 1H), 8.69-8.78 (m, J=8.3 Hz, 2H), 8.41 (s, 1H), 8.36 (d, J=4.4 Hz, 1H), 8.24 (d, J=4.4 Hz, 1H), 7.91-7.99 (m, J=8.3 Hz, 2H), 4.86-4.97 (m, 1H), 1.94-2.09 (m, 4H), 1.83-1.94 (m, 2H), 1.75 (d, J=12.7 Hz, 1H), 1.46-1.62 (m, 2H), 1.25-1.41 (m, 1H). MS(M+1): 517. Yellow-brown solid.

### Compound 10-25

### N-(1-cyclohexyl-6-(6-(trifluoromethyl)pyridin-3-yl)-1H-pyrazolo[3,4-d]pyrimidin-4-yl)-5-nitrothiophene-2-carboxamide

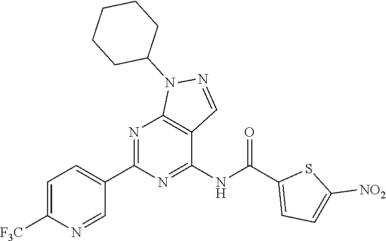 [SMILES: O=C(Nc1nc(-c2ccc(C(F)(F)F)nc2)nc2c1cnn2C1CCCCC1)c1ccc([N+](=O)[O-])s1]

¹H NMR (400 Hz, DMSO-d₆): δ 12.01 (br. s., 1H), 9.72-9.83 (m, 1H), 9.03 (dd, J=8.1, 1.7 Hz, 1H), 8.41-8.49 (m, 1H), 8.36 (d, J=4.4 Hz, 1H), 8.24 (d, J=4.4 Hz, 1H), 8.10-8.16 (m, 1H), 4.83-5.00 (m, 1H), 1.95-2.11 (m, 4H), 1.90 (d, J=13.2 Hz, 2H), 1.75 (d, J=12.7 Hz, 1H), 1.45-1.62 (m, 2H), 1.25-1.39 (m, 1H). MS(M+1): 518. Khaki solid.

### Compound 10-26

### N-(1-cyclohexyl-6-(4-(trifluoromethoxy)phenyl)-1H-pyrazolo[3,4-d]pyrimidin-4-yl)-5-nitrothiophene-2-carboxamide

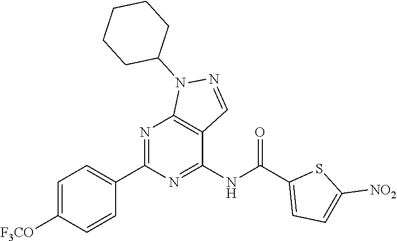 [SMILES: O=C(Nc1nc(-c2ccc(OC(F)(F)F)cc2)nc2c1cnn2C1CCCCC1)c1ccc([N+](=O)[O-])s1]

¹H NMR (400 MHz, DMSO-d₆): δ 11.94 (br. s., 1H), 8.59-8.68 (m, 2H), 8.30-8.40 (m, 2H), 8.23 (d, J=4.4 Hz, 1H), 7.49-7.63 (m, J=8.3 Hz, 2H), 4.81-4.95 (m, 1H), 1.94-2.09 (m, 4H), 1.89 (d, J=12.7 Hz, 2H), 1.69-1.82 (m, 1H), 1.44-1.61 (m, 2H), 1.23-1.37 (m, 1H). MS(M+1): 533. Khaki solid.

### Compound 10-27

### N-(1-cyclohexyl-6-(4-formylphenyl)-1H-pyrazolo[3,4-d]pyrimidin-4-yl)-5-nitrothiophene-2-carboxamide

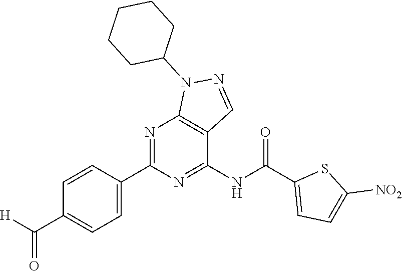 [SMILES: O=Cc1ccc(-c2nc(NC(=O)c3ccc([N+](=O)[O-])s3)c3cnn(C4CCCCC4)c3n2)cc1]

¹H NMR (400 MHz, DMSO-d₆): δ 11.98 (br. s., 1H), 10.13 (s, 1H), 8.66-8.79 (m, J=8.3 Hz, 2H), 8.34-8.47 (m, 2H), 8.25 (d, J=4.4 Hz, 1H), 8.03-8.20 (m, J=8.3 Hz, 2H), 4.79-5.03 (m, 1H), 1.96-2.09 (m, 4H), 1.83-1.96 (m, 2H), 1.75 (d, J=13.2 Hz, 1H), 1.43-1.68 (m, 2H), 1.21-1.41 (m, 1H). MS(M+1): 477. Yellow solid.

### Compound 10-28

### N-(6-(4-acetylphenyl)-1-cyclohexyl-1H-pyrazolo[3,4-d]pyrimidin-4-yl)-5-nitrothiophene-2-carboxamide

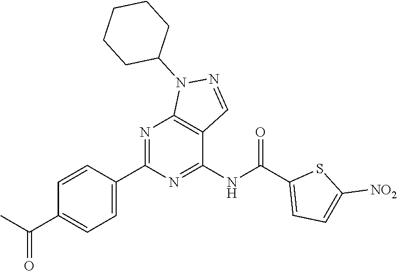 [SMILES: CC(=O)c1ccc(-c2nc(NC(=O)c3ccc([N+](=O)[O-])s3)c3cnn(C4CCCCC4)c3n2)cc1]

¹H NMR (DMSO-d₆, 400 MHz): δ 11.97 (brs., 1H), 8.56-8.74 (m, J=8.3 Hz, 2H), 8.31-8.45 (m, 2H), 8.25 (d, J=4.4 Hz, 1H), 8.05-8.20 (m, J=8.3 Hz, 2H), 4.83-4.99 (m, 1H), 2.66 (s, 3H), 1.95-2.06 (m, 4H), 1.91 (d, J=12.7 Hz, 2H), 1.75 (d, J=13.2 Hz, 1H), 1.43-1.66 (m, 2H), 1.21-1.40 (m, 1H). MS(M+1): 491. Yellow solid.

### Compound 10-29

### N-(1-cyclohexyl-6-(4-((dimethylamino)methyl)phenyl)-1H-pyrazolo[3,4-d]pyrimidin-4-yl)-5-nitrothiophene-2-carboxamide

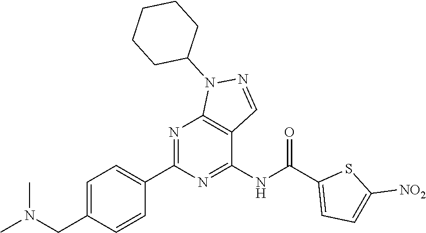 [SMILES: CN(C)Cc1ccc(-c2nc(NC(=O)c3ccc([N+](=O)[O-])s3)c3cnn(C4CCCCC4)c3n2)cc1]

¹H NMR (400 MHz, DMSO-d₆): δ 11.93 (br. s., 1H), 8.53-8.66 (m, J=8.3 Hz, 2H), 8.32-8.49 (m, 2H), 8.26 (d, J=4.4 Hz, 1H), 7.65-7.79 (m, J=7.8 Hz, 2H), 4.91 (dt, J=15.3, 7.8 Hz, 1H), 4.32 (br. s., 2H), 2.71 (s, 6H), 1.94-2.08 (m, 4H), 1.90 (d, J=13.2 Hz, 2H), 1.75 (d, J=13.2 Hz, 1H), 1.43-1.63 (m, 2H), 1.26-1.41 (m, 1H). MS(M+1): 506. Khaki solid.

### Compound 10-30

### N-(1-cyclohexyl-6-(4-(methoxymethyl)phenyl)-1H-pyrazolo[3,4-d]pyrimidin-4-yl)-5-nitrothiophene-2-carboxamide

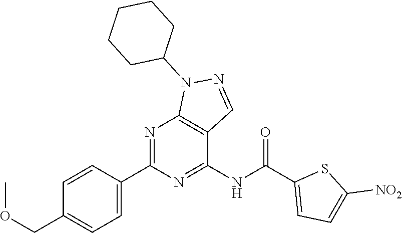 [SMILES: COCc1ccc(-c2nc(NC(=O)c3ccc([N+](=O)[O-])s3)c3cnn(C4CCCCC4)c3n2)cc1]

¹H NMR (400 MHz, DMSO-d₆): δ 11.91 (br. s., 1H), 8.48-8.59 (m, J=8.3 Hz, 2H), 8.33-8.41 (m, 2H), 8.24 (d, J=4.4 Hz, 1H), 7.44-7.57 (m, J=8.3 Hz, 2H), 4.82-4.99 (m, 1H), 4.52 (s, 2H), 3.35 (s, 3H), 1.93-2.09 (m, 4H), 1.90 (d, J=13.2 Hz, 2H), 1.71-1.81 (m, 1H), 1.47-1.62 (m, 2H), 1.26-1.37 (m, 1H). MS(M+1): 493. Khaki solid

### Compound 10-31

### methyl 3-(1-cyclohexyl-4-(5-nitrothiophene-2-carboxamido)-1H-pyrazolo[3,4-d]pyrimidin-6-yl)benzoate

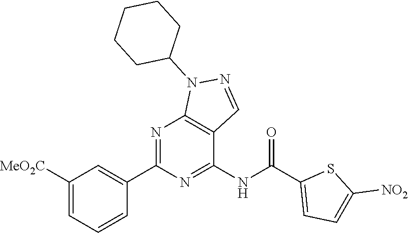 [SMILES: COC(=O)c1cccc(-c2nc(NC(=O)c3ccc([N+](=O)[O-])s3)c3cnn(C4CCCCC4)c3n2)c1]

¹H NMR (400 MHz, DMSO-d₆): δ 12.01 (br. s., 1H), 9.10 (t, J=1.7 Hz, 1H), 8.73-8.84 (m, 1H), 8.31-8.42 (m, 2H), 8.24 (d, J=4.9 Hz, 1H), 8.10-8.17 (m, 1H), 7.73 (t, J=7.8 Hz, 1H), 4.85-4.98 (m, 1H), 3.93 (s, 3H), 1.96-2.12 (m, 4H), 1.90 (d, J=13.2 Hz, 2H), 1.75 (d, J=12.7 Hz, 1H), 1.46-1.64 (m, 2H), 1.25-1.39 (m, 1H).

MS(M+1) 507. Khaki solid.

### Compound 10-32

### methyl 4-(1-cyclohexyl-4-(5-nitrothiophene-2-carboxamido)-1H-pyrazolo[3,4-d] pyrmidin-6-yl)benzoate

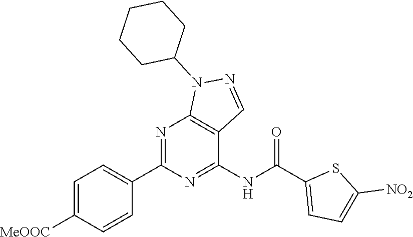 [SMILES: COC(=O)c1ccc(-c2nc(NC(=O)c3ccc([N+](=O)[O-])s3)c3cnn(C4CCCCC4)c3n2)cc1]

¹H NMR (400 MHz, DMSO-d₆): δ 12.02 (br. s., 1H), 8.61-4.71 (m, J=8.3 Hz, 2H), 8.38-8.45 (m, 1H), 8.34 (d, J=4.4 Hz, 1H), 8.24 (d, J=4.4 Hz, 1H), 8.10-8.20 (m, 2H), 4.83-5.01 (m, 1H), 3.82-3.98 (m, 3H), 1.95-2.07 (m, 4H), 1.84-1.95 (m, 2H), 1.75 (d, J=13.2 Hz, 1H), 1.48-1.63 (m, 2H), 1.23-1.39 (m, 1H). MS(M+1): 507. Light khaki solid.

### Compound 10-33

### ethyl 4-(1-cyclohexyl-4-(5-nitrothiophene-2-carboxamido)-1H-pyrazolo[3,4-d] pyrimidin-6-yl)benzoate 1285

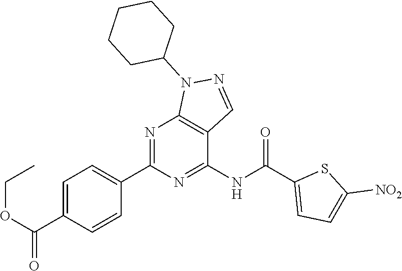 [SMILES: CCOC(=O)c1ccc(-c2nc(NC(=O)c3ccc([N+](=O)[O-])s3)c3cnn(C4CCCCC4)c3n2)cc1]

¹H NMR (400 MHz, DMSO-d₆): δ 12.00 (br. s., 1H), 8.61-8.72 (m, J=8.3 Hz, 2H), 8.39 (s, 1H), 8.35 (d, J=4.4 Hz, 1H), 8.24 (d, J=4.4 Hz, 1H), 8.11-8.20 (m, J=8.3 Hz, 2H), 4.86-4.98 (m, 1H), 4.37 (q, J=7.2 Hz, 2H), 1.96-2.14 (m, 4H), 1.90 (d, J=13.2 Hz, 2H), 1.75 (d, J=13.2 Hz, 1H), 1.47-1.65 (m, 2H), 1.36 (t, J=7.1 Hz, 3H), 1.24-1.34 (m, 1H). MS(M+1): 521. Yellow solid.

### Compound 10-34

### N-(1-cyclohexyl-6-(4-(methylsulfonyl)phenyl)-1H-pyrazolo[3,4-d]pyrimidin-4-yl)-5-nitrothiophene-2-carboxamide

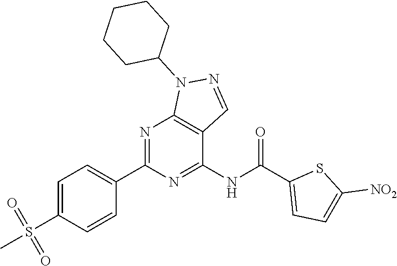 [SMILES: CS(=O)(=O)c1ccc(-c2nc(NC(=O)c3ccc([N+](=O)[O-])s3)c3cnn(C4CCCCC4)c3n2)cc1]

¹H NMR (400 MHz, DMSO-d₆): δ 12.00 (br. s., 1H), 8.67-8.80 (m, 2H), 8.40 (s, 1H), 8.36 (d, J=4.4 Hz, 1H), 8.24 (d, J=4.4 Hz, 1H), 8.10-8.18 (m, 2H), 4.85-4.96 (m, 1H), 3.30 (s, 3H), 1.95-2.09 (m, 4H), 1.90 (d, J=13.2 Hz, 2H), 1.68-1.82 (m, 1H), 1.47-1.64 (m, 2H), 1.23-1.40 (m, 1H). MS(M+1): 527. Pale yellow solid.

### Compound 10-35

### N-(6-(4-(tert-butyl)phenyl)-1-cyclohexyl-1H-pyrazolo[3,4,d]pyrimidin-4-yl)-5-nitrothiophene-2-carboxamide

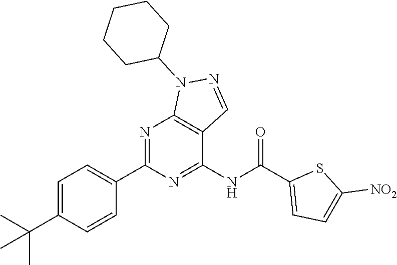 [SMILES: CC(C)(C)c1ccc(-c2nc(NC(=O)c3ccc([N+](=O)[O-])s3)c3cnn(C4CCCCC4)c3n2)cc1]

¹H NMR (400 MHz, DMSO-d₆): δ 11.89 (br. s., 1H), 8.45 (d, J=8.3 Hz, 2H), 8.35-8.39 (m, 1H), 8.34 (s, 1H), 8.23 (d, J=4.4 Hz, 1H), 7.53-7.63 (m, 2H), 4.86 (dt, J=15.3, 7.8 Hz, 1H), 1.94-2.09 (m, 4H), 1.90 (d, J=13.2 Hz, 2H), 1.68-1.81 (m, 1H), 1.46-1.60 (m, 2H), 1.31-1.40 (m, 9H), 1.23-1.31 (m, 1H). MS(M+1): 505. Pale yellow solid.

### Compound 10-36

### N-(6-(benzo[d][1,3]dioxol-5-yl)-1-cyclohexyl-1H-pyrazolo[3,4-d]pyrimidin-4-yl)-5-nitrothiophene-2-carboxamide

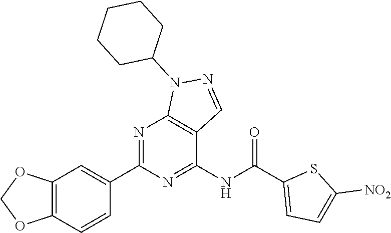 [SMILES: O=C(Nc1nc(-c2ccc3c(c2)OCO3)nc2c1cnn2C1CCCCC1)c1ccc([N+](=O)[O-])s1]

¹H NMR (400 MHz, DMSO-d₆): δ 11.78 (br. s., 1H), 8.27-8.39 (m, 2H), 8.24 (d, J=4.4 Hz, 1H), 8.15 (dd, J=8.1, 1.7 Hz, 1H), 8.02 (d, J=1.5 Hz, 1H), 7.10 (d, J=8.3 Hz, 1H), 6.14 (s, 2H), 4.81-4.94 (m, 1H), 1.94-2.06 (m, 4H), 1.89 (d, J=13.2 Hz, 2H), 1.70-1.81 (m, 1H), 1.45-1.62 (m, 2H), 1.23-1.37 (m, 1H). MS(M+1): 493. Light orange solid.

### Compound 10-37

### N-(1-cyclohexyl-6-(2,3-dihydrobenzo[b][1.4]dioxin-6-yl)-1H-pyrazolo[3,4-d]pyrimidin-4-yl)-5-nitrothiophene-2-carboxamide

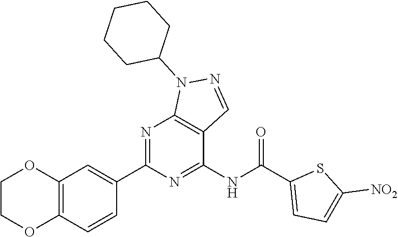 [SMILES: O=C(Nc1nc(-c2ccc3c(c2)OCCO3)nc2c1cnn2C1CCCCC1)c1ccc([N+](=O)[O-])s1]

¹H NMR (400 MHz, DMSO-d₆): δ 11.80 (br. s., 1H), 8.30-8.40 (m, 2H), 8.24 (d, J=4.4 Hz, 1H), 8.01-8.08 (m, 2H), 7.00-7.06 (m, 1H), 4.85 (dt, J=15.3, 7.8 Hz, 1H), 4.27-4.40 (m, 4H), 1.95-2.05 (m, 4H), 1.89 (d, J=13.2 Hz, 2H), 1.70-1.79 (nm, 1H), 1.45-1.64 (m, 2H), 1.19-1.39 (nm, 1H). MS(M+1): 507. Yellow solid.

### Compound 10-38

### N-(1-cyclohexyl-6-(6-morpholinopyridin-3-yl)-1H-pyrazolo[3,4-d]pyrimidin-4-yl)-5-nitrothiophene-2-carboxamide

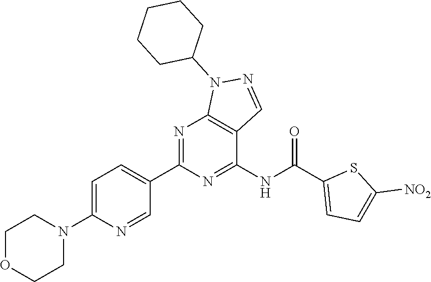 [SMILES: O=C(Nc1nc(-c2ccc(N3CCOCC3)nc2)nc2c1cnn2C1CCCCC1)c1ccc([N+](=O)[O-])s1]

¹H NMR (400 MHz, DMSO-d₆): δ 11.82 (br. s., 1H), 9.26 (d, J=2.4 Hz, 1H), 8.55 (dd, J=8.8, 2.4 Hz, 1H), 8.28-8.38 (m, 2H), 8.23 (d, J=4.4 Hz, 1H), 6.98 (d, J=8.8 Hz, 1H), 4.76-4.91 (m, 1H), 3.66-3.82 (m, 4H), 3.55-3.66 (m, 4H), 1.92-2.08 (m, 4H), 1.89 (d, J=12.7 Hz, 2H), 1.69-1.81 (m, 1H), 1.46-1.61 (m, 2H), 1.22-1.40 (m, 1H). MS(M+1): 535. Yellow solid.

### Compound 10-39

### N-(1-cyclohexyl-6-(2-(4-difluorophenyl)-1H-pyrazolo[3,4,d]pyrimidin-4-yl)-5-nitrothiophene-2-carboxamide

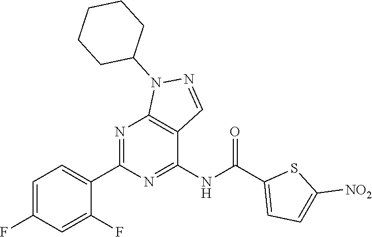 [SMILES: O=C(Nc1nc(-c2ccc(F)cc2F)nc2c1cnn2C1CCCCC1)c1ccc([N+](=O)[O-])s1]

¹H NMR (400 MHz, DMSO-d₆): δ 12.05 (br. s., 1H), 8.39 (s, 1H), 8.33 (d, J=3.9 Hz, 1H), 8.10-8.25 (m, 2H), 7.41 (id, J=10.3, 2.4 Hz, 1H), 7.22-7.34 (m, 1H), 4.79 (dt, J=15.3, 7.8 Hz, 1H), 1.92-2.05 (m, 4H), 1.88 (d, J=13.2 Hz, 2H), 1.65-1.77 (m, 1H), 1.39-1.61 (m, 2H), 1.17-1.35 (m, 1H). MS(M+1): 485. Khaki solid.

### Compound 10-40

### N-(1-cyclohexyl-6-(3,4-difluorophenyl)-1H-pyrazolo[3,4-d]pyrimidin-4-yl)-5-nitrothiophene-2-carboxamide

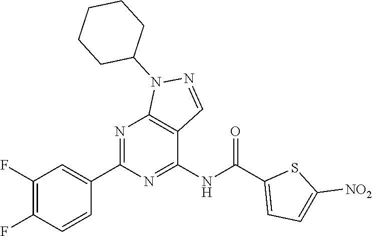 [SMILES: O=C(Nc1nc(-c2ccc(F)c(F)c2)nc2c1cnn2C1CCCCC1)c1ccc([N+](=O)[O-])s1]

¹H NMR (400 MHz, DMSO-d₆): δ 11.90 (br. s., 1H), 8.47 (ddd, J=12.0, 8.1, 2.0 Hz, 1H), 8.35-8.43 (m, 2H), 8.32 (d, J=4.4 Hz, 1H), 8.25 (d, J=4.4 Hz, 1H), 7.65 (dt, J=10.4, 8.5 Hz, 1H), 4.83-4.98 (m, 1H), 1.94-2.04 (m, 4H), 1.90 (d, J=12.7 Hz, 2H), 1.74 (d, J=13.2 Hz, 1H), 1.47-1.61 (m, 2H), 1.25-1.36 (m, 1H). MS(M+1): 485. Light yellow solid.

### Compound 10-41

### N-(1-cyclohexyl-1-(3,5-difluorophenyl)-1H-pyrazolo[3,4-d]pyrimidin-4-yl)-5-nitrothiophene-2-carboxamide

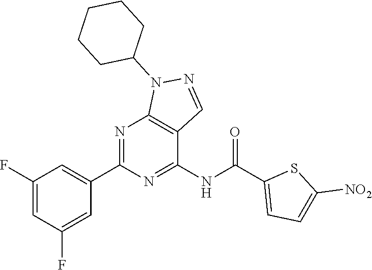 [SMILES: O=C(Nc1nc(-c2cc(F)cc(F)c2)nc2c1cnn2C1CCCCC1)c1ccc([N+](=O)[O-])s1]

¹H NMR (400 MHz, DMSO-d₆): δ 11.87 (br. s., 1H), 8.42 (s, 1H), 8.34 (d, J=4.4 Hz, 1H), 8.25 (d, J=4.4 Hz, 1H), 8.12-8.21 (m, 2H), 7.47 (tt, J=9.0, 2.4 Hz, 1H), 4.89-5.00 (m, 1H), 1.95-2.06 (m, 4H), 1.89 (d, J=12.7 Hz, 2H), 1.74 (d, J=13.2 Hz, 1H), 1.50-1.63 (m, 2H), 1.24-1.37 (m, 1H). MS(M+1): 485. Khaki solid.

### Compound 10-42

### N-(1-cyclohexyl-6-(2-(4-dichlorophenyl)-1H-pyrazolo[3,4-d]pyrimidin-4-yl)-5-nitrothiophene-2-carboxamide

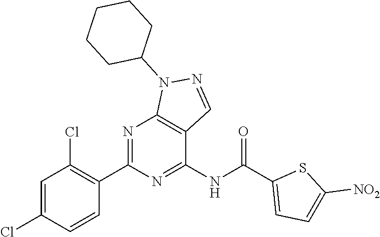 [SMILES: O=C(Nc1nc(-c2ccc(Cl)cc2Cl)nc2c1cnn2C1CCCCC1)c1ccc([N+](=O)[O-])s1]

¹H NMR (400 MHz, DMSO-d₆): δ 12.17 (br. s., 1H), 8.43 (s, 1H), 8.33 (d, J=4.4 Hz, 1H), 8.20 (d, J=4.4 Hz, 1H), 7.85 (d, J=8.3 Hz, 1H), 7.78 (d, J=2.4 Hz, 1H), 7.60 (dd, J=8.3, 2.0 Hz, 1H), 4.64-4.85 (m, 1H), 1.91-2.06 (m, 4H), 1.86 (d, J=13.2 Hz, 2H), 1.61-1.74 (m, 1H), 1.34-1.59 (m, 2H), 1.17-1.34 (m, 1H). MS(M+1): 517. Yellow solid.

### Compound 10-43

### N-(1-cyclohexyl-6-(3,4-dichlorophenyl)-1H-pyrazolo[3,4,d]pyrimidin-4-yl)-5-nitrothiophene-2-carboxamide

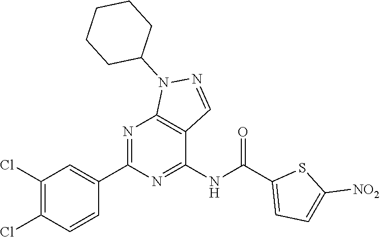 [SMILES: O=C(Nc1nc(-c2ccc(Cl)c(Cl)c2)nc2c1cnn2C1CCCCC1)c1ccc([N+](=O)[O-])s1]

¹H NMR (400 MHz, DMSO-d₆): δ 11.91 (br. s., 1H), 8.69 (d, J=2.0 Hz, 1H), 8.47 (dd, J=8.3, 2.0 Hz, 1H), 8.40 (s, 1H), 8.35 (d, J=4.4 Hz, 1H), 8.24 (d, J=4.9 Hz, 1H), 7.78-7.91 (m, 1H), 4.82-4.97 (m, 1H), 1.92-2.09 (m, 4H), 1.89 (d, J=13.2 Hz, 2H), 1.74 (d, J=12.7 Hz, 1H), 1.47-1.65 (m, 2H), 1.26-1.40 (m, 1H). MS(M+1): 517. Yellow solid.

### Compound 10-44

### N-(3-chloro-4-ethoxyphenyl)-1-cyclohexyl-1H-pyrazolo[3,4-d]pyrimidin-4-yl)-5-nitrothiophene-2-carboxamide

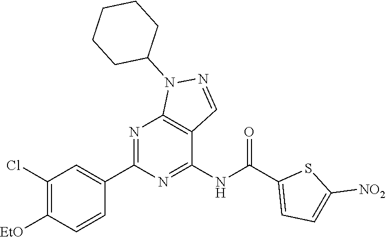 [SMILES: CCOc1ccc(-c2nc(NC(=O)c3ccc([N+](=O)[O-])s3)c3cnn(C4CCCCC4)c3n2)cc1Cl]

¹H NMR (400 MHz, DMSO-d₆): δ 11.81 (br. s., 1H), 8.54 (d, J=2.0 Hz, 1H), 8.44 (dd, J=8.6, 2.2 Hz, 1H), 8.30-8.39 (m, 2H), 8.23 (d, J=4.4 Hz, 1H), 7.31 (d, J=8.8 Hz, 1H), 4.82-4.94 (m, 1H), 4.23 (q, J=6.8 Hz, 2H), 1.93-2.10 (m, 4H), 1.89 (d, J=13.2 Hz, 2H), 1.69-1.79 (m, 1H), 1.47-1.62 (m, 2H), 1.41 (t, J=6.8 Hz, 3H), 1.23-1.36 (m, 1H). MS(M+1): 527. Light khaki solid.

### Compound 10-45

### N-(6-(4-chloro-2-fluorophenyl)-1-cyclohexyl-1H-pyrazolo[3,4-d]pyrimidin-4-yl)-5-nitrothiophene-2-carboxamide

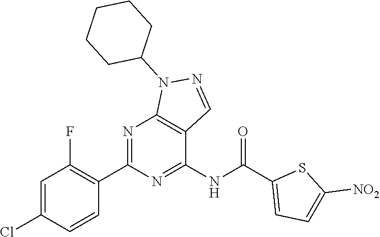 [SMILES: O=C(Nc1nc(-c2ccc(Cl)cc2F)nc2c1cnn2C1CCCCC1)c1ccc([N+](=O)[O-])s1]

¹H NMR (400 MHz, DMSO-d₆): δ 12.08 (br. s., 1H), 8.40 (s, 1H), 8.32 (d, J=4.4 Hz, 1H), 8.22 (d, J=4.4 Hz, 1H), 8.12-8.19 (m, 1H), 7.61 (dd, J=10.8, 2.0 Hz, 1H), 7.49 (dd, J=8.3, 2.0 Hz, 1H), 4.79 (dt, J=15.3, 7.8 Hz, 1H), 1.94-2.07 (m, 4H), 1.88 (d, J=12.7 Hz, 2H), 1.72 (d, J=12.7 Hz, 1H), 1.42-1.58 (m, 2H), 1.23-1.39 (m, 1H). MS(M+1): 501. Light yellow solid.

### Compound 10-46

### N-(6-(2-chlor-4-fluorophenyl)-1-cyclohexyl-1H-pyrazolo[3,4-d]pyrimidin-4-yl)-5-nitrothiophene-2-carboxamide

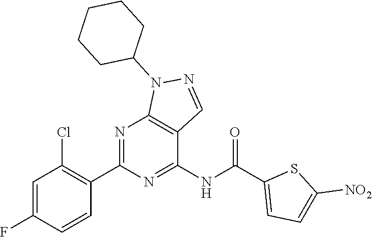 [SMILES: O=C(Nc1nc(-c2ccc(F)cc2Cl)nc2c1cnn2C1CCCCC1)c1ccc([N+](=O)[O-])s1]

¹H NMR (400 MHz, DMSO-d₆): δ 12.17 (br. s., 1H), 8.42 (s, 1H), 8.32 (d, J=4.4 Hz, 1H), 8.20 (d, J=4.4 Hz, 1H), 7.87 (dd, J=8.6, 6.1 Hz, 1H), 7.53-7.67 (m, 1H), 7.39 (td, J=8.4, 2.7 Hz, 1H), 4.78 (t, J=7.1 Hz, 1H), 1.92-2.05 (m, 4H), 1.86 (d, J=12.7 Hz, 2H), 1.65-1.76 (m, 1H), 1.38-1.58 (m, 2H), 1.21-1.38 (m, 1H). MS(M+1): 501. Light yellow solid.

### Compound 10-47

### N-(6-(5-chloro-2-fluorophenyl)-1-cyclohexyl-1H-pyrazolo[3,4-d]pyrimidin-4-yl)-5-nitrothiophene-2-carboxamide

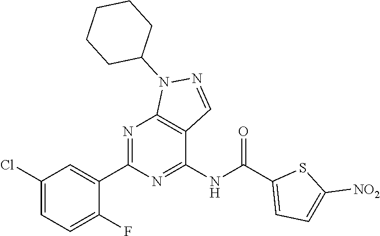 [SMILES: O=C(Nc1nc(-c2cc(Cl)ccc2F)nc2c1cnn2C1CCCCC1)c1ccc([N+](=O)[O-])s1]

¹H NMR (400 MHz, DMSO-d₆): δ 12.01 (br s., 1H), 8.40 (s, 1H), 8.33 (d, J=4.4 Hz, 1H), 8.21 (d, J=4.4 Hz, 1H), 8.17 (dd, J=6.4, 2.9 Hz, 1H), 7.60-7.68 (m, 1H), 7.44 (dd, J=10.3, 8.8 Hz, 1H), 4.72-4.86 (m, 1H), 1.93-2.05 (m, 4H), 1.87 (d, J=13.2 Hz, 2H), 1.66-1.77 (m, 1H), 1.40-1.57 (m, 2H), 1.20-1.36 (m, 1H). MS(M+1): 501. Yellow solid.

### Compound 10-48

### N-(1-cyclohexyl-6-(3,4-dimethoxyphenyl)-1H-pyrazolo[3,4-d]pyrimidin-4-yl)-5-nitrothiophene-2-carboxamide

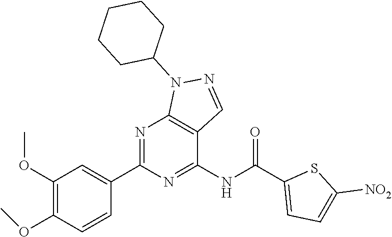 [SMILES: COc1ccc(-c2nc(NC(=O)c3ccc([N+](=O)[O-])s3)c3cnn(C4CCCCC4)c3n2)cc1OC]

¹H NMR (400 MHz, DMSO-d₆): δ 11.81 (br. s., 1H), 8.34 (d, J=4.4 Hz, 1H), 8.32 (s, 1H), 8.22 (d, J=4.4 Hz, 1H), 8.15 (dd, J=8.6, 1.7 Hz, 1H), 8.09 (d, J=2.0 Hz, 1H), 7.13 (d, J=8.8 Hz, 1H), 4.78-4.92 (m, 1H), 3.90 (s, 3H), 3.86 (s, 3H), 1.94-2.07 (m, 4H), 1.89 (d, J=13.2 Hz, 2H), 1.68-1.79 (m, 1H), 1.43-1.63 (m, 2H), 1.22-1.36 (m, 1H). MS(M+1): 509. Yellow solid.

### Compound 10-49

### N-(1-cyclohexyl-6-(6-(2,2,2-trifluoroethoxy)pyridin-3-yl)-1H-pyrazolo[3,4-d]pyrimidin-4-yl)-5-nitrothiophene-2-carboxamide

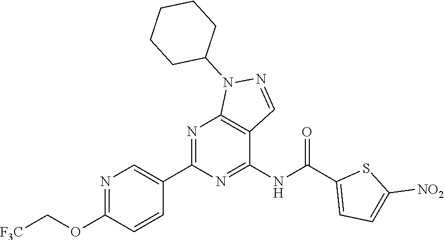 [SMILES: O=C(Nc1nc(-c2ccc(OCC(F)(F)F)nc2)nc2c1cnn2C1CCCCC1)c1ccc([N+](=O)[O-])s1]

¹H NMR (400 MHz, DMSO-d₆): δ 11.89 (br. s., 1H), 9.29 (d, J=2.4 Hz, 1H), 8.79 (dd, J=8.8, 2.4 Hz, 1H), 8.30-8.44 (m, 2H), 8.25 (d, J=4.4 Hz, 1H), 7.19 (d, J=8.3 Hz, 1H), 5.11 (q, J=8.8 Hz, 2H), 4.79-4.97 (m, 1H), 1.94-2.08 (m, 4H), 1.89 (d, J=12.2 Hz, 2H), 1.74 (d, J=12.7 Hz, 1H), 1.46-1.66 (m, 2H), 1.25-1.39 (m, 1H). MS(M+1): 548. Yellow solid.

### Compound 10-50

### N-(1-cyclohexyl-6-(4-(2-methoxyethoxy)phenyl)-1H-pyrazolo[3,4-d]pyrimidin-4-yl)-5-nitrothiophene-2-carboxamide

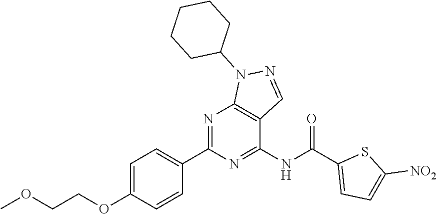 [SMILES: COCCOc1ccc(-c2nc(NC(=O)c3ccc([N+](=O)[O-])s3)c3cnn(C4CCCCC4)c3n2)cc1]

¹H NMR (400 MHz, DMSO-d₆): δ 11.83 (br. s., 1H), 8.45-8.53 (m, J=8.3 Hz, 2H), 8.37 (d, J=3.9 Hz, 1H), 8.34 (s, 1H), 8.25 (d, J=4.4 Hz, 1H), 7.09-7.17 (m, J=8.8 Hz, 2H), 4.87 (dt, J=15.9, 7.7 Hz, 1H), 4.20 (dd, J=5.6, 3.7 Hz, 2H), 3.68-3.75 (m, 2H), 1.95-2.04 (m, 4H), 1.86-1.95 (m, 2H), 1.74 (d, J=13.2 Hz, 1H), 1.46-1.60 (m, 2H), 1.28-1.37 (m, 1H). MS(M+1) 523. Yellow solid.

### Compound 10-51

### N-(1-cyclohexyl-6-(6-(2-methoxyethoxy)pyridin-3-yl)-1H-pyrazolo[3,4-d]pyrimidin-4-yl)-5-nitrothiophene-2-carboxamide

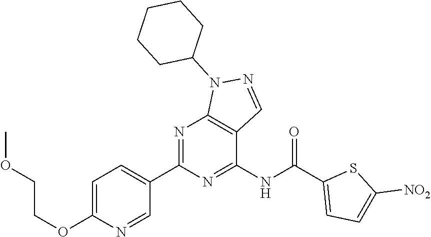 [SMILES: COCCOc1ccc(-c2nc(NC(=O)c3ccc([N+](=O)[O-])s3)c3cnn(C4CCCCC4)c3n2)cn1]

¹H NMR (400 MHz, DMSO-d₆): δ 11.85 (br. s., 1H), 9.25 (d, J=2.0 Hz, 1H), 8.69 (dd, J=8.8, 2.4 Hz, 1H), 8.35 (t, J=2.2 Hz, 2H), 8.23 (d, J=4.4 Hz, 1H), 7.01 (d, J=8.8 Hz, 1H), 4.81-4.94 (m, 1H), 4.39-4.54 (m, 2H), 3.70 (dd, J=5.4, 3.9 Hz, 2H), 3.32 (s, 5H), 1.94-2.07 (m, 4H), 1.89 (d, J=13.2 Hz, 2H), 1.74 (d, J=13.2 Hz, 1H), 1.46-1.61 (m, 2H), 1.24-1.37 (m, 1H). MS(M+1): 524. Yellow solid.

### Compound 10-52

### N-(1-cyclohexyl-6-(6-(2,2,3,3-tetrafluoropropoxy)pyridin-3-yl)-1H-pyrazolo[3,4-d]pyrimidin-4-yl)-5-nitrothiophene-2-carboxamide

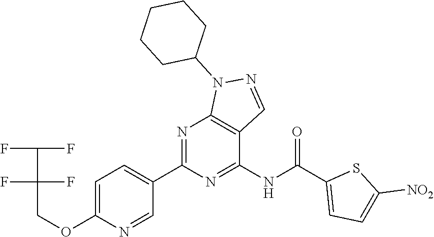 [SMILES: O=C(Nc1nc(-c2ccc(OCC(F)(F)C(F)F)nc2)nc2c1cnn2C1CCCCC1)c1ccc([N+](=O)[O-])s1]

¹H NMR (400 MHz, DMSO-d₆): δ 11.94 (br. s., 1H), 9.29 (d, J=2.0 Hz, 1H), 8.79 (dd, J=8.6, 2.2 Hz, 1H), 8.37 (s, 1H), 8.29 (d, J=4.4 Hz, 1H), 8.24 (d, J=4.4 Hz, 1H), 7.16 (d, J=8.8 Hz, 1H), 6.71 (t, J=5.4 Hz, 1H), 4.92-5.05 (m, 2H), 4.81-4.92 (m, 1H), 1.95-2.08 (m, 4H), 1.90 (d, J=13.2 Hz, 2H), 1.74 (d, J=12.7 Hz, 1H), 1.45-1.61 (m, 2H), 1.30 (q. J=13.0 Hz, 1H). MS(M+1): 580. Light yellow solid.

### Compound 10-53

### N-(1-cyclohexyl-6-(6-(2-(2-ethoxyethoxy)ethoxy)pyridin-3-yl)-1H-pyrazolo[3,4-d]pyrimidin-4-yl)-5-nitrothiophene-2-carboxamide

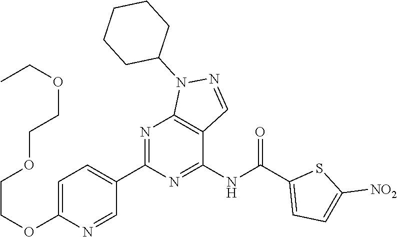 [SMILES: CCOCCOCCOc1ccc(-c2nc(NC(=O)c3ccc([N+](=O)[O-])s3)c3cnn(C4CCCCC4)c3n2)cn1]

¹H NMR (400 MHz, DMSO-d₆): δ 11.88 (br. s., 1H), 9.27 (d, J=2.4 Hz, 1H), 8.71 (dd, J=8.8, 2.4 Hz, 1H), 8.29-8.39 (m, 2H), 8.24 (d, J=4.4 Hz, 1H), 7.02 (d, J=8.8 Hz, 1H), 4.82-4.92 (m, 1H), 4.48 (dd, J=5.6, 4.2 Hz, 2H), 3.76-3.83 (m, 2H), 3.57-3.64 (m, 2H), 3.46-3.53 (m, 2H), 3.39-3.46 (m, 2H), 1.94-2.08 (m, 4H), 1.89 (d, J=12.7 Hz, 2H), 1.69-1.79 (m, 1H), 1.46-1.62 (m, 2H), 1.23-1.36 (m, 1H), 1.05-1.13 (m, 3H). MS(M+1): 582. Yellow solid.

### Compound 10-54

### N-(1-(4,4-difluorocyclohexyl)-6-(4-fluorphenyl)-1H-pyrazolo[3,4-d]pyrimidin-4-yl)-5-nitrothiophene-2-carboxamide

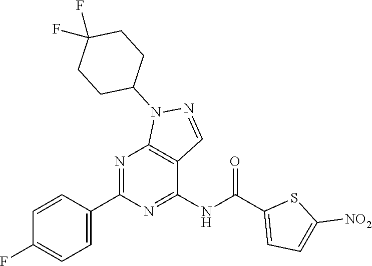 [SMILES: O=C(Nc1nc(-c2ccc(F)cc2)nc2c1cnn2C1CCC(F)(F)CC1)c1ccc([N+](=O)[O-])s1]

¹H NMR (400 MHz, DMSO-d₆): δ 11.93 (br. s., 1H), 8.53-8.66 (m, 2H), 8.40 (s, 1H), 8.35 (d, J=4.4 Hz, 1H), 8.25 (d, J=4.4 Hz, 1H), 7.33-7.47 (m, 2H), 5.06-5.23 (m, 1H), 2.15-2.36 (m, 6H), 2.09 (br. s., 2H). MS(M+1): 503. Pale yellow solid.

### Compound 10-55

### N-(1-(4,4-difluorocyclohexyl)-6-(6-fluoropyridin-3-yl)-1H-pyrazolo[3,4-d]pyrimidin-4-yl)-5-nitrothiophene-2-carboxamide

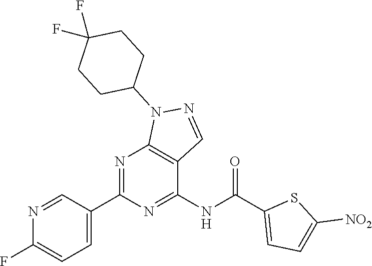

¹H NMR (400 MHz, DMSO-d₆): δ 11.90 (s, 1H), 9.30 (d, J=2.4 Hz, 1H), 8.93 (td, J=8.3, 2.4 Hz, 1H), 8.40 (s, 1H), 8.34 (d, J=4.4 Hz, 1H), 8.22 (d, J=4.4 Hz, 1H), 7.40 (dd, J=8.3, 2.4 Hz, 1H), 5.03-5.25 (m, 1H), 2.15-2.34 (m, 6H), 2.01-2.15 (m, 2H) MS(M+1): 504. White solid.

### Compound 10-56

### N-(6-(4-chlorophenyl)-1-(4,4-difluorocyclohexyl)-1H-pyrazolo[3,4,d]pyrimidin-4-yl)-5-nitrothiophene-2-carboxamide

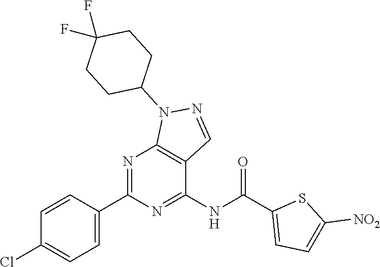

¹H NMR (400 MHz, DMSO-d₆): δ 11.93 (br. s., 1H), 8.49-8.62 (m, 2H), 8.29-8.42 (m, 2H), 8.23 (d, J=4.4 Hz, 1H), 7.59-7.71 (m, 2H), 5.15 (d, J=3.9 Hz, 1H), 2.15-2.34 (m, 6H), 2.09 (br. s., 2H). MS(M+1): 519. White solid.

### Compound 10-57

### N-(6-(6-chloropyridin-3-yl)-1-(4,4-difluorocyclohexyl)-TH-pyrazolo[3,4-d]pyrimidin-4-yl)-5-nitrothiophene-2-carboxamide

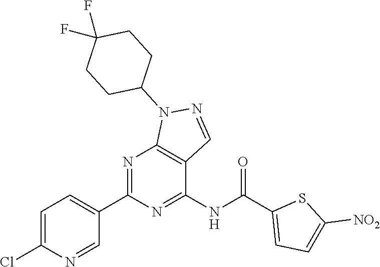

¹H NMR (400 MHz, DMSO-d₆): δ 11.99 (br. s., 1H), 9.47 (d, J=2.0 Hz, 1H), 8.83 (dd, J=8.3, 2.4 Hz, 1H), 8.41-8.48 (m, 1H), 8.35 (d, J=4.4 Hz, 1H), 8.25 (d, J=4.4 Hz, 1H), 7.77 (d, J=8.3 Hz, 1H), 5.12-5.25 (m, 1H), 2.16-2.1 (m, 8H). MS(M+1): 520. Rosy brown solid.

### Compound 10-58

### N-(1-(4,4-difluorocyclohexyl)-6-(4-(trifluoromethyl)phenyl)-1H-pyrazolo[3,4-d]pyrimidin-4-yl)-5-nitrothiophene-2-carboxamide

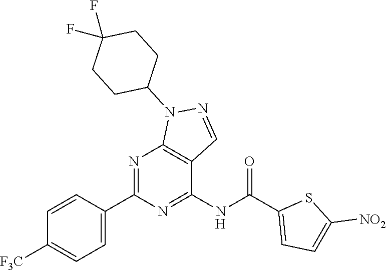

¹H NMR (400 MHz, DMSO-d₆): δ 12.02 (br. s., 1H), 8.65-8.81 (m, J=8.3 Hz, 2H), 8.43 (s, 1H), 8.36 (d, J=4.4 Hz, 1H), 8.24 (d, J=4.4 Hz, 1H), 7.86-8.02 (m, J=8.3 Hz, 2H), 5.05-5.28 (m, 1H), 2.16-2.36 (m, 6H), 2.11 (br. s., 2H). MS(M+1): 553. White solid.

### Compound 10-59

### N-(1-(4,4<fluorocyclohexyl)-4-(4-ethoxyphenyl)-1H-pyrazolo[3,4-d]pyrimidin-4-yl)-5-nitrothiophene-2-carboxamide

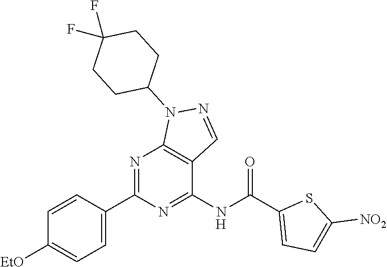

¹H NMR (400 MHz, DMSO-d₆): δ 11.87 (br. s., 1H), 8.42 8.54 (m, J=8.8 Hz, 2H), 8.29-8.39 (m, 2H), 8.24 (d, J=4.4 Hz, 1H), 7.02-7.15 (m, 2H), 5.04-5.20 (m, 1H), 4.14 (q, J=6.8 Hz, 2H), 2.15-2.37 (m, 6H), 2.08 (br. s., 2H), 1.37 (t, J=6.8 Hz, 3H). MS(M+1): 529. Bright yellow solid.

### Compound 10-60

### N-(1-(4,4-difluorocyclohexyl)-6-(4-propoxyphenyl)-1H-pyrazolo[3,4-d]pyrimidin-4-yl)-5-nitrothiophene-2-carboxamide

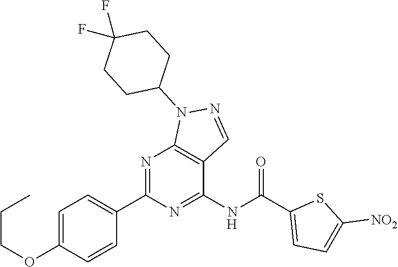

¹H NMR (400 MHz, DMSO-d₆): δ 11.89 (br. s., 1H), 8.49 (d, J=8.8 Hz, 2H), 8.28-8.38 (m, 2H), 8.24 (d, J=4.4 Hz, 1H), 7.02-7.19 (m, 2H), 5.05-5.20 (m, 1H), 4.04 (t, J=6.6 Hz, 2H), 2.14-2.37 (m, 6H), 2.08 (br. s., 2H), 1.68-1.84 (m, 2H), 1.01 (4, J=7.6 Hz, 3H). MS(M+1): 543. Bright yellow solid.

### Compound 10-61

### N-(1-(4,4-dimethylcyclohexyl)-4-(4-fluorophenyl)-1H-pyrazolo[3,4-d]pyrimidin-4-yl)-5-nitrothiophene-2-carboxamide

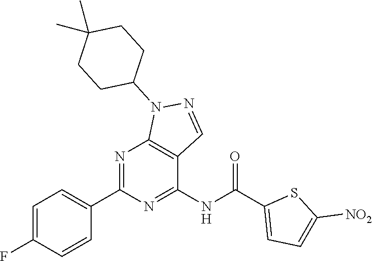

¹H NMR (4007 MHz, DMSO-d₆): δ 11.90 (br s., 1H), 8.48-8.64 (m, 2H), 8.29-8.40 (m, 2H), 8.24 (d, J=4.4 Hz, 1H), 7.32-7.49 (m, 2H), 4.74-4.90 (m, 1H), 2.23 (qd, J=12.5, 4.6 Hz, 2H), 1.73-1.86 (mi, 2H), 1.43-1.62 (m, 4H), 1.04-1.12 (m, 3H), 1.01 (s, 3H).

MS(M+1): 495. Bright yellow solid.

### Compound 10-62

### N-(1-(4,4-dimethylcyclohexyl)-6-(6-fluoropyridin-3-yl)-1H-pyrazolo[3,4-d]pyrimidin-4-yl)-5-nitrothiophene-2-carboxamide

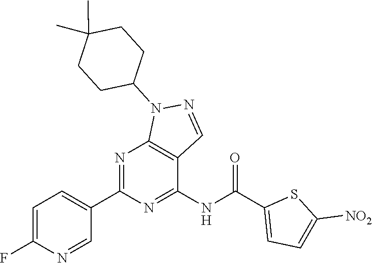

¹H NMR (400 MHz, DMSO-d₆): δ 11.96 (s, 1H), 9.30 (d, J=2.4 Hz, 1H), 8.94 (t, J=8.3, 1H), 8.41 (s, 1H), 8.34 (d, J=4.4 Hz, 1H), 8.23 (d, J=4.4 Hz, 1H), 7.40 (dd, J=8.3, 2.7 Hz, 1H), 4.89-4.82 (m, 1H), 2.27-2.20 (m, 2H), 1.82-1.80 (m, 2H), 1.54-1.48 (m, 4H), 1.07 (s, 3H), 1.01 (s, 3H). MS(M+1): 496. Yellow solid.

### Compound 10-63

### N-(6(4-chlorophenyl)-1-(4,4-dimethylcyclohexyl)-1H-pyrazolo[3,4-d]pyrimidin-4-yl)-5-nitrothiophene-2-carboxamide

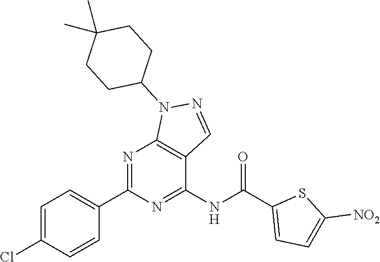

¹H NMR (400 MHz, DMSO-d₆): δ 11.89 (br. s., 1H), 8.45-8.58 (m, 2H), 8.30-8.40 (m, 2H), 8.22 (d, J=4.4 Hz, 1H), 7.54-7.71 (m, 2H), 4.72-4.89 (m, 1H), 2.09-2.31 (m, 2H), 1.70-1.86 (m, 2H), 1.42-1.59 (m, 4H), 1.03-1.11 (m, 3H), 1.00 (s, 3H). MS(M+1): 511. Bright yellow solid.

Compound 10-64 N-(6-(6-chloropyridin-3-yl)-1-(4,4-dimethylcyclohexyl)-1H-pyrazolo[3,4-d]pyrimidin-4-yl)-5-nitrothiophene-2-carboxamide

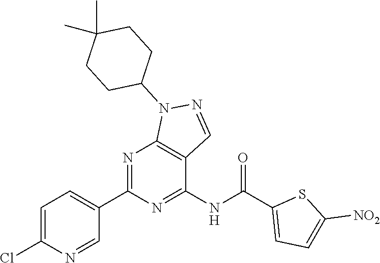

¹H NMR (400 MHz, DMSO-d₆): δ 11.96 (s, 1H), 9.44 (d, J=1.7 Hz, 1H), 8.81 (dd, J=8.8, 2.4 Hz, 1H), 8.41 (s, 1H), 8.35 (d, J=4.4 Hz, 1H), 8.24 (d, J=4.4 Hz, 1H), 7.74 (d, J=8.8 Hz, 1H), 4.88-4.80 (m, 1H), 2.26-2.16 (m, 2H), 1.82-1.79 (m, 2H), 1.54-1.48 (m, 4H), 1.06 (s, 3H), 1.01 (s, 3H). MS(M+1): 512. Yellow solid.

### Compound 10-65

### N-(6-(4-chloro-2-fluorophenyl)-1-(4,4-dimethylcyclohexyl)-1H-pyrazolo[3,4-d]pyrimidin-4-yl)-5-nitrothiophene-2-carboxamide

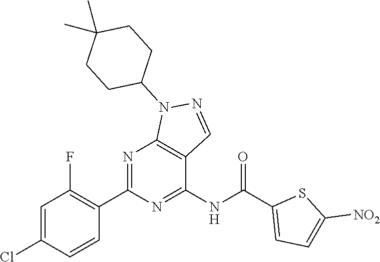

¹H NMR (400 MHz, DMSO-d₆): δ 12.05 (br. s., 1H), 8.37-8.46 (m, 1H), 8.34 (d, J=4.4 Hz, 1H), 8.09-8.27 (m, 2H), 7.61 (dd, J=10.8, 2.0 Hz, 1H), 7.49 (dd, J=8.3, 2.0 Hz, 1H), 4.73 (tt, J=11.9, 4.3 Hz, 1H), 2.24 (qd, J=12.7, 3.9 Hz, 2H), 1.73-1.87 (m, 2H), 1.39-1.60 (m), 4H), 1.05 (s, 3H), 0.99 (s, 3H). MS(M+1) 529. Yellow solid.

### Compound 10-66

### N-(6-(benzo[d][1,3]dioxol-5-yl)-1-(4,4-dimethylcyclohexyl)-1H-pyrazolo[3,4-d]pyrimidin-4-yl)-5-nitrothiophene-2-carboxamide

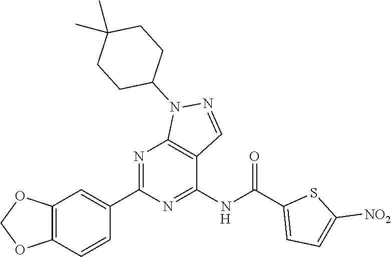

¹H NMR (400 MHz, DMSO-d_{b}): δ 11.76 (br. s., 1H), 8.27-8.40 (m, 2H), 8.23 (d, J=4.4 Hz, 1H), 8.14 (dd, J=8.3, 1.5 Hz, 1H), 8.01 (d, J=1.5 Hz, 1H), 7.09 (d, J=8.3 Hz, 1H), 6.13 (s, 2H), 4.72-4.85 (m, 1H), 2.22 (qd, J=12.2, 5.4 Hz, 2H), 1.71-1.86 (m, 2H), 1.42-1.61 (m, 4H), 1.07 (s, 3H), 1.00 (s, 3H). MS(M+1): 521. Yellow solid.

### Compound 10-67

### N-(1-(3,5-dimethylcyclohexyl)-6-(4-fluorophenyl)-1H-pyrazolo[3,4-d]pyrimidin-4-yl)-5-nitrothiophene-2-carboxamide

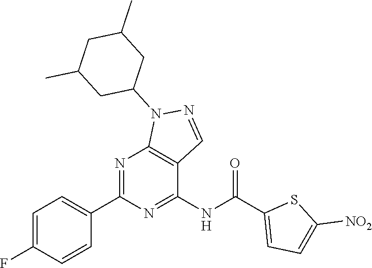

¹H NMR (400 MHz, DMSO-d₆): δ 11.88 (br. s., 1H), 8.52-8.66 (m, 2H), 8.32-8.40 (m, 2H), 8.24 (d, J=4.4 Hz, 1H), 7.32-7.46 (m, 2H), 4.97 (tt, J=11.7, 3.9 Hz, 1H), 1.94 (d, J=11.7 Hz, 2H), 1.68-1.86 (m, 3H), 1.60 (q, J=12.1 Hz, 2H), 0.97 (d, J=6.4 Hz, 6H), 0.64-0.80 (m, 1H). MS(M+1): 495. Light green solid.

### Compound 10-68

### N-(1-(3,5-dimethylcyclohexyl)-6-(6-fluoropyridin-3-yl)-1H-pyrazolo[3,4-d]pyrimidin-4-yl)-5-nitrothiophene-2-carboxamide

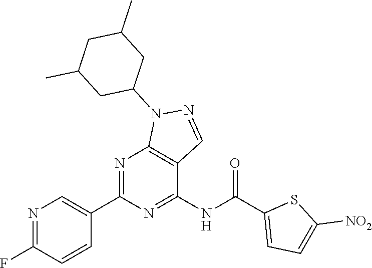

¹H NMR (400 MHz, DMSO-d₆): δ 11.94 (br. s., 1H), 9.31 (d, J=2.4 Hz, 1H), 8.96 (td, J=8.3, 2.4 Hz, 1H), 8.41 (s, 1H), 8.34 (d, J=4.4 Hz, 1H), 8.25 (d, J=4.4 Hz, 1H), 7.41 (dd, J=8.8, 2.4 Hz, 1H), 4.90-5.08 (m, 1H), 1.86-2.05 (m, 2H), 1.74 (t, J=9.3 Hz, 3H), 1.61 (q, J=11.9 Hz, 2H), 0.97 (d, J=6.4 Hz, 6H), 0.74 (d, J=11.7 Hz, 1H)

MS(M+1): 496. Light yellow solid.

### Compound 10-69

### N-(6-(4-chlorophenyl)-1-(3,5-dimethylcyclohexyl)-1H-pyrazolo[3,4-d]pyrimidin-4-yl)-5-nitrothiophene-2-carboxamide

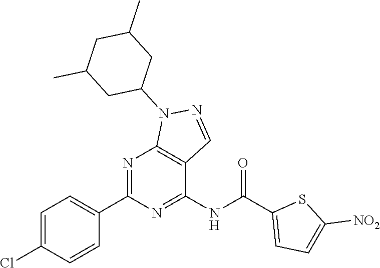

¹H NMR (400 MHz, DMSO-d₆): δ 11.90 (br. s., 1H), 8.47-8.56 (m, J=8.3 Hz, 2H), 8.29-8.42 (m, 2H), 8.24 (d, J=4.4 Hz, 1H), 7.56-7.73 (m, J=8.8 Hz, 2H), 4.84-5.08 (m, 1H), 1.93 (d, J=12.2 Hz, 2H), 1.72 (d, J=11.2 Hz, 3H), 1.60 (q. J=12.1 Hz, 2H), 0.96 (d, J=6.4 Hz, 6H), 0.65-0.80 (m, 1H). MS(M+1): 511. Light yellow solid.

### Compound 10-70

### N-(6-(6-chloropyridin-3-yl)-1-(3,5-dimethylcyclohexyl)-1H-pyrazolo[3,4-d]pyrimidin-4-yl)-5-nitrothiophene-2-carboxamide

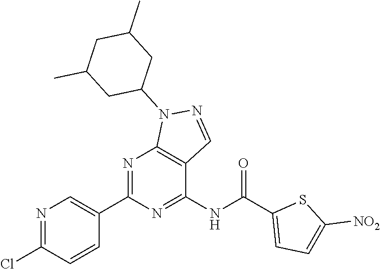 [SMILES: CC1CC(C)CC(n2ncc3c(NC(=O)c4ccc([N+](=O)[O-])s4)nc(-c4ccc(Cl)nc4)nc32)C1]

¹H NMR (400 MHz, DMSO-d₆): δ 11.94 (br. s., 1H), 9.44 (d, J=2.4 Hz, 1H), 8.80 (dd, J=8.3, 2.4 Hz, 1H), 8.40 (s, 1H), 8.34 (d, J=4.4 Hz, 1H), 8.24 (d, J=4.4 Hz, 1H), 7.60-7.81 (m, 1H), 4.98 (tt, J=11.7, 3.9 Hz, 1H), 1.94 (d, J=12.2 Hz, 2H), 1.68-1.82 (m, 3H), 1.60 (q, J=12.2 Hz, 2H), 0.97 (d, J=6.4 Hz, 6H), 0.66-0.79 (m, 1H). MS(M+1): 512. Light yellow solid.

### Compound 10-71

### methyl 4-(1-(3,5-dimethylcyclohexyl)-4-(5-nitrothiophene-2-carboxamido)-1H-pyrazolo[3,4-d]pyrimidin-6-yl)benzoate

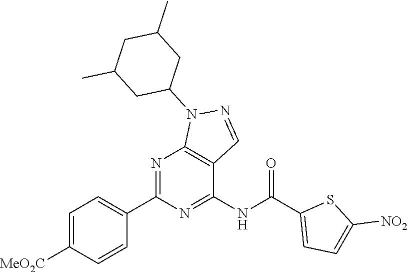 [SMILES: COC(=O)c1ccc(-c2nc(NC(=O)c3ccc([N+](=O)[O-])s3)c3cnn(C4CC(C)CC(C)C4)c3n2)cc1]

¹H NMR (400 MHz, DMSO-d₆): δ 12.00 (br. s., 1H), 8.57-8.71 (m, J=7.8 Hz, 2H), 8.31-8.44 (m, 2H), 8.25 (d, J=4.4 Hz, 1H), 8.09-8.21 (m, J=7.8 Hz, 2H), 5.01 (t, J=11.2 Hz, 1H), 3.91 (s, 3H), 1.95 (d, J=11.7 Hz, 2H), 1.68-1.84 (m, 3H), 1.49-1.68 (m, 2H), 0.98 (d, J=5.9 Hz, 6H), 0.74 (d, J=12.2 Hz, 1H). MS(M+1): 535. Light yellow solid.

### Compound 10-72

### N-(6-(4-chloro-2-fluorophenyl)-1-(3,5-dimethylcyclohexyl)-1H-pyrazolo[3,4-d]pyrimidin-4-yl)-5-nitrothiophene-2-carboxamide

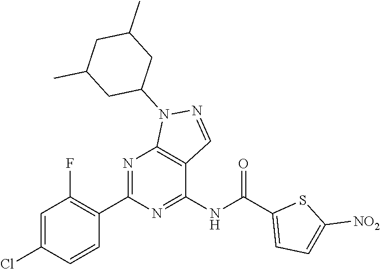 [SMILES: CC1CC(C)CC(n2ncc3c(NC(=O)c4ccc([N+](=O)[O-])s4)nc(-c4ccc(Cl)cc4F)nc32)C1]

¹H NMR (400 MHz, DMSO-d₆): δ 12.08 (br s., 1H), 8.36-8.45 (m, 1H), 8.32 (d, J=4.4 Hz, 1H), 8.22 (d, J=4.4 Hz, 1H), 8.15 (t, J=8.3 Hz, 1H), 7.61 (dd, J=10.8, 2.0 Hz, 1H), 7.49 (dd, J=8.6, 1.7 Hz, 1H), 4.78-4.97 (m, 1H), 1.93 (d, J=11.7 Hz, 2H), 1.52-1.81 (m, 5H), 0.95 (d, J=5.9 Hz, 6H), 0.61-0.83 (m, 1H). MS(M+1): 529. Light yellow solid.

Shown in Tables 11 are in vitro activities of exemplary compounds of formula (I). The results indicate that the compounds of the present disclosure indeed have efficacy for inhibiting the growth of various tumor cells.

TABLE 11

| | SW480 | NCI-H460 |
|---|---|---|
| | LC₅₀ | LC₅₀ |
| Compound | (μM) | (μM) |
| 1-4 | 6.19 | 3.56 |
| 1-5 | 4.32 | 2.85 |
| 1-11 | 2.24 | 2.62 |
| 1-22 | 5.38 | 2.18 |
| 1-27 | 4.14 | 1.99 |
| 4-2 | 10.06 | 1.65 |
| 4-5 | 3.51 | 2.45 |
| 5-6 | 1.96 | 1.46 |
| 5-18 | 2.43 | 0.46 |
| 5-28 | 0.59 | 0.28 |
| 5-29 | 0.66 | 0.95 |
| 5-30 | 2.69 | 0.54 |
| 5-31 | 0.86 | 0.41 |
| 5-32 | 2.77 | 1.16 |
| 5-33 | 0.60 | 1.80 |
| 5-35 | 1.11 | 0.65 |
| 5-39 | >10C | 1.25 |
| 5-40 | 5.42 | 0.93 |
| 5-42 | 0.89 | 1.67 |
| 5-44 | 1.62 | 4.64 |
| 5-45 | 1.11 | 2.18 |
| 5-47 | 0.61 | 0.29 |
| 5-49 | 3.75 | 1.22 |
| 5-56 | 0.73 | 1.10 |
| 6-4 | 1.51 | 0.68 |
| 6-5 | 0.65 | 0.91 |
| 6-11 | 0.67 | 0.33 |
| 6-18 | 1.24 | 0.27 |
| 6-19 | 0.45 | 0.22 |
| 6-28 | 3.44 | 0.68 |
| 6-35 | 0.52 | 0.50 |
| 6-36 | 2.80 | 0.67 |
| 6-39 | 3.18 | 1.07 |
| 6-45 | 0.58 | 0.57 |
| 6-53 | 1.04 | 1.09 |
| 6-54 | 0.34 | 0.33 |
| 6-57 | 1.24 | 2.75 |
| 6-58 | 4.09 | 3.97 |
| 6-66 | >10 | 0.41 |
| 6-69 | 0.89 | 2.76 |
| 6-71 | 0.98 | 0.44 |
| 6-75 | 5.48 | 1.28 |
| 6-85 | 0.68 | 0.56 |
| 6-89 | 0.81 | 0.71 |
| 6-90 | 0.54 | 0.56 |
| 6-104 | 0.65 | 0.86 |
| 6-106 | 0.50 | 1.65 |
| 6-110 | 1.25 | 1.41 |
| 6-111 | >4 | 1.87 |
| 6-112 | 0.81 | 0.97 |
| 6-114 | 0.84 | 0.35 |
| 6-115 | 1.91 | 1.13 |
| 6-116 | 0.55 | 0.50 |
| 6-117 | 0.91 | 0.60 |
| 6-118 | 2.18 | 0.98 |
| 6-119 | 0.69 | 0.53 |
| 6-120 | 0.51 | 1.34 |
| 6-121 | 0.63 | 0.48 |
| 6-125 | 2.28 | 0.35 |
| 6-127 | 1.06 | 0.45 |
| 6-128 | 0.57 | 0.31 |
| 6-130 | 2.90 | 3.81 |
| 6-133 | 0.43 | 0.49 |
| 6-135 | 0.69 | 0.56 |
| 7-1 | >20 | 8.63 |
| 7-3 | 0.53 | 0.40 |
| 9-3 | 2.72 | 1.22 |
| 9-5 | 2.78 | 1.30 |
| 10-13 | 1.73 | 2.62 |
| 10-14 | 1.71 | 0.91 |
| 10-18 | 0.95 | 0.85 |
| 10-23 | 1.66 | 3.01 |
| 10-32 | 1.21 | 1.72 |
| 10-40 | 1.04 | 1.16 |
| 10-55 | 1.06 | 0.94 |
| 10-57 | 1.34 | 1.30 |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A compound of formula (I): 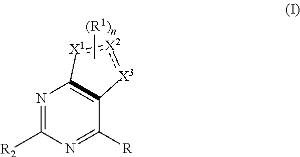
or a stereoisomer, a tautomer, or a pharmaceutically acceptable salt thereof,
wherein
each of X₁, X₂ and X₃ independently is C, N, O or S, with the proviso that no more than two of X₁, X₂ and X₃ are N, O or S;
each of R₁ independently is selected from the group consisting of hydrogen, halogen, alkyl, alkyloxy, cycloalkyl, heterocycloalkyl, aryl or heteroaryl, wherein each of alkyl, alkyloxy, cycloalkyl, heterocycloalkyl, aryl and heteroaryl is optionally substituted with one to three moieties selected from the group consisting of halogen, hydroxyl, nitro, cyano, —NRₐR_{b}, —C(═O)R_{c}, —C(═O)OR_{d}, heterocycloalkyl optionally substituted with one to four halogen, aryl optionally substituted with one to three halogen or —NRₐR_{b}, alkyl optionally substituted with one to three halogen, and alkyloxy optionally substituted with one to three halogen or alkyloxy, in which each of Rₐ and R_{b} independently is hydrogen, alkyl or acrylamide, and each of R_{c} and R_{d} independently is hydrogen, alkenyl, or alkyl optionally substituted with one to three halogen or alkyloxy optionally further substituted with alkyloxy;
one of R and R₂ is 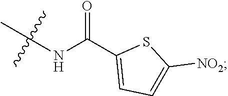 [SMILES: *NC(=O)c1ccc([N+](=O)[O-])s1]
the other of R and R₂ is —OR₃, —NHR₄, —SR₅, alkenyl, alkynyl, aryl, heteroaryl, cycloalkyl, heterocycloalkyl, aryl fused with heterocycloalkyl, cycloalkenyl or cycloamine, wherein each of alkenyl, alkynyl, aryl, heteroaryl, cycloalkyl, heterocycloalkyl and cycloalkenyl is optionally substituted with one to three moieties selected from the group consisting of halogen, hydroxyl, nitro, cyano, —NRₑR_{f}, —C(═O)R_{g}, —C(═O)ORₕ, —SO₂Rᵢ, —OSiRⱼ, alkenyl, heterocycloalkyl, aryl optionally substituted with alkyl optionally substituted with one to three halogen or alkyloxy optionally substituted with one to three halogen, alkyl optionally substituted with one to three halogen, —NRₑR_{f} or —ORₖ, and alkyloxy optionally substituted with one to four halogen, hydroxyl, —NRₑR_{f} or alkyloxy optionally further substituted with alkyloxy, in which each of Rₑ and R_{f} independently is hydrogen or alkyl optionally substituted with alkyloxy, each of R_{g} and Rₕ independently is hydrogen, alkyl or alkenyl, Rᵢ is alkyl, Rⱼ is alkyl, and Rₖ is hydrogen, aryl, or alkyl optionally substituted with alkyloxy optionally further substituted with alkyloxy;
each of R₃ and R₅ independently is alkyl, cycloalkyl or aryl, wherein alkyl, cycloalkyl or aryl is optionally substituted with one to four moieties selected from the group consisting of halogen, alkyl optionally substituted with one to four halogen, alkyloxy optionally substituted with one to three halogen or alkyloxy, —NRₗRₘ, and heterocycloalkyl, in which each of Rₗ and Rₘ independently is hydrogen or alkyl;
R₄ is alkyl, cycloalkyl or —SO₂Rₙ, in which Rₙ is hydrogen, alkyl or aryl, and each of alkyl or aryl is optionally substituted with one to three halogen; and
n is 1, 2 or 3.

2. The compound of claim 1, wherein is 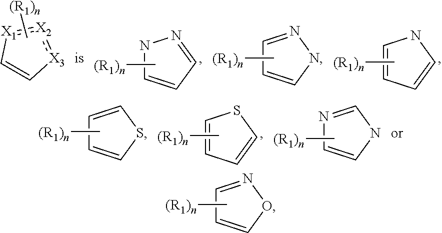
in which n is 1 or 2.

3. The compound of claim 1, wherein 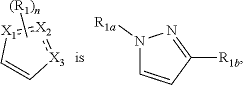
in which each of R₁ₐ and R_{1b} independently is selected from the group consisting of hydrogen, halogen, alkyl, alkyloxy, cycloalkyl, heterocycloalkyl, aryl or heteroaryl, wherein each of alkyl, alkyloxy, cycloalkyl, heterocycloalkyl, aryl and heteroaryl is optionally substituted with one to three moieties selected from the group consisting of halogen, hydroxyl, nitro, cyano, —NRₐR_{b}, —C(═O)R_{c}, —C(═O)OR_{d}, heterocycloalkyl optionally substituted with one to four halogen, aryl optionally substituted with one to three halogen or —NRₐR_{b}, alkyl optionally substituted with one to three halogen, and alkyloxy optionally substituted with one to three halogen or alkyloxy, in which each of Rₐ and R_{b} independently is hydrogen, alkyl or acrylamide, and each of R_{c} and R_{d} independently is hydrogen, alkenyl, or alkyl optionally substituted with one to three halogen or alkyloxy optionally further substituted with alkyloxy.

4. The compound of claim 3, wherein R_{1b} is hydrogen or alkyl.

5. The compound of claim 4, wherein R_{1b} is hydrogen.

6. The compound of claim 5, wherein R₁ₐ is alkyl, aryl, heteroaryl, cycloalkyl or heterocycloalkyl, wherein each of alkyl, cycloalkyl, heterocycloalkyl, aryl and heteroaryl is optionally substituted with one to three moieties selected from the group consisting of F, Cl, hydroxyl, cyano, —NRₐR_{b}, —C(═O)R_{c}, —C(═O)OR_{d}, heterocycloalkyl optionally substituted with one to four F or Cl, aryl optionally substituted with one to three F, Cl or —NRₐR_{b}, alkyl optionally substituted with one to three F, Cl, and alkyloxy optionally substituted with one to three F, Cl or alkyloxy, in which Rₐ is hydrogen or alkyl, R_{b} is alkyl or acrylamide, R_{c} is alkyl or alkenyl, R_{d} is alkyl optionally substituted with one to three F, Cl or alkyloxy optionally further substituted with alkyloxy.

7. The compound of claim 6, wherein R₁ₐ is alkyl, benzyl, phenyl, pyridinyl, pyrrolidinyl, cyclopentyl, cyclohexyl, cycloheptyl or piperidinyl; wherein alkyl is optionally substituted with hydroxyl, cyano, dialkylamino, heterocycloalkyl optionally substituted with one or two F, or alkyloxy optionally substituted with alkyloxy; benzyl is optionally substituted with one F; phenyl is optionally substituted with one or two F, Cl, —NRₐR_{b}, alkyl optionally substituted with three F, or alkyloxy optionally substituted with one to three F; pyridinyl is optionally substituted with alkyl optionally substituted with three F; pyrrolidinyl is substituted with alkyl, —C(═O)R_{c} or —C(═O)OR_{d}; cyclopentyl, cyclohexyl and cycloheptyl is optionally substituted with one or two F, Cl or alkyl; and piperidinyl is optionally substituted with alkyl, —C(═O)R_{c}, or —C(═O)OR_{d}, wherein Rₐ is hydrogen, R_{b} is acrylamide, R_{c} is alkyl or alkenyl, R_{d} is alkyl optionally substituted with one to three F, Cl or alkyloxy optionally further substituted with alkyloxy.

8. The compound of claim 7, wherein R₁ₐ is benzyl, phenyl, pyridinyl, pyrrolidinyl, cyclopentyl, cyclohexyl, cycloheptyl or piperidinyl; wherein benzyl is optionally substituted with one F; phenyl is optionally substituted with one or two F, Cl, —NRₐR_{b}, alkyl optionally substituted with three F, or alkyloxy optionally substituted with one to three F; pyridinyl is optionally substituted with alkyl optionally substituted with three F; pyrrolidinyl is substituted with alkyl, —C(═O)R_{c} or —C(═O)OR_{d}; cyclopentyl, cyclohexyl and cycloheptyl is optionally substituted with one or two F or alkyl; and piperidinyl is optionally substituted with alkyl, —C(═O)R_{c}, or —C(═O)OR_{d}; wherein R₁ is hydrogen, R_{b} is acrylamide, R_{c}; is alkyl or alkenyl, R_{d} is alkyl optionally substituted with one to three F or alkyloxy optionally further substituted with alkyloxy.

9. The compound of claim 3, wherein R is

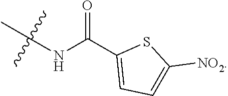 [SMILES: *NC(=O)c1ccc([N+](=O)[O-])s1]

10. The compound of claim 9, wherein R₂ is —OR₃, —NHR₄, —SR₅, alkenyl, alkynyl, aryl, heteroaryl, cycloalkyl, heterocycloalkyl, aryl fused with heterocycloalkyl or cycloalkenyl, wherein each of alkenyl, alkynyl, aryl, heteroaryl, cycloalkyl, heterocycloalkyl and cycloalkenyl is optionally substituted with one to three moieties selected from the group consisting of F, Cl, hydroxyl, cyano, —NRₑR_{f}, —C(═O)R_{g}, —C(═O)ORₕ, —SO₂Rᵢ, —OSiRⱼ, alkenyl, heterocycloalkyl, aryl optionally substituted with alkyl optionally substituted with one to three F, Cl or alkyloxy optionally substituted with one to three F, Cl, alkyl optionally substituted with one to three F, Cl, —NRₑR_{f} or —ORₖ, and alkyloxy optionally substituted with one to four F, Cl, hydroxyl, —NRₑR_{f} or alkyloxy optionally further substituted with alkyloxy, in which each of Rₑ and R_{f} independently is hydrogen or alkyl optionally substituted with alkyloxy, each of R_{g} and Rₕ independently is hydrogen, alkyl or alkenyl, Rᵢ is alkyl, Rⱼ is alkyl, and Rₖ is hydrogen, aryl, or alkyl optionally substituted with alkyloxy optionally further substituted with alkyloxy;
each of R₃ and R₅ independently is alkyl, cycloalkyl or aryl, wherein alkyl, cycloalkyl or aryl is optionally substituted with one to four moieties selected from the group consisting of F, Cl, alkyl optionally substituted with one to four F or Cl, alkyloxy optionally substituted with one to three F, Cl or alkyloxy, —NRₗRₘ and heterocycloalkyl, in which each of Rₗ and Rₘ independently is hydrogen or alkyl; and
R₄ is alkyl, cycloalkyl or —SO₂Rₙ, in which Rₙ is hydrogen, alkyl or aryl, and each of alkyl or aryl is optionally substituted with one to three F or Cl.

11. The compound of claim 10, wherein R₂ is —OR₃, —NHR₄, —SR₅, styryl, phenylethyl, cyclohexyl, cyclohexenyl, phenyl, benzodioxolyl, benzodioxinyl, benzofuranyl, furanyl, thiophenyl, xazolyl, imidazole, pyrazolyl, oxadiazolyl, azetidinyl, pyrrolidinyl, piperidinyl, morpholinyl, piperazinyl, pyrimidinyl, or pyridinyl, wherein styryl is optionally substituted with alkyl substituted with one to three F or alkoxy; phenylethynyl is optionally substituted with alkyl optionally substituted with one to three F or alkoxy optionally substituted with one to three F; cyclohexyl is optionally substituted with one to three alkyl; cyclohexenyl is optionally substituted with one to three alkyl; phenyl is optionally substituted with one to three moieties selected from the group consisting of F, Cl, cyano, dialkylamino, —C(═O)R_{g}, —C(═O)ORₕ, —SO₂Rᵢ, alkyl optionally substituted with one to three F, dialkylamin or alkyloxy, and alkyloxy optionally substituted with one to three F or alkyloxy optionally further substituted with alkyloxy; thiophenyl is optionally substituted with Cl or alkyl; xazolyl is optionally substituted with one or two alkyl; pyrazolyl is optionally substituted with alkyl optionally substituted with one to three F; oxadiazolyl is optionally substituted with alkyl; azetidinyl is optionally substituted with one or two F or —OSiRⱼ; pyrrolidinyl is optionally substituted with hydroxyl, cyano, alkenyl, —C(═O)ORₕ, one to two F, or alkyl optionally substituted with hydroxyl, phenoxy or alkyloxy optionally substituted with alkyloxy optionally further substituted with alkyloxy; piperidinyl is optionally substituted with alkyl, one or two F, —C(═O)R_{g}; morpholinyl is optionally substituted with one to three alkyl; piperazinyl is optionally substituted with alkyl, and pyridinyl is optionally substituted with one or two moieties selected from the group consisting of F, Cl, cyano, piperidinyl, morpholinyl, —NRₑR_{f}, alkyl optionally substituted with three to four F, and alkyloxy optionally substituted with one to four F, hydroxyl, dialkylamino or alkyloxy optionally further substituted with alkyloxy; in which Rₑ is alkyl, R_{f} is alkyl optionally substituted with alkyloxy, R_{g} is hydrogen, alkyl or alkenyl, Rₕ is hydrogen or alkyl, Rᵢ is alkyl, and Rⱼ is alkyl;
R₃ is phenyl, cycloalkyl or alkyl, wherein phenyl is optionally substituted with F, morpholinyl, alkyloxy optionally substituted with one to three F, or alkyl optionally substituted with one to three F; and alkyl is optionally substituted with one to four F, morpholinyl, dialkylamino, or alkyloxy optionally substituted with alkyloxy;
R₄ is alkyl, cycloalkyl or —SO₂Rₙ, in which Rₙ is phenyl optionally substituted with one to three Cl; and
R₅ is phenyl optionally substituted with Cl.

12. The compound of claim 3, wherein R₂ is

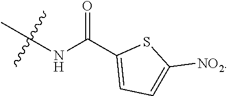 [SMILES: *NC(=O)c1ccc([N+](=O)[O-])s1]

13. The compound of claim 12, wherein R is phenyl or morpholinyl, and phenyl is optionally substituted with alkyl or alkyloxy optionally substituted with one to three F.

14. The compound of claim 1, wherein 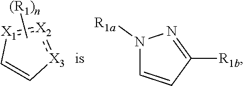
in which R₁ₐ is alkyl and R_{1b} is hydrogen;
R is 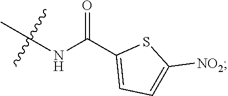 [SMILES: *NC(=O)c1ccc([N+](=O)[O-])s1]
and
R₂ is styryl or phenyl, in which phenyl is substituted with alkyl substituted with one to three F or alkyloxy substituted with one to three F; and styryl is substituted with alkyl substituted with one to three F.

15. The compound of claim 1, wherein 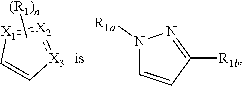
in which R_{1b} is hydrogen, and R₁ₐ is phenyl optionally substituted with one or two F, Cl, acrylamido, alkyl optionally substituted with three F or alkyloxy optionally substituted with three F;
R is 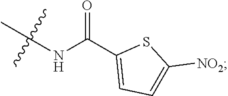 [SMILES: *NC(=O)c1ccc([N+](=O)[O-])s1]
and
R₂ is —OR₃, phenyl, benzodioxolyl, thiophenyl, pyrazolyl, pyrrolidinyl, pyrimidinyl, or pyridinyl, wherein phenyl is optionally substituted with one or two moieties selected from the group consisting of F, Cl, cyano, alkyl substituted with one to three F, and alkyloxy substituted with one to three F; pyrazolyl is optionally substituted with alkyl; pyrrolidinyl is substituted with —C(═O)ORₕ, one to two F, or alkyl substituted with hydroxyl, phenoxy or alkyloxy; pyridinyl is optionally substituted with one or two moieties selected from the group consisting of F, Cl, cyano, piperidinyl, alkyl, and alkyloxy optionally substituted with dialkylamino or alkyloxy optionally further substituted with alkyloxy; R₃ is alkyl optionally substituted with dialkylamino, and Rₕ is alkyl.

16. The compound of claim 1, wherein 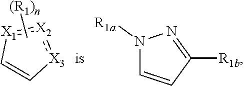
in which R_{1b} is hydrogen, and R₁ₐ is pyridinyl substituted with alkyl substituted with one to three F;
R is 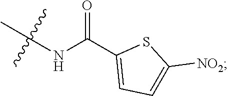 [SMILES: *NC(=O)c1ccc([N+](=O)[O-])s1]
and
R₂ is phenyl substituted with one or two F or Cl.

17. The compound of claim 1, wherein 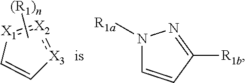
in which R_{1b} is hydrogen, R₁ₐ is piperidinyl substituted with —C(═O)OR_{d}, and R_{d} is alkyl;
R is 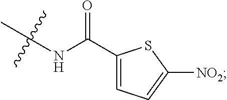 [SMILES: *NC(=O)c1ccc([N+](=O)[O-])s1]
and
R₂ is phenyl substituted with Cl or —C(═O)ORₕ, in which Rₕ is alkyl.

18. The compound of claim 1, wherein 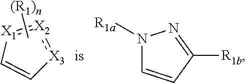
in which R_{1b} is hydrogen, and R₁ₐ is cyclohexyl optionally substituted with one or two F or alkyl;
R is 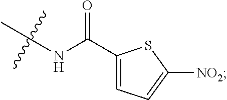 [SMILES: *NC(=O)c1ccc([N+](=O)[O-])s1]
and
R₂ is phenyl or pyridinyl, wherein phenyl is substituted with one or two F, Cl, or —C(═O)ORₕ, in which Rₕ is alkyl; and pyridinyl is substituted with F, Cl or alkyloxy.

19. The compound of claim 1, wherein 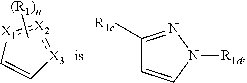
in which R_{1c} and R_{1d} are respectively alkyl.

20. The compound of claim 19, wherein R is 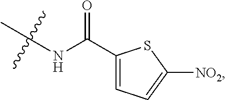 [SMILES: *NC(=O)c1ccc([N+](=O)[O-])s1]
and R₂ is phenyl, wherein phenyl is optionally substituted with F, alkyl optionally substituted with one to three F or alkyloxy optionally substituted with one to three F.

21. The compound of claim 1, wherein 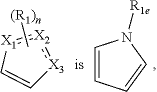
in which R₁ₑ is alkyl.

22. The compound of claim 21, wherein R is 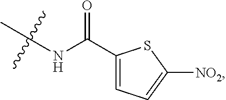 [SMILES: *NC(=O)c1ccc([N+](=O)[O-])s1]
and R₂ is phenyl optionally substituted with alkyl.

23. The compound of claim 1, wherein 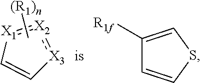
in which R_{1f} is alkyl or aryl optionally substituted with halogen or alkyl.

24. The compound of claim 23, wherein R_{1f} is alkyl, R₂ is 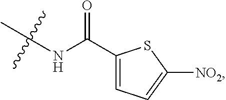 [SMILES: *NC(=O)c1ccc([N+](=O)[O-])s1]
and R is phenyl, in which phenyl is optionally substituted with alkoxy optionally substituted with one to three F.

25. The compound of claim 23, wherein R_{1f} is alkyl or phenyl optionally substituted with F or alkyl, R is 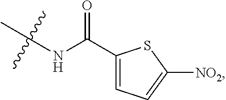 [SMILES: *NC(=O)c1ccc([N+](=O)[O-])s1]
and R₂ is —OR₃, pyrrolidinyl or phenyl, in which pyrrolidinyl is optionally substituted with one or two F, phenyl is optionally substituted with one to three moieties selected from the group consisting of F, Cl dialkylamino, alkyl optionally substituted with one to three F, and alkoxy optionally substituted with one to three F, and R₃ is phenyl optionally substituted with alkyl.

26. The compound of claim 1, wherein 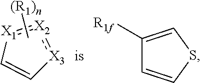
in which R_{1f} is alkyl; R is 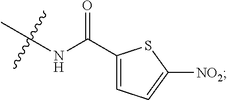 [SMILES: *NC(=O)c1ccc([N+](=O)[O-])s1]
and R₂ is phenyl, in which phenyl is substituted with alkyl substituted with one to three F or alkyloxy substituted with one to three F.

27. The compound of claim 1, wherein 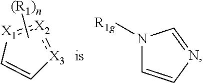
in which R_{1g} is cycloalkyl or heterocycloalkyl optionally substituted with —C(═O)OR_{d}, and R_{d} is alkyl.

28. The compound of claim 27, wherein R_{1g} is piperidinyl or cyclohexyl, R is 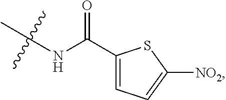 [SMILES: *NC(=O)c1ccc([N+](=O)[O-])s1]
and R₂ is phenyl optionally substituted with F or pyridinyl optionally substituted with F or alkyloxy.

29. The compound claim 1, wherein the compound is any one selected from the group consisting of Compounds 1-1 to 1-33, Compounds 2-1 to 2-8, Compounds 3-1 to 3-12, Compounds 4-1 to 4-22, Compounds 5-1 to 5-57, Compounds 6-1 to 6-145, Compounds 7-1 to 7-19, Compounds 8-1 to 8-16, Compounds 9-1 to 9-13 and Compounds 10-1 to 10-72.

30. The compound claim 1, wherein the compound is any one selected from the group consisting of compounds 14 to 1-5, compound 1-11, compound 1-22, compound 1-27, compound 4-2, compound 4-5, compound 5-6, compound 5-18, compounds 5-28 to 5-33, compound 5-35, compound 5-39, compound 5-40, compound 5-42, compound 5-44, compound 5-45, compound 5-47, compound 5-49, compound 5-56, compound 6-4, compound 6-11, compound 6-18, compound 6-19, compounds 6-24 to 6-28, compound 6-35, compound 6-36, compound 6-39, compound 6-43, compound 6-45, compound 6-46, compounds 6-50 to 6-54, compounds 6-57 to 6-58, compound 6-66, compound 6-69, compound 6-71, compound 6-75, compound 6-85, compounds 6-89 to 6-91, compound 6-104, compound 6-106, compound 6-108, compounds 6-110 to 6-112, compounds 6-114 to 6-121, compound 6-125, compounds 6-127 to 6-128, compound 6-130, compound 6-133, compound 6-135, compound 6-137, compound 7-1, compound 7-3, compound 7-6, compound 7-13, compound 7-19, compound 9-3, compound 9-5, compound 10-13, compound 10-14, compound 10-18, compound 10-23, compound 10-32, compound 10-40, compound 10-55, compound 10-57, and compound 10-64.

31. A pharmaceutical composition comprising:
a compound of claim 1 or a stereoisomer, a tautomer, or a pharmaceutically acceptable salt thereof; and
a pharmaceutically acceptable carrier.

32. A method for treating a cancer, comprising: administering to a subject in need thereof an effective amount of a compound of claim 1 or a stereoisomer, a tautomer, or a pharmaceutically acceptable salt thereof.

33. The method of claim 32, wherein the cancer is selected from the group consisting of gastric cancer, colon cancer, colorectal cancer, breast cancer, lung cancer, prostate cancer, bladder cancer, pancreatic cancer, liver cancer, uterine cancer, cervical caner, endometrial cancer, esophageal cancer, leukemia, lymphoma, kidney cancer, osteosarcoma, ovarian cancer, skin cancer, small intestine cancer, thymus cancer, thyroid cancer, nervous system cancers, bone cancer, brain cancer, and head and neck cancer.
